(19) United States
(12) Patent Application Publication
(10) Pub. No.: US 2017/0327512 A1
(43) Pub. Date: Nov. 16, 2017

(54) MODULATORS OF VASOPRESSIN RECEPTORS WITH THERAPEUTIC POTENTIAL

(71) Applicant: The Scripps Research Institute, La Jolla, CA (US)

(72) Inventors: Edward Roberts, Fallbrook, CA (US); Gopi Kumar Mittapalli, San Diego, CA (US); Yuanhua Wang, San Diego, CA (US); Jun Yang, La Jolla, CA (US); Marion Toussaint, La Jolla, CA (US); Olga Habarova, La Jolla, CA (US); Hugh Rosen, La Jolla, CA (US)

(21) Appl. No.: 15/418,579

(22) Filed: Jan. 27, 2017

Related U.S. Application Data
(63) Continuation of application No. 14/768,735, filed on Aug. 18, 2015, now abandoned.
(60) Provisional application No. 61/765,968, filed on Feb. 18, 2013.
(60) Provisional application No. 61/765,956, filed on Feb. 18, 2013.

(51) Int. Cl. C07D 491/107; C07D 207/16; C07D 417/14; C07D 413/14; C07D 413/06; C07D 409/14; C07D 409/06; C07D 405/14; C07D 401/14; C07D 401/12; C07D 401/06; C07D 401/04; C07D 307/14; C07D 295/205; C07D 295/20; C07D 277/82; C07D 267/20; C07D 263/58; C07D 249/08; C07D 241/54; C07D 241/44; C07D 239/94; C07D 239/42; C07D 235/30; C07D 213/74; C07D 211/62; C07D 498/10; C07D 519/00

(52) U.S. Cl. CPC C07D 491/107; C07D 519/00; C07D 207/16; C07D 417/14; C07D 413/14; C07D 413/06; C07D 409/14; C07D 409/06; C07D 405/14; C07D 401/14; C07D 401/12; C07D 401/06; C07D 401/04; C07D 307/14; C07D 295/205; C07D 295/20; C07D 277/82; C07D 267/20; C07D 263/58; C07D 249/08; C07D 241/54; C07D 241/44; C07D 239/94; C07D 239/42; C07D 235/30; C07D 213/74; C07D 211/62; C07D 498/10

(57) ABSTRACT

Compounds comprising piperazines, piperidines, spiro-furanopiperidines, and analogs thereof are provided that are modulators, such as positive allosteric modulators, of one or more subclasses of vasopressin receptors. The compounds can be selective modulators of one or more subclasses of vasopressin receptors. Compounds of the invention can be used in the treatment of a condition wherein modulating a vasopressin receptor is medically indicated for treatment of the condition.

### CROSS-REFERENCE TO RELATED APPLICATIONS

This application claims the priority of U.S. provisional applications Ser. No. 61/765,956, filed Feb. 18, 2013, and Ser. No. 61/765,968, filed Feb. 18, 2013, the disclosures of which are incorporated by reference herein in their entireties.

### STATEMENT OF GOVERNMENT SUPPORT

This invention was made with government support under grant number MH082878, awarded by the National Institutes of Health. The U.S. government has certain rights in the invention.

### BACKGROUND

Argenine Vasopressin (AVP) receptors belong to the G-protein coupled receptor family characterized by having seven transmembrane helices. For recent reviews on AVP receptors see Jard, 1998; Barberis et al., 1999; Gimpl and Fahrenholz, 2001; Holmes et al., 2003, 2004.

AVP is believed to mediate its actions at least through three known receptor subtypes: V1A, V1B (or V3) and V2 and a putative vasodilating V1C receptor.

Vasopressin 1a (V1A or V1a) receptors are expressed in the liver, vascular smooth muscle cells, brain and in many other tissues (Jard, 1998; Barberis et al., 1999; Holmes et al., 2003, 2004; Treschan and Peters, 2006). In the vasculature, V1A receptors mediate the pressor actions of AVP by a phospholipase C mediated pathway. In brain, V1A receptors mediate the anxiety/social responses (Ring, 2005) and aggression (Ferris et al., 2006) producing responses to AVP. V1A Receptors have been implicated in circadian rhythm and jet Lag (Li et al Am J Physiol Regul Integr Comp Physiol 296: R824-R830, 2009; Hastings, Science 342, 52 (2013); Foster et al Current Opinion in Neurobiology 2013, 23:888-894; Progress in Molecular Biology and Translational Science, Volume 119 #2013, ISSN 1877-1173, http://dx.doi.org/10.1016/B978-0-12-396971-2.00011-7; Tse Nat Rev Drug Discov 2013 December; 12(12):903).

Vasopressin 1b (V1B or V1b) receptors, present in the anterior pituitary, mediate the ACTH releasing effects of AVP, also by a phospholipase C mediating pathway (Jard, 1998). In a number of publications (Robert et al., 2005), the V1B receptor is also referred to as the V3 receptor. Evidence for the presence of V1B receptors in extra-pituitary tissues such as brain, the kidney and the adrenal medulla has also been reported. Recently the V1B receptor has been shown to mediate anxiety and stress in rats and in humans (Landgraf, 2006).

V2 receptors, present in the collecting duct of the kidney, mediate the antidiuretic action of AVP by an adenylate cyclase mediated pathway (Jard, 1998; Barbcris et al., 1999; Holmes et al., 2003, 2004). The pain response to AVP in the rat appears to be modulated by V2 receptors (Yang et al., 2006).

Besides its antidiuretic and vasoconstrictor properties, AVP can also cause vasodilation (Liard, 1989; Hirsch et al., 1989; Walker et al., 1989; Tagawa et al., 1995; Van Lieburg et al., 1995). The receptor subtype that mediates the vasodilating actions of AVP has to date not been characterized. Studies aimed at doing so have been hampered by the lack of specific vasodilating AVP agonists or antagonists.

Besides their value as pharmacological tools and radioligands, AVP antagonists are of potential clinical value. Non-peptide AVP V2 antagonists have potential therapeutic value for the treatment of the hyponatremia caused by the syndrome of inappropriate secretion of the antidiuretic hormone (SIADH) (Serradeil-Le Gal et al., 2002b; Hays, 2006; Palm et al., 2006; Schrier et al., 2006; Streefkerk and van Zwieten, 2006; Verbalis, 2006; Cawley, 2007; Chen et al., 2007; Gines, 2007; Munger, 2007; Parashar et al., 2007). The non-selective non-peptide AVP V2/V1A antagonist, Conivaptan (YM-087) (Tahara et al., 1997, 1998) under the tradename “Vaprisol” was approved for the treatment, by i.v. only, of euvolemic hyponatremia by the FDA in 2005 (Ghali et al., 2006; Verbalis, 2006). Later, it received FDA approval for the treatment of hypervolemic hyponatremia. Non-peptide V2 and V2/V1A antagonists may also have value for the treatment of heart failure (Abraham et al., 2006; Schwarz and Sanghi, 2006). They also have potential as pharmacochaperones for the treatment of X-linked nephrogenic diabetes insipidus (NDI) (Bernier et al., 2006; Robben et al., 2007). Three selective non-peptide V2 antagonists are currently in clinical trial (Verbalis, 2006): Tolvaptan (OPC 41061) (Yamamura et al., 1998), Satavaptan (SR 121 463) (Serradeil-Le Gal et al., 1996) and Lixivaptan (VPA-985) (Albright et al., 1998; Schrier et al., 2006; Soupart et al., 2006).

Antagonists of the vascular responses (V1A receptor) to AVP may have clinical potential for the treatment of those patients with hypertension or congestive heart failure (CHF) with concomitant elevated plasma AVP levels (Thibonnier et al., 2001). They may also be of value as “serenics” in the management of anger (Ferris et al., 2006). However, with the exception of the V1A antagonist SRX-251 (Ferris et al., 2006; Guillon et al., 2007a, b), and the Roche compound RG3714, none are currently in clinical trial. Non-peptide AVP V1B antagonists could be of value as diagnostic agents and as therapeutic agents for the treatment of ACTH secreting tumours (Serradeil-Le Gal et al., 2002a, b, 2007) and for treating anxiety and stress (Griebel et al., 2002; Craighead and MacSweeney, 2008).

Evidence from preclinical and human studies demonstrate that arginine vasopressin (AVP) and vasopressin receptor 1a (V1A) plays a crucial role in the pathophysiology of psychiatric disorders. AVP and corticotropin releasing factor (CRF) modulate the activity of the hypothalamic-pituitary-adrenal (HPA) axis. They are released from the hypothalamus and activate the secretion of adrenocorticotrophic hormone (ACTH) from the pituitary, which in turn releases the glucocorticoids (cortisol, human and corticosterone, rats) from the adrenal cortex. The glucocorticoids bind to their respective receptors which are responsible for the feedback loop inhibiting further release of AVP and CRF from the pituitary. The HPA axis does not only regulate peripheral functions such as immunity and metabolism, but also has significant actions in the CNS, having profound effects in psychiatric disorders such as major depression, PTSD and behaviors related to autism. Recent preclinical and clinical studies show that AVP, not CRF, is primarily responsible for regulating HPA function during chronic psychological stress. Significantly increased levels of cortisol have been observed in the saliva, plasma and urine of depressed patients as well as increased size and activity of the pituitary and adrenal glands. Indeed, the cortisol response to psychological stress appears under the regulation of AVP but not CRF in anxious healthy human volunteers. In war veterans with PTSD, elevated plasma levels of AVP were measured.

In particular, the V1A receptor is extensively expressed and the predominant vasopressin receptor subtype in the brain particularly in limbic areas like the amygdala, lateral septum, hypothalamus and hippocampus, which plays an important role in the regulation of anxiety. In the brain vasopressin is elevated in the amygdala during stress. Indeed, V1A knock-out mice show a reduction in anxious behavior in the plus-maze, open field and light-dark box and in marble burying test. The down regulation of V1A receptor with antisense oligonucleotide injected into the septum also causes a reduction in anxious behavior. Wistar rats bred for high anxiety/depression related behaviors (HAB) exhibit significantly overexpression of vasopressin mRNA and excessive vasopressin release in the paraventricular nucleus (PVN), where the V1A receptor is the predominant subtype. Administration of a selective, peptidic V1A receptor antagonist d(CH₂)₅Tyr(Me)AVP directly into the PVN decreases the depression-related behaviors in HAB rats. Clinically, patients with major depression show elevated levels of vasopressin in the PVN. The brain penetrant, non-peptidic V1A receptor antagonists JNJ-17308616 and SRX-251 show anxiolytic activity and block aggressive behavior in animal models, respectively. The V1A receptor antagonist of Hoffmann-La Roche, RG-3714, is in phase-II clinical trials for treating behaviors associated with autism spectrum disorders which may include ADHD, OCD, anxiety, etc.

V1A receptors have been implicated in circadian rhythm and jet lag (Li et al Am J Physiol Regul Integr Comp Physiol 296: R824-R830, 2009; Hastings, Science 342, 52 (2013); Foster et al Current Opinion in Neurobiology 2013, 23:888-894; Progress in Molecular Biology and Translational Science, Volume 119 #2013, ISSN 1877-1173, http://dx.doi.org/10.1016/B978-0-12-396971-2.00011-7; Tse Nat Rev Drug Discov 2013 December; 12(12):903).

Vasopressin has additionally been implicated in bone resorption (Tamm et al, Proc. Natl. Acad. Sci. U.S.A. 2013 Nov. 12; 110(46):18644-9); treatment of Traumatic brain injury (Plesnila et al JOURNAL OF NEUROTRAUMA 30:1442-1448 (Aug. 15, 2013), Marmarou et al Acta Neurochir (2013) 155:151-164); immulological disorders (Hu et al, Journal of Neuroimmunology 135 (2003) 72-81); and metabolic disorders (Aoyagi et al European Journal of Pharmacology 622 (2009) 32-36).

### SUMMARY

The present invention is directed to compounds and methods of treatment which in various embodiments enable the modulation of the bioactivity of one or more G-protein coupled receptor whose natural ligand is vasopressin. As discussed below, such receptors are promising targets for therapeutic targets for the treatment of a range of medical conditions including those for which there is presently little or nothing that can be done to cure them or to palliate symptoms. Certain mental/cognitive medical conditions are included in the spectrum of disease states that may be amenable to treatment with vasopressin receptor modulators of the present invention, including such difficult to reach conditions as anxiety disorders, autism, and post-traumatic stress disorder (PTSD).

In addition to the potential therapeutic areas mentioned above, the present disclosure relates to use of a compound(s) as outlined below for treatment of one or more medical conditions, or for the manufacture of a medicament for treatment of one or more medical conditions, or for medical use for conditions, such as compromised lactation conditions, labor induction impairment, uterine atony conditions, excessive bleeding, inflammation and pain including abdominal and back pain, male or female sexual dysfunction, irritable bowel syndrome, constipation and gastrointestinal obstruction, autism, stress, anxiety disorder, depression, post-traumatic stress syndrome, surgical blood loss, post-partum hemorrhage, defective wound healing, infection, mastitis, placenta delivery impairment, placental insufficiency, osteoporosis, or cancer; or septic shock, polycystic kidney disease, pulmonary hypertension, vasodilation/constriction, cardiopulmonary resuscitation, pediatric shock, cardiac arrest, wound healing disorders, metabolic disorders, diabetes, obesity, substance abuse, nicotine or alcohol abuse, circadian rhythm disorders, jet lag, disorders of the immune system, metabolic disorders, traumatic brain injury, cerebral infarction or stroke. Herein, the term anxiety includes anxiety disorders. Anxiety disorders includes the sub-indications generalized anxiety disorder, panic disorder, agoraphobia, phobias, social anxiety disorder, obsessive-compulsive disorder, post-traumatic stress disorder, and separation anxiety.

These compounds may exert their effects in the modulation of receptors as agonists, antagonists, inverse agonists, allosteric agonists, positive allosteric modulators (PAMs) or negative allosteric modulators.

The present disclosed subject matter is directed in various embodiments to compounds that at an effective concentration in vivo in a patient can modulate the action of a receptor of vasopressin, to pharmaceutical formulations and combinations of the compounds, to use of the compounds for modulating vasopressin receptors, and to treatment of conditions in patients wherein modulation of a vasopressin receptor is medically indicated.

In various embodiments, the invention provides a compound formula (I) or of formula (V), as defined below, in any of the various embodiments disclosed and claimed herein. A compound of any of these formulas can be a modulator, such as an antagonist, of one or more types of a vasopressin receptor.

In various embodiments, the invention provides a pharmaceutical composition comprising a compound formula (I) or of formula (V), as defined below, with a pharmaceutically acceptable excipient.

In various embodiments, the invention provides a method of treatment of a condition in a patient afflicted therewith, comprising administering to the patient an effective amount of a compound formula (I) or of formula (V), as defined below, at a frequency and for a duration to provide a beneficial effect to the patient. In various embodiments, the condition can be such that modulating a vasopressin receptor is medically indicated for treatment of the condition. For example, the condition can include compromised lactation conditions, labor induction impairment, uterine atony conditions, excessive bleeding, inflammation and pain including abdominal and back pain, sexual dysfunction, both male and female, irritable bowel syndrome, constipation and gastrointestinal obstruction, autism, stress, anxiety disorder, depression, surgical blood loss, post-partum hemorrhage, defective wound healing, infection, mastitis, placenta delivery impairment, placental insufficiency, osteoporosis, or cancer; or septic shock, polycystic kidney disease, pulmonary hypertension, vasodilation/constriction, cardiopulmonary resuscitation, pediatric shock, cardiac arrest, wound healing disorders, metabolic disorders, diabetes, obesity, substance abuse, nicotine or alcohol abuse, circadian rhythm disorders, jet lag, disorders of the immune system, metabolic disorders, use in treatment of traumatic brain injury, cerebral infarction and stroke.

### DETAILED DESCRIPTION

### Definitions

As used in the specification and the appended claims, the singular forms “a,” “an” and “the” include plural referents unless the context clearly dictates otherwise.

The term “about” as used herein, when referring to a numerical value or range, allows for a degree of variability in the value or range, for example, within 10%, or within 5% of a stated value or of a stated limit of a range.

All percent compositions are given as weight-percentages, unless otherwise stated.

All average molecular weights of polymers are weight-average molecular weights, unless otherwise specified.

As used herein, “individual” (as in the subject of the treatment) means both mammals and non-mammals. Mammals include, for example, humans; non-human primates, e.g. apes and monkeys; and non-primates, e.g. dogs, cats, cattle, horses, sheep, and goats. Non-mammals include, for example, fish and birds.

The term “disease” or “disorder” or “condition” are used interchangeably, and are used to refer to diseases or conditions wherein a vasopressin receptor plays a role in the biochemical mechanisms involved in the disease or condition such that a therapeutically beneficial effect can be achieved by acting on, i.e., “modulating” the vasopressin receptor. Modulating a vasopressin receptor can include binding to the receptor either at a ligand binding site or allosterically, and/or inhibiting the bioactivity of a vasopressin receptor.

Autism is defined by the guidelines given in DSM IV (Diagnostic and Statistical Manual of Mental Disorders (DSM), version 4). This includes Aspergers and Retts Syndrome under the umbrella of Autistic spectrum disorders. We are aware that Version 5 (DSMV) has recently been published (May 2013), but that this has yet to receive wide acceptance since it separates autism from Aspergers Syndrome amongst other changes that have severe socio-economical impacts. Thus for clarity we include Aspergers, Retts and others in the definition of Autism or autistic spectrum disorders as described in DSM IV.

The expression “effective amount”, when used to describe therapy to an individual suffering from a disorder, refers to the amount of a compound of the invention that is effective to modulate (e.g., act as an agonist or antagonist, or alter the response of the receptor to an agonist or antagonist, such as an endogenous agonist or antagonist) or otherwise act on a vasopressin receptor in the individual's tissues wherein the receptor involved in the disorder is active, wherein such inhibition or other action occurs to an extent sufficient to produce a beneficial therapeutic effect.

“Substantially” as the term is used herein means completely or almost completely; for example, a composition that is “substantially free” of a component either has none of the component or contains such a trace amount that any relevant functional property of the composition is unaffected by the presence of the trace amount, or a compound is “substantially pure” is there are only negligible traces of impurities present.

“Treating” or “treatment” within the meaning herein refers to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result. A therapeutically effective amount is also one in which any toxic or detrimental effects of compounds of the invention are outweighed by the therapeutically beneficial effects.

By “chemically feasible” is meant a bonding arrangement or a compound where the generally understood rules of organic structure are not violated; for example a structure within a definition of a claim that would contain in certain situations a pentavalent carbon atom that would not exist in nature would be understood to not be within the claim. The structures disclosed herein, in all of their embodiments are intended to include only “chemically feasible” structures, and any recited structures that are not chemically feasible, for example in a structure shown with variable atoms or groups, are not intended to be disclosed or claimed herein.

When a substituent is specified to be an atom or atoms of specified identity, “or a bond”, a configuration is referred to when the substituent is “a bond” that the groups that are immediately adjacent to the specified substituent are directly connected to each other in a chemically feasible bonding configuration.

When a group is recited, wherein the group can be present in more than a single orientation within a structure resulting in more than single molecular structure, e.g., a carboxamide group C(═O)NR, it is understood that the group can be present in any possible orientation, e.g., X—C(═O)N(R)—Y or X—N(R)C(═O)—Y, unless the context clearly limits the orientation of the group within the molecular structure.

All chiral, diastereomeric, racemic forms of a structure are intended, unless a particular stereochemistry or isomeric form is specifically indicated. Compounds used in the present invention can include enriched or resolved optical isomers at any or all asymmetric atoms as are apparent from the depictions, at any degree of enrichment. Both racemic and diastereomeric mixtures, as well as the individual optical isomers can be isolated or synthesized so as to be substantially free of their enantiomeric or diastereomeric partners, and these are all within the scope of the invention.

The inclusion of an isotopic form of one or more atoms in a molecule that is different from the naturally occurring isotopic distribution of the atom in nature is referred to as an “isotopically labeled form” of the molecule. All isotopic forms of atoms are included as options in the composition of any molecule, unless a specific isotopic form of an atom is indicated. For example, any hydrogen atom or set thereof in a molecule can be any of the isotopic forms of hydrogen, i.e., protium (¹H), deuterium (²H), or tritium (³H) in any combination. Similarly, any carbon atom or set thereof in a molecule can be any of the isotopic form of carbons, such as ¹¹C, ¹²C, ¹³C, or ¹⁴C, or any nitrogen atom or set thereof in a molecule can be any of the isotopic forms of nitrogen, such as ¹³N, ¹⁴N, or ¹⁵N. A molecule can include any combination of isotopic forms in the component atoms making up the molecule, the isotopic form of every atom forming the molecule being independently selected. In a multi-molecular sample of a compound, not every individual molecule necessarily has the same isotopic composition. For example, a sample of a compound can include molecules containing various different isotopic compositions, such as in a tritium or ¹⁴C radiolabeled sample where only some fraction of the set of molecules making up the macroscopic sample contains a radioactive atom. It is also understood that many elements that are not artificially isotopically enriched themselves are mixtures of naturally occurring isotopic forms, such as ¹⁴N and ¹⁵N, ³²S and ³⁴S, and so forth. A molecule as recited herein is defined as including isotopic forms of all its constituent elements at each position in the molecule. As is well known in the art, isotopically labeled compounds can be prepared by the usual methods of chemical synthesis, except substituting an isotopically labeled precursor molecule. The isotopes, radiolabeled or stable, can be obtained by any method known in the art, such as generation by neutron absorption of a precursor nuclide in a nuclear reactor, by cyclotron reactions, or by isotopic separation such as by mass spectrometry. The isotopic forms are incorporated into precursors as required for use in any particular synthetic route. For example, ¹⁴C and ³H can be prepared using neutrons generated in a nuclear reactor. Following nuclear transformation, ¹⁴C and ³H are incorporated into precursor molecules, followed by further elaboration as needed.

In general, “substituted” refers to an organic group as defined herein in which one or more bonds to a hydrogen atom contained therein are replaced by one or more bonds to a non-hydrogen atom such as, but not limited to, a halogen (i.e., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, N-oxides, hydrazides, azides, and enamines; and other heteroatoms in various other groups. Non-limiting examples of substituents J that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein R can be hydrogen or a carbon-based moiety, and wherein the carbon-based moiety can itself be further substituted; for example, wherein R can be hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl or R can be independently mono- or multi-substituted with J or with some or all of the above-listed functional groups, or with other functional groups; or wherein two R groups bonded to a nitrogen atom or to adjacent nitrogen atoms can together with the nitrogen atom or atoms form a heterocyclyl, which can be mono- or independently multi-substituted with J, or with some or all of the above-listed functional groups, or with other functional groups.

When a substituent is monovalent, such as, for example, F or Cl, it is bonded to the atom it is substituting by a single bond. When a substituent is more than monovalent, such as O, which is divalent, it can be bonded to the atom it is substituting by more than one bond, i.e., a divalent substituent is bonded by a double bond; for example, a C substituted with O forms a carbonyl group, C═O, which can also be written as “CO”, “C(O)”, or “C(═O)”, wherein the C and the O are double bonded. When a carbon atom is substituted with a double-bonded oxygen (═O) group, the oxygen substituent is termed an “oxo” group. When a divalent substituent such as NR is double-bonded to a carbon atom, the resulting C(═NR) group is termed an “imino” group. When a divalent substituent such as S is double-bonded to a carbon atom, the results C(═S) group is termed a “thiocarbonyl” group.

Alternatively, a divalent substituent such as O, S, C(O), S(O), or S(O)₂ can be connected by two single bonds to two different carbon atoms. For example, O, a divalent substituent, can be bonded to each of two adjacent carbon atoms to provide an epoxide group, or the O can form a bridging ether group, termed an “oxy” group, between adjacent or non-adjacent carbon atoms, for example bridging the 1,4-carbons of a cyclohexyl group to form a [2.2.1]-oxabicyclo system. Further, any substituent can be bonded to a carbon or other atom by a linker, such as (CH₂)ₙ or (CR₂)ₙ wherein n is 1, 2, 3, or more, and each R is independently selected.

C(O) and S(O)₂ groups can also be bound to one or two heteroatoms, such as nitrogen or oxygen, rather than to a carbon atom. For example, when a C(O) group is bound to one carbon and one nitrogen atom, the resulting group is called an “amide” or “carboxamide.” When a C(O) group is bound to two nitrogen atoms, the functional group is termed a “urea.” When a C(O) is bonded to one oxygen and one nitrogen atom, the resulting group is termed a “carbamate” or “urethane.” When a S(O)₂ group is bound to one carbon and one nitrogen atom, the resulting unit is termed a “sulfonamide.” When a S(O)₂ group is bound to two nitrogen atoms, the resulting unit is termed a “sulfamate.”

Substituted alkyl, alkenyl, alkynyl, cycloalkyl, and cycloalkenyl groups as well as other substituted groups also include groups in which one or more bonds to a hydrogen atom are replaced by one or more bonds, including double or triple bonds, to a carbon atom, or to a heteroatom such as, but not limited to, oxygen in carbonyl (oxo), carboxyl, ester, amide, imide, urethane, and urea groups; and nitrogen in imines, hydroxyimines, oximes, hydrazones, amidines, guanidines, and nitriles.

Substituted ring groups such as substituted cycloalkyl, aryl, heterocyclyl and heteroaryl groups also include rings and fused ring systems in which a bond to a hydrogen atom is replaced with a bond to a carbon atom. Therefore, substituted cycloalkyl, aryl, heterocyclyl and heteroaryl groups can also be substituted with alkyl, alkenyl, and alkynyl groups as defined herein.

By a “ring system” as the term is used herein is meant a moiety comprising one, two, three or more rings, which can be substituted with non-ring groups or with other ring systems, or both, which can be fully saturated, partially unsaturated, fully unsaturated, or aromatic, and when the ring system includes more than a single ring, the rings can be fused, bridging, or spirocyclic. By “spirocyclic” is meant the class of structures wherein two rings are fused at a single tetrahedral carbon atom, as is well known in the art.

As to any of the groups described herein, which contain one or more substituents, it is understood, of course, that such groups do not contain any substitution or substitution patterns which are sterically impractical and/or synthetically non-feasible. In addition, the compounds of this disclosed subject matter include all stereochemical isomers arising from the substitution of these compounds.

Alkyl groups include straight chain and branched alkyl groups and cycloalkyl groups having from 1 to about 20 carbon atoms, and typically from 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, ncopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed above, for example, amino, hydroxy, cyano, carboxy, nitro, thio, alkoxy, and halogen groups.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The terms “carbocyclic,” “carbocyclyl,” and “carbocycle” denote a ring structure wherein the atoms of the ring are carbon, such as a cycloalkyl group or an aryl group. In some embodiments, the carbocycle has 3 to 8 ring members, whereas in other embodiments the number of ring carbon atoms is 4, 5, 6, or 7. Unless specifically indicated to the contrary, the carbocyclic ring can be substituted with as many as N−1 substituents wherein N is the size of the carbocyclic ring with, for example, alkyl, alkenyl, alkynyl, amino, aryl, hydroxy, cyano, carboxy, heteroaryl, heterocyclyl, nitro, thio, alkoxy, and halogen groups, or other groups as are listed above. A carbocyclyl ring can be a cycloalkyl ring, a cycloalkenyl ring, or an aryl ring. A carbocyclyl can be monocyclic or polycyclic, and if polycyclic each ring can be independently be a cycloalkyl ring, a cycloalkenyl ring, or an aryl ring.

(Cycloalkyl)alkyl groups, also denoted cycloalkylalkyl, are alkyl groups as defined above in which a hydrogen or carbon bond of the alkyl group is replaced with a bond to a cycloalkyl group as defined above.

Alkenyl groups include straight and branched chain and cyclic alkyl groups as defined above, except that at least one double bond exists between two carbon atoms. Thus, alkenyl groups have from 2 to about 20 carbon atoms, and typically from 2 to 12 carbons or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

Cycloalkenyl groups include cycloalkyl groups having at least one double bond between 2 carbons. Thus for example, cycloalkenyl groups include but are not limited to cyclohexenyl, cyclopentenyl, and cyclohexadienyl groups. Cycloalkenyl groups can have from 3 to about 8-12 ring members, whereas in other embodiments the number of ring carbon atoms range from 3 to 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like, provided they include at least one double bond within a ring. Cycloalkenyl groups also include rings that are substituted with straight or branched chain alkyl groups as defined above.

(Cycloalkenyl)alkyl groups are alkyl groups as defined above in which a hydrogen or carbon bond of the alkyl group is replaced with a bond to a cycloalkenyl group as defined above.

Alkynyl groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂CH₂—OH, —CH₂—CH₂—NH—CH₃, —CH₂—S—CH₂—CH₃, —CH₂CH₂—S(═O)—CH₃, and —CH₂CH₂—O—CH₂CH₂—O—CH₃. Up to two heteroatoms may be consecutive, such as, for example, —CH₂—NH—OCH₃, or —CH₂—CH₂—S—S—CH₃.

A “cycloheteroalkyl” ring is a cycloalkyl ring containing at least one heteroatom. A cycloheteroalkyl ring can also be termed a “heterocyclyl,” described below.

The term “heteroalkenyl” by itself or in combination with another term means, unless otherwise stated, a stable straight or branched chain monounsaturated or di-unsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, —CH₂—CH═CH—CH₂—SH, and —CH═CH—O—CH₂CH₂—O—CH₃.

Aryl groups are cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined above. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed above.

Aralkyl groups are alkyl groups as defined above in which a hydrogen or carbon bond of an alkyl group is replaced with a bond to an aryl group as defined above. Representative aralkyl groups include benzyl and phenylethyl groups and fused (cycloalkylaryl)alkyl groups such as 4-ethyl-indanyl. Aralkenyl group are alkenyl groups as defined above in which a hydrogen or carbon bond of an alkyl group is replaced with a bond to an aryl group as defined above.

Heterocyclyl groups or the term “heterocyclyl” includes aromatic and non-aromatic ring compounds containing 3 or more ring members, of which, one or more is a heteroatom such as, but not limited to, N, O, and S. Thus a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms sums up to equal the total number of ring atoms. A heterocyclyl ring can also include one or more double bonds. A heteroaryl ring is an embodiment of a heterocyclyl group. The phrase “heterocyclyl group” includes fused ring species including those comprising fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed above. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed above.

Heteroaryl groups are aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed above. Representative substituted heteroaryl groups can be substituted one or more times with groups such as those listed above.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

Heterocyclylalkyl groups are alkyl groups as defined above in which a hydrogen or carbon bond of an alkyl group as defined above is replaced with a bond to a heterocyclyl group as defined above. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

Heteroarylalkyl groups are alkyl groups as defined above in which a hydrogen or carbon bond of an alkyl group is replaced with a bond to a heteroaryl group as defined above.

The term “alkoxy” refers to an oxygen atom connected to an alkyl group, including a cycloalkyl group, as are defined above. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include one to about 12-20 carbon atoms bonded to the oxygen atom, and can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structures are substituted therewith.

The terms “halo” or “halogen” or “halide” by themselves or as part of another substituent mean, unless otherwise stated, a fluorine, chlorine, bromine, or iodine atom, preferably, fluorine, chlorine, or bromine.

A “haloalkyl” group includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

A “haloalkoxy” group includes mono-halo alkoxy groups, poly-halo alkoxy groups wherein all halo atoms can be the same or different, and per-halo alkoxy groups, wherein all hydrogen atoms are replaced by halogen atoms, such as fluoro. Examples of haloalkoxy include trifluoromethoxy, 1,1-dichloroethoxy, 1,2-dichloroethoxy, 1,3-dibromo-3,3-difluoropropoxy, perfluorobutoxy, and the like.

The term “(Cₓ-C_{y})perfluoroalkyl,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkyl, more preferred is —(C₁-C₃)perfluoroalkyl, most preferred is —CF₃.

The term “(Cₓ-C_{y})perfluoroalkylene,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkylene, more preferred is —(C₁-C₃)perfluoroalkylene, most preferred is —CF₂—.

The terms “aryloxy” and “arylalkoxy” refer to, respectively, an aryl group bonded to an oxygen atom and an aralkyl group bonded to the oxygen atom at the alkyl moiety. Examples include but are not limited to phenoxy, naphthyloxy, and benzyloxy.

An “acyl” group as the term is used herein refers to a group containing a carbonyl moiety wherein the group is bonded via the carbonyl carbon atom. The carbonyl carbon atom is also bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-20 additional carbon atoms bonded to the carbonyl group. An acyl group can include double or triple bonds within the meaning herein. An acryloyl group is an example of an acyl group. An acyl group can also include heteroatoms within the meaning here. A nicotinoyl group (pyridyl-3-carbonyl) group is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “amine” includes primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

An “amino” group is a substituent of the form —NH₂, —NHR, —NR₂, —NR₃ wherein each R is independently selected, and protonated forms of each, except for —NR₃ which cannot be protonated. Accordingly, any compound substituted with an amino group can be viewed as an amine. An “amino group” within the meaning herein can be a primary, secondary, tertiary or quaternary amino group. An “alkylamino” group includes a monoalkylamino, dialkylamino, and trialkylamino group.

An “ammonium” ion includes the unsubstituted ammonium ion NH₄ but unless otherwise specified, it also includes any protonated or quaternarized forms of amines. Thus, trimethylammonium hydrochloride and tetramethylammonium chloride are both ammonium ions, and amines, within the meaning herein.

The term “amide” (or “amido”) includes C- and N-amide groups, i.e., —C(O)NR₂, and —NRC(O)R groups, respectively. Amide groups therefore include but are not limited to primary carboxamide groups (—C(O)NH₂) and formamide groups (—NHC(O)H). A “carboxamido” group is a group of the formula C(O)NR₂, wherein R can be H, alkyl, aryl, etc.

The term “azido” refers to an N₃ group. An “azide” can be an organic azide or can be a salt of the azide (N₃⁻) anion. The term “nitro” refers to an NO₂ group bonded to an organic moiety. The term “nitroso” refers to an NO group bonded to an organic moiety. The term nitrate refers to an ONO₂ group bonded to an organic moiety or to a salt of the nitrate (NO₃⁻) anion.

The term “urethane” (“carbamoyl” or “carbamyl”) includes N- and O-urethane groups, i.e., —NRC(O)OR and —OC(O)NR₂ groups, respectively.

The term “sulfonamide” (or “sulfonamido”) includes S- and N-sulfonamide groups, i.e., —SO₂NR₂ and —NRSO₂R groups, respectively. Sulfonamide groups therefore include but are not limited to sulfamoyl groups (—SO₂NH₂). An organosulfur structure represented by the formula —S(O)(NR)— is understood to refer to a sulfoximine, wherein both the oxygen and the nitrogen atoms are bonded to the sulfur atom, which is also bonded to two carbon atoms.

The term “amidine” or “amidino” includes groups of the formula —C(NR)NR₂. Typically, an amidino group is —C(NH)NH₂.

The term “guanidine” or “guanidino” includes groups of the formula —NRC(NR)NR₂. Typically, a guanidino group is —NHC(NH)NH₂.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH₄⁺ or the cations of various amines, including tetraalkyl ammonium salts such as tetramethylammonium, or other cations such as trimethylsulfonium, and the like. A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein. The compounds of the present invention may take the form of salts. The term “salts” embraces addition salts of free acids or free bases which are compounds of the invention. Salts can be “pharmaceutically-acceptable salts.” The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention.

Suitable pharmaceutically-acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafluoroborates.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. Although pharmaceutically unacceptable salts are not generally useful as medicaments, such salts may be useful, for example as intermediates in the synthesis of Formula (I) compounds, for example in their purification by recrystallization. All of these salts may be prepared by conventional means from the corresponding compound according to Formula (I) by reacting, for example, the appropriate acid or base with the compound according to Formula (I). The term “pharmaceutically acceptable salts” refers to nontoxic inorganic or organic acid and/or base addition salts, see, for example, Lit et al., Salt Selection for Basic Drugs (1986), Int J. Pharm., 33, 201-217, incorporated by reference herein.

A “hydrate” is a compound that exists in a composition with water molecules. The composition can include water in stoichiometric quantities, such as a monohydrate or a dihydrate, or can include water in random amounts. As the term is used herein a “hydrate” refers to a solid form, i.e., a compound in water solution, while it may be hydrated, is not a hydrate as the term is used herein.

A “solvate” is a similar composition except that a solvent other that water replaces the water. For example, methanol or ethanol can form an “alcoholate”, which can again be stoichiometric or non-stoichiometric. As the term is used herein a “solvate” refers to a solid form, i.e., a compound in solution in a solvent, while it may be solvated, is not a solvate as the term is used herein.

A “prodrug” as is well known in the art is a substance that can be administered to a patient where the substance is converted in vivo by the action of biochemicals within the patients body, such as enzymes, to the active pharmaceutical ingredient. Examples of prodrugs include esters of carboxylic acid groups, which can be hydrolyzed by endogenous esterases as are found in the bloodstream of humans and other mammals. Conventional procedures for the selection and preparation of suitable prodrug derivatives are described, for example, in “Design of Prodrugs”, ed. H. Bundgaard, Elsevier, 1985.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

If a value of a variable that is necessarily an integer, e.g., the number of carbon atoms in an alkyl group or the number of substituents on a ring, is described as a range, e.g., 0-4, what is meant is that the value can be any integer between 0 and 4 inclusive, i.e., 0, 1, 2, 3, or 4.

In various embodiments, the compound or set of compounds, such as are used in the inventive methods, can be any one of any of the combinations and/or sub-combinations of the above-listed embodiments.

In various embodiments, a compound as shown in any of the Examples, or among the exemplary compounds, is provided. Provisos may apply to any of the disclosed categories or embodiments wherein any one or more of the other above disclosed embodiments or species may be excluded from such categories or embodiments.

The present invention further embraces isolated compounds according to formula (I) or of formula (V). The expression “isolated compound” refers to a preparation of a compound of formula (I) or (V), or a mixture of compounds according to formula (I) or (V), wherein the isolated compound has been separated from the reagents used, and/or byproducts formed, in the synthesis of the compound or compounds. “Isolated” does not mean that the preparation is technically pure (homogeneous), but it is sufficiently pure to compound in a form in which it can be used therapeutically. Preferably an “isolated compound” refers to a preparation of a compound of formula (I) or (V) or a mixture of compounds according to formula (I) or (V), which contains the named compound or mixture of compounds according to formula (I) or (V) in an amount of at least 10 percent by weight of the total weight. Preferably the preparation contains the named compound or mixture of compounds in an amount of at least 50 percent by weight of the total weight; more preferably at least 80 percent by weight of the total weight; and most preferably at least 90 percent, at least 95 percent or at least 98 percent by weight of the total weight of the preparation.

The compounds of the invention and intermediates may be isolated from their reaction mixtures and purified by standard techniques such as filtration, liquid-liquid extraction, solid phase extraction, distillation, recrystallization or chromatography, including flash column chromatography, or HPLC.

### Isomerism and Tautomerism in Compounds of the Invention

### Tautomerism

Within the present invention it is to be understood that a compound of the formula (I) or (V) or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound. It is to be understood that the formulae drawings within this specification can represent only one of the possible tautomeric forms. However, it is also to be understood that the invention encompasses any tautomeric form, and is not to be limited merely to any one tautomeric form utilized within the formulae drawings. The formulae drawings within this specification can represent only one of the possible tautomeric forms and it is to be understood that the specification encompasses all possible tautomeric forms of the compounds drawn not just those forms which it has been convenient to show graphically herein. For example, tautomerism may be exhibited by a pyrazolyl group bonded as indicated by the wavy line. While both substituents would be termed a 4-pyrazolyl group, it is evident that a different nitrogen atom bears the hydrogen atom in each structure.

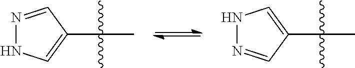 [SMILES: *c1cn[nH]c1 ; *c1cn[nH]c1]

Such tautomerism can also occur with substituted pyrazoles such as 3-methyl, 5-methyl, or 3,5-dimethylpyrazoles, and the like. Another example of tautomerism is amido-imido (lactam-lactim when cyclic) tautomerism, such as is seen in heterocyclic compounds bearing a ring oxygen atom adjacent to a ring nitrogen atom. For example, the equilibrium:

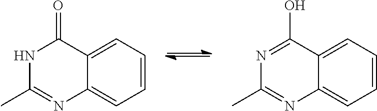

is an example of tautomerism. Accordingly, a structure depicted herein as one tautomer is intended to also include the other tautomer.

### Optical Isomerism

It will be understood that when compounds of the present invention contain one or more chiral centers, the compounds may exist in, and may be isolated as pure enantiomeric or diastereomeric forms or as racemic mixtures. The present invention therefore includes any possible enantiomers, diastereomers, racemates or mixtures thereof of the compounds of the invention.

The isomers resulting from the presence of a chiral center comprise a pair of non-superimposable isomers that are called “enantiomers.” Single enantiomers of a pure compound are optically active, i.e., they are capable of rotating the plane of plane polarized light. Single enantiomers are designated according to the Cahn-Ingold-Prelog system. The priority of substituents is ranked based on atomic weights, a higher atomic weight, as determined by the systematic procedure, having a higher priority ranking Once the priority ranking of the four groups is determined, the molecule is oriented so that the lowest ranking group is pointed away from the viewer. Then, if the descending rank order of the other groups proceeds clockwise, the molecule is designated as having an (R) absolute configuration, and if the descending rank of the other groups proceeds counterclockwise, the molecule is designated as having an (5) absolute configuration. In the example in the Scheme below, the Cahn-Ingold-Prelog ranking is A>B>C>D. The lowest ranking atom, D is oriented away from the viewer. The solid wedge indicates that the atom bonded thereby projects toward the viewer out of the plane of the paper, and a dashed wedge indicates that the atom bonded thereby projects away from the viewer out of the plan of the paper, i.e., the plane “of the paper” being defined by atoms A, C, and the chiral carbon atom for the (R) configuration shown below.

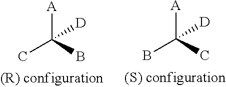

A carbon atom bearing the A-D atoms as shown above is known as a “chiral” carbon atom, and the position of such a carbon atom in a molecule is termed a “chiral center.” Compounds of the invention may contain more than one chiral center, and the configuration at each chiral center is described in the same fashion.

There are various conventions for depicting chiral structures using solid and dashed wedges. For example, for the (R) configuration shown above, the following two depictions are equivalent:

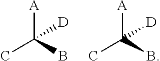

The present invention is meant to encompass diastereomers as well as their racemic and resolved, diastereomerically and enantiomerically pure forms and salts thereof. Diastereomeric pairs may be resolved by known separation techniques including normal and reverse phase chromatography, and crystallization.

“Isolated optical isomer” means a compound which has been substantially purified from the corresponding optical isomer(s) of the same formula. Preferably, the isolated isomer is at least about 80%, more preferably at least 90% pure, even more preferably at least 98% pure, most preferably at least about 99% pure, by weight.

Isolated optical isomers may be purified from racemic mixtures by well-known chiral separation techniques. According to one such method, a racemic mixture of a compound of the invention, or a chiral intermediate thereof, is separated into 99% wt. % pure optical isomers by HPLC using a suitable chiral column, such as a member of the series of DAICEL® CHIRALPAK® family of columns (Daicel Chemical Industries, Ltd., Tokyo, Japan). The column is operated according to the manufacturer's instructions.

### Rotational Isomerism

It is understood that due to chemical properties (i.e., resonance lending some double bond character to the C—N bond) of restricted rotation about the amide bond linkage (as illustrated below) it is possible to observe separate rotamer species and even, under some circumstances, to isolate such species (see below). It is further understood that certain structural elements, including steric bulk or substituents on the amide nitrogen, may enhance the stability of a rotamer to the extent that a compound may be isolated as, and exist indefinitely, as a single stable rotamer. The present invention therefore includes any possible stable rotamers of formula (I) which are biologically active in the treatment of cancer or other proliferative disease states.

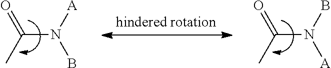

### Regioisoinerism

The preferred compounds of the present invention have a particular spatial arrangement of substituents on the aromatic rings, which is related to the structure activity relationship demonstrated by the compound class. Often such substitution arrangement is denoted by a numbering system; however, numbering systems are often not consistent between different ring systems. In six-membered aromatic systems, the spatial arrangements are specified by the common nomenclature “para” for 1,4-substitution, “meta” for 1,3-substitution and “ortho” for 1,2-substitution as shown below.

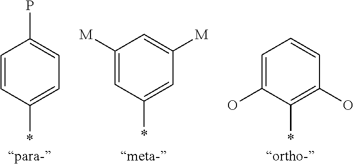

In various embodiments, the compound or set of compounds, such as are among the inventive compounds or are used in the inventive methods, can be any one of any of the combinations and/or sub-combinations of the above-listed embodiments.

### Detailed Description

In various embodiments, the invention provides a compound of formula (I)

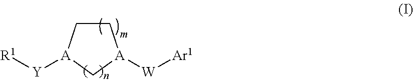

wherein

each A is independently N or CR, provided that at least one A is N;

m and n are independently 0, 1, 2, or 3;

wherein the ring comprising A optionally comprises a double bond; and,

the ring comprising A is substituted with 0-3 J; W and Y are each independently a bond, (CHR)₁₋₄, (CH₂)₀₋₂O, (CH₂)₀₋₂C(O)(CH₂)₀₋₂, (CH₂)₀₋₂CR₂(CH₂)₀₋₂, (CH₂)₀₋₂C(OR)(R)(CH₂)₀₋₂, O(CR₂)₁₋₄O, (CH₂)₀₋₂N(R)(CH₂)₀₋₂, (CH₂)₀₋₂S(CH₂)₀₋₂, (CH₂)₀₋₂SO(CH₂)₀₋₂, (CH₂)₀₋₂SO₂(CH₂)₀₋₂, (CH₂)₀₋₂SO₂N(R)(CH₂)₀₋₂, (CH₂)₀₋₂SO₃(CH₂)₀₋₂, (CH₂)₀₋₂C(O)C(O) (CH₂)₀₋₂, (CH₂)₀₋₂C(O)CH₂C(O)(CH₂)₀₋₂, (CH₂)₀₋₂C(S)(CH₂)₀₋₂, (CH₂)₀₋₂C(O)O(CH₂)₀₋₂, (CH₂)₀₋₂OC(O)(CH₂)₀₋₂, (CH₂)₀₋₂OC(O)O(CH₂)₀₋₂, (CH₂)₀₋₂C(O)N(R)(CH₂)₀₋₂, (CH₂)₀₋₂OC(O)N(R)(CH₂)₀₋₂, (CH₂)₀₋₂C(S)N(R)(CH₂)₀₋₂, (CH₂)₀₋₂NHC(O) (CH₂)₀₋₂, (CH₂)₀₋₂N(R)N(R)C(O)(CH₂)₀₋₂, (CH₂)₀₋₂N(R)N(R)C(O)O(CH₂)₀₋₂, (CH₂)₀₋₂N(R)N(R)CON(R)(CH₂)₀₋₂, (CH₂)₀₋₂N(R)SO₂(CH₂)₀₋₂, (CH₂)₀₋₂N(R)SO₂N(R)(CH₂)₀₋₂, (CH₂)₀₋₂N(R)C(O)O(CH₂)₀₋₂, (CH₂)₀₋₂N(R)C(O)(CH₂)₀₋₂, (CH₂)₀₋₂N(R)C(S)(CH₂)₀₋₂, (CH₂)₀₋₂N(R)C(O)N(R)(CH₂)₀₋₂, (CH₂)₀₋₂N(R)C(S)N(R)(CH₂)₀₋₂, (CH₂)₀₋₂N(COR)CO(CH₂)₀₋₂, (CH₂)₀₋₂N(OR)(CH₂)₀₋₂, (CH₂)₀₋₂C(═NH)N(R)(CH₂)₀₋₂, (CH₂)₀₋₂C(O)N(OR)(CH₂)₀₋₂, or (CH₂)₀₋₂C(═NOR)(CH₂)₀₋₂;

wherein R is independently at each occurrence hydrogen or an alkyl, heteroalkyl, acyl, cycloalkyl, cycloalkylalkyl, aryl, aralkyl, heterocyclyl, heterocyclylalkyl, heteroaryl, or heteroarylalkyl, wherein any alkyl, heteroalkyl, acyl, cycloalkyl, cycloalkylalkyl, aryl, aralkyl, heterocyclyl, heterocyclylalkyl, heteroaryl, or heteroarylalkyl is substituted with 0-3 J; or wherein two R groups together with a nitrogen atom or with two adjacent nitrogen atoms to which they are bonded can together form a 3-8 membered heterocyclyl substituted with 0-3 J, optionally further comprising 1-3 additional heteroatoms selected from the group consisting of O, NR, S, S(O) and S(O)₂;

R¹ is hydrogen, alkyl, cycloalkyl, cycloalkylalkyl, aryl, aralkyl, heterocyclyl, heterocyclylalkyl, heteroaryl, or heteroarylalkyl, wherein any alkyl, cycloalkyl, cycloalkyl alkyl, aryl, aralkyl, heterocyclyl, heterocyclyl alkyl, heteroaryl, or heteroarylalkyl is substituted with 0-3 J;

Ar¹ is cycloalkyl, aryl, heterocyclyl, or heteroaryl, wherein any cycloalkyl, aryl, heterocyclyl, or heteroaryl, is mono- or independently multi-substituted with J, (C₁-C₆)alkyl, (C₂-C₆)alkenyl, (C₂-C₆) alkynyl, (C₁-C₆)haloalkyl, (C₁-C₆)alkoxy, (C₁-C₆)haloalkoxy, cycloalkyl-(C₀-C₆)alkyl, heterocyclyl-(C₀-C₆)alkyl, aryl-(C₀-C₆)alkyl, or heteroaryl-(C₀-C₆)alkyl, wherein any alkyl, alkenyl, alkynyl, haloalkyl, alkoxy, haloalkoxy, cycloalkyl, heterocyclyl, aryl, or heteroaryl is optionally mono- or independently multi-substituted with J;

J is independently at each occurrence F, Cl, Br, I, OR^{J}, CN, CF₃, OCF₃, O, S, C(O), S(O), methylenedioxy, ethylenedioxy, N(R^{J})₂, SR^{J}, SOR^{J}, SO₂R^{J}, SO₂N(R^{J})₂, SO₃R^{J}, C(O)R^{J}, C(O)C(O)R^{J}, C(O)CH₂C(O)R^{J}, C(S)R^{J}, C(O)OR^{J}, OC(O)R^{J}, OC(O)OR^{J}, C(O)N(R^{J})₂, OC(O)N(R^{J})₂, C(S)N(R^{J})₂, (CH₂)₀₋₂NHC(O)R^{J}, N(R^{J})N(R^{J})C(O)R^{J}, N(R^{J})N(R^{J})C(O)OR^{J}, N(R^{J})N(R^{J})CON(R^{J})₂, N(R^{J})SO₂R^{J}, N(R^{J})SO₂N(R^{J})₂, N(R^{J})C(O)OR^{J}, N(R^{J})C(O)R^{J}, N(R^{J})C(S)R^{J}, N(R^{J})C(O)N(R^{J})₂, N(R^{J})C(S)N(R^{J})₂, N(COR^{J})COR^{J}, N(OR)R^{J}, C(═NH)N(R^{J})₂, C(O)N(OR^{J})R^{J}, or C(═NOR^{J})R; wherein R^{J} is hydrogen, alkyl, cycloalkyl, cycloalkylalkyl, aryl, aralkyl, heterocyclyl, heterocyclylalkyl, heteroaryl, or heteroarylalkyl; or J is Ar² wherein Ar² is cycloalkyl, aryl, heterocyclyl, or heteroaryl, wherein any cycloalkyl, aryl, heterocyclyl, or heteroaryl, is mono- or independently multi-substituted with J, (C₁-C₆)alkyl, (C₂-C₆)alkenyl, (C₂-C₆) alkynyl, (C₁-C₆)haloalkyl, (C₁-C₆)alkoxy, (C₁-C₆)haloalkoxy, cycloalkyl-(C₀-C₆)alkyl, heterocyclyl-(C₀-C₆)alkyl, aryl-(C₀-C₆)alkyl, or heteroaryl-(C₀-C₆)alkyl, wherein any alkyl, alkenyl, alkynyl, haloalkyl, alkoxy, haloalkoxy, cycloalkyl, heterocyclyl, aryl, or heteroaryl is optionally mono- or independently multi-substituted with J;

wherein any cycloalkyl, aryl, heterocyclyl, or heteroaryl comprised by formula (I) can be fused, bridged, or in a spiro configuration with one or more additional optionally substituted cycloalkyl, aryl, heterocyclyl, and heteroaryl, monocyclic, bicyclic or polycyclic, saturated, partially unsaturated, or aromatic rings; and wherein any heterocyclyl or heteroaryl comprising nitrogen can be an N-oxide or N-metho salt thereof;

or any salt thereof.

A compound of formula (I) can be a vasopressin receptor modulatory compound, useful for carrying out methods of treatment and medical uses as described and claimed herein.

In various embodiments, the compound of the invention can comprise a compound of formula (IA)

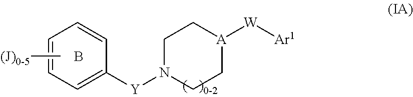

wherein Y is a bond, (CHR)₁₋₄, (CH₂)₀₋₂C(O)(CH₂)₀₋₂, or (CH₂)₀₋₂OC(O)(CH₂)₀₋₂, and ring B can comprise one or two N atoms, and wherein A, J, W, and Ar¹ are as defined above for a compound of formula (I).

In various embodiments, the compound of the invention can comprise a compound of formula (IB)

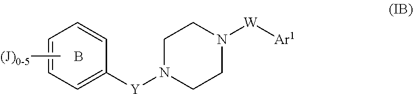

wherein Y is a bond, (CHR)₁₋₄, (CH₂)₀₋₂C(O)(CH₂)₀₋₂, or (CH₂)₀₋₂OC(O)(CH₂)₀₋₂, and ring B can comprise one or two N atoms, and wherein J, W, and Ar¹ are as defined above for a compound of formula (I).

In various embodiments, the compound of the invention can comprise a compound of formula (IC)

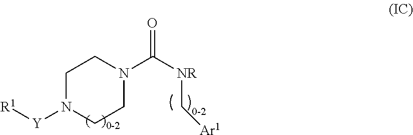

wherein R, R¹, Y, and Ar¹ are as defined above for a compound of formula (I).

In various embodiments, the compound of the invention can comprise a compound of formula (ID)

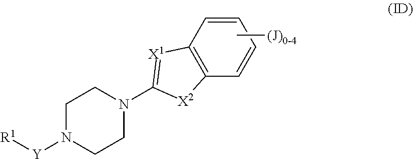

wherein J, R¹, and Y are as defined above for a compound of formula (I), and
wherein X¹ is CR or N, and X² is NR, S, or O.

In various embodiments, the compound of the invention can comprise a compound of formula (I) wherein Ar¹ is any of the following

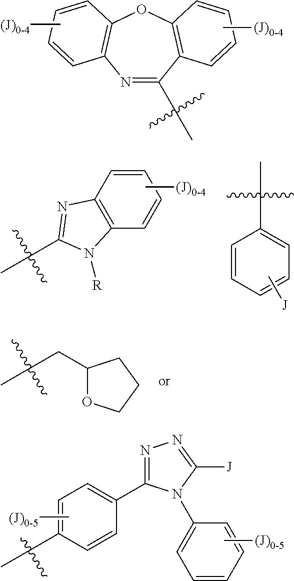

wherein J is as defined above and a wavy line indicates a point of attachment.

In various embodiments, the compound of the invention can comprise a compound of formula (I) wherein R¹ is unsubstituted or substituted phenyl or pyridyl.

In various embodiments, the compound of the invention can comprise a compound of formula (I) wherein W is C(O) or C(O)NR, or wherein Y is a bond, or both.

In various embodiments, the compound of the invention can comprise a compound of formula (I) wherein the group of formula

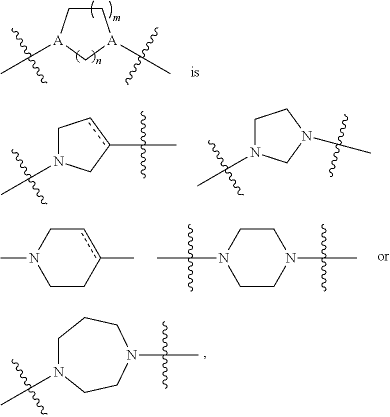

wherein a wavy line indicates a point of attachment, and wherein a dashed line indicates that a single or a double bond can be present.

In various embodiments, the compound of the invention can comprise any of the exemplary compounds of formula (I) as shown below in the list of specific compounds of the invention.

In various embodiments, a compound of the invention can be any of the exemplary compounds shown below.

TABLE 1
Exemplary compounds of the invention of Formula (I)

| | |
|---|---|
| 1 | 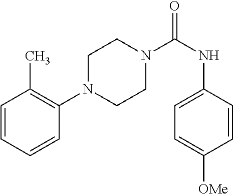 [SMILES: COc1ccc(NC(=O)N2CCN(c3ccccc3C)CC2)cc1] |
| 2 | 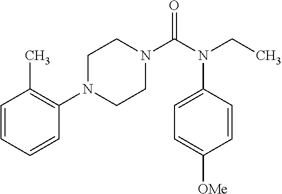 [SMILES: CCN(C(=O)N1CCN(c2ccccc2C)CC1)c1ccc(OC)cc1] |
| 3 | 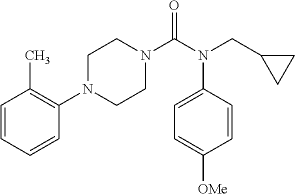 [SMILES: COc1ccc(N(CC2CC2)C(=O)N2CCN(c3ccccc3C)CC2)cc1] |
| 4 | 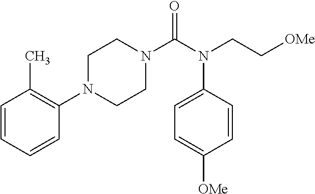 [SMILES: COCCN(C(=O)N1CCN(c2ccccc2C)CC1)c1ccc(OC)cc1] |
| 5 | 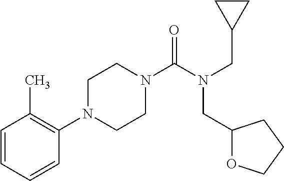 [SMILES: Cc1ccccc1N1CCN(C(=O)N(CC2CC2)CC2CCCO2)CC1] |
| 6 | 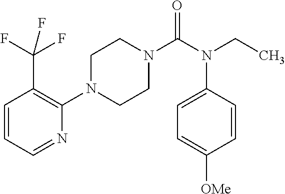 [SMILES: CCN(C(=O)N1CCN(c2ncccc2C(F)(F)F)CC1)c1ccc(OC)cc1] |
| 7 | 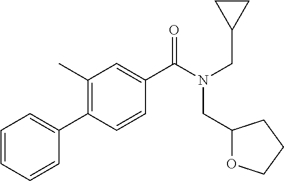 [SMILES: Cc1cc(C(=O)N(CC2CC2)CC2CCCO2)ccc1-c1ccccc1] |
| 8 | 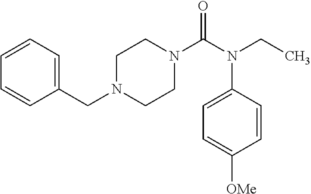 [SMILES: CCN(C(=O)N1CCN(Cc2ccccc2)CC1)c1ccc(OC)cc1] |
| 9 | 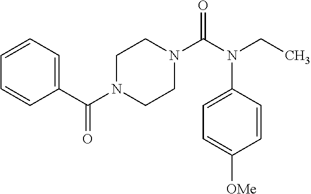 [SMILES: CCN(C(=O)N1CCN(C(=O)c2ccccc2)CC1)c1ccc(OC)cc1] |
| 10 | 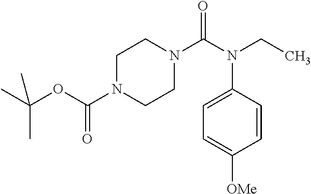 [SMILES: CCN(C(=O)N1CCN(C(=O)OC(C)(C)C)CC1)c1ccc(OC)cc1] |
| 11 | 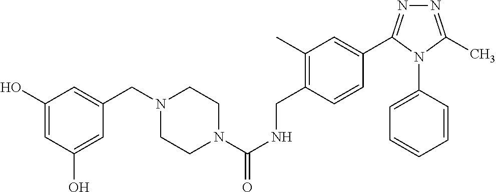 [SMILES: Cc1cc(-c2nnc(C)n2-c2ccccc2)ccc1CNC(=O)N1CCN(Cc2cc(O)cc(O)c2)CC1] |
| 12 | 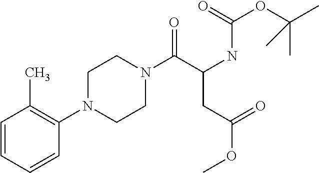 [SMILES: COC(=O)CC(NC(=O)OC(C)(C)C)C(=O)N1CCN(c2ccccc2C)CC1] |
| 13 | 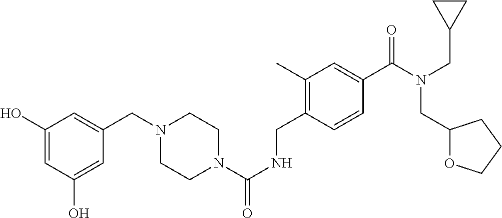 [SMILES: Cc1cc(C(=O)N(CC2CC2)CC2CCCO2)ccc1CNC(=O)N1CCN(Cc2cc(O)cc(O)c2)CC1] |
| 14 | 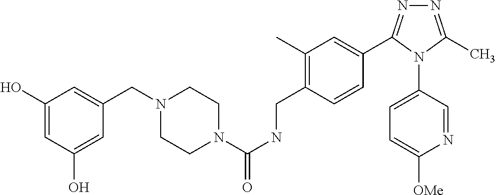 [SMILES: COc1ccc(-n2c(C)nnc2-c2ccc(CNC(=O)N3CCN(Cc4cc(O)cc(O)c4)CC3)c(C)c2)cn1] |
| 15 | 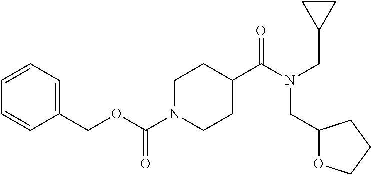 [SMILES: O=C(OCc1ccccc1)N1CCC(C(=O)N(CC2CC2)CC2CCCO2)CC1] |
| 16 | 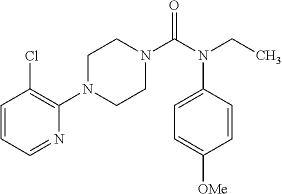 [SMILES: CCN(C(=O)N1CCN(c2ncccc2Cl)CC1)c1ccc(OC)cc1] |
| 17 | 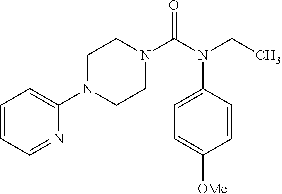 [SMILES: CCN(C(=O)N1CCN(c2ccccn2)CC1)c1ccc(OC)cc1] |
| 18 | 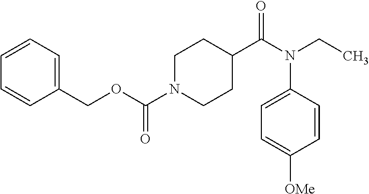 [SMILES: CCN(C(=O)C1CCN(C(=O)OCc2ccccc2)CC1)c1ccc(OC)cc1] |
| 19 | 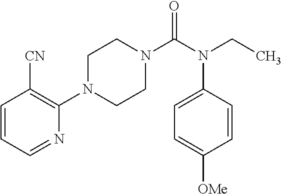 [SMILES: CCN(C(=O)N1CCN(c2ncccc2C#N)CC1)c1ccc(OC)cc1] |
| 20 | 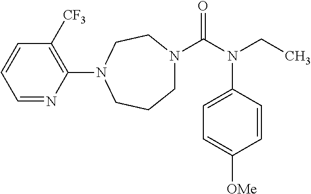 [SMILES: CCN(C(=O)N1CCCN(c2ncccc2C(F)(F)F)CC1)c1ccc(OC)cc1] |
| 21 | 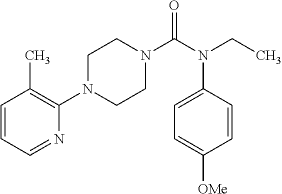 [SMILES: CCN(C(=O)N1CCN(c2ncccc2C)CC1)c1ccc(OC)cc1] |
| 22 | 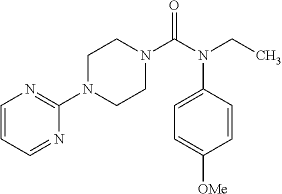 [SMILES: CCN(C(=O)N1CCN(c2ncccn2)CC1)c1ccc(OC)cc1] |
| 23 | 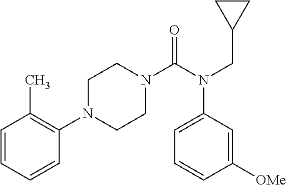 [SMILES: COc1cccc(N(CC2CC2)C(=O)N2CCN(c3ccccc3C)CC2)c1] |
| 24 | 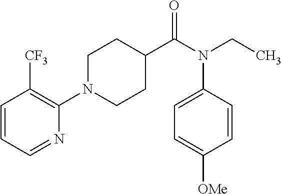 [SMILES: CCN(C(=O)C1CCN(c2ncccc2C(F)(F)F)CC1)c1ccc(OC)cc1] |
| 25 | 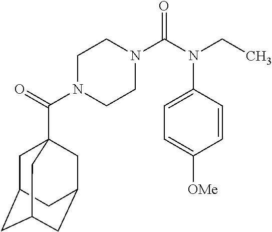 [SMILES: CCN(C(=O)N1CCN(C(=O)C23CC4CC(CC(C4)C2)C3)CC1)c1ccc(OC)cc1] |
| 26 | 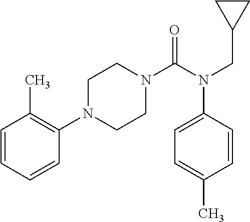 [SMILES: Cc1ccc(N(CC2CC2)C(=O)N2CCN(c3ccccc3C)CC2)cc1] |
| 27 | 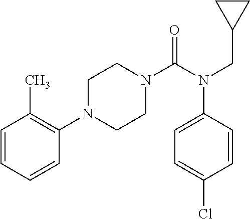 [SMILES: Cc1ccccc1N1CCN(C(=O)N(CC2CC2)c2ccc(Cl)cc2)CC1] |
| 28 | 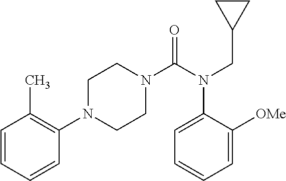 [SMILES: COc1ccccc1N(CC1CC1)C(=O)N1CCN(c2ccccc2C)CC1] |
| 29 | 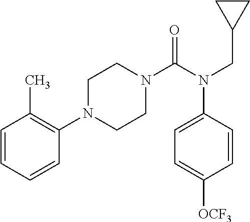 [SMILES: Cc1ccccc1N1CCN(C(=O)N(CC2CC2)c2ccc(OC(F)(F)F)cc2)CC1] |
| 30 | 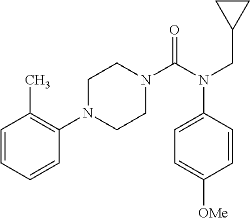 [SMILES: COc1ccc(N(CC2CC2)C(=O)N2CCN(c3ccccc3C)CC2)cc1] |
| 31 | 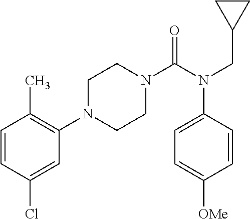 [SMILES: COc1ccc(N(CC2CC2)C(=O)N2CCN(c3cc(Cl)ccc3C)CC2)cc1] |
| 32 | 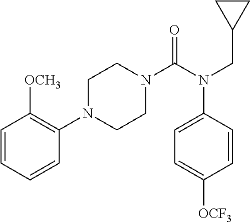 [SMILES: COc1ccccc1N1CCN(C(=O)N(CC2CC2)c2ccc(OC(F)(F)F)cc2)CC1] |
| 33 | 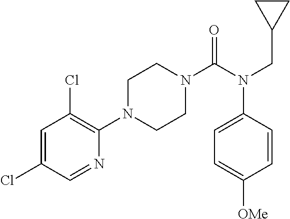 [SMILES: COc1ccc(N(CC2CC2)C(=O)N2CCN(c3ncc(Cl)cc3Cl)CC2)cc1] |
| 34 | 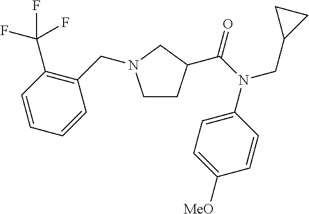 [SMILES: COc1ccc(N(CC2CC2)C(=O)C2CCN(Cc3ccccc3C(F)(F)F)C2)cc1] |
| 35 | 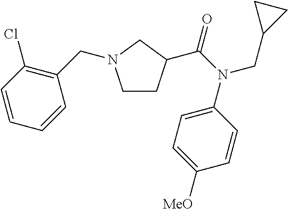 [SMILES: COc1ccc(N(CC2CC2)C(=O)C2CCN(Cc3ccccc3Cl)C2)cc1] |
| 36 | 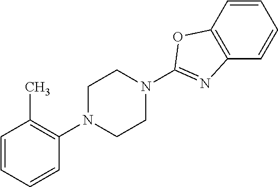 [SMILES: Cc1ccccc1N1CCN(c2nc3ccccc3o2)CC1] |
| 37 | 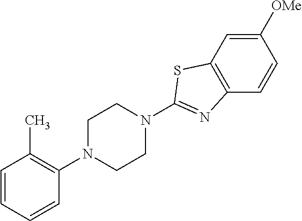 [SMILES: COc1ccc2nc(N3CCN(c4ccccc4C)CC3)sc2c1] |
| 38 | 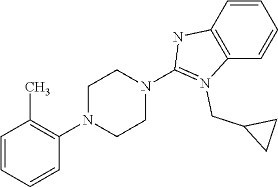 |
| 39 | 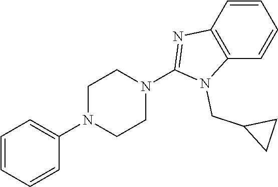 [SMILES: c1ccc(N2CCN(c3nc4ccccc4n3CC3CC3)CC2)cc1] |
| 40 | 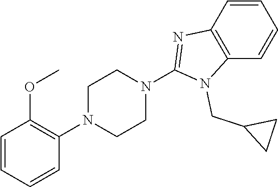 [SMILES: COc1ccccc1N1CCN(c2nc3ccccc3n2CC2CC2)CC1] |
| 41 | 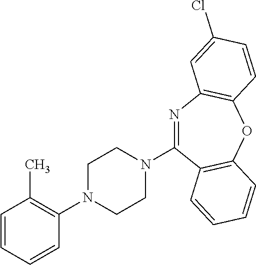 [SMILES: Cc1ccccc1N1CCN(C2=Nc3cc(Cl)ccc3Oc3ccccc32)CC1] |
| 42 | 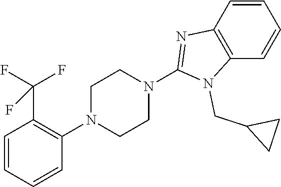 [SMILES: FC(F)(F)c1ccccc1N1CCN(c2nc3ccccc3n2CC2CC2)CC1] |
| 43 | 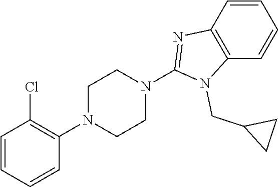 [SMILES: Clc1ccccc1N1CCN(c2nc3ccccc3n2CC2CC2)CC1] |
| 44 | 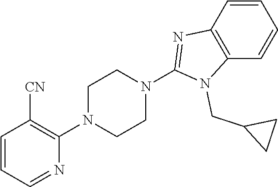 [SMILES: N#Cc1cccnc1N1CCN(c2nc3ccccc3n2CC2CC2)CC1] |
| 45 | 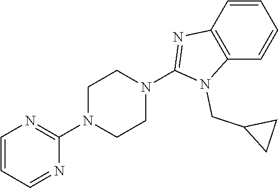 [SMILES: c1cnc(N2CCN(c3nc4ccccc4n3CC3CC3)CC2)nc1] |
| 46 | 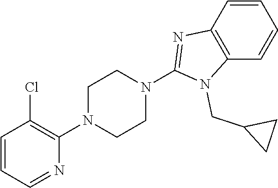 [SMILES: Clc1cccnc1N1CCN(c2nc3ccccc3n2CC2CC2)CC1] |
| 47 | 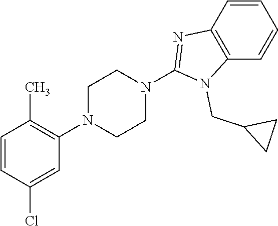 [SMILES: Cc1ccc(Cl)cc1N1CCN(c2nc3ccccc3n2CC2CC2)CC1] |
| 48 | 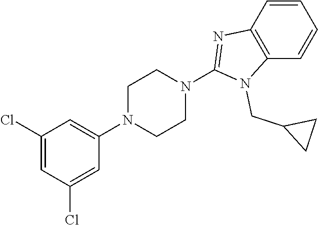 [SMILES: Clc1cc(Cl)cc(N2CCN(c3nc4ccccc4n3CC3CC3)CC2)c1] |
| 49 | 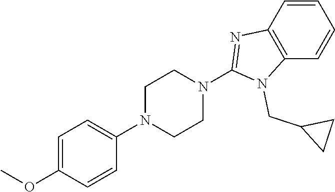 [SMILES: COc1ccc(N2CCN(c3nc4ccccc4n3CC3CC3)CC2)cc1] |
| 50 | 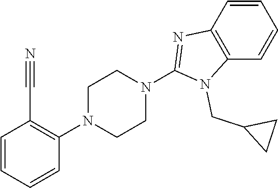 [SMILES: N#Cc1ccccc1N1CCN(c2nc3ccccc3n2CC2CC2)CC1] |
| 51 | 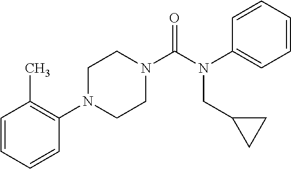 [SMILES: Cc1ccccc1N1CCN(C(=O)N(CC2CC2)c2ccccc2)CC1] |
| 52 | 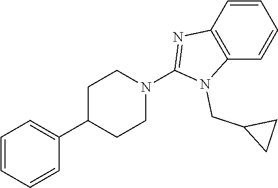 [SMILES: c1ccc(C2CCN(c3nc4ccccc4n3CC3CC3)CC2)cc1] |
| 53 | 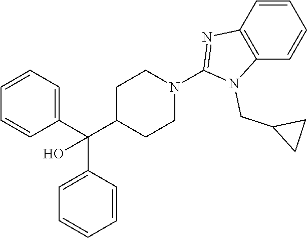 [SMILES: OC(c1ccccc1)(c1ccccc1)C1CCN(c2nc3ccccc3n2CC2CC2)CC1] |
| 54 | 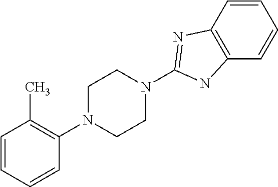 [SMILES: Cc1ccccc1N1CCN(c2nc3ccccc3[nH]2)CC1] |
| 55 | 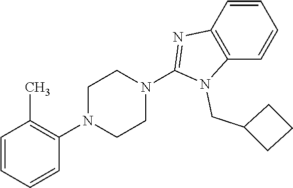 [SMILES: Cc1ccccc1N1CCN(c2nc3ccccc3n2CC2CCC2)CC1] |
| 56 | 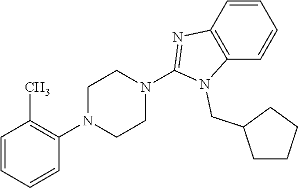 [SMILES: Cc1ccccc1N1CCN(c2nc3ccccc3n2CC2CCCC2)CC1] |
| 57 | 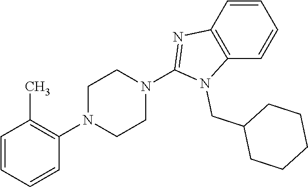 [SMILES: Cc1ccccc1N1CCN(c2nc3ccccc3n2CC2CCCCC2)CC1] |
| 58 | 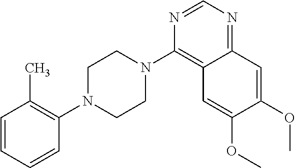 [SMILES: COc1cc2ncnc(N3CCN(c4ccccc4C)CC3)c2cc1OC] |
| 59 | 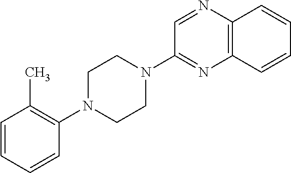 [SMILES: Cc1ccccc1N1CCN(c2cnc3ccccc3n2)CC1] |
| 60 | 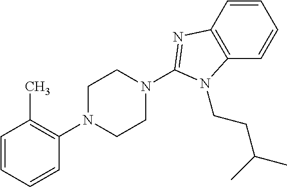 [SMILES: Cc1ccccc1N1CCN(c2nc3ccccc3n2CCC(C)C)CC1] |
| 61 | 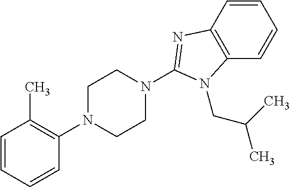 [SMILES: Cc1ccccc1N1CCN(c2nc3ccccc3n2CC(C)C)CC1] |
| 62 | 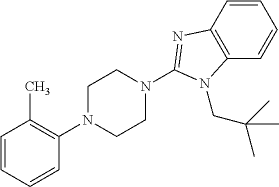 [SMILES: Cc1ccccc1N1CCN(c2nc3ccccc3n2CC(C)(C)C)CC1] |
| 63 | 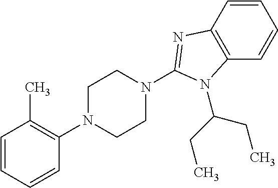 [SMILES: CCC(CC)n1c(N2CCN(c3ccccc3C)CC2)nc2ccccc21] |
| 64 | 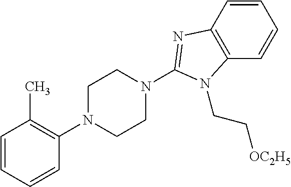 [SMILES: CCOCCn1c(N2CCN(c3ccccc3C)CC2)nc2ccccc21] |
| 65 | 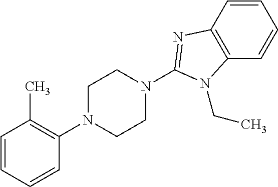 [SMILES: CCn1c(N2CCN(c3ccccc3C)CC2)nc2ccccc21] |
| 66 | 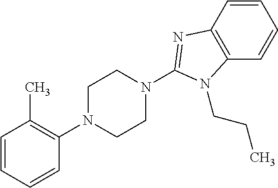 [SMILES: CCCn1c(N2CCN(c3ccccc3C)CC2)nc2ccccc21] |
| 67 | 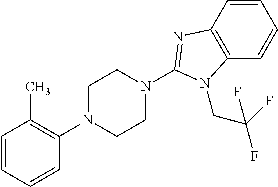 [SMILES: Cc1ccccc1N1CCN(c2nc3ccccc3n2CC(F)(F)F)CC1] |
| 68 | 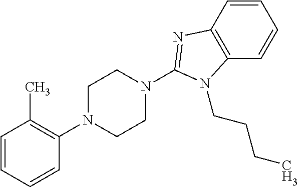 [SMILES: CCCCn1c(N2CCN(c3ccccc3C)CC2)nc2ccccc21] |
| 69 | 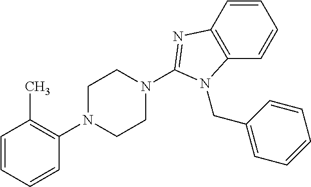 [SMILES: Cc1ccccc1N1CCN(c2nc3ccccc3n2Cc2ccccc2)CC1] |
| 70 | 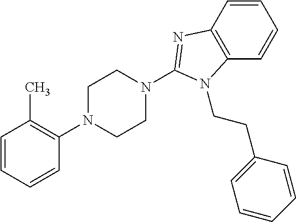 [SMILES: Cc1ccccc1N1CCN(c2nc3ccccc3n2CCc2ccccc2)CC1] |
| 71 | 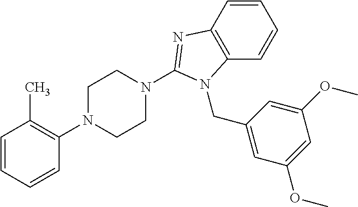 [SMILES: COc1cc(Cn2c(N3CCN(c4ccccc4C)CC3)nc3ccccc32)cc(OC)c1] |
| 72 | 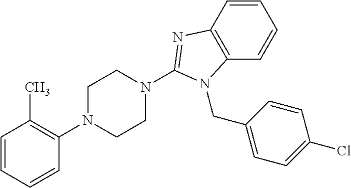 [SMILES: Cc1ccccc1N1CCN(c2nc3ccccc3n2Cc2ccc(Cl)cc2)CC1] |
| 73 | 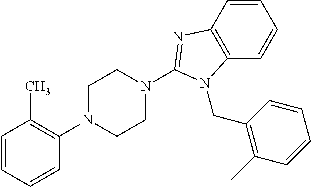 [SMILES: Cc1ccccc1Cn1c(N2CCN(c3ccccc3C)CC2)nc2ccccc21] |
| 74 | 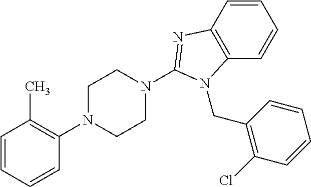 [SMILES: Cc1ccccc1N1CCN(c2nc3ccccc3n2Cc2ccccc2Cl)CC1] |
| 75 | 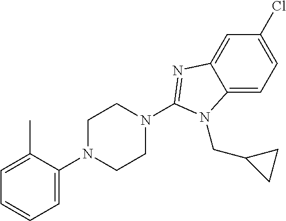 [SMILES: Cc1ccccc1N1CCN(c2nc3cc(Cl)ccc3n2CC2CC2)CC1] |
| 76 | 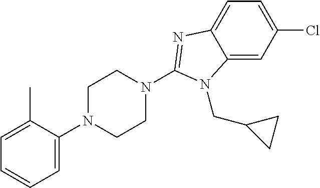 [SMILES: Cc1ccccc1N1CCN(c2nc3ccc(Cl)cc3n2CC2CC2)CC1] |
| 77 | 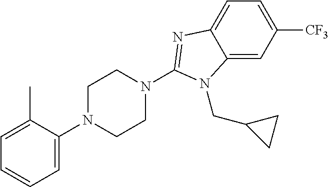 [SMILES: Cc1ccccc1N1CCN(c2nc3ccc(C(F)(F)F)cc3n2CC2CC2)CC1] |
| 78 | 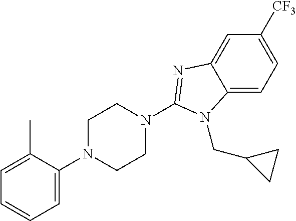 [SMILES: Cc1ccccc1N1CCN(c2nc3cc(C(F)(F)F)ccc3n2CC2CC2)CC1] |
| 79 | 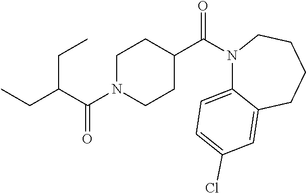 [SMILES: CCC(CC)C(=O)N1CCC(C(=O)N2CCCCc3cc(Cl)ccc32)CC1] |
| 80 | 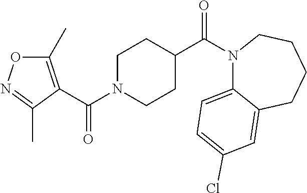 [SMILES: Cc1noc(C)c1C(=O)N1CCC(C(=O)N2CCCCc3cc(Cl)ccc32)CC1] |
| 81 | 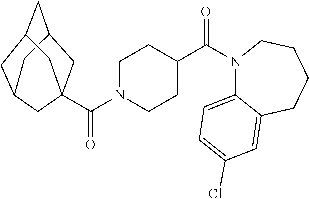 [SMILES: O=C(C1CCN(C(=O)C23CC4CC(CC(C4)C2)C3)CC1)N1CCCCc2cc(Cl)ccc21] |
| 82 | 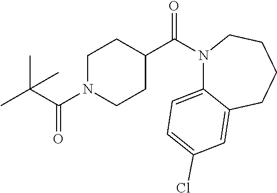 [SMILES: CC(C)(C)C(=O)N1CCC(C(=O)N2CCCCc3cc(Cl)ccc32)CC1] |
| 83 | 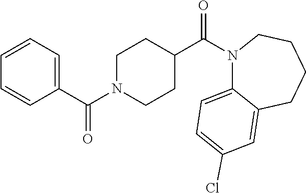 [SMILES: O=C(c1ccccc1)N1CCC(C(=O)N2CCCCc3cc(Cl)ccc32)CC1] |
| 84 | 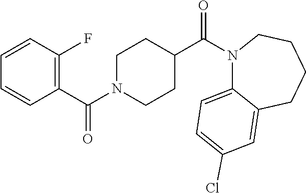 [SMILES: O=C(c1ccccc1F)N1CCC(C(=O)N2CCCCc3cc(Cl)ccc32)CC1] |
| 85 | 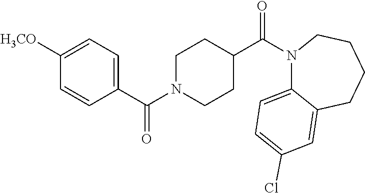 [SMILES: COc1ccc(C(=O)N2CCC(C(=O)N3CCCCc4cc(Cl)ccc43)CC2)cc1] |
| 86 | 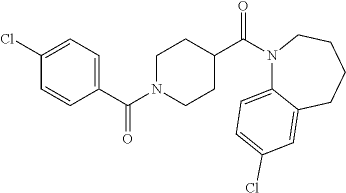 [SMILES: O=C(c1ccc(Cl)cc1)N1CCC(C(=O)N2CCCCc3cc(Cl)ccc32)CC1] |
| 87 | 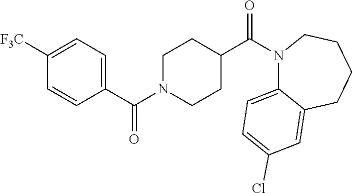 [SMILES: O=C(c1ccc(C(F)(F)F)cc1)N1CCC(C(=O)N2CCCCc3cc(Cl)ccc32)CC1] |
| 88 | 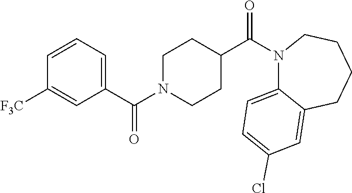 [SMILES: O=C(c1cccc(C(F)(F)F)c1)N1CCC(C(=O)N2CCCCc3cc(Cl)ccc32)CC1] |
| 89 | 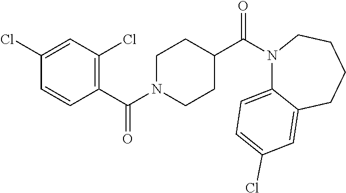 [SMILES: O=C(c1ccc(Cl)cc1Cl)N1CCC(C(=O)N2CCCCc3cc(Cl)ccc32)CC1] |
| 90 | 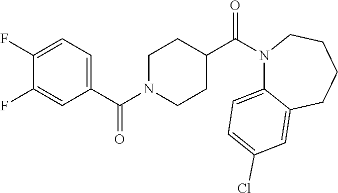 [SMILES: O=C(c1ccc(F)c(F)c1)N1CCC(C(=O)N2CCCCc3cc(Cl)ccc32)CC1] |
| 91 | 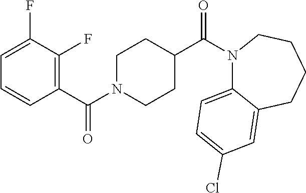 [SMILES: O=C(c1cccc(F)c1F)N1CCC(C(=O)N2CCCCc3cc(Cl)ccc32)CC1] |
| 92 | 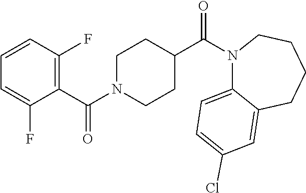 [SMILES: O=C(c1c(F)cccc1F)N1CCC(C(=O)N2CCCCc3cc(Cl)ccc32)CC1] |
| 93 | 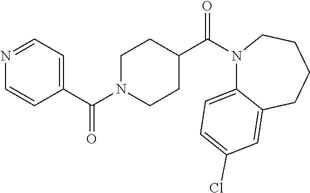 [SMILES: O=C(c1ccncc1)N1CCC(C(=O)N2CCCCc3cc(Cl)ccc32)CC1] |
| 94 | 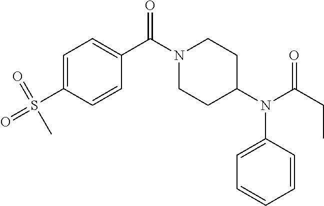 [SMILES: CCC(=O)N(c1ccccc1)C1CCN(C(=O)c2ccc(S(C)(=O)=O)cc2)CC1] |
| 95 | 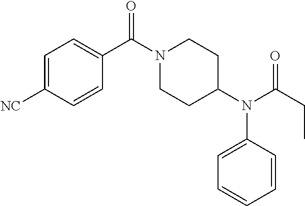 [SMILES: CCC(=O)N(c1ccccc1)C1CCN(C(=O)c2ccc(C#N)cc2)CC1] |
| 96 | 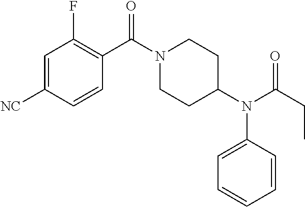 [SMILES: CCC(=O)N(c1ccccc1)C1CCN(C(=O)c2ccc(C#N)cc2F)CC1] |
| 97 | 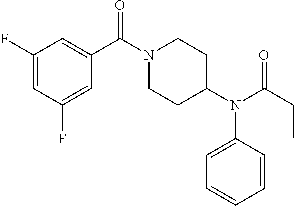 [SMILES: CCC(=O)N(c1ccccc1)C1CCN(C(=O)c2cc(F)cc(F)c2)CC1] |
| 98 | 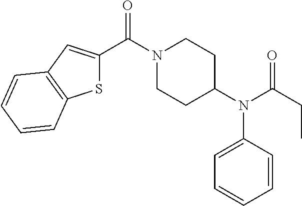 [SMILES: CCC(=O)N(c1ccccc1)C1CCN(C(=O)c2cc3ccccc3s2)CC1] |
| 99 | 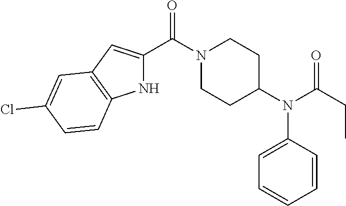 [SMILES: CCC(=O)N(c1ccccc1)C1CCN(C(=O)c2cc3cc(Cl)ccc3[nH]2)CC1] |
| 100 | 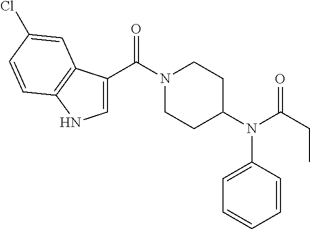 [SMILES: CCC(=O)N(c1ccccc1)C1CCN(C(=O)c2c[nH]c3ccc(Cl)cc23)CC1] |
| 101 | 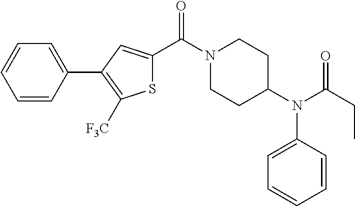 [SMILES: CCC(=O)N(c1ccccc1)C1CCN(C(=O)c2cc(-c3ccccc3)c(C(F)(F)F)s2)CC1] |
| 102 | 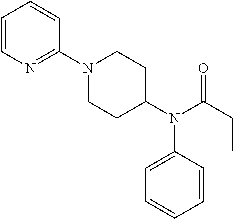 [SMILES: CCC(=O)N(c1ccccc1)C1CCN(c2ccccn2)CC1] |
| 103 | 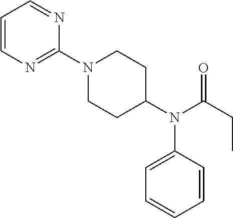 [SMILES: CCC(=O)N(c1ccccc1)C1CCN(c2ncccn2)CC1] |
| 104 | 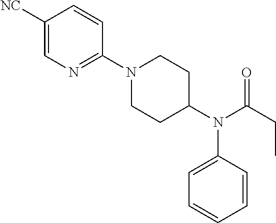 [SMILES: CCC(=O)N(c1ccccc1)C1CCN(c2ccc(C#N)cn2)CC1] |
| 105 | 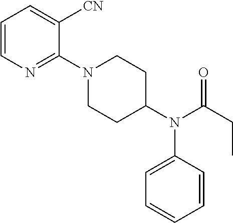 [SMILES: CCC(=O)N(c1ccccc1)C1CCN(c2ncccc2C#N)CC1] |
| 106 | 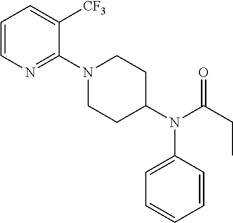 [SMILES: CCC(=O)N(c1ccccc1)C1CCN(c2ncccc2C(F)(F)F)CC1] |
| 107 | 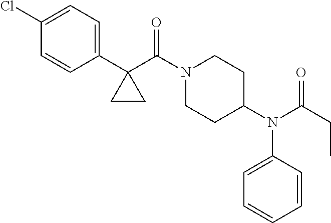 [SMILES: CCC(=O)N(c1ccccc1)C1CCN(C(=O)C2(c3ccc(Cl)cc3)CC2)CC1] |
| 108 | 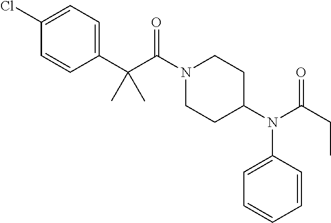 [SMILES: CCC(=O)N(c1ccccc1)C1CCN(C(=O)C(C)(C)c2ccc(Cl)cc2)CC1] |
| 109 | 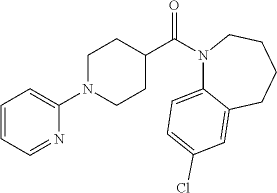 [SMILES: O=C(C1CCN(c2ccccn2)CC1)N1CCCCc2cc(Cl)ccc21] |
| 110 | 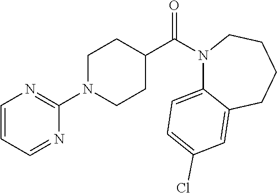 [SMILES: O=C(C1CCN(c2ncccn2)CC1)N1CCCCc2cc(Cl)ccc21] |
| 111 | 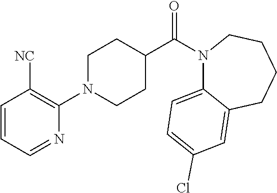 [SMILES: N#Cc1cccnc1N1CCC(C(=O)N2CCCCc3cc(Cl)ccc32)CC1] |
| 112 | 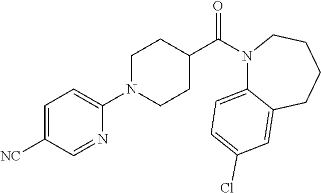 [SMILES: N#Cc1ccc(N2CCC(C(=O)N3CCCCc4cc(Cl)ccc43)CC2)nc1] |
| 113 | 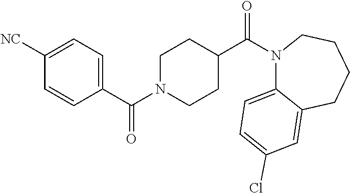 [SMILES: N#Cc1ccc(C(=O)N2CCC(C(=O)N3CCCCc4cc(Cl)ccc43)CC2)cc1] |
| 114 | 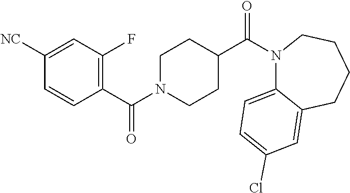 [SMILES: N#Cc1ccc(C(=O)N2CCC(C(=O)N3CCCCc4cc(Cl)ccc43)CC2)c(F)c1] |
| 115 | 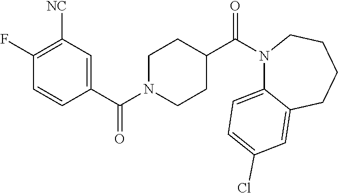 [SMILES: N#Cc1cc(C(=O)N2CCC(C(=O)N3CCCCc4cc(Cl)ccc43)CC2)ccc1F] |
| 116 | 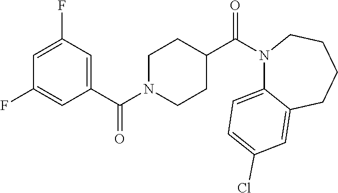 [SMILES: O=C(c1cc(F)cc(F)c1)N1CCC(C(=O)N2CCCCc3cc(Cl)ccc32)CC1] |
| 117 | 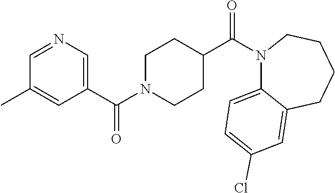 [SMILES: Cc1cncc(C(=O)N2CCC(C(=O)N3CCCCc4cc(Cl)ccc43)CC2)c1] |
| 118 | 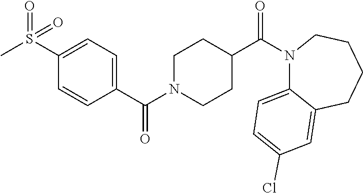 [SMILES: CS(=O)(=O)c1ccc(C(=O)N2CCC(C(=O)N3CCCCc4cc(Cl)ccc43)CC2)cc1] |
| 119 | 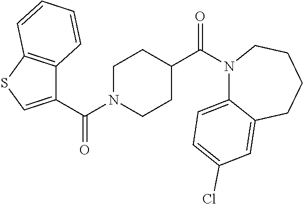 [SMILES: O=C(c1csc2ccccc12)N1CCC(C(=O)N2CCCCc3cc(Cl)ccc32)CC1] |
| 120 | 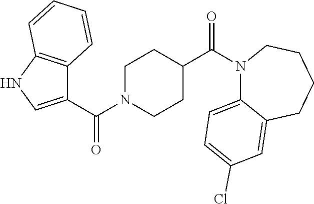 [SMILES: O=C(c1c[nH]c2ccccc12)N1CCC(C(=O)N2CCCCc3cc(Cl)ccc32)CC1] |
| 121 | 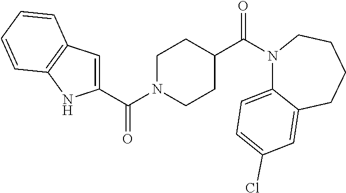 [SMILES: O=C(c1cc2ccccc2[nH]1)N1CCC(C(=O)N2CCCCc3cc(Cl)ccc32)CC1] |
| 122 | 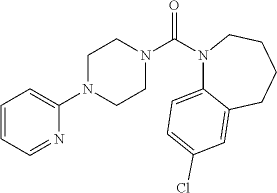 [SMILES: O=C(N1CCN(c2ccccn2)CC1)N1CCCCc2cc(Cl)ccc21] |
| 123 | 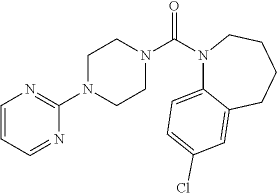 [SMILES: O=C(N1CCN(c2ncccn2)CC1)N1CCCCc2cc(Cl)ccc21] |
| 124 | 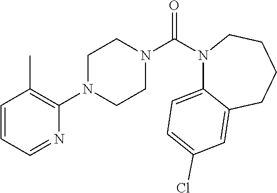 [SMILES: Cc1cccnc1N1CCN(C(=O)N2CCCCc3cc(Cl)ccc32)CC1] |
| 125 | 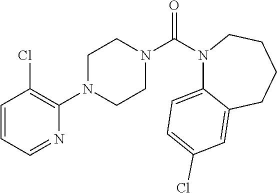 [SMILES: O=C(N1CCN(c2ncccc2Cl)CC1)N1CCCCc2cc(Cl)ccc21] |
| 126 | 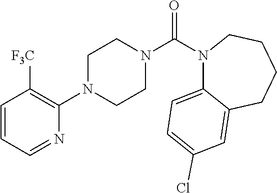 [SMILES: O=C(N1CCN(c2ncccc2C(F)(F)F)CC1)N1CCCCc2cc(Cl)ccc21] |
| 127 | 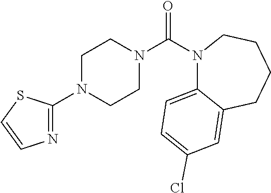 [SMILES: O=C(N1CCN(c2nccs2)CC1)N1CCCCc2cc(Cl)ccc21] |
| 128 | 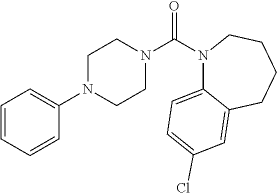 [SMILES: O=C(N1CCN(c2ccccc2)CC1)N1CCCCc2cc(Cl)ccc21] |
| 129 | 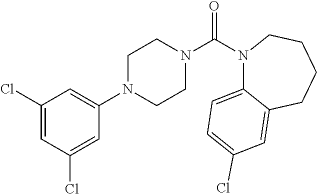 [SMILES: O=C(N1CCN(c2cc(Cl)cc(Cl)c2)CC1)N1CCCCc2cc(Cl)ccc21] |
| 130 | 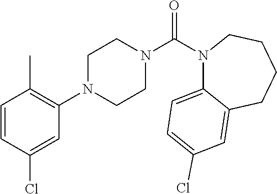 [SMILES: Cc1ccc(Cl)cc1N1CCN(C(=O)N2CCCCc3cc(Cl)ccc32)CC1] |
| 131 | 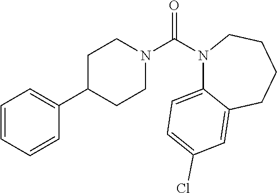 [SMILES: O=C(N1CCC(c2ccccc2)CC1)N1CCCCc2cc(Cl)ccc21] |
| 132 | 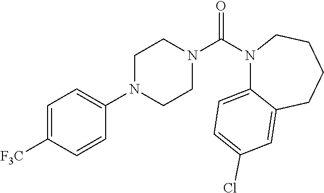 [SMILES: O=C(N1CCN(c2ccc(C(F)(F)F)cc2)CC1)N1CCCCc2cc(Cl)ccc21] |
| 133 | 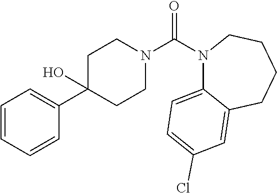 [SMILES: O=C(N1CCC(O)(c2ccccc2)CC1)N1CCCCc2cc(Cl)ccc21] |
| 134 | 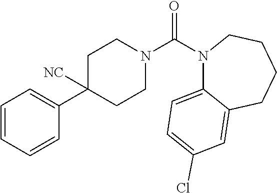 [SMILES: N#CC1(c2ccccc2)CCN(C(=O)N2CCCCc3cc(Cl)ccc32)CC1] |
| 135 | 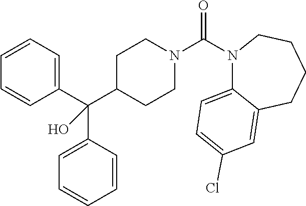 [SMILES: O=C(N1CCC(C(O)(c2ccccc2)c2ccccc2)CC1)N1CCCCc2cc(Cl)ccc21] |
| 136 | 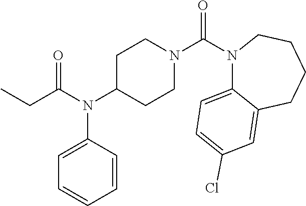 [SMILES: CCC(=O)N(c1ccccc1)C1CCN(C(=O)N2CCCCc3cc(Cl)ccc32)CC1] |
| 139 | 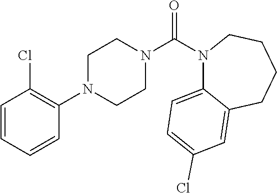 [SMILES: O=C(N1CCN(c2ccccc2Cl)CC1)N1CCCCc2cc(Cl)ccc21] |
| 140 | 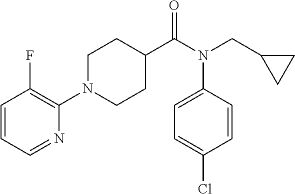 [SMILES: O=C(C1CCN(c2ncccc2F)CC1)N(CC1CC1)c1ccc(Cl)cc1] |
| 141 | 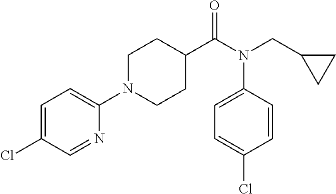 [SMILES: O=C(C1CCN(c2ccc(Cl)cn2)CC1)N(CC1CC1)c1ccc(Cl)cc1] |
| 142 | 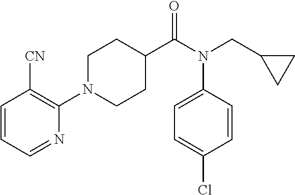 [SMILES: N#Cc1cccnc1N1CCC(C(=O)N(CC2CC2)c2ccc(Cl)cc2)CC1] |
| 143 | 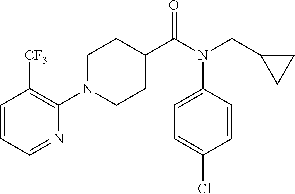 [SMILES: O=C(C1CCN(c2ncccc2C(F)(F)F)CC1)N(CC1CC1)c1ccc(Cl)cc1] |
| 144 | 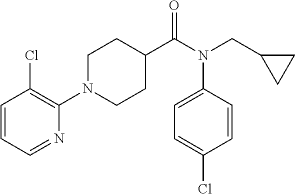 [SMILES: O=C(C1CCN(c2ncccc2Cl)CC1)N(CC1CC1)c1ccc(Cl)cc1] |
| 145 | 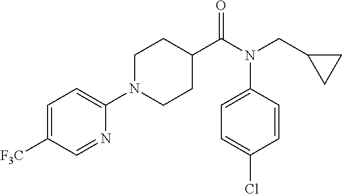 [SMILES: O=C(C1CCN(c2ccc(C(F)(F)F)cn2)CC1)N(CC1CC1)c1ccc(Cl)cc1] |
| 146 | 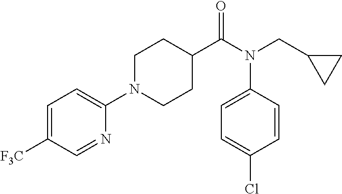 [SMILES: O=C(C1CCN(c2ccc(C(F)(F)F)cn2)CC1)N(CC1CC1)c1ccc(Cl)cc1] |
| 147 | 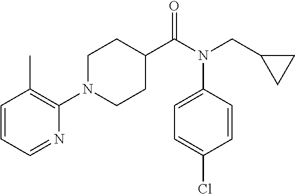 [SMILES: Cc1cccnc1N1CCC(C(=O)N(CC2CC2)c2ccc(Cl)cc2)CC1] |
| 148 | 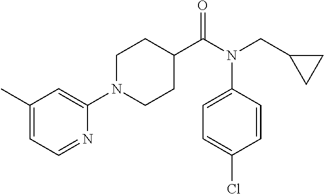 [SMILES: Cc1ccnc(N2CCC(C(=O)N(CC3CC3)c3ccc(Cl)cc3)CC2)c1] |
| 149 | 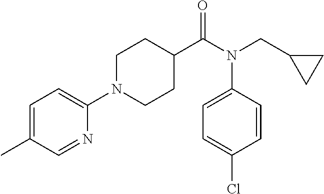 [SMILES: Cc1ccc(N2CCC(C(=O)N(CC3CC3)c3ccc(Cl)cc3)CC2)nc1] |
| 150 | 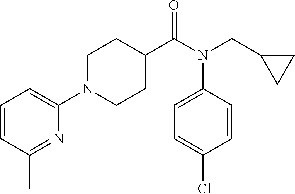 [SMILES: Cc1cccc(N2CCC(C(=O)N(CC3CC3)c3ccc(Cl)cc3)CC2)n1] |
| 151 | 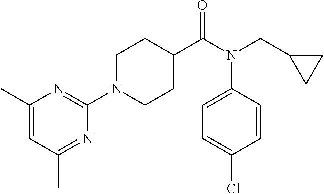 [SMILES: Cc1cc(C)nc(N2CCC(C(=O)N(CC3CC3)c3ccc(Cl)cc3)CC2)n1] |
| 152 | 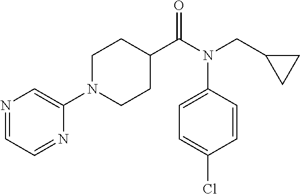 [SMILES: O=C(C1CCN(c2cnccn2)CC1)N(CC1CC1)c1ccc(Cl)cc1] |
| 153 | 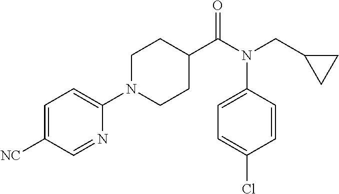 [SMILES: N#Cc1ccc(N2CCC(C(=O)N(CC3CC3)c3ccc(Cl)cc3)CC2)nc1] |
| 154 | 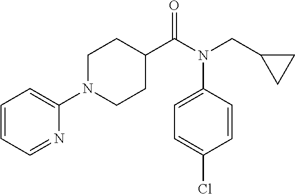 [SMILES: O=C(C1CCN(c2ccccn2)CC1)N(CC1CC1)c1ccc(Cl)cc1] |
| 155 | 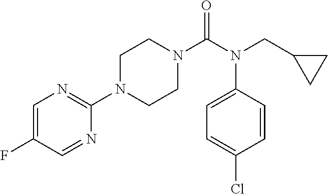 [SMILES: O=C(N1CCN(c2ncc(F)cn2)CC1)N(CC1CC1)c1ccc(Cl)cc1] |
| 156 | 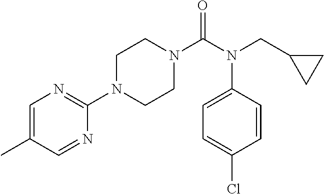 [SMILES: Cc1cnc(N2CCN(C(=O)N(CC3CC3)c3ccc(Cl)cc3)CC2)nc1] |
| 157 | 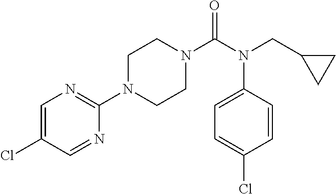 [SMILES: O=C(N1CCN(c2ncc(Cl)cn2)CC1)N(CC1CC1)c1ccc(Cl)cc1] |
| 158 | 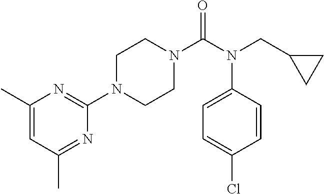 [SMILES: Cc1cc(C)nc(N2CCN(C(=O)N(CC3CC3)c3ccc(Cl)cc3)CC2)n1] |
| 159 | 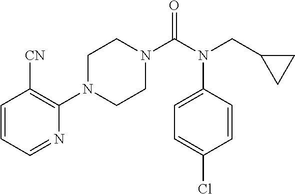 [SMILES: N#Cc1cccnc1N1CCN(C(=O)N(CC2CC2)c2ccc(Cl)cc2)CC1] |
| 160 | 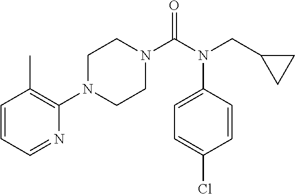 [SMILES: Cc1cccnc1N1CCN(C(=O)N(CC2CC2)c2ccc(Cl)cc2)CC1] |
| 161 | 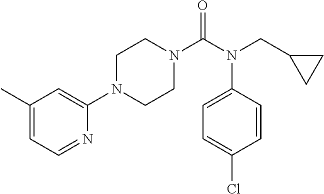 [SMILES: Cc1ccnc(N2CCN(C(=O)N(CC3CC3)c3ccc(Cl)cc3)CC2)c1] |
| 162 | 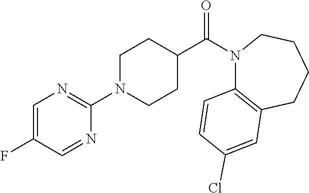 [SMILES: O=C(C1CCN(c2ncc(F)cn2)CC1)N1CCCCc2cc(Cl)ccc21] |
| 163 | 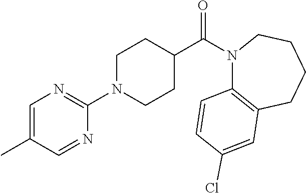 [SMILES: Cc1cnc(N2CCC(C(=O)N3CCCCc4cc(Cl)ccc43)CC2)nc1] |
| 164 | 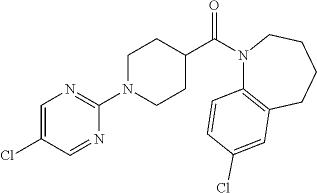 [SMILES: O=C(C1CCN(c2ncc(Cl)cn2)CC1)N1CCCCc2cc(Cl)ccc21] |
| 165 | 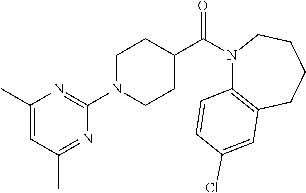 [SMILES: Cc1cc(C)nc(N2CCC(C(=O)N3CCCCc4cc(Cl)ccc43)CC2)n1] |
| 166 | 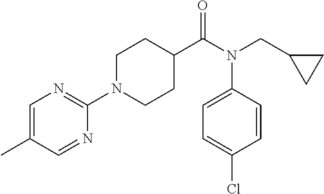 [SMILES: Cc1cnc(N2CCC(C(=O)N(CC3CC3)c3ccc(Cl)cc3)CC2)nc1] |
| 167 | 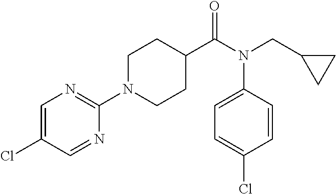 [SMILES: O=C(C1CCN(c2ncc(Cl)cn2)CC1)N(CC1CC1)c1ccc(Cl)cc1] |
| 168 | 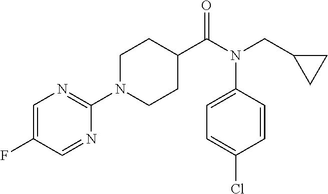 [SMILES: O=C(C1CCN(c2ncc(F)cn2)CC1)N(CC1CC1)c1ccc(Cl)cc1] |
| 169 | 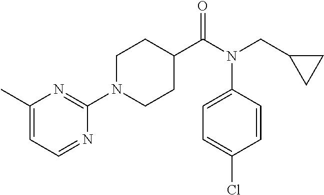 [SMILES: Cc1ccnc(N2CCC(C(=O)N(CC3CC3)c3ccc(Cl)cc3)CC2)n1] |
| 170 | 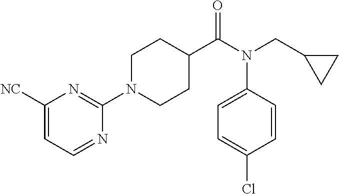 [SMILES: N#Cc1ccnc(N2CCC(C(=O)N(CC3CC3)c3ccc(Cl)cc3)CC2)n1] |
| 171 | 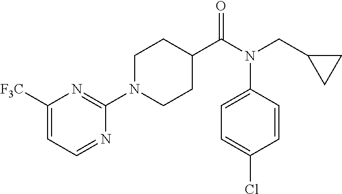 [SMILES: O=C(C1CCN(c2nccc(C(F)(F)F)n2)CC1)N(CC1CC1)c1ccc(Cl)cc1] |
| 172 | 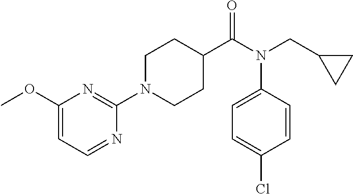 [SMILES: COc1ccnc(N2CCC(C(=O)N(CC3CC3)c3ccc(Cl)cc3)CC2)n1] |
| 173 | 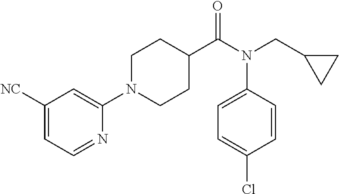 [SMILES: N#Cc1ccnc(N2CCC(C(=O)N(CC3CC3)c3ccc(Cl)cc3)CC2)c1] |
| 174 | 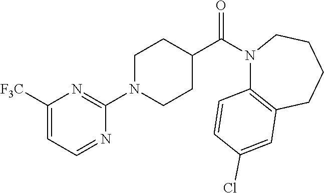 [SMILES: O=C(C1CCN(c2nccc(C(F)(F)F)n2)CC1)N1CCCCc2cc(Cl)ccc21] |
| 175 | 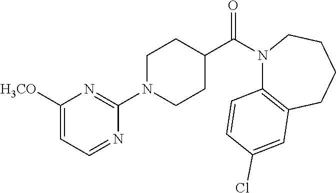 [SMILES: COc1ccnc(N2CCC(C(=O)N3CCCCc4cc(Cl)ccc43)CC2)n1] |
| 176 | 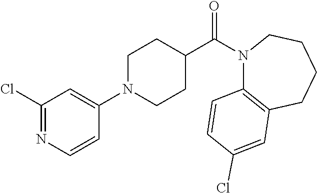 [SMILES: O=C(C1CCN(c2ccnc(Cl)c2)CC1)N1CCCCc2cc(Cl)ccc21] |
| 177 | 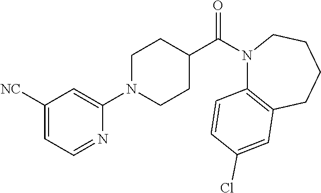 [SMILES: N#Cc1ccnc(N2CCC(C(=O)N3CCCCc4cc(Cl)ccc43)CC2)c1] |
| 178 | 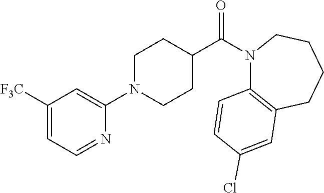 [SMILES: O=C(C1CCN(c2cc(C(F)(F)F)ccn2)CC1)N1CCCCc2cc(Cl)ccc21] |
| 179 | 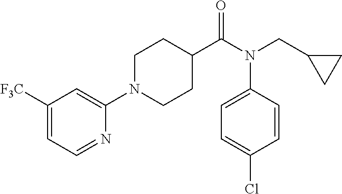 [SMILES: O=C(C1CCN(c2cc(C(F)(F)F)ccn2)CC1)N(CC1CC1)c1ccc(Cl)cc1] |
| 180 | 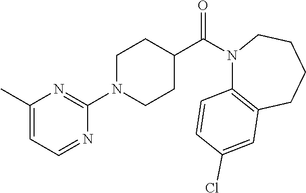 [SMILES: Cc1ccnc(N2CCC(C(=O)N3CCCCc4cc(Cl)ccc43)CC2)n1] |
| 181 | 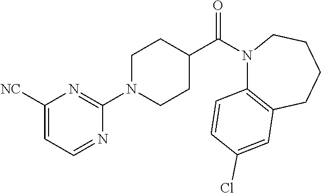 [SMILES: N#Cc1ccnc(N2CCC(C(=O)N3CCCCc4cc(Cl)ccc43)CC2)n1] |
| 182 | 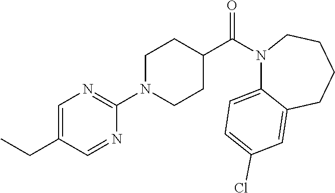 [SMILES: CCc1cnc(N2CCC(C(=O)N3CCCCc4cc(Cl)ccc43)CC2)nc1] |
| 183 | 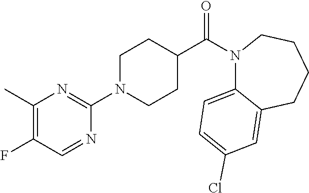 [SMILES: Cc1nc(N2CCC(C(=O)N3CCCCc4cc(Cl)ccc43)CC2)ncc1F] |
| 184 | 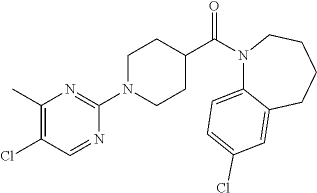 [SMILES: Cc1nc(N2CCC(C(=O)N3CCCCc4cc(Cl)ccc43)CC2)ncc1Cl] |
| 185 | 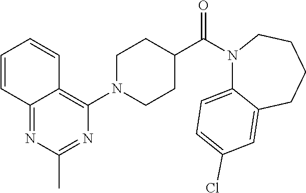 [SMILES: Cc1nc(N2CCC(C(=O)N3CCCCc4cc(Cl)ccc43)CC2)c2ccccc2n1] |
| 186 | 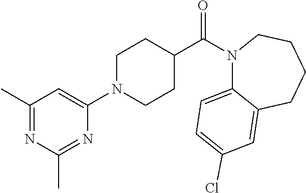 [SMILES: Cc1cc(N2CCC(C(=O)N3CCCCc4cc(Cl)ccc43)CC2)nc(C)n1] |
| 187 | 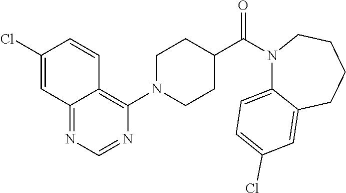 [SMILES: O=C(C1CCN(c2ncnc3cc(Cl)ccc23)CC1)N1CCCCc2cc(Cl)ccc21] |
| 188 | 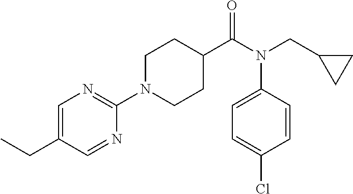 [SMILES: CCc1cnc(N2CCC(C(=O)N(CC3CC3)c3ccc(Cl)cc3)CC2)nc1] |
| 189 | 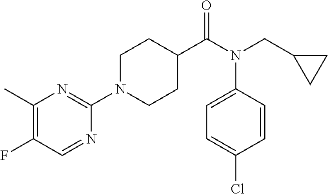 [SMILES: Cc1nc(N2CCC(C(=O)N(CC3CC3)c3ccc(Cl)cc3)CC2)ncc1F] |
| 190 | 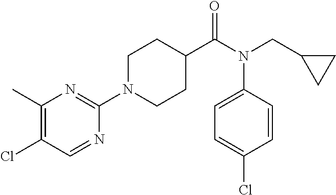 [SMILES: Cc1nc(N2CCC(C(=O)N(CC3CC3)c3ccc(Cl)cc3)CC2)ncc1Cl] |
| 191 | 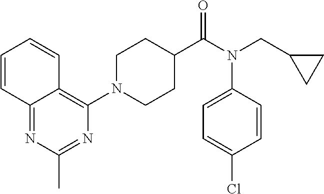 [SMILES: Cc1nc(N2CCC(C(=O)N(CC3CC3)c3ccc(Cl)cc3)CC2)c2ccccc2n1] |
| 192 | 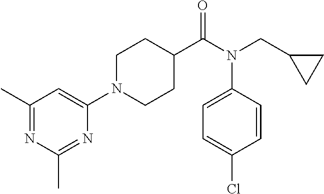 [SMILES: Cc1cc(N2CCC(C(=O)N(CC3CC3)c3ccc(Cl)cc3)CC2)nc(C)n1] |
| 193 | 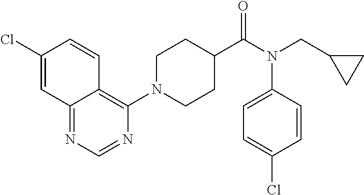 [SMILES: O=C(C1CCN(c2ncnc3cc(Cl)ccc23)CC1)N(CC1CC1)c1ccc(Cl)cc1] |
| 194 | 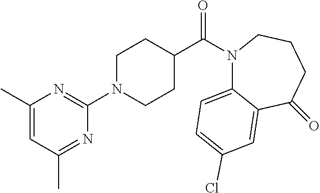 [SMILES: Cc1cc(C)nc(N2CCC(C(=O)N3CCCC(=O)c4cc(Cl)ccc43)CC2)n1] |
| 195 | 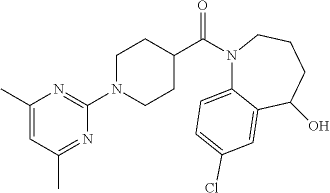 [SMILES: Cc1cc(C)nc(N2CCC(C(=O)N3CCCC(O)c4cc(Cl)ccc43)CC2)n1] |
| 196 | 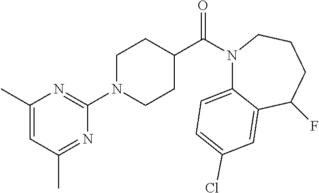 [SMILES: Cc1cc(C)nc(N2CCC(C(=O)N3CCCC(F)c4cc(Cl)ccc43)CC2)n1] |
| 197 | 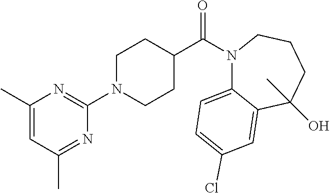 |
| 198 | 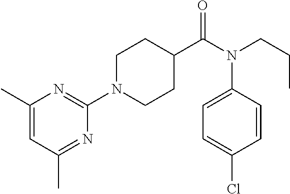 [SMILES: CCCN(C(=O)C1CCN(c2nc(C)cc(C)n2)CC1)c1ccc(Cl)cc1] |
| 199 | 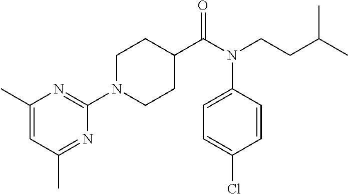 [SMILES: Cc1cc(C)nc(N2CCC(C(=O)N(CCC(C)C)c3ccc(Cl)cc3)CC2)n1] |
| 200 | 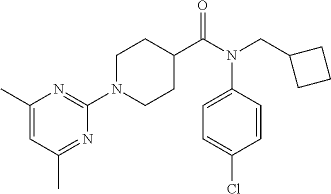 [SMILES: Cc1cc(C)nc(N2CCC(C(=O)N(CC3CCC3)c3ccc(Cl)cc3)CC2)n1] |
| 201 | 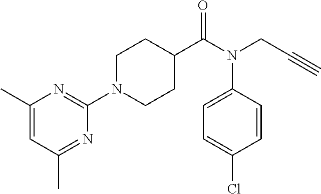 [SMILES: C#CCN(C(=O)C1CCN(c2nc(C)cc(C)n2)CC1)c1ccc(Cl)cc1] |
| 202 | 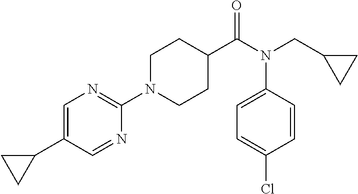 [SMILES: O=C(C1CCN(c2ncc(C3CC3)cn2)CC1)N(CC1CC1)c1ccc(Cl)cc1] |
| 203 | 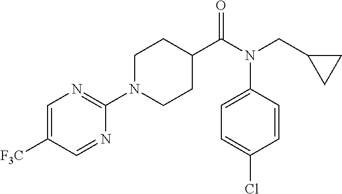 [SMILES: O=C(C1CCN(c2ncc(C(F)(F)F)cn2)CC1)N(CC1CC1)c1ccc(Cl)cc1] |
| 204 | 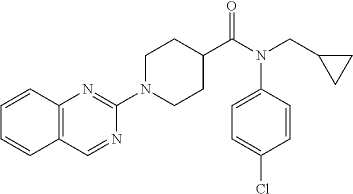 [SMILES: O=C(C1CCN(c2ncc3ccccc3n2)CC1)N(CC1CC1)c1ccc(Cl)cc1] |
| 205 | 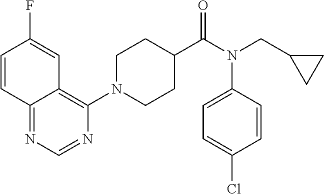 [SMILES: O=C(C1CCN(c2ncnc3ccc(F)cc23)CC1)N(CC1CC1)c1ccc(Cl)cc1] |
| 206 | 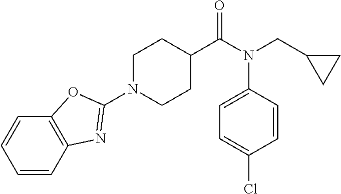 [SMILES: O=C(C1CCN(c2nc3ccccc3o2)CC1)N(CC1CC1)c1ccc(Cl)cc1] |
| 207 | 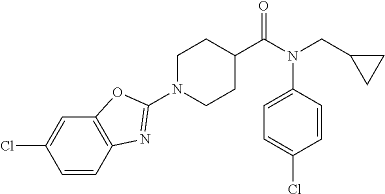 [SMILES: O=C(C1CCN(c2nc3ccc(Cl)cc3o2)CC1)N(CC1CC1)c1ccc(Cl)cc1] |
| 208 | 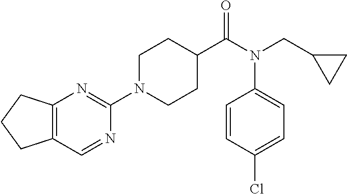 [SMILES: O=C(C1CCN(c2ncc3c(n2)CCC3)CC1)N(CC1CC1)c1ccc(Cl)cc1] |
| 209 | 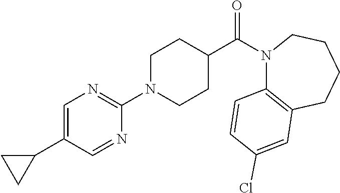 [SMILES: O=C(C1CCN(c2ncc(C3CC3)cn2)CC1)N1CCCCc2cc(Cl)ccc21] |
| 210 | 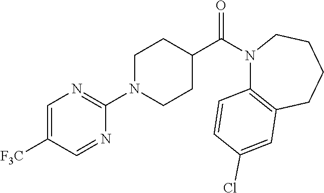 [SMILES: O=C(C1CCN(c2ncc(C(F)(F)F)cn2)CC1)N1CCCCc2cc(Cl)ccc21] |
| 211 | 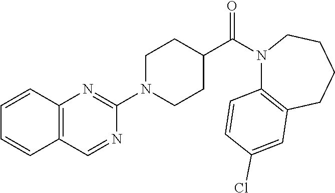 [SMILES: O=C(C1CCN(c2ncc3ccccc3n2)CC1)N1CCCCc2cc(Cl)ccc21] |
| 212 | 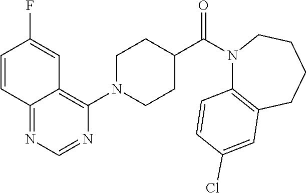 [SMILES: O=C(C1CCN(c2ncnc3ccc(F)cc23)CC1)N1CCCCc2cc(Cl)ccc21] |
| 213 | 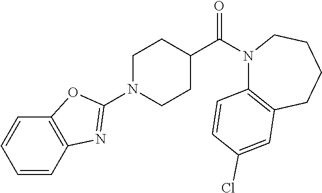 [SMILES: O=C(C1CCN(c2nc3ccccc3o2)CC1)N1CCCCc2cc(Cl)ccc21] |
| 214 | 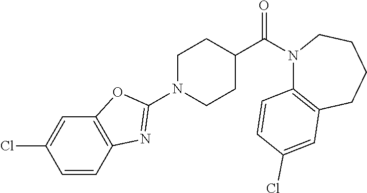 [SMILES: O=C(C1CCN(c2nc3ccc(Cl)cc3o2)CC1)N1CCCCc2cc(Cl)ccc21] |
| 215 | 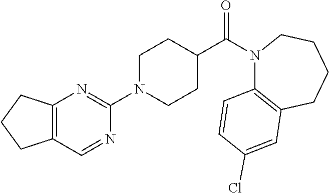 [SMILES: O=C(C1CCN(c2ncc3c(n2)CCC3)CC1)N1CCCCc2cc(Cl)ccc21] |
| 216 | 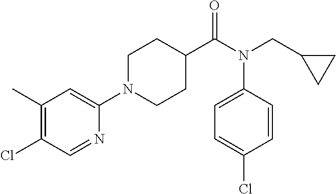 [SMILES: Cc1cc(N2CCC(C(=O)N(CC3CC3)c3ccc(Cl)cc3)CC2)ncc1Cl] |
| 217 | 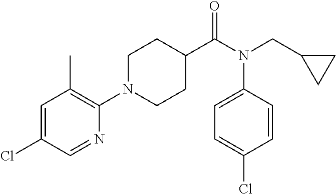 [SMILES: Cc1cc(Cl)cnc1N1CCC(C(=O)N(CC2CC2)c2ccc(Cl)cc2)CC1] |
| 218 | 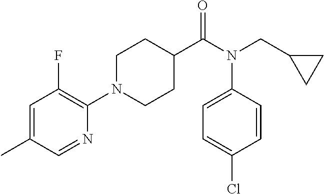 [SMILES: Cc1cnc(N2CCC(C(=O)N(CC3CC3)c3ccc(Cl)cc3)CC2)c(F)c1] |
| 219 | 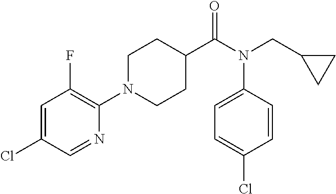 [SMILES: O=C(C1CCN(c2ncc(Cl)cc2F)CC1)N(CC1CC1)c1ccc(Cl)cc1] |
| 220 | 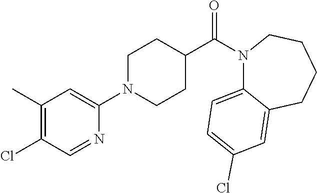 [SMILES: Cc1cc(N2CCC(C(=O)N3CCCCc4cc(Cl)ccc43)CC2)ncc1Cl] |
| 221 | 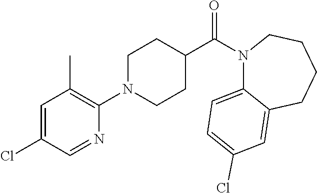 [SMILES: Cc1cc(Cl)cnc1N1CCC(C(=O)N2CCCCc3cc(Cl)ccc32)CC1] |
| 222 | 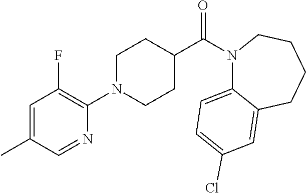 [SMILES: Cc1cnc(N2CCC(C(=O)N3CCCCc4cc(Cl)ccc43)CC2)c(F)c1] |
| 223 | 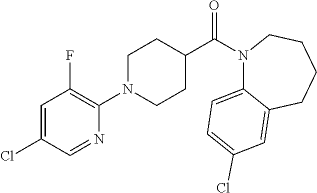 [SMILES: O=C(C1CCN(c2ncc(Cl)cc2F)CC1)N1CCCCc2cc(Cl)ccc21] |
| 224 | 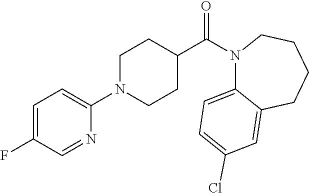 [SMILES: O=C(C1CCN(c2ccc(F)cn2)CC1)N1CCCCc2cc(Cl)ccc21] |
| 225 | 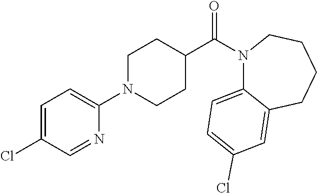 [SMILES: O=C(C1CCN(c2ccc(Cl)cn2)CC1)N1CCCCc2cc(Cl)ccc21] |
| 226 | 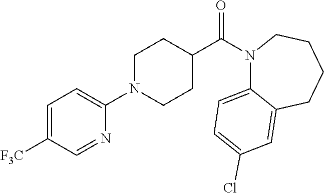 [SMILES: O=C(C1CCN(c2ccc(C(F)(F)F)cn2)CC1)N1CCCCc2cc(Cl)ccc21] |
| 227 | 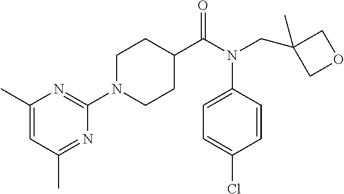 [SMILES: Cc1cc(C)nc(N2CCC(C(=O)N(CC3(C)COC3)c3ccc(Cl)cc3)CC2)n1] |
| 228 | 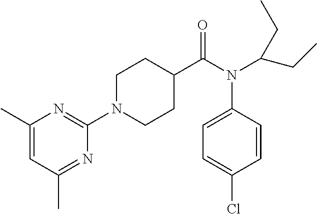 [SMILES: CCC(CC)N(C(=O)C1CCN(c2nc(C)cc(C)n2)CC1)c1ccc(Cl)cc1] |
| 229 | 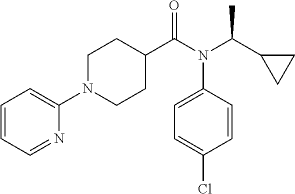 [SMILES: C[C@@H](C1CC1)N(C(=O)C1CCN(c2ccccn2)CC1)c1ccc(Cl)cc1] |
| 230 | 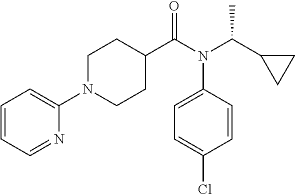 [SMILES: C[C@H](C1CC1)N(C(=O)C1CCN(c2ccccn2)CC1)c1ccc(Cl)cc1] |
| 231 | 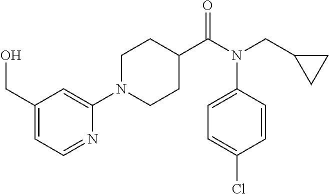 [SMILES: O=C(C1CCN(c2cc(CO)ccn2)CC1)N(CC1CC1)c1ccc(Cl)cc1] |
| 232 | 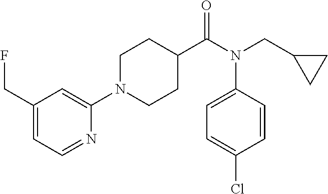 [SMILES: O=C(C1CCN(c2cc(CF)ccn2)CC1)N(CC1CC1)c1ccc(Cl)cc1] |
| 233 | 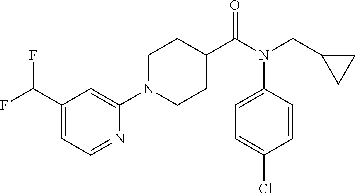 [SMILES: O=C(C1CCN(c2cc(C(F)F)ccn2)CC1)N(CC1CC1)c1ccc(Cl)cc1] |
| 234 | 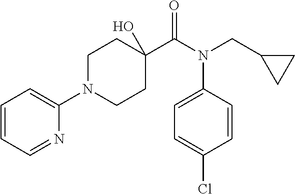 [SMILES: O=C(N(CC1CC1)c1ccc(Cl)cc1)C1(O)CCN(c2ccccn2)CC1] |
| 235 | 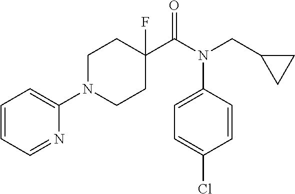 [SMILES: O=C(N(CC1CC1)c1ccc(Cl)cc1)C1(F)CCN(c2ccccn2)CC1] |
| 236 | 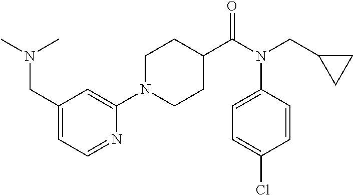 [SMILES: CN(C)Cc1ccnc(N2CCC(C(=O)N(CC3CC3)c3ccc(Cl)cc3)CC2)c1] |
| 237 | 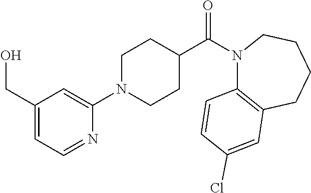 [SMILES: O=C(C1CCN(c2cc(CO)ccn2)CC1)N1CCCCc2cc(Cl)ccc21] |
| 238 | 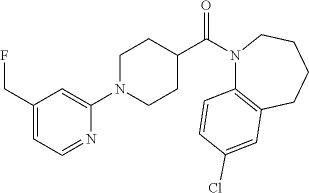 [SMILES: O=C(C1CCN(c2cc(CF)ccn2)CC1)N1CCCCc2cc(Cl)ccc21] |
| 239 | 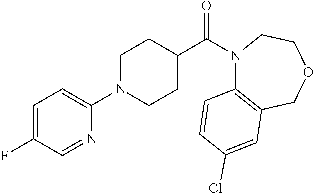 [SMILES: O=C(C1CCN(c2ccc(F)cn2)CC1)N1CCOCc2cc(Cl)ccc21] |
| 240 | 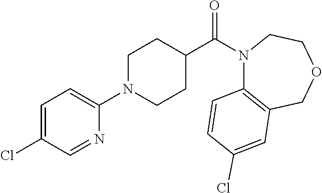 [SMILES: O=C(C1CCN(c2ccc(Cl)cn2)CC1)N1CCOCc2cc(Cl)ccc21] |
| 241 | 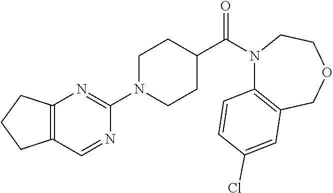 [SMILES: O=C(C1CCN(c2ncc3c(n2)CCC3)CC1)N1CCOCc2cc(Cl)ccc21] |
| 242 | 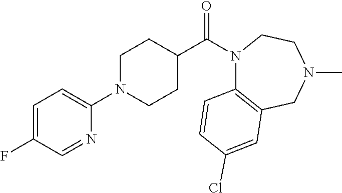 [SMILES: CN1CCN(C(=O)C2CCN(c3ccc(F)cn3)CC2)c2ccc(Cl)cc2C1] |
| 243 | 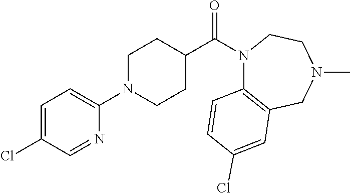 [SMILES: CN1CCN(C(=O)C2CCN(c3ccc(Cl)cn3)CC2)c2ccc(Cl)cc2C1] |
| 244 | 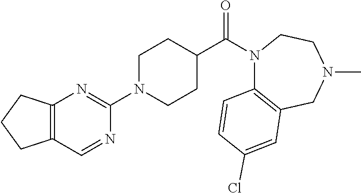 [SMILES: CN1CCN(C(=O)C2CCN(c3ncc4c(n3)CCC4)CC2)c2ccc(Cl)cc2C1] |
| 245 | 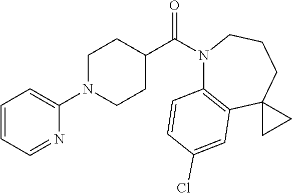 [SMILES: O=C(C1CCN(c2ccccn2)CC1)N1CCCC2(CC2)c2cc(Cl)ccc21] |
| 246 | 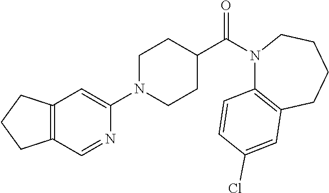 [SMILES: O=C(C1CCN(c2cc3c(cn2)CCC3)CC1)N1CCCCc2cc(Cl)ccc21] |
| 247 | 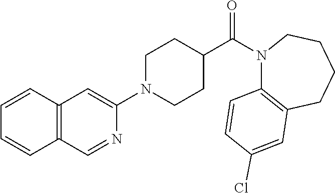 [SMILES: O=C(C1CCN(c2cc3ccccc3cn2)CC1)N1CCCCc2cc(Cl)ccc21] |
| 248 | 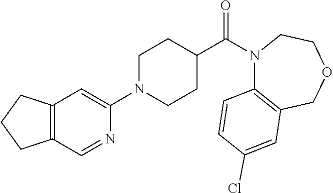 [SMILES: O=C(C1CCN(c2cc3c(cn2)CCC3)CC1)N1CCOCc2cc(Cl)ccc21] |
| 249 | 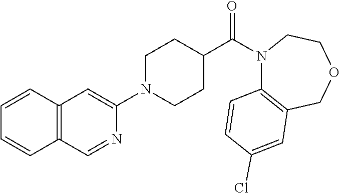 [SMILES: O=C(C1CCN(c2cc3ccccc3cn2)CC1)N1CCOCc2cc(Cl)ccc21] |
| 250 | 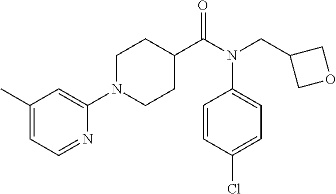 [SMILES: Cc1ccnc(N2CCC(C(=O)N(CC3COC3)c3ccc(Cl)cc3)CC2)c1] |

In various embodiments, the compound of the invention can comprise a compound of formula (V):

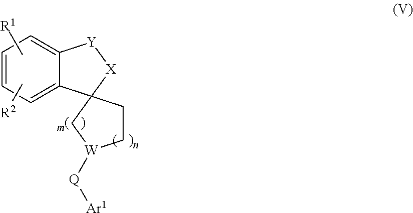

wherein:

X and Y are independently NR³, CR⁴R⁵, O, S, SO, SO₂, CO, or CO₂; provided that when X and Y are both CR⁴R⁵, one each of R⁵ on respective X and Y can optionally be absent and a double bond can optionally be present between X and Y; and provided that when one of X and Y is NR³ and the other of X and Y is CR⁴R⁵, R³ and R⁵ on respective X and Y can optionally be absent and a double bond can optionally be present between X and Y; and when X and Y are both NR³, both R³ can optionally be absent and a double bond can optionally be present between X and Y;

m and n are both independently 0, 1, 2, or 3;

W is N or CR⁴;

Ar¹ and Ar² are each independently cycloalkyl, aryl, heterocyclyl, or heteroaryl, wherein any cycloalkyl, aryl, heterocyclyl, or heteroaryl is optionally mono- or independently multi-substituted with J, (C₁-C₆)alkyl, (C₂-C₆)alkenyl, (C₂-C₆)alkynyl, (C₁-C₆)acyl, (C₁-C₆)haloalkyl, (C₁-C₆)alkoxy, (C₁-C₆)haloalkoxy, cycloalkyl(C₀-C₆)alkyl, heterocyclyl(C₀-C₆) alkyl, aryl(C₀-C₆)alkyl, heteroaryl(C₀-C₆)alkyl, or -Q²-Ar²; wherein any alkyl, alkenyl, alkynyl, acyl, haloalkyl, alkoxy, haloalkoxy, cycloalkyl, aryl, heterocyclyl, or heteroaryl can be further optionally substituted with 1-5 J and/or with -Q²-Ar²;

Q and Q² each independently is a bond, (CH₂)₀₋₂O, (CH₂)₀₋₂C(O)(CH₂)₀₋₂, (CH₂)₀₋₂C(O)CR═CR(CH₂)₀₋₂, (CH₂)₀₋₂CR₂(CH₂)₀₋₂, (CH₂)₀₋₂C(OR)(R)(CH₂)₀₋₂, O(CR₂)₁₋₄O, (CH₂)₀₋₂N(R)(CH₂)₀₋₂, (CH₂)₀₋₂S(CH₂)₀₋₂, (CH₂)₀₋₂SO(CH₂)₀₋₂, (CH₂)₀₋₂SO₂(CH₂)₀₋₂, (CH₂)₀₋₂SO₂N(R)(CH₂)₀₋₂, (CH₂)₀₋₂SO₃(CH₂)₀₋₂, (CH₂)₀₋₂C(O)C(O) (CH₂)₀₋₂, (CH₂)₀₋₂C(O)CH₂C(O)(CH₂)₀₋₂, (CH₂)₀₋₂C(S)(CH₂)₀₋₂, (CH₂)₀₋₂C(O)O(CH₂)₀₋₂, (CH₂)₀₋₂OC(O)(CH₂)₀₋₂, (CH₂)₀₋₂OC(O)O(CH₂)₀₋₂, (CH₂)₀₋₂C(O)N(R)(CH₂)₀₋₂, (CH₂)₀₋₂OC(O)N(R)(CH₂)₀₋₂, (CH₂)₀₋₂C(S)N(R)(CH₂)₀₋₂, (CH₂)₀₋₂NHC(O) (CH₂)₀₋₂, (CH₂)₀₋₂N(R)N(R)C(O)(CH₂)₀₋₂, (CH₂)₀₋₂N(R)N(R)C(O)O(CH₂)₀₋₂, (CH₂)₀₋₂N(R)N(R)CON(R)(CH₂)₀₋₂, (CH₂)₀₋₂N(R)SO₂(CH₂)₀₋₂, (CH₂)₀₋₂N(R)SO₂N(R)(CH₂)₀₋₂, (CH₂)₀₋₂N(R)C(O)O(CH₂)₀₋₂, (CH₂)₀₋₂N(R)C(O)(CH₂)₀₋₂, (CH₂)₀₋₂N(R)C(S)(CH₂)₀₋₂, (CH₂)₀₋₂N(R)C(O)N(R)(CH₂)₀₋₂, (CH₂)₀₋₂N(R)C(S)N(R)(CH₂)₀₋₂, (CH₂)₀₋₂N(COR)CO(CH₂)₀₋₂, (CH₂)₀₋₂N(OR)(CH₂)₀₋₂, (CH₂)₀₋₂C(═NH)N(R)(CH₂)₀₋₂, (CH₂)₀₋₂C(O)N(OR)(CH₂)₀₋₂, or (CH₂)₀₋₂C(═NOR)(CH₂)₀₋₂;

R², R³, R⁴, and R⁵ are each independently H, J, (C₁-C₆)alkyl, (C₂-C₆)alkenyl, (C₂-C₆)alkynyl, (C₁-C₆)acyl, (C₁-C₆)haloalkyl, (C₁-C₆)haloalkoxy, cycloalkyl(C₁-C₆)alkyl, heterocyclyl(C₁-C₆)alkyl, aryl(C₁-C₆)alkyl, or heteroaryl(C₁-C₆)alkyl, wherein any alkyl, alkenyl, alkynyl, acyl, haloalkyl, haloalkoxy, cycloalkyl, heterocyclyl, aryl, or heteroaryl can be mono- or independently multi-substituted with J; or J;

or R¹ and R² taken together can be methylenedioxy or ethylenedioxy; J is independently at each occurrence F, Cl, Br, I, OR, CN, CF₃, OCF₃, R, O, S, C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, OC(O)OR, C(O)N(R)₂, OC(O)N(R)₂, C(S)N(R)₂, (CH₂)₀₋₂NHC(O)R, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R;

wherein R is independently at each occurrence hydrogen or an alkyl, heteroalkyl, cycloalkyl, cycloalkylalkyl, aryl, aralkyl, heterocyclyl, heterocyclylalkyl, heteroaryl, or heteroarylalkyl, wherein any alkyl, heteroalkyl, cycloalkyl, cycloalkylalkyl, aryl, aralkyl, heterocyclyl, heterocyclylalkyl, heteroaryl, or heteroarylalkyl is substituted with 0-3 J; or wherein two R groups together with a nitrogen atom or with two adjacent nitrogen atoms to which they are bonded can together form a 3-8 membered heterocyclyl substituted with 0-3 J; optionally further comprising 1-3 additional heteroatoms selected from the group consisting of O, NR, S, S(O) and S(O)₂;

wherein any cycloalkyl, aryl, heterocyclyl, or heteroaryl of formula (V) throughout can be fused, bridged, or in a Spiro configuration with one or more additional optionally substituted cycloalkyl, aryl, heterocyclyl, and heteroaryl, monocyclic, bicyclic or polycyclic, saturated, partially unsaturated, or aromatic rings; and wherein any heterocyclyl or heteroaryl comprising nitrogen can be an N-oxide or N-metho salt thereof;

or a salt thereof.

In various embodiments, the compound of the invention can comprise a compound of formula (VA)

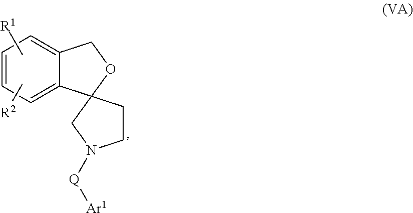

### or (VB)

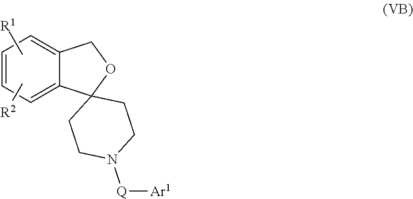

wherein R¹, R², Q, and Ar¹ are as defined in above for a compound of formula (V).

Alternatively, in various embodiments, the compound of the invention is of formula (VC)

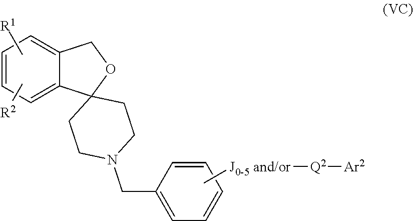

### or (VD)

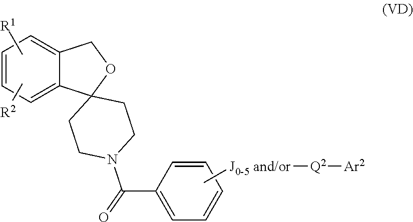

### or (VE)

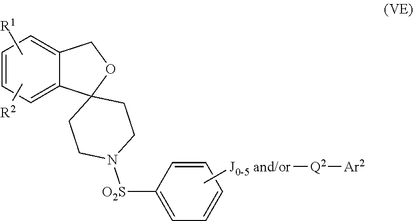

### or (VF)

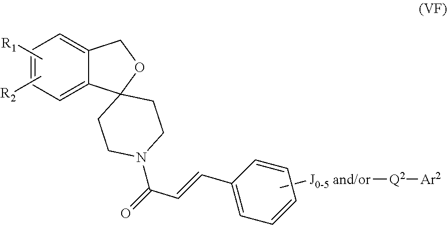

wherein R¹, R², J, Q² and Ar² are as defined above for a compound of formula (V).

In various embodiments, Q can be a bond, CH₂, C(O), or SO₂ in any of formulas (V), or (VA)-(VF).

In various embodiments, Ar¹ can be unsubstituted or substituted phenyl, or can comprise a pyrazole, benzimidazole, benzthiophene, oxazepine or diazepine in any of formulas (V), or (VA)-(VF).

In various embodiments, the compound of the invention can comprise any of the exemplary compounds of formula (V) as shown below in the list of specific compounds of the invention.

In various embodiments, a compound of the invention can be any of the exemplary compounds shown below.

### Exemplary Compound of Formula (V)

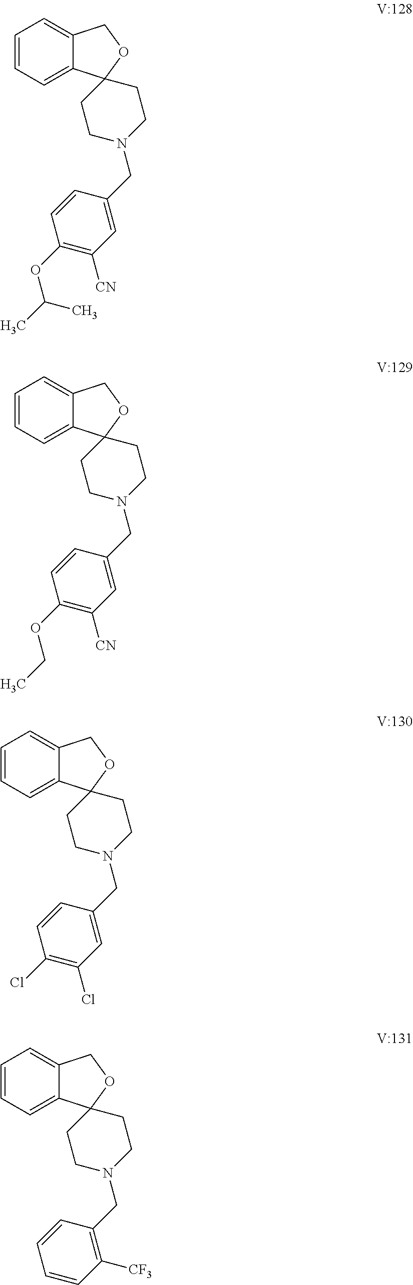 [SMILES: CC(C)Oc1ccc(CN2CCC3(CC2)OCc2ccccc23)cc1C#N ; CCOc1ccc(CN2CCC3(CC2)OCc2ccccc23)cc1C#N ; Clc1ccc(CN2CCC3(CC2)OCc2ccccc23)cc1Cl ; FC(F)(F)c1ccccc1CN1CCC2(CC1)OCc1ccccc12]

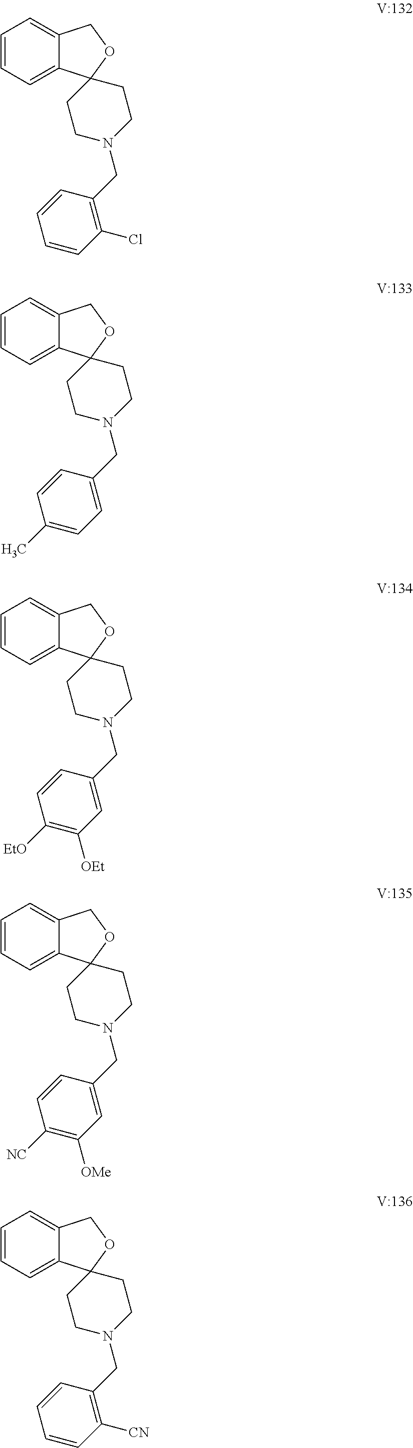

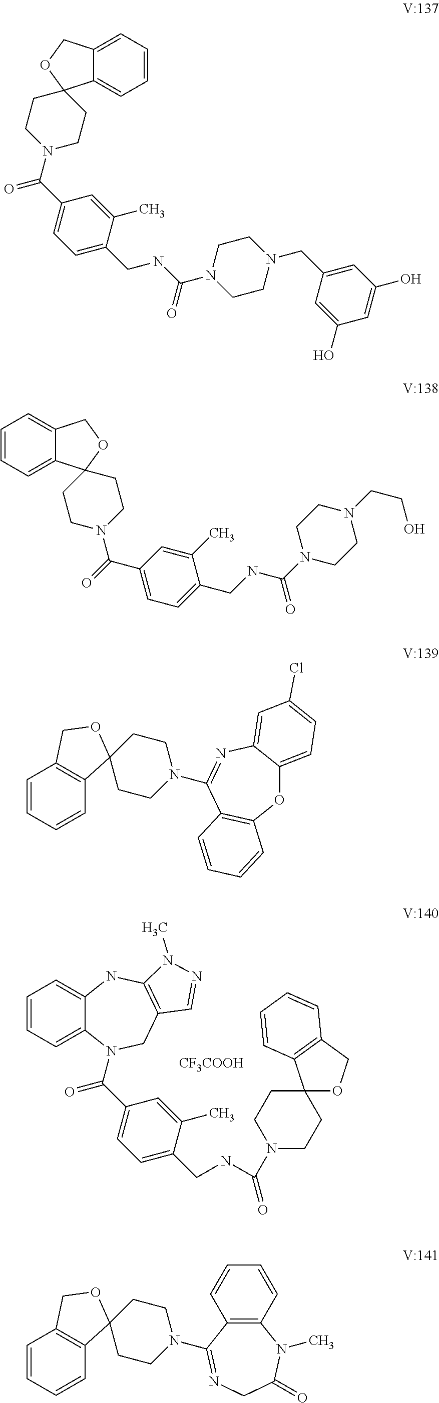

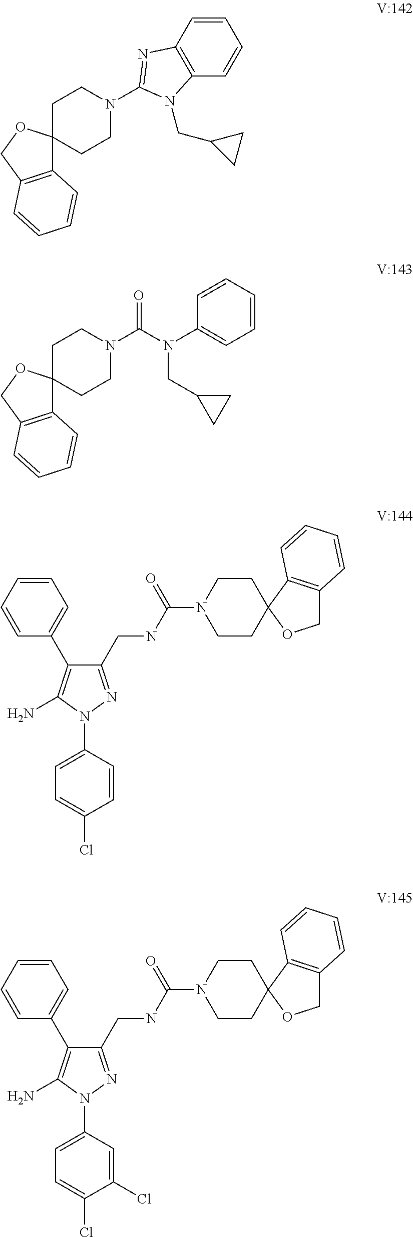 [SMILES: c1ccc2c(c1)COC21CCN(c2nc3ccccc3n2CC2CC2)CC1 ; O=C(N1CCC2(CC1)OCc1ccccc12)N(CC1CC1)c1ccccc1 ; Nc1c(-c2ccccc2)c(CNC(=O)N2CCC3(CC2)OCc2ccccc23)nn1-c1ccc(Cl)cc1 ; Nc1c(-c2ccccc2)c(CNC(=O)N2CCC3(CC2)OCc2ccccc23)nn1-c1ccc(Cl)c(Cl)c1]

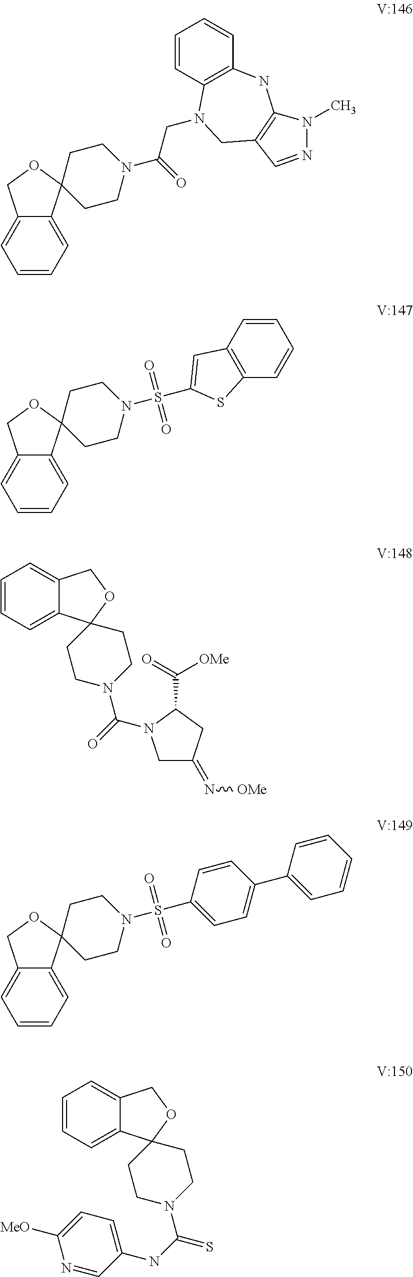 [SMILES: Cn1ncc2c1Nc1ccccc1N(CC(=O)N1CCC3(CC1)OCc1ccccc13)C2 ; O=S(=O)(c1cc2ccccc2s1)N1CCC2(CC1)OCc1ccccc12 ; CON=C1C[C@@H](C(=O)OC)N(C(=O)N2CCC3(CC2)OCc2ccccc23)C1 ; O=S(=O)(c1ccc(-c2ccccc2)cc1)N1CCC2(CC1)OCc1ccccc12 ; COc1ccc(NC(=S)N2CCC3(CC2)OCc2ccccc23)cn1]

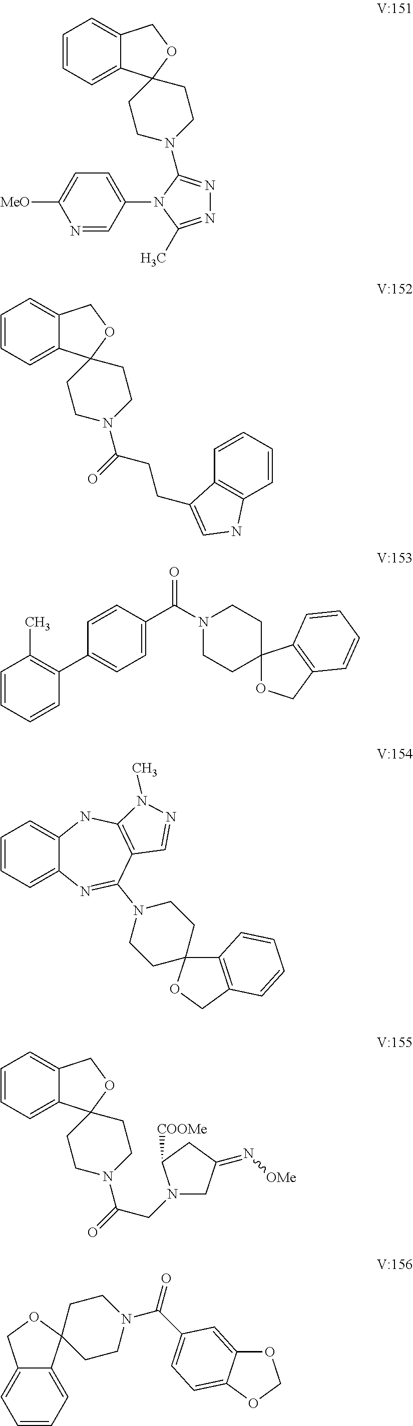

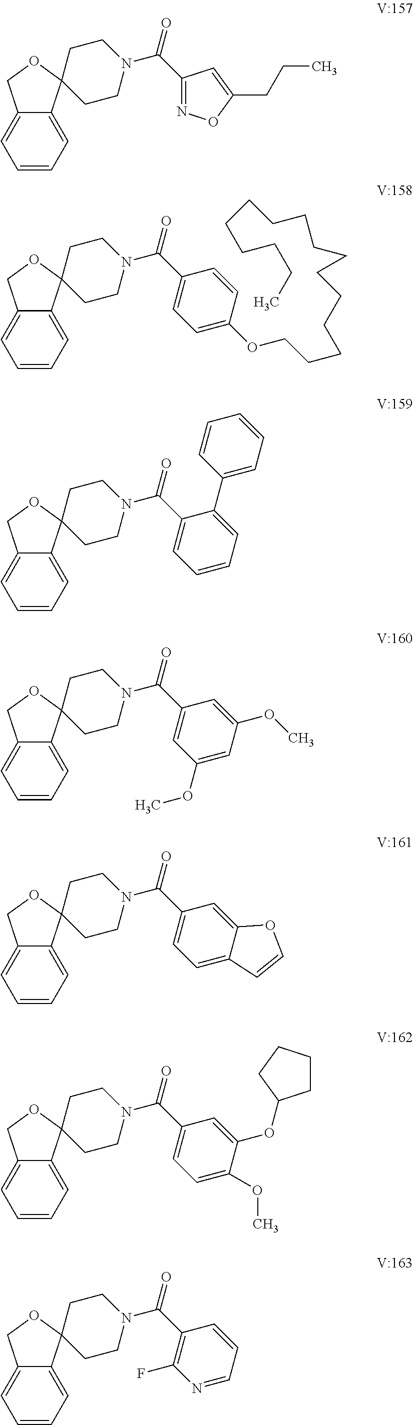

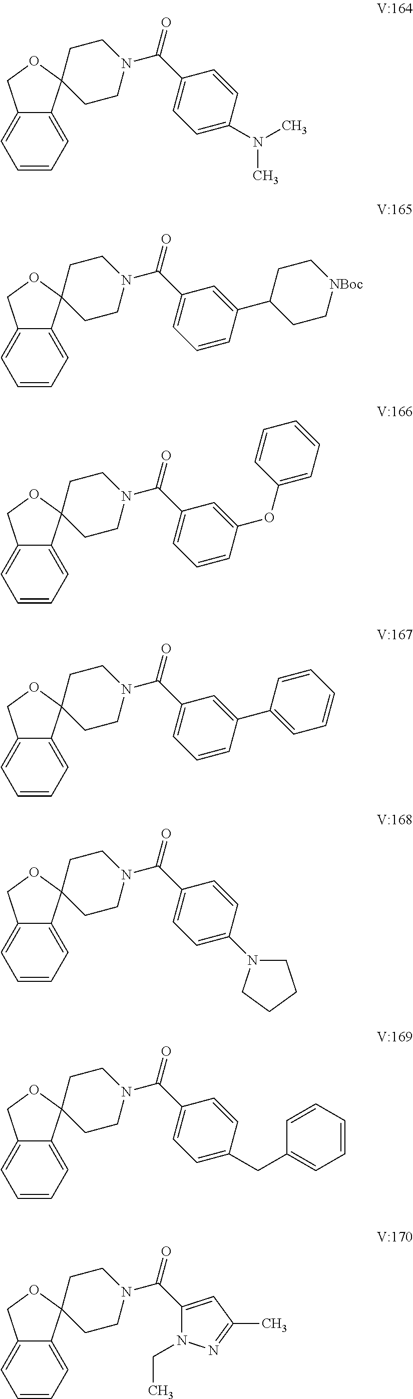

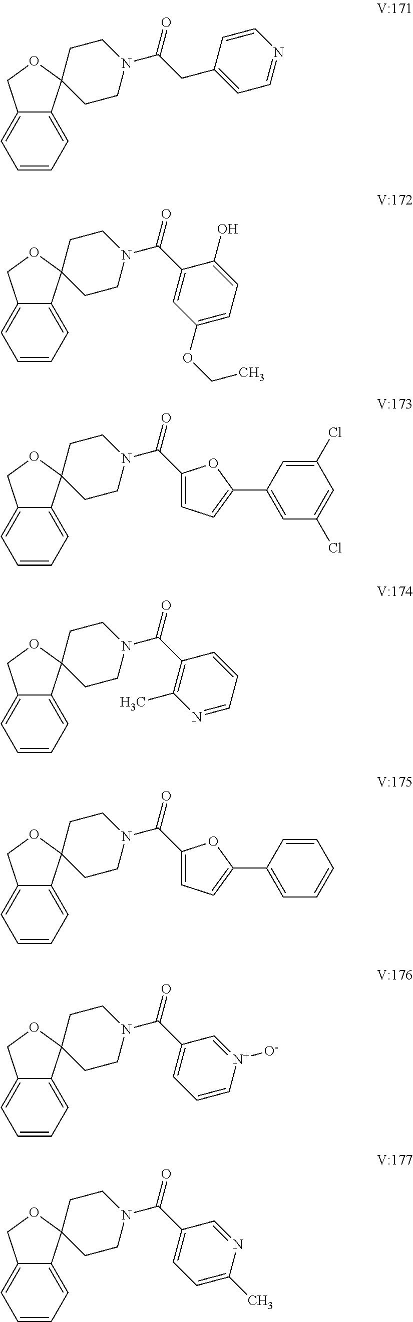

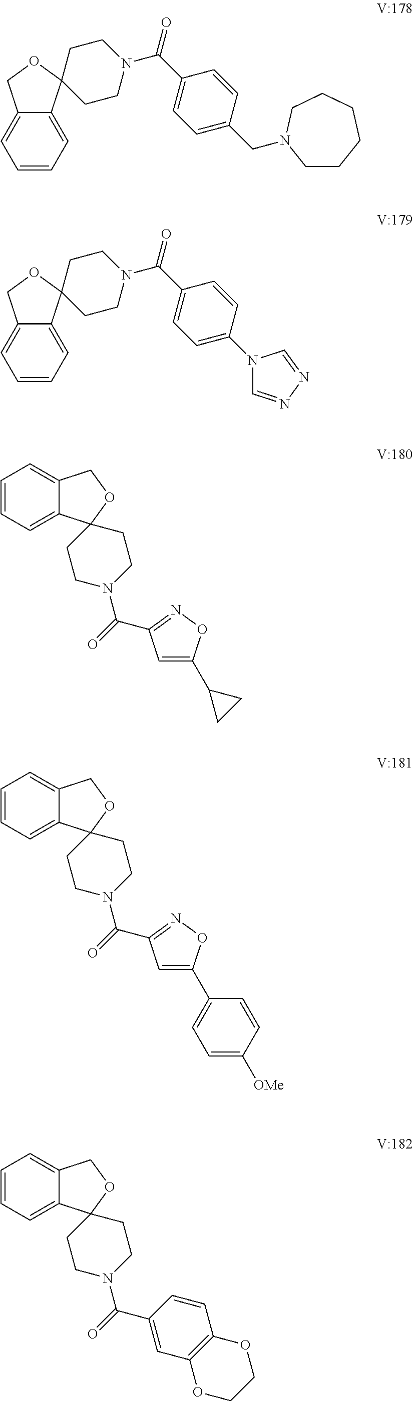 [SMILES: O=C(c1ccc(CN2CCCCCC2)cc1)N1CCC2(CC1)OCc1ccccc12 ; O=C(c1ccc(-n2cnnc2)cc1)N1CCC2(CC1)OCc1ccccc12 ; O=C(c1cc(C2CC2)on1)N1CCC2(CC1)OCc1ccccc12 ; COc1ccc(-c2cc(C(=O)N3CCC4(CC3)OCc3ccccc34)no2)cc1 ; O=C(c1ccc2c(c1)OCCO2)N1CCC2(CC1)OCc1ccccc12]

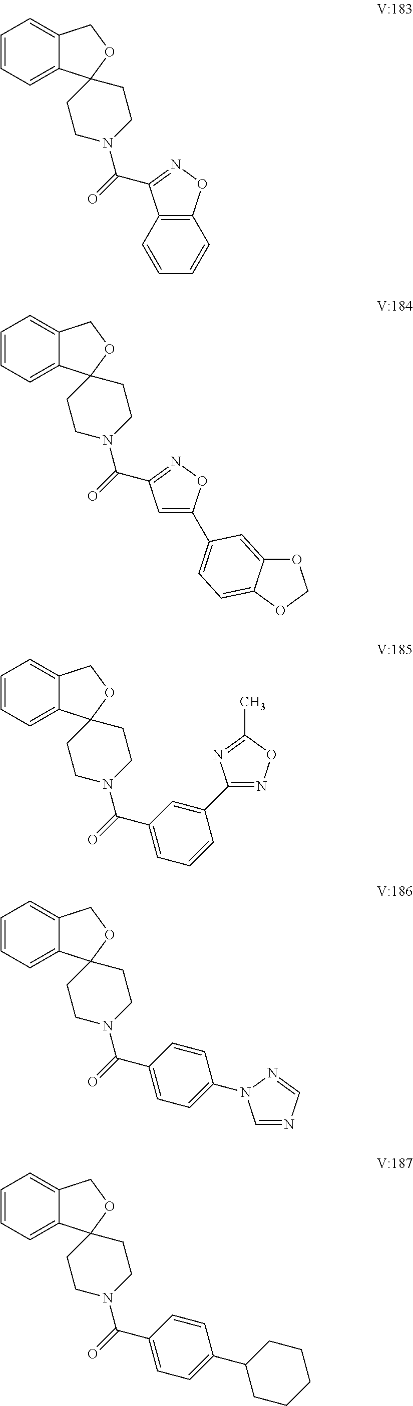 [SMILES: O=C(c1noc2ccccc12)N1CCC2(CC1)OCc1ccccc12 ; O=C(c1cc(-c2ccc3c(c2)OCO3)on1)N1CCC2(CC1)OCc1ccccc12 ; Cc1nc(-c2cccc(C(=O)N3CCC4(CC3)OCc3ccccc34)c2)no1 ; O=C(c1ccc(-n2cncn2)cc1)N1CCC2(CC1)OCc1ccccc12 ; O=C(c1ccc(C2CCCCC2)cc1)N1CCC2(CC1)OCc1ccccc12]

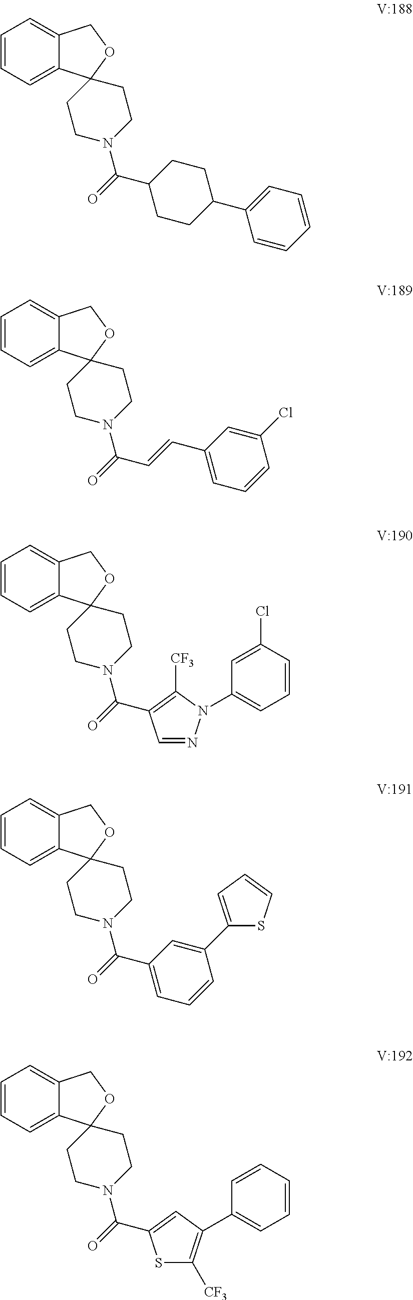 [SMILES: O=C(C1CCC(c2ccccc2)CC1)N1CCC2(CC1)OCc1ccccc12 ; O=C(/C=C/c1cccc(Cl)c1)N1CCC2(CC1)OCc1ccccc12 ; O=C(c1cnn(-c2cccc(Cl)c2)c1C(F)(F)F)N1CCC2(CC1)OCc1ccccc12 ; O=C(c1cccc(-c2cccs2)c1)N1CCC2(CC1)OCc1ccccc12 ; O=C(c1cc(-c2ccccc2)c(C(F)(F)F)s1)N1CCC2(CC1)OCc1ccccc12]

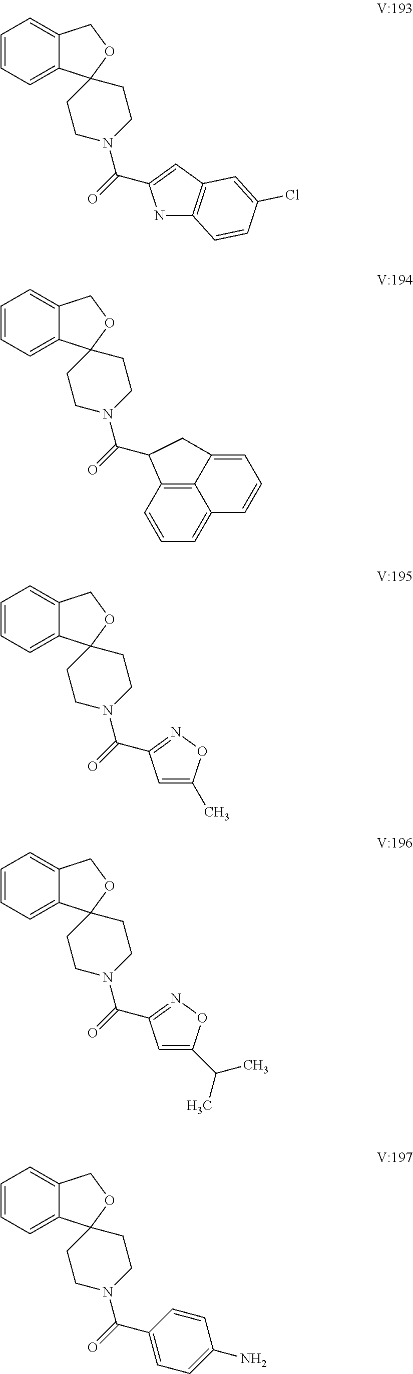 [SMILES: O=C(c1cc2cc(Cl)ccc2[nH]1)N1CCC2(CC1)OCc1ccccc12 ; O=C(C1Cc2cccc3cccc1c23)N1CCC2(CC1)OCc1ccccc12 ; Cc1cc(C(=O)N2CCC3(CC2)OCc2ccccc23)no1 ; CC(C)c1cc(C(=O)N2CCC3(CC2)OCc2ccccc23)no1 ; Nc1ccc(C(=O)N2CCC3(CC2)OCc2ccccc23)cc1]

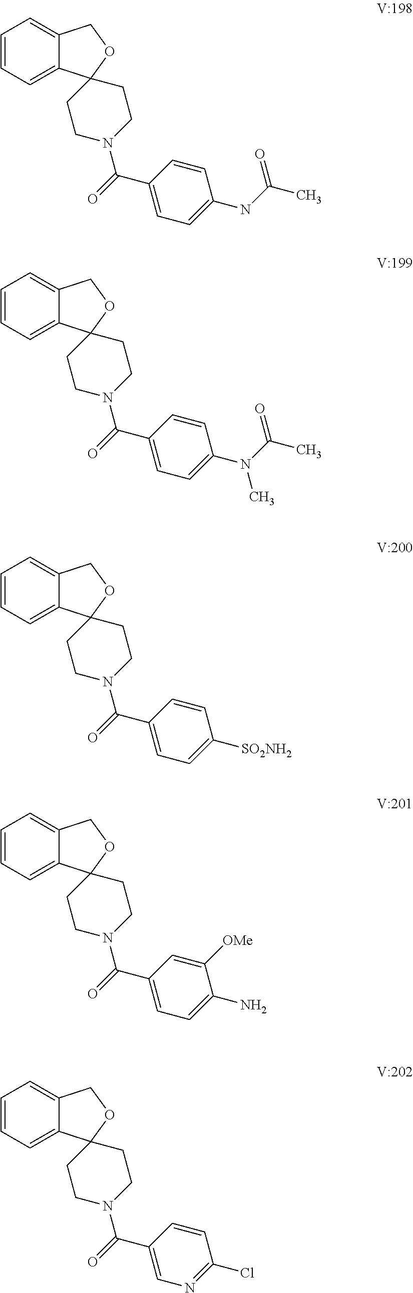 [SMILES: CC(=O)Nc1ccc(C(=O)N2CCC3(CC2)OCc2ccccc23)cc1 ; CC(=O)N(C)c1ccc(C(=O)N2CCC3(CC2)OCc2ccccc23)cc1 ; NS(=O)(=O)c1ccc(C(=O)N2CCC3(CC2)OCc2ccccc23)cc1 ; COc1cc(C(=O)N2CCC3(CC2)OCc2ccccc23)ccc1N ; O=C(c1ccc(Cl)nc1)N1CCC2(CC1)OCc1ccccc12]

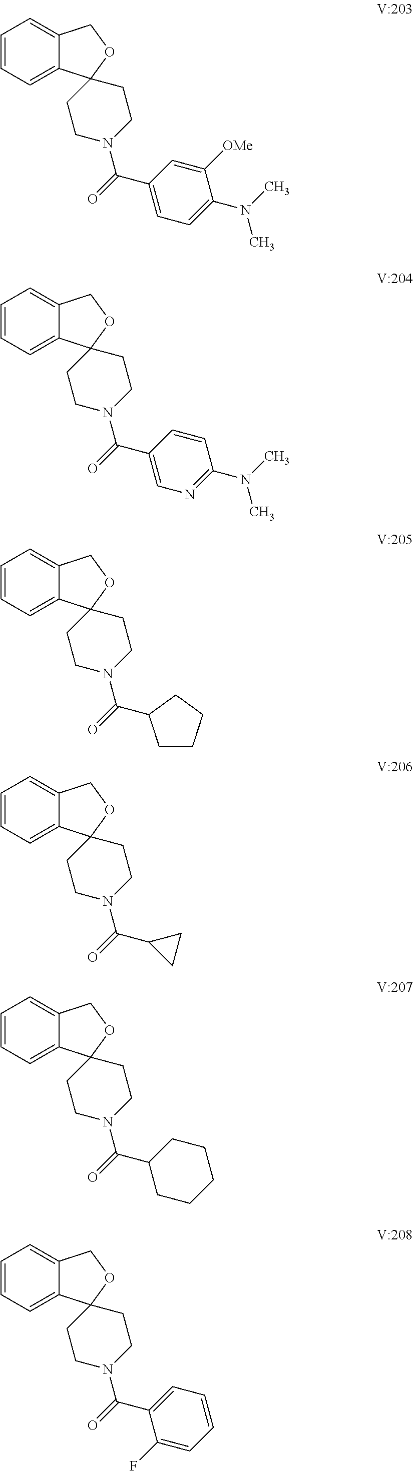

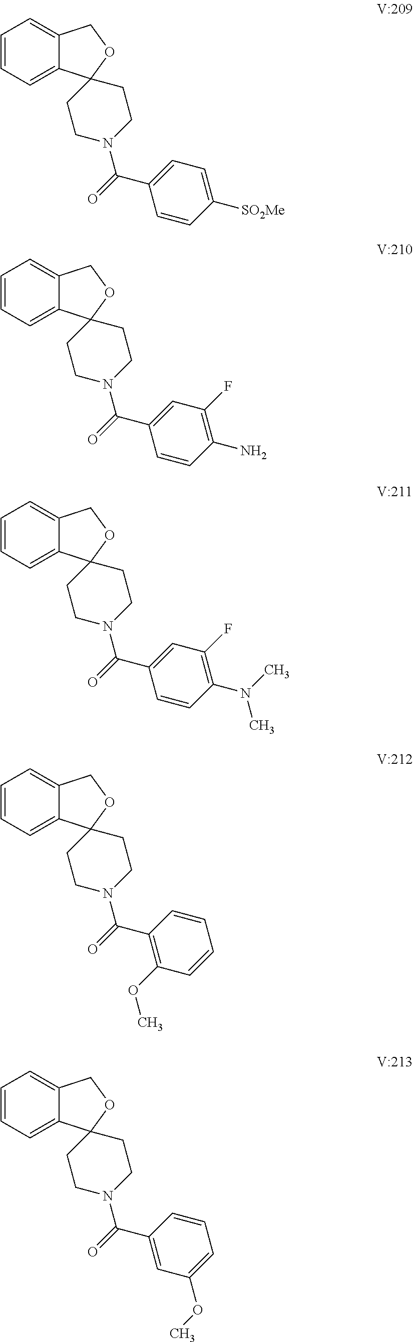 [SMILES: CS(=O)(=O)c1ccc(C(=O)N2CCC3(CC2)OCc2ccccc23)cc1 ; Nc1ccc(C(=O)N2CCC3(CC2)OCc2ccccc23)cc1F ; CN(C)c1ccc(C(=O)N2CCC3(CC2)OCc2ccccc23)cc1F ; COc1ccccc1C(=O)N1CCC2(CC1)OCc1ccccc12 ; COc1cccc(C(=O)N2CCC3(CC2)OCc2ccccc23)c1]

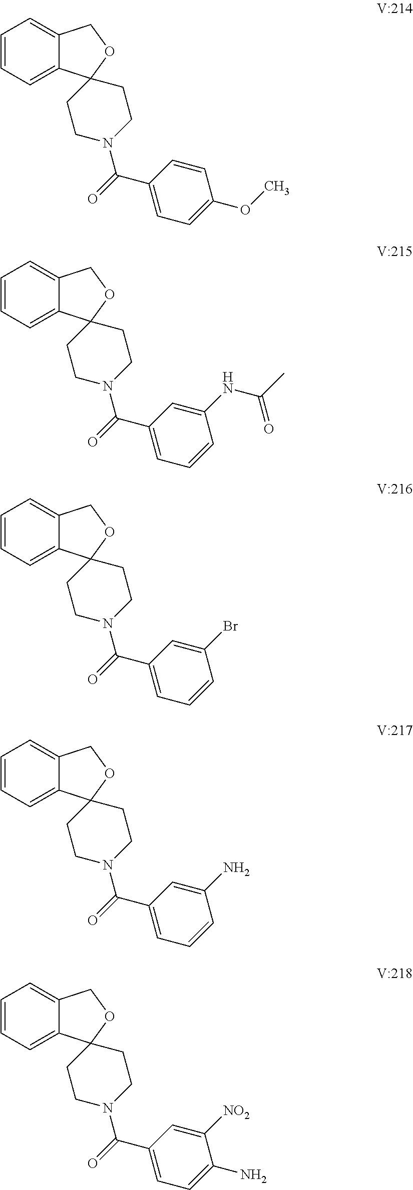 [SMILES: COc1ccc(C(=O)N2CCC3(CC2)OCc2ccccc23)cc1 ; CC(=O)Nc1cccc(C(=O)N2CCC3(CC2)OCc2ccccc23)c1 ; O=C(c1cccc(Br)c1)N1CCC2(CC1)OCc1ccccc12 ; Nc1cccc(C(=O)N2CCC3(CC2)OCc2ccccc23)c1 ; Nc1ccc(C(=O)N2CCC3(CC2)OCc2ccccc23)cc1[N+](=O)[O-]]

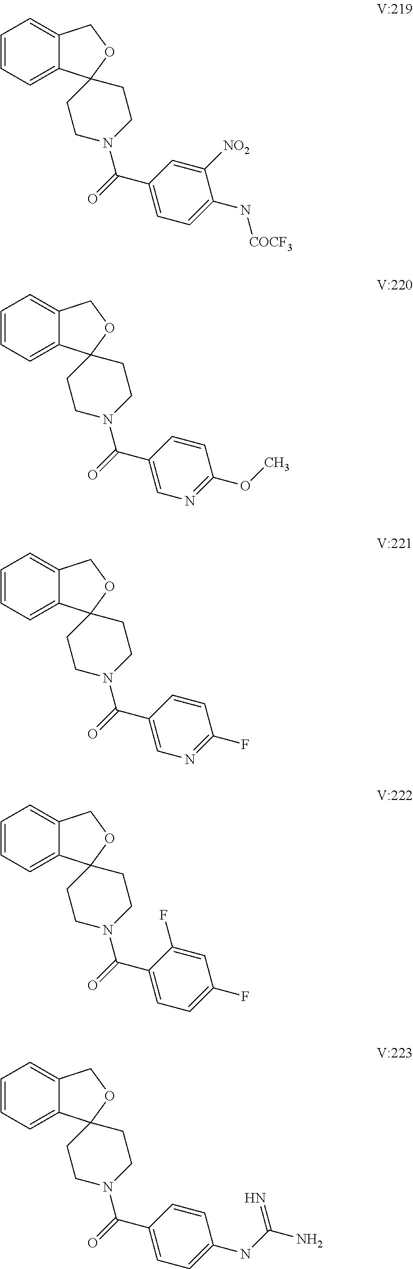

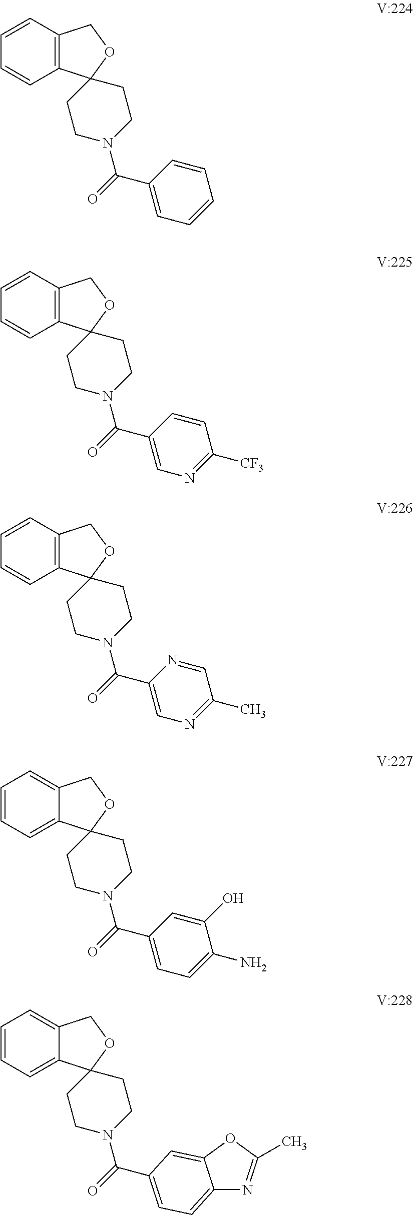 [SMILES: O=C(c1ccccc1)N1CCC2(CC1)OCc1ccccc12 ; O=C(c1ccc(C(F)(F)F)nc1)N1CCC2(CC1)OCc1ccccc12 ; Cc1cnc(C(=O)N2CCC3(CC2)OCc2ccccc23)cn1 ; Nc1ccc(C(=O)N2CCC3(CC2)OCc2ccccc23)cc1O ; Cc1nc2ccc(C(=O)N3CCC4(CC3)OCc3ccccc34)cc2o1]

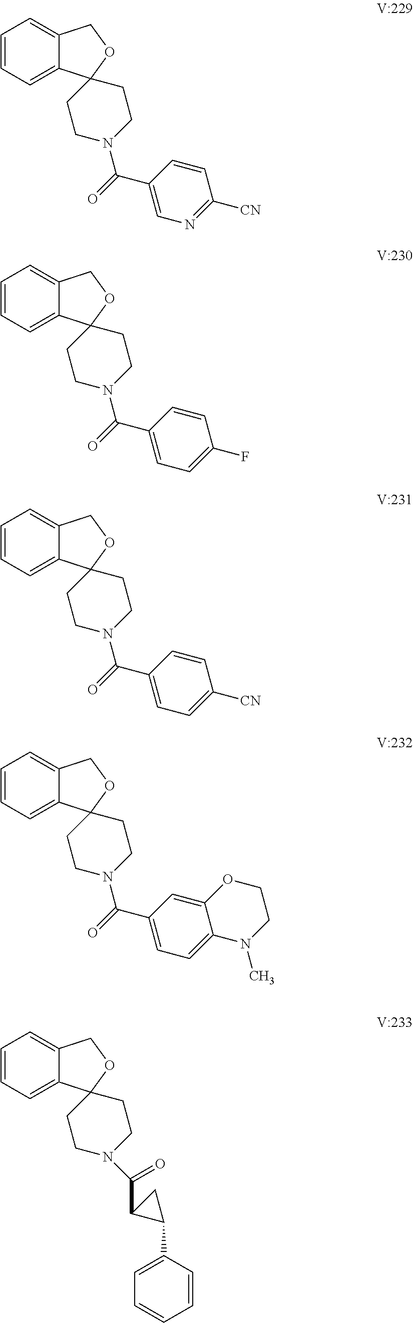

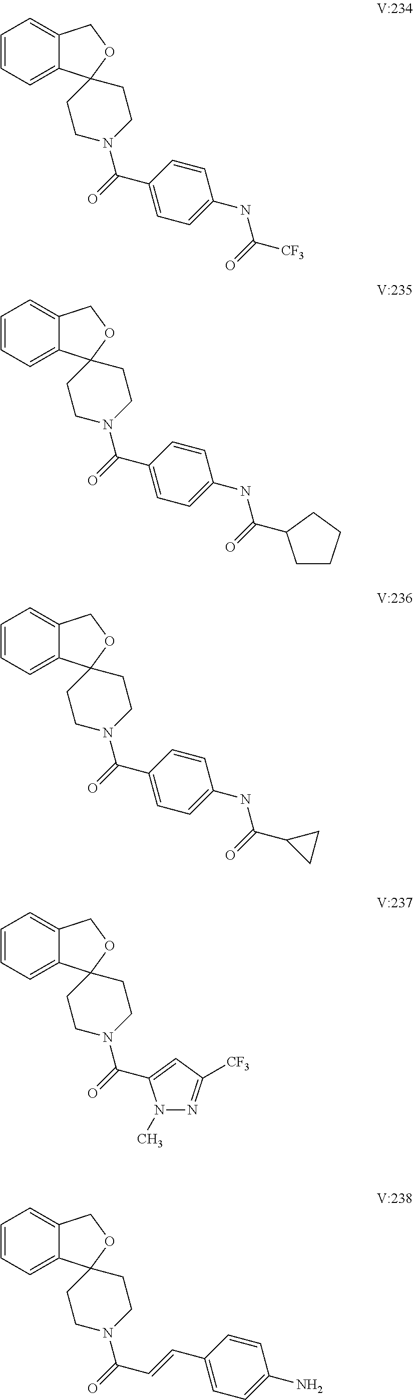 [SMILES: O=C(c1ccc(NC(=O)C(F)(F)F)cc1)N1CCC2(CC1)OCc1ccccc12 ; O=C(Nc1ccc(C(=O)N2CCC3(CC2)OCc2ccccc23)cc1)C1CCCC1 ; O=C(Nc1ccc(C(=O)N2CCC3(CC2)OCc2ccccc23)cc1)C1CC1 ; Cn1nc(C(F)(F)F)cc1C(=O)N1CCC2(CC1)OCc1ccccc12 ; Nc1ccc(/C=C/C(=O)N2CCC3(CC2)OCc2ccccc23)cc1]

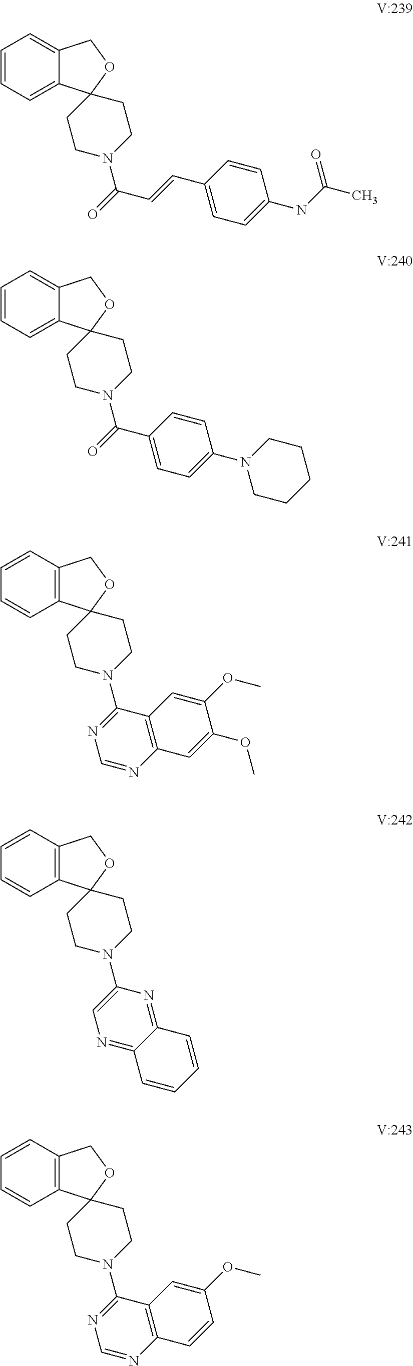 [SMILES: CC(=O)Nc1ccc(/C=C/C(=O)N2CCC3(CC2)OCc2ccccc23)cc1 ; O=C(c1ccc(N2CCCCC2)cc1)N1CCC2(CC1)OCc1ccccc12 ; COc1cc2ncnc(N3CCC4(CC3)OCc3ccccc34)c2cc1OC ; c1ccc2c(c1)COC21CCN(c2cnc3ccccc3n2)CC1 ; COc1ccc2ncnc(N3CCC4(CC3)OCc3ccccc34)c2c1]

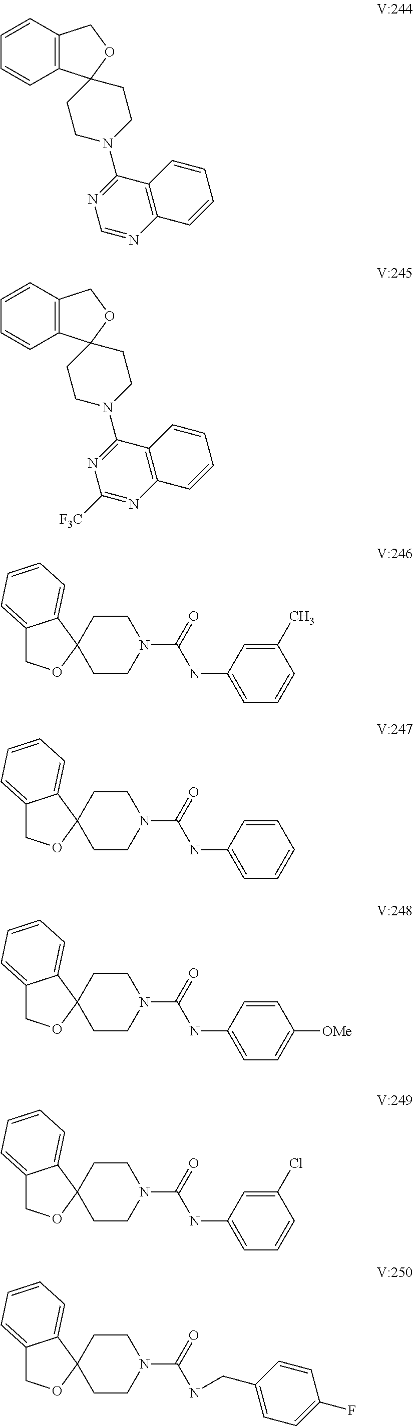

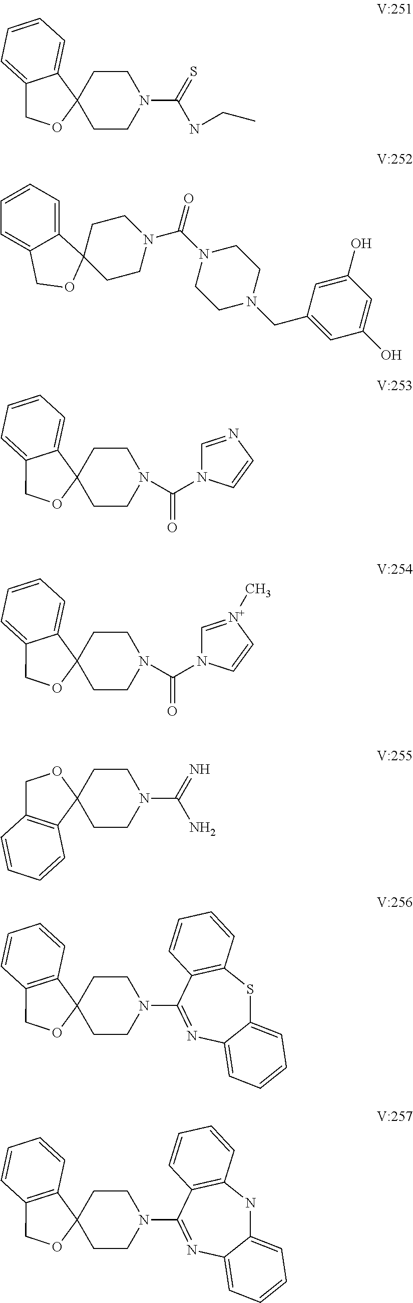

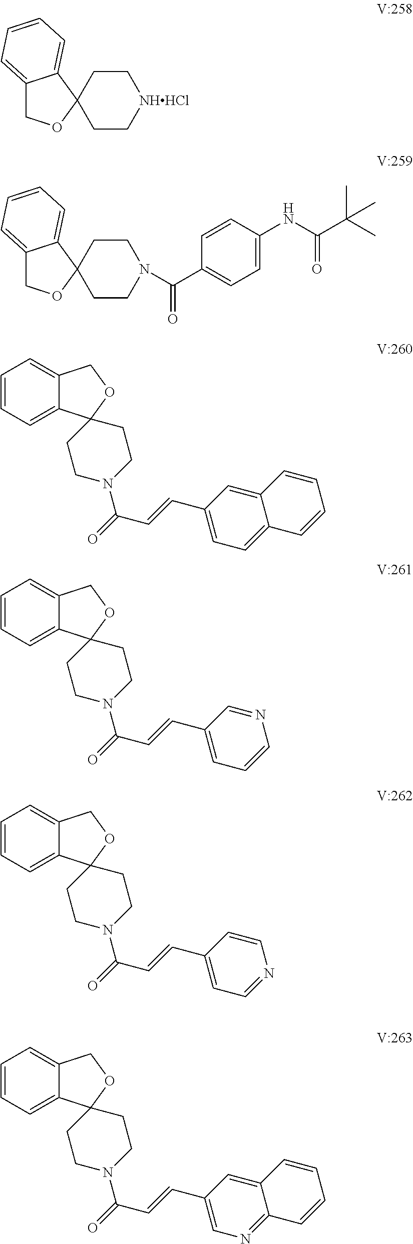

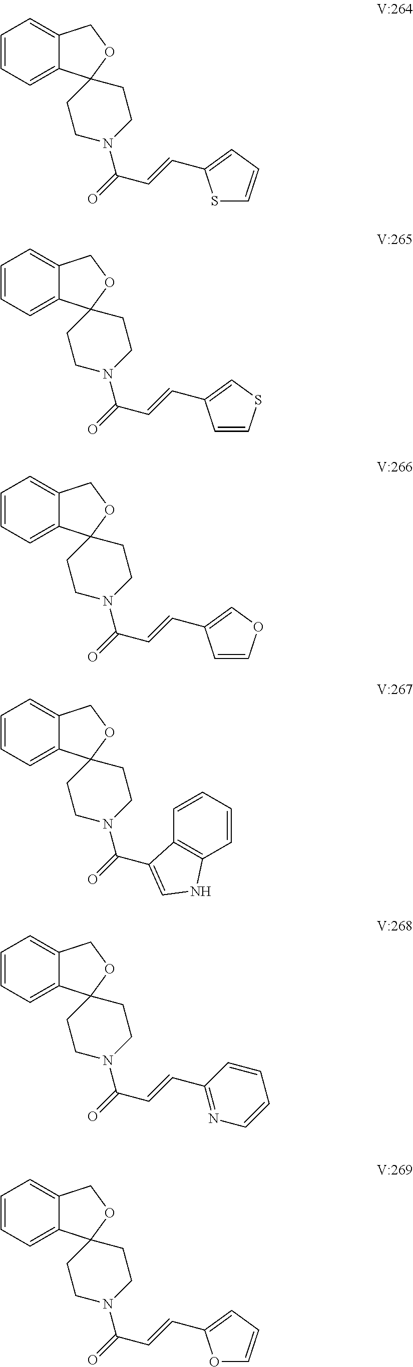 [SMILES: O=C(/C=C/c1cccs1)N1CCC2(CC1)OCc1ccccc12 ; O=C(/C=C/c1ccsc1)N1CCC2(CC1)OCc1ccccc12 ; O=C(/C=C/c1ccoc1)N1CCC2(CC1)OCc1ccccc12 ; O=C(c1c[nH]c2ccccc12)N1CCC2(CC1)OCc1ccccc12 ; O=C(/C=C/c1ccccn1)N1CCC2(CC1)OCc1ccccc12 ; O=C(/C=C/c1ccco1)N1CCC2(CC1)OCc1ccccc12]

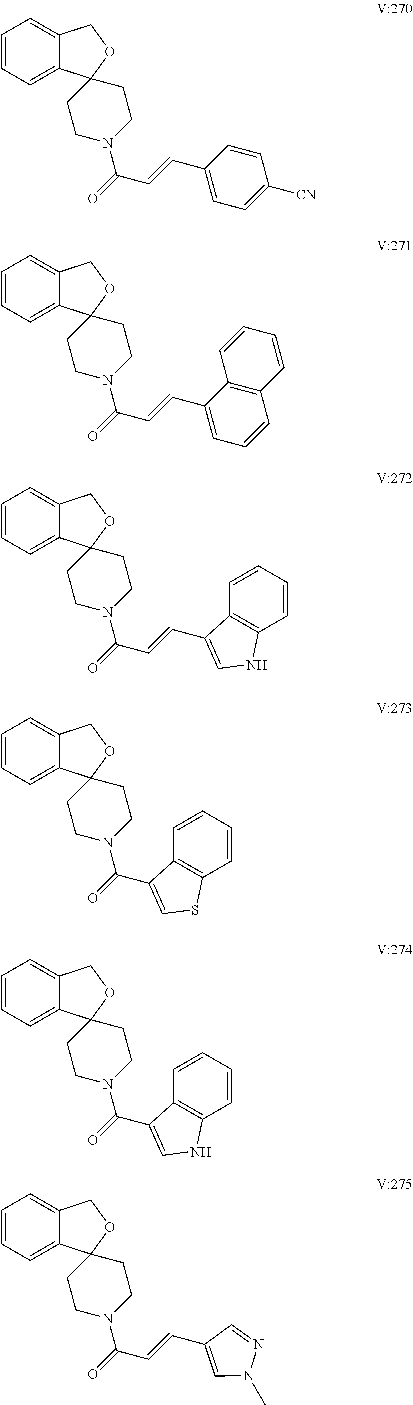 [SMILES: N#Cc1ccc(/C=C/C(=O)N2CCC3(CC2)OCc2ccccc23)cc1 ; O=C(/C=C/c1cccc2ccccc12)N1CCC2(CC1)OCc1ccccc12 ; O=C(/C=C/c1c[nH]c2ccccc12)N1CCC2(CC1)OCc1ccccc12 ; O=C(c1csc2ccccc12)N1CCC2(CC1)OCc1ccccc12 ; O=C(c1c[nH]c2ccccc12)N1CCC2(CC1)OCc1ccccc12 ; Cn1cc(/C=C/C(=O)N2CCC3(CC2)OCc2ccccc23)cn1]

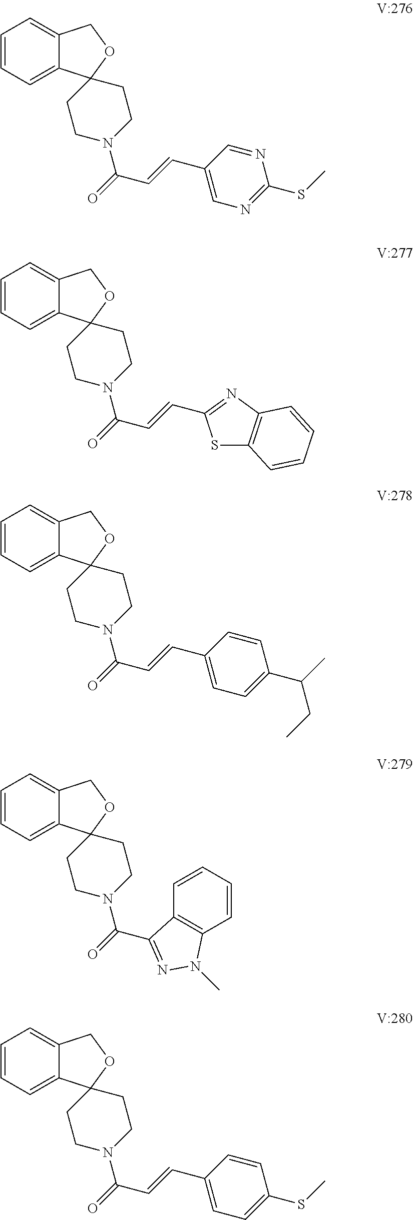 [SMILES: CSc1ncc(/C=C/C(=O)N2CCC3(CC2)OCc2ccccc23)cn1 ; O=C(/C=C/c1nc2ccccc2s1)N1CCC2(CC1)OCc1ccccc12 ; CCC(C)c1ccc(/C=C/C(=O)N2CCC3(CC2)OCc2ccccc23)cc1 ; Cn1nc(C(=O)N2CCC3(CC2)OCc2ccccc23)c2ccccc21 ; CSc1ccc(/C=C/C(=O)N2CCC3(CC2)OCc2ccccc23)cc1]

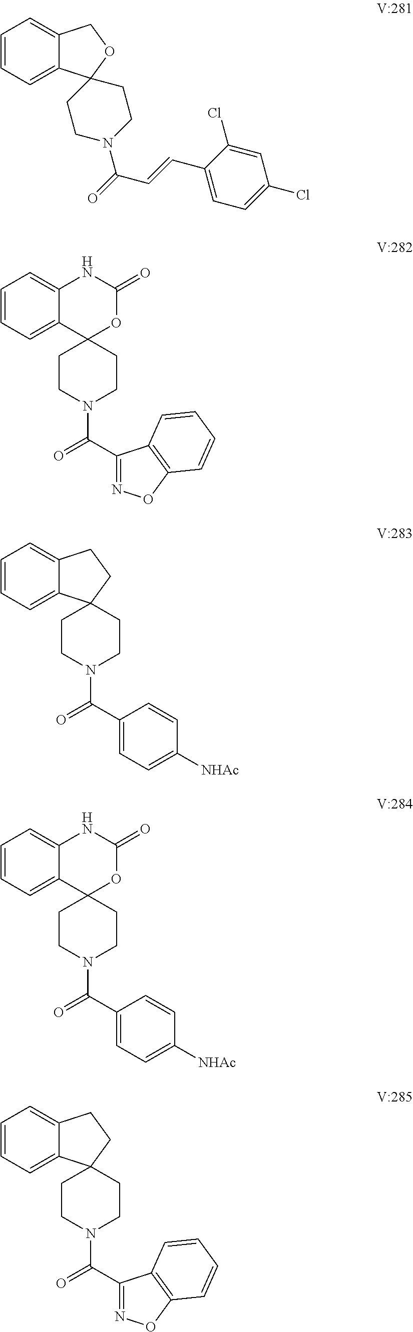 [SMILES: O=C(/C=C/c1ccc(Cl)cc1Cl)N1CCC2(CC1)OCc1ccccc12 ; O=C1Nc2ccccc2C2(CCN(C(=O)c3noc4ccccc34)CC2)O1 ; CC(=O)Nc1ccc(C(=O)N2CCC3(CCc4ccccc43)CC2)cc1 ; CC(=O)Nc1ccc(C(=O)N2CCC3(CC2)OC(=O)Nc2ccccc23)cc1 ; O=C(c1noc2ccccc12)N1CCC2(CCc3ccccc32)CC1]

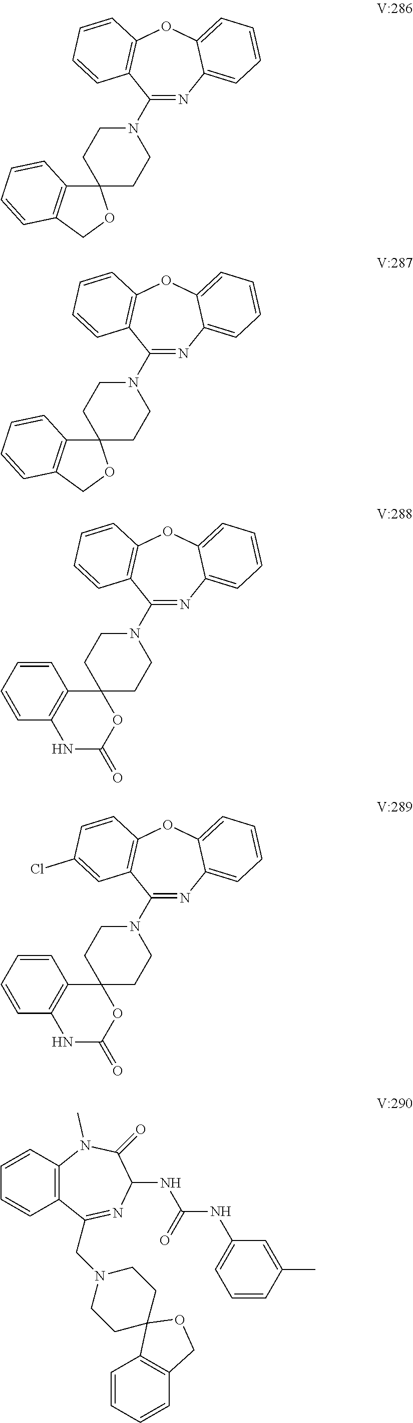

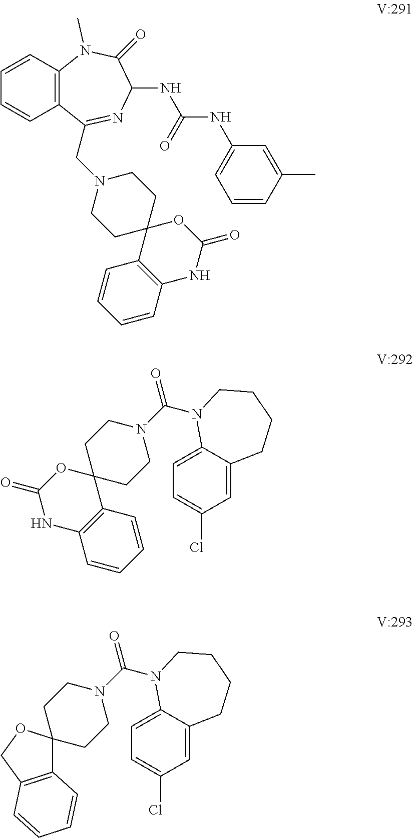 [SMILES: Cc1cccc(NC(=O)NC2N=C(CN3CCC4(CC3)OC(=O)Nc3ccccc34)c3ccccc3N(C)C2=O)c1 ; O=C1Nc2ccccc2C2(CCN(C(=O)N3CCCCc4cc(Cl)ccc43)CC2)O1 ; O=C(N1CCC2(CC1)OCc1ccccc12)N1CCCCc2cc(Cl)ccc21]

### Pharmaceutical Compositions

In various embodiments, the invention provides pharmaceutical compositions comprising a compound of the invention and a pharmaceutically acceptable excipient, optionally in combination with another medicament. As set forth herein, compounds of the invention include stereoisomers, tautomers, solvates, prodrugs, pharmaceutically acceptable salts and mixtures thereof. Compositions containing a compound of the invention can be prepared by conventional techniques, e.g. as described in Remington: The Science and Practice of Pharmacy, 19th Ed., 1995, or later versions thereof, incorporated by reference herein. The compositions can appear in conventional forms, for example capsules, tablets, aerosols, solutions, suspensions or topical applications.

Typical compositions include a compound of the invention and a pharmaceutically acceptable excipient which can be a carrier or a diluent. For example, the active compound will usually be mixed with a carrier, or diluted by a carrier, or enclosed within a carrier which can be in the form of an ampoule, capsule, sachet, paper, or other container. When the active compound is mixed with a carrier, or when the carrier serves as a diluent, it can be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent can include any sustained release material known in the art, such as glyceryl monostearate or glyceryl distearate, alone or mixed with a wax.

The formulations can be mixed with auxiliary agents which do not deleteriously react with the active compounds. Such additives can include wetting agents, emulsifying and suspending agents, salt for influencing osmotic pressure, buffers and/or coloring substances preserving agents, sweetening agents or flavoring agents. The compositions can also be sterilized if desired.

The route of administration can be any route which effectively transports the active compound of the invention to the appropriate or desired site of action, such as oral, nasal, pulmonary, buccal, subdermal, intradermal, transdermal or parenteral, e.g., rectal, depot, subcutaneous, intravenous, intraurethral, intramuscular, intranasal, ophthalmic solution or an ointment, the oral route being preferred.

If a solid carrier is used for oral administration, the preparation can be tableted, placed in a hard gelatin capsule in powder or pellet form or it can be in the form of a troche or lozenge. If a liquid carrier is used, the preparation can be in the form of a syrup, emulsion, soft gelatin capsule or sterile injectable liquid such as an aqueous or non-aqueous liquid suspension or solution.

Injectable dosage forms generally include aqueous suspensions or oil suspensions which can be prepared using a suitable dispersant or wetting agent and a suspending agent Injectable forms can be in solution phase or in the form of a suspension, which is prepared with a solvent or diluent. Acceptable solvents or vehicles include sterilized water, Ringer's solution, or an isotonic aqueous saline solution. Alternatively, sterile oils can be employed as solvents or suspending agents. Preferably, the oil or fatty acid is non-volatile, including natural or synthetic oils, fatty acids, mono-, di- or tri-glycerides.

For injection, the formulation can also be a powder suitable for reconstitution with an appropriate solution as described above. Examples of these include, but are not limited to, freeze dried, rotary dried or spray dried powders, amorphous powders, granules, precipitates, or particulates. For injection, the formulations can optionally contain stabilizers, pH modifiers, surfactants, bioavailability modifiers and combinations of these. The compounds can be formulated for parenteral administration by injection such as by bolus injection or continuous infusion. A unit dosage form for injection can be in ampoules or in multi-dose containers.

The formulations of the invention can be designed to provide quick, sustained, or delayed release of the active ingredient after administration to the patient by employing procedures well known in the art. Thus, the formulations can also be formulated for controlled release or for slow release.

Compositions contemplated by the present invention can include, for example, micelles or liposomes, or some other encapsulated form, or can be administered in an extended release form to provide a prolonged storage and/or delivery effect. Therefore, the formulations can be compressed into pellets or cylinders and implanted intramuscularly or subcutaneously as depot injections. Such implants can employ known inert materials such as silicones and biodegradable polymers, e.g., polylactide-polyglycolide. Examples of other biodegradable polymers include poly(orthoesters) and poly(anhydrides).

For nasal administration, the preparation can contain a compound of the invention, dissolved or suspended in a liquid carrier, preferably an aqueous carrier, for aerosol application. The carrier can contain additives such as solubilizing agents, e.g., propylene glycol, surfactants, absorption enhancers such as lecithin (phosphatidylcholine) or cyclodextrin, or preservatives such as parabens.

For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

A typical tablet that can be prepared by conventional tabletting techniques can contain:

| Core: | | |
|---|---|---|
| Active compound (as free compound or salt thereof) | 250 | mg |
| Colloidal silicon dioxide (Aerosil) ® | 1.5 | mg |
| Cellulose, microcryst. (Avicel) ® | 70 | mg |
| Modified cellulose gum (Ac-Di-Sol) ® | 7.5 | mg |
| Magnesium stearate | Ad. | |
| Coating: | | |
| HPMC approx. | 9 | mg |
| *Mywacett 9-40 T approx. | 0.9 | mg |

*Acylated monoglyceride used as plasticizer for film coating.

A typical capsule for oral administration contains compounds of the invention (250 mg), lactose (75 mg) and magnesium stearate (15 mg). The mixture is passed through a 60 mesh sieve and packed into a No. 1 gelatin capsule. A typical injectable preparation is produced by aseptically placing 250 mg of compounds of the invention into a vial, aseptically freeze-drying and sealing. For use, the contents of the vial are mixed with 2 mL of sterile physiological saline, to produce an injectable preparation.

The compounds of the invention can be administered to a mammal, especially a human in need of such treatment, prevention, elimination, alleviation or amelioration of a condition. Such mammals include also animals, both domestic animals, e.g. household pets, farm animals, and non-domestic animals such as wildlife.

The compounds of the invention are effective over a wide dosage range. For example, in the treatment of adult humans, dosages from about 0.05 to about 5000 mg, preferably from about 1 to about 2000 mg, and more preferably between about 2 and about 2000 mg per day can be used. A typical dosage is about 10 mg to about 1000 mg per day. In choosing a regimen for patients it can frequently be necessary to begin with a higher dosage and when the condition is under control to reduce the dosage. The exact dosage will depend upon the activity of the compound, mode of administration, on the therapy desired, form in which administered, the subject to be treated and the body weight of the subject to be treated, and the preference and experience of the physician or veterinarian in charge.

Generally, the compounds of the invention are dispensed in unit dosage form including from about 0.05 mg to about 1000 mg of active ingredient together with a pharmaceutically acceptable carrier per unit dosage.

Usually, dosage forms suitable for oral, nasal, pulmonal or transdermal administration include from about 125 μg to about 1250 mg, preferably from about 250 μg to about 500 mg, and more preferably from about 2.5 mg to about 250 mg, of the compounds admixed with a pharmaceutically acceptable carrier or diluent.

Dosage forms can be administered daily, or more than once a day, such as twice or thrice daily. Alternatively dosage forms can be administered less frequently than daily, such as every other day, or weekly, if found to be advisable by a prescribing physician.

### Pharmaceutical Uses

In various embodiments, the invention provides a use of a compound of the invention for treatment of a disease or condition. For example, the disease or condition can be one wherein modulation of a vasopressin receptor is medically indicated. More specifically, the disease or condition can include any of compromised lactation conditions, labor induction impairment, uterine atony conditions, excessive bleeding, inflammation and pain including abdominal and back pain, sexual dysfunction, both male and female, irritable bowel syndrome, constipation and gastrointestinal obstruction, autism, stress, anxiety including anxiety disorders, depression, surgical blood loss, post-partum hemorrhage, defective wound healing, infection, mastitis, placenta delivery impairment, placental insufficiency, osteoporosis, cancer, nicotine or alcohol abuse, circadian rhythm disorders, jet lag, disorders of the immune system, metabolic disorders, traumatic brain injury, cerebral infarction or stroke.

In various embodiments, the invention provides a method of modulating a vasopressin receptor, comprising contacting the receptor with an effective amount or concentration of a compound of the invention. The modulation can take place in vivo, as in the treatment of a human or non-human patient, or can take place in vitro, as in carrying out a biochemical assay or evaluation.

In various embodiments, the invention provides a method of treatment of a condition in a patient afflicted therewith, comprising administering to the patient an effective amount of a compound of the invention at a frequency and for a duration to provide a benefit to the patient. For example, the condition can be such that modulating a vasopressin receptor is medically indicated for treatment of the condition. Examples of such conditions can include any of compromised lactation conditions, labor induction impairment, uterine atony conditions, excessive bleeding, inflammation and pain including abdominal and back pain, sexual dysfunction, both male and female, irritable bowel syndrome, constipation and gastrointestinal obstruction, autism, stress, anxiety including anxiety disorders, depression, surgical blood loss, post-partum hemorrhage, defective wound healing, infection, mastitis, placenta delivery impairment, placental insufficiency, osteoporosis, or cancer; or septic shock, polycystic kidney disease, pulmonary hypertension, vasodilation/cinstriction, cardiopulmonary resuscitation, pediatric shock, cardiac arrest, wound healing disorders, metabolic disorders, diabetes, obesity, substance abuse, nicotine or alcohol abuse, circadian rhythm disorders, jet lag, disorders of the immune system, metabolic disorders, traumatic brain injury, cerebral infarction or stroke. More specifically, anxiety disorder can comprise any of generalized anxiety disorder, panic disorder, agoraphobia, phobias, social anxiety disorder, obsessive-compulsive disorder, post-traumatic stress disorder, or separation anxiety.

### Biological Data

### Compounds of Formula (I)

Data represent Ki values (nM) or mean % inhibition (indicated as %) for exemplary compounds of formula (I) tested versus receptor subtypes.

The default concentration for primary binding experiments was 10 μM. Each row shows data for a single compound. See Examples for further details.

TABLE 2
Receptor Binding Constants for Exemplary Compounds of Formula (I):
Each row represents data for a single compound.

| | OT binding | V1A binding | V2 binding |
|---|---|---|---|
| Example # | Ki (nM) | Ki (nM) | Ki (nM) |
| 1 | 12.8% at | 34.1% at | NA |
| | 10 μM | 10 μM | |
| 2 | 13 | 10 | 33.6% at |
| | | | 10 μM |
| 3 | 1 | 0.9 | 708 |
| 4 | 21 | 10 | 24.5% at |
| | | | 10 μM |
| 5 | 326 | 785 | 17.7% at |
| | | | 10 μM |
| 6 | 45 | 113 | 33% at 10 μM |
| 8 | 1178 | 35.1% at | NA |
| | | 10 μM | |
| 9 | 22.3% at | NA | NA |
| | 10 μM | | |
| 10 | 17.1% at | 2.2% at | NA |
| | 10 μM | 10 μM | |
| 11 | 14.8% at | NA | NA |
| | 10 μM | | |
| 12 | 10.6% at | NA | NA |
| | 10 μM | | |
| 13 | 16.1% at | NA | 13.6% at |
| | 10 μM | | 10 μM |
| 14 | 19.6% at | NA | 5.8% at 10 μM |
| | 10 μM | | |
| 15 | 2.8% at | 19% at | 12.8% at |
| | 10 μM | 10 μM | 10 μM |
| 16 | 2317 | 1159 | 12.7% at |
| | | | 10 μM |
| 17 | 118 | 79 | 17.9% at |
| | | | 10 μM |
| 18 | 2.8% at | 19% at | 12.8% at |
| | 10 μM | 10 μM | 10 μM |
| 19 | 757 | 880 | 5.7% at 10 μM |
| 20 | 329 | 851 | 33% at 10 μM |
| 21 | 204 | 116 | 24.3% at |
| | | | 10 μM |
| 22 | 27.7% at | 1590 | 6.6% at 10 μM |
| | 10 μM | | |
| 23 | 47 | 15 | 407 |
| 24 | 265 | 1166 | 23.3% at |
| | | | 10 μM |
| 25 | 40.7% at | 3880 | 40.4% at |
| | 10 μM | | 10 μM |
| 26 | 4.8 | 3 | 1855 |
| 27 | 2.3 | 1 | 3781 |
| 28 | 51 | 107 | 5006 |
| 29 | 61 | 12 | 159 |
| 30 | 2.3 | 11 | 75 |
| 31 | 6 | 5 | 7338 |
| 32 | 2.3 | 11 | 1699 |
| 33 | 4.1 | 3 | 205 |
| 24 | 24.5 nM | 19 nM | 541.6 nM |
| 35 | 37.4 nM | 71 nM | <50% @ 10 |
| | | | uM |

TABLE 3
Functional data for Exemplary Compounds of Formula (I)
Each row represents data for a single compound. Data are provided for
compounds 55-78.

| | OT Antagonist- | V1a Antgonist- |
|---|---|---|
| Compound No. | 10 μM | 10 μM |
| 55 | −8.7 | −67.7 |
| 56 | −34.6 | −27.8 |
| 57 | −36.6 | −79.5 |
| 58 | 25.1 | −5.7 |
| 59 | 98.8 | 54.9 |
| 60 | 11.2 | −36.7 |
| 61 | 13.2 | −8.6 |
| 62 | 86.8 | 43.1 |
| 63 | 86.8 | 51.9 |
| 64 | −76.4 | −38.2 |
| 65 | −58.5 | −46.3 |
| 66 | 23.1 | −16.7 |
| 67 | 27.1 | −24.1 |
| 68 | 31.1 | −25.6 |
| 69 | 78.9 | −10.1 |
| 70 | 17.2 | −14.5 |
| 71 | 16.5 | −1.5 |
| 72 | −54.5 | 0 |
| 73 | −42.5 | 0.5 |
| 74 | 16.5 | −0.1 |
| 75 | −31.3 | −0.1 |
| 76 | −2.6 | 17.1 |
| 77 | −61.8 | −0.1 |
| 78 | −21.7 | −0.1 |

TABLE 4
Chemical and Bioactivity Data for Compounds 79-228

| | | V1a-R | |
|---|---|---|---|
| Cpd. | | IC₅₀ | Observed |
| # | Structure | (μM) | LC-MS |
| 79 | 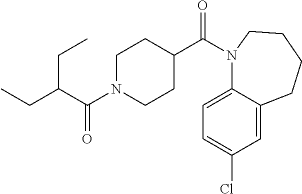 [SMILES: CCC(CC)C(=O)N1CCC(C(=O)N2CCCCc3cc(Cl)ccc32)CC1] | 5.403 | 391.0 |
| 80 | 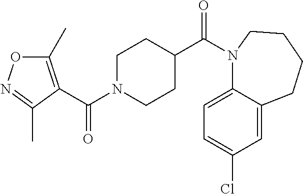 [SMILES: Cc1noc(C)c1C(=O)N1CCC(C(=O)N2CCCCc3cc(Cl)ccc32)CC1] | 15.102 | 416.0 |
| 81 | 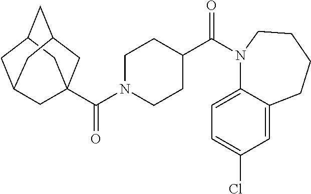 [SMILES: O=C(C1CCN(C(=O)C23CC4CC(CC(C4)C2)C3)CC1)N1CCCCc2cc(Cl)ccc21] | 8.077 | 455.0 |
| 82 | 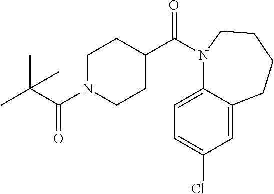 [SMILES: CC(C)(C)C(=O)N1CCC(C(=O)N2CCCCc3cc(Cl)ccc32)CC1] | 2.486 | 377.0 |
| 83 | 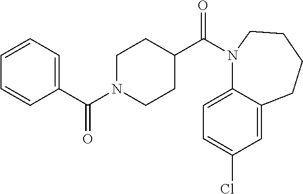 [SMILES: O=C(c1ccccc1)N1CCC(C(=O)N2CCCCc3cc(Cl)ccc32)CC1] | 1.825 | 397.0 |
| 84 | 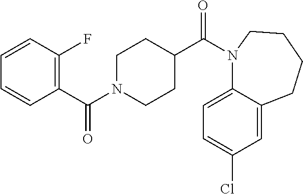 [SMILES: O=C(c1ccccc1F)N1CCC(C(=O)N2CCCCc3cc(Cl)ccc32)CC1] | 1.494 | 415.0 |
| 85 | 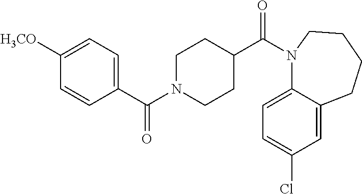 [SMILES: COc1ccc(C(=O)N2CCC(C(=O)N3CCCCc4cc(Cl)ccc43)CC2)cc1] | 1.779 | 427.0 |
| 86 | 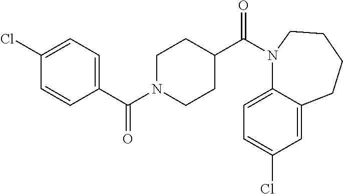 [SMILES: O=C(c1ccc(Cl)cc1)N1CCC(C(=O)N2CCCCc3cc(Cl)ccc32)CC1] | 0.835 | 432.0 |
| 87 | 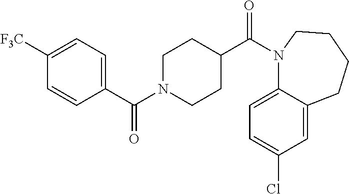 [SMILES: O=C(c1ccc(C(F)(F)F)cc1)N1CCC(C(=O)N2CCCCc3cc(Cl)ccc32)CC1] | 1.869 | 465.0 |
| 88 | 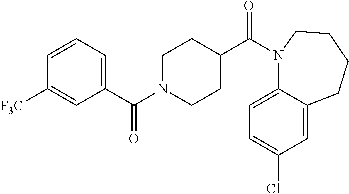 [SMILES: O=C(c1cccc(C(F)(F)F)c1)N1CCC(C(=O)N2CCCCc3cc(Cl)ccc32)CC1] | 0.595 | 465.0 |
| 89 | 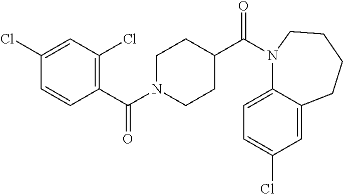 [SMILES: O=C(c1ccc(Cl)cc1Cl)N1CCC(C(=O)N2CCCCc3cc(Cl)ccc32)CC1] | 1.796 | 465.8 |
| 90 | 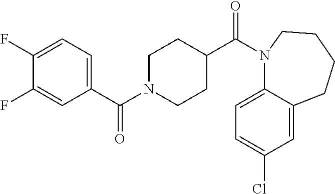 [SMILES: O=C(c1ccc(F)c(F)c1)N1CCC(C(=O)N2CCCCc3cc(Cl)ccc32)CC1] | 1.515 | 433.0 |
| 91 | 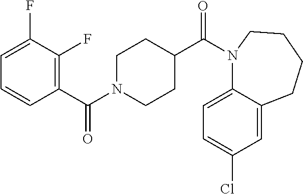 [SMILES: O=C(c1cccc(F)c1F)N1CCC(C(=O)N2CCCCc3cc(Cl)ccc32)CC1] | 1.855 | 433.0 |
| 92 | 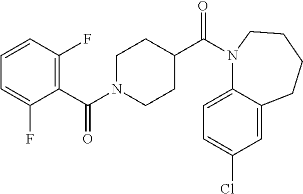 [SMILES: O=C(c1c(F)cccc1F)N1CCC(C(=O)N2CCCCc3cc(Cl)ccc32)CC1] | 1.716 | 433.0 |
| 93 | 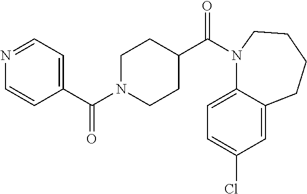 [SMILES: O=C(c1ccncc1)N1CCC(C(=O)N2CCCCc3cc(Cl)ccc32)CC1] | 5.810 | 398.0 |
| 94 | 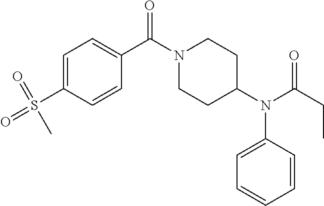 [SMILES: CCC(=O)N(c1ccccc1)C1CCN(C(=O)c2ccc(S(C)(=O)=O)cc2)CC1] | >50 | 415.0 |
| 95 | 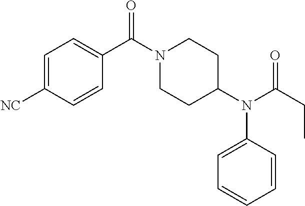 [SMILES: CCC(=O)N(c1ccccc1)C1CCN(C(=O)c2ccc(C#N)cc2)CC1] | >50 | 362.0 |
| 96 | 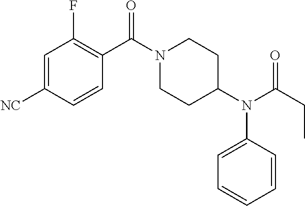 [SMILES: CCC(=O)N(c1ccccc1)C1CCN(C(=O)c2ccc(C#N)cc2F)CC1] | >50 | 380.0 |
| 97 | 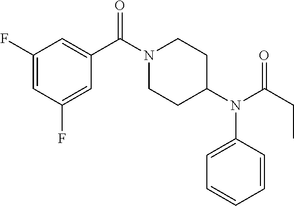 [SMILES: CCC(=O)N(c1ccccc1)C1CCN(C(=O)c2cc(F)cc(F)c2)CC1] | >50 | 373.0 |
| 98 | 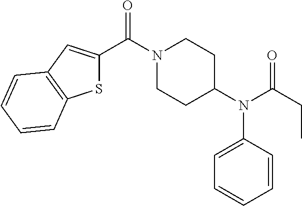 [SMILES: CCC(=O)N(c1ccccc1)C1CCN(C(=O)c2cc3ccccc3s2)CC1] | >50 | 393.0 |
| 99 | 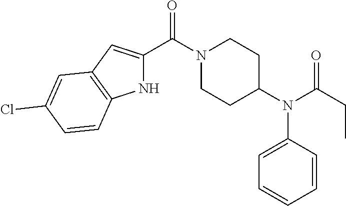 [SMILES: CCC(=O)N(c1ccccc1)C1CCN(C(=O)c2cc3cc(Cl)ccc3[nH]2)CC1] | 36.902 | 410.0 |
| 101 | 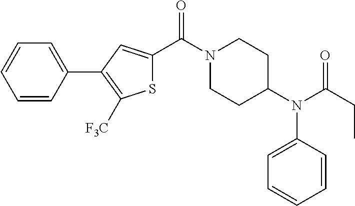 [SMILES: CCC(=O)N(c1ccccc1)C1CCN(C(=O)c2cc(-c3ccccc3)c(C(F)(F)F)s2)CC1] | >50 | 487.0 |
| 102 | 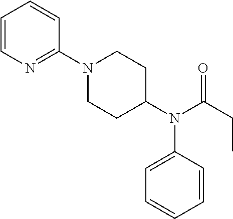 [SMILES: CCC(=O)N(c1ccccc1)C1CCN(c2ccccn2)CC1] | >50 | 310.0 |
| 103 | 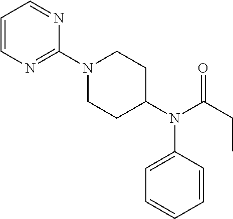 [SMILES: CCC(=O)N(c1ccccc1)C1CCN(c2ncccn2)CC1] | >50 | 311.0 |
| 104 | 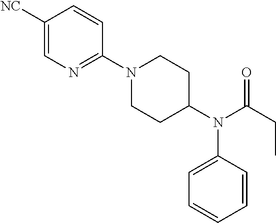 [SMILES: CCC(=O)N(c1ccccc1)C1CCN(c2ccc(C#N)cn2)CC1] | >50 | 335.0 |
| 105 | 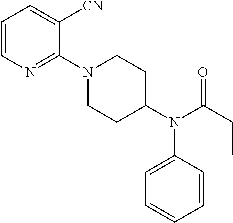 [SMILES: CCC(=O)N(c1ccccc1)C1CCN(c2ncccc2C#N)CC1] | >50 | 335.0 |
| 106 | 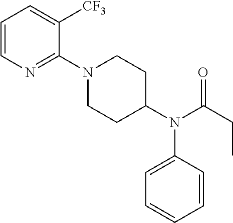 [SMILES: CCC(=O)N(c1ccccc1)C1CCN(c2ncccc2C(F)(F)F)CC1] | 18.503 | 378.0 |
| 107 | 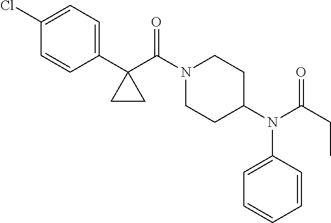 [SMILES: CCC(=O)N(c1ccccc1)C1CCN(C(=O)C2(c3ccc(Cl)cc3)CC2)CC1] | 29.545 | 411.0 |
| 108 | 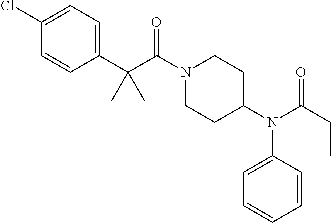 [SMILES: CCC(=O)N(c1ccccc1)C1CCN(C(=O)C(C)(C)c2ccc(Cl)cc2)CC1] | >50 | 413.0 |
| 109 | 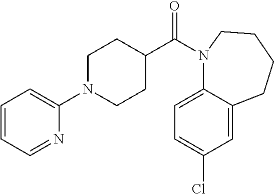 [SMILES: O=C(C1CCN(c2ccccn2)CC1)N1CCCCc2cc(Cl)ccc21] | 0.005 | 370.0 |
| 110 | 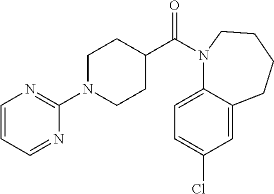 [SMILES: O=C(C1CCN(c2ncccn2)CC1)N1CCCCc2cc(Cl)ccc21] | 0.207 | 371.0 |
| 111 | 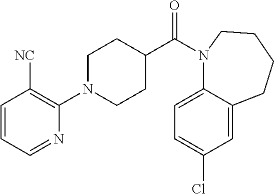 [SMILES: N#Cc1cccnc1N1CCC(C(=O)N2CCCCc3cc(Cl)ccc32)CC1] | 0.130 | 395.0 |
| 112 | 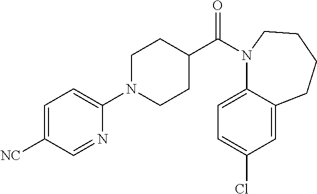 [SMILES: N#Cc1ccc(N2CCC(C(=O)N3CCCCc4cc(Cl)ccc43)CC2)nc1] | 1.234 | 395.0 |
| 113 | 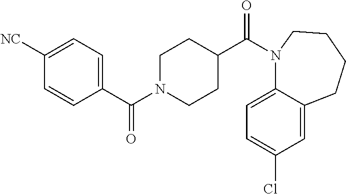 [SMILES: N#Cc1ccc(C(=O)N2CCC(C(=O)N3CCCCc4cc(Cl)ccc43)CC2)cc1] | 5.760 | 422.0 |
| 114 | 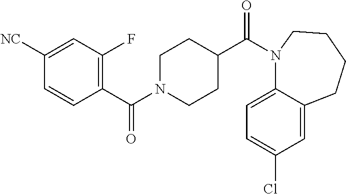 [SMILES: N#Cc1ccc(C(=O)N2CCC(C(=O)N3CCCCc4cc(Cl)ccc43)CC2)c(F)c1] | 9.480 | 440.0 |
| 115 | 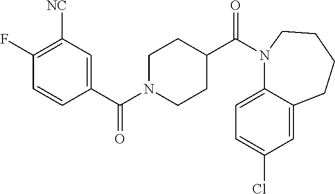 [SMILES: N#Cc1cc(C(=O)N2CCC(C(=O)N3CCCCc4cc(Cl)ccc43)CC2)ccc1F] | 5.281 | 440.0 |
| 116 | 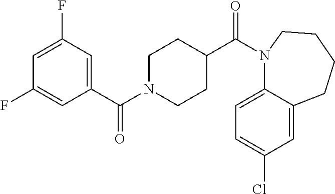 [SMILES: O=C(c1cc(F)cc(F)c1)N1CCC(C(=O)N2CCCCc3cc(Cl)ccc32)CC1] | 1.600 | 433.0 |
| 117 | 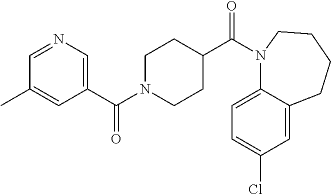 [SMILES: Cc1cncc(C(=O)N2CCC(C(=O)N3CCCCc4cc(Cl)ccc43)CC2)c1] | 1.155 | 412.0 |
| 118 | 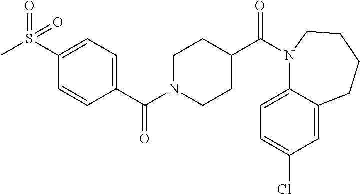 [SMILES: CS(=O)(=O)c1ccc(C(=O)N2CCC(C(=O)N3CCCCc4cc(Cl)ccc43)CC2)cc1] | 12.935 | 475.0 |
| 119 | 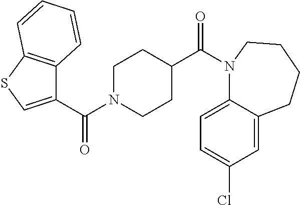 [SMILES: O=C(c1csc2ccccc12)N1CCC(C(=O)N2CCCCc3cc(Cl)ccc32)CC1] | 0.621 | 453.0 |
| 120 | 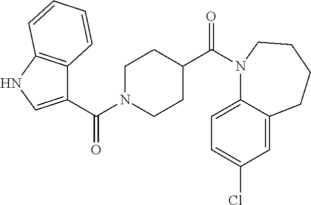 [SMILES: O=C(c1c[nH]c2ccccc12)N1CCC(C(=O)N2CCCCc3cc(Cl)ccc32)CC1] | 0.249 | 436.0 |
| 121 | 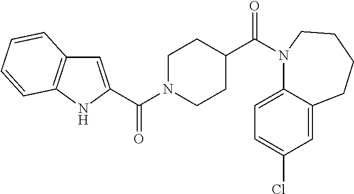 [SMILES: O=C(c1cc2ccccc2[nH]1)N1CCC(C(=O)N2CCCCc3cc(Cl)ccc32)CC1] | 0.446 | 436.0 |
| 122 | 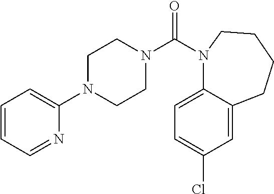 [SMILES: O=C(N1CCN(c2ccccn2)CC1)N1CCCCc2cc(Cl)ccc21] | 0.080 | 371.0 |
| 123 | 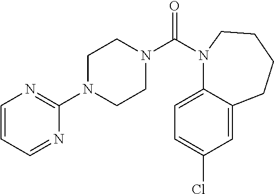 [SMILES: O=C(N1CCN(c2ncccn2)CC1)N1CCCCc2cc(Cl)ccc21] | 0.256 | 372.0 |
| 124 | 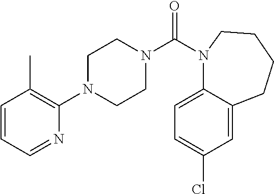 [SMILES: Cc1cccnc1N1CCN(C(=O)N2CCCCc3cc(Cl)ccc32)CC1] | 0.040 | 385.0 |
| 125 | 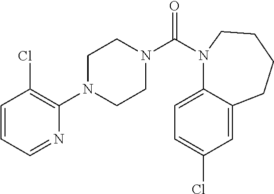 [SMILES: O=C(N1CCN(c2ncccc2Cl)CC1)N1CCCCc2cc(Cl)ccc21] | 0.057 | 405.0 |
| 126 | 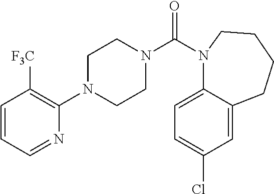 [SMILES: O=C(N1CCN(c2ncccc2C(F)(F)F)CC1)N1CCCCc2cc(Cl)ccc21] | 0.054 | 439.0 |
| 127 | 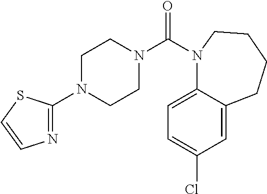 [SMILES: O=C(N1CCN(c2nccs2)CC1)N1CCCCc2cc(Cl)ccc21] | 0.267 | 377.0 |
| 128 | 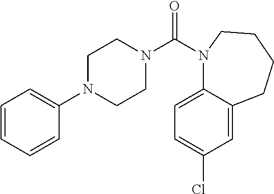 [SMILES: O=C(N1CCN(c2ccccc2)CC1)N1CCCCc2cc(Cl)ccc21] | 0.027 | 370.0 |
| 129 | 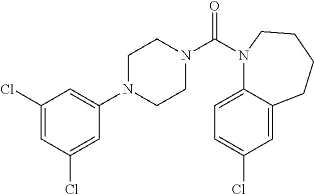 [SMILES: O=C(N1CCN(c2cc(Cl)cc(Cl)c2)CC1)N1CCCCc2cc(Cl)ccc21] | 0.205 | 438.0 |
| 130 | 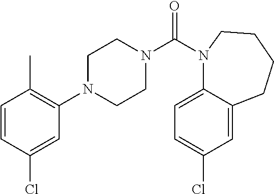 [SMILES: Cc1ccc(Cl)cc1N1CCN(C(=O)N2CCCCc3cc(Cl)ccc32)CC1] | 0.279 | 418.0 |
| 131 | 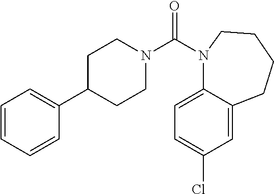 [SMILES: O=C(N1CCC(c2ccccc2)CC1)N1CCCCc2cc(Cl)ccc21] | 0.083 | 369.0 |
| 132 | 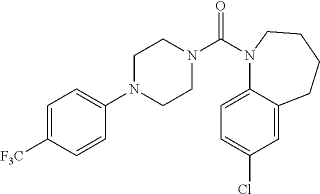 [SMILES: O=C(N1CCN(c2ccc(C(F)(F)F)cc2)CC1)N1CCCCc2cc(Cl)ccc21] | 0.029 | 438.0 |
| 133 | 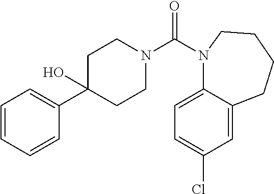 [SMILES: O=C(N1CCC(O)(c2ccccc2)CC1)N1CCCCc2cc(Cl)ccc21] | 0.137 | 385.0 |
| 134 | 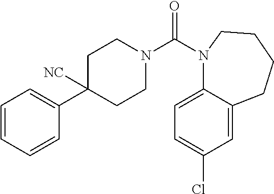 [SMILES: N#CC1(c2ccccc2)CCN(C(=O)N2CCCCc3cc(Cl)ccc32)CC1] | 0.358 | 394.0 |
| 136 | 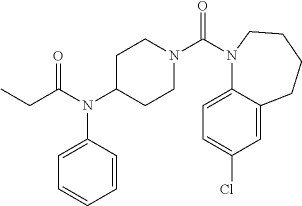 [SMILES: CCC(=O)N(c1ccccc1)C1CCN(C(=O)N2CCCCc3cc(Cl)ccc32)CC1] | 0.194 | 440.0 |
| 139 | 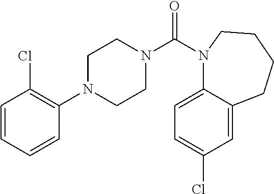 [SMILES: O=C(N1CCN(c2ccccc2Cl)CC1)N1CCCCc2cc(Cl)ccc21] | 0.035 | 404.0 |
| 140 | 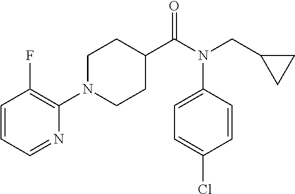 [SMILES: O=C(C1CCN(c2ncccc2F)CC1)N(CC1CC1)c1ccc(Cl)cc1] | 0.015 | 388.0 |
| 141 | 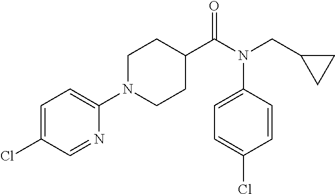 [SMILES: O=C(C1CCN(c2ccc(Cl)cn2)CC1)N(CC1CC1)c1ccc(Cl)cc1] | 0.040 | 404.0 |
| 142 | 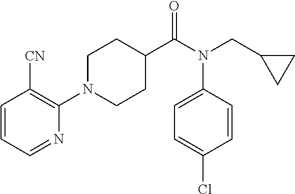 [SMILES: N#Cc1cccnc1N1CCC(C(=O)N(CC2CC2)c2ccc(Cl)cc2)CC1] | 0.067 | 395.0 |
| 143 | 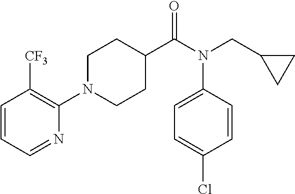 [SMILES: O=C(C1CCN(c2ncccc2C(F)(F)F)CC1)N(CC1CC1)c1ccc(Cl)cc1] | 0.084 | 438.0 |
| 144 | 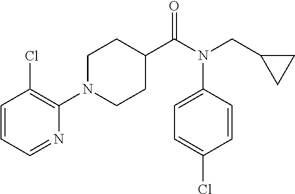 [SMILES: O=C(C1CCN(c2ncccc2Cl)CC1)N(CC1CC1)c1ccc(Cl)cc1] | 0.023 | 404.0 |
| 145 | 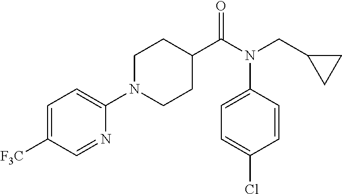 [SMILES: O=C(C1CCN(c2ccc(C(F)(F)F)cn2)CC1)N(CC1CC1)c1ccc(Cl)cc1] | 0.123 | 438.0 |
| 146 | 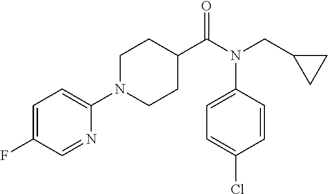 [SMILES: O=C(C1CCN(c2ccc(F)cn2)CC1)N(CC1CC1)c1ccc(Cl)cc1] | 0.024 | 388.0 |
| 147 | 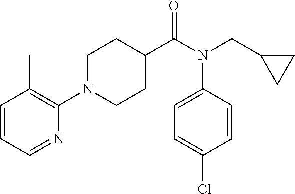 [SMILES: Cc1cccnc1N1CCC(C(=O)N(CC2CC2)c2ccc(Cl)cc2)CC1] | 0.007 | 384.0 |
| 148 | 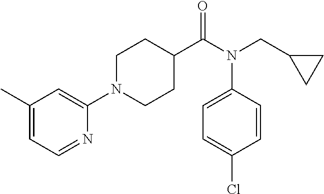 [SMILES: Cc1ccnc(N2CCC(C(=O)N(CC3CC3)c3ccc(Cl)cc3)CC2)c1] | 0.003 | 384.0 |
| 149 | 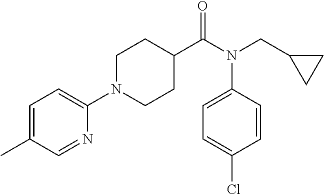 [SMILES: Cc1ccc(N2CCC(C(=O)N(CC3CC3)c3ccc(Cl)cc3)CC2)nc1] | 0.020 | 384.0 |
| 150 | 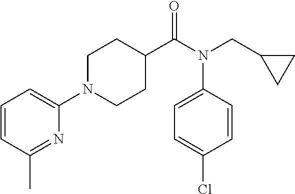 [SMILES: Cc1cccc(N2CCC(C(=O)N(CC3CC3)c3ccc(Cl)cc3)CC2)n1] | 0.023 | 384.0 |
| 151 | 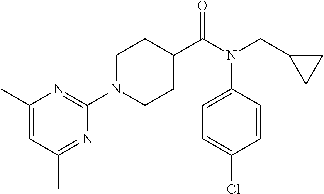 [SMILES: Cc1cc(C)nc(N2CCC(C(=O)N(CC3CC3)c3ccc(Cl)cc3)CC2)n1] | 0.008 | 399.0 |
| 152 | 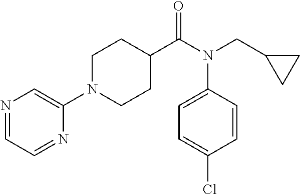 [SMILES: O=C(C1CCN(c2cnccn2)CC1)N(CC1CC1)c1ccc(Cl)cc1] | 0.045 | 371.0 |
| 153 | 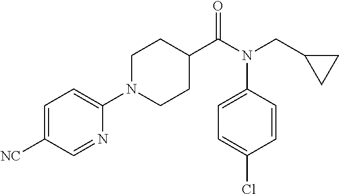 [SMILES: N#Cc1ccc(N2CCC(C(=O)N(CC3CC3)c3ccc(Cl)cc3)CC2)nc1] | 0.800 | 395.0 |
| 154 | 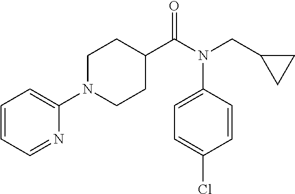 [SMILES: O=C(C1CCN(c2ccccn2)CC1)N(CC1CC1)c1ccc(Cl)cc1] | 0.015 | 370.0 |
| 155 | 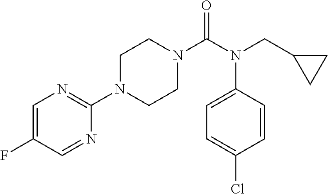 [SMILES: O=C(N1CCN(c2ncc(F)cn2)CC1)N(CC1CC1)c1ccc(Cl)cc1] | 0.106 | 390.0 |
| 156 | 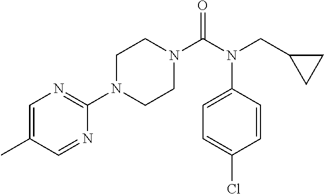 [SMILES: Cc1cnc(N2CCN(C(=O)N(CC3CC3)c3ccc(Cl)cc3)CC2)nc1] | 0.017 | 386.0 |
| 157 | 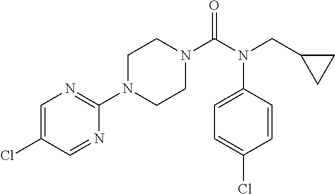 [SMILES: O=C(N1CCN(c2ncc(Cl)cn2)CC1)N(CC1CC1)c1ccc(Cl)cc1] | 0.206 | 406.0 |
| 158 | 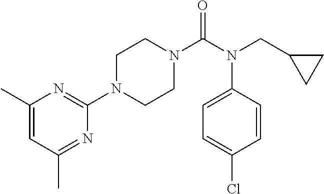 [SMILES: Cc1cc(C)nc(N2CCN(C(=O)N(CC3CC3)c3ccc(Cl)cc3)CC2)n1] | 0.024 | 400.0 |
| 159 | 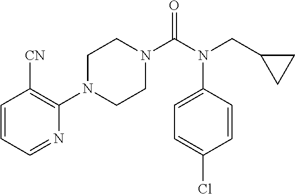 [SMILES: N#Cc1cccnc1N1CCN(C(=O)N(CC2CC2)c2ccc(Cl)cc2)CC1] | 0.081 | 396.0 |
| 160 | 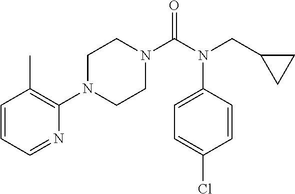 [SMILES: Cc1cccnc1N1CCN(C(=O)N(CC2CC2)c2ccc(Cl)cc2)CC1] | 0.008 | 385.0 |
| 161 | 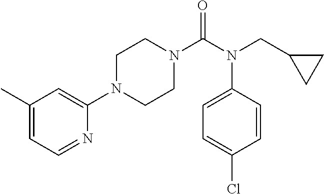 [SMILES: Cc1ccnc(N2CCN(C(=O)N(CC3CC3)c3ccc(Cl)cc3)CC2)c1] | 0.016 | 385.0 |
| 162 | 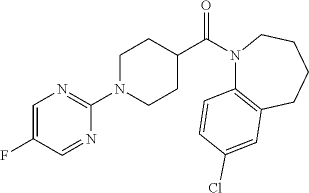 [SMILES: O=C(C1CCN(c2ncc(F)cn2)CC1)N1CCCCc2cc(Cl)ccc21] | 0.035 | 389.0 |
| 163 | 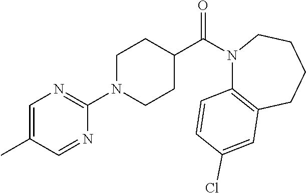 [SMILES: Cc1cnc(N2CCC(C(=O)N3CCCCc4cc(Cl)ccc43)CC2)nc1] | 0.008 | 385.0 |
| 164 | 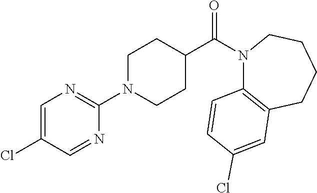 [SMILES: O=C(C1CCN(c2ncc(Cl)cn2)CC1)N1CCCCc2cc(Cl)ccc21] | 0.056 | 405.0 |
| 165 | 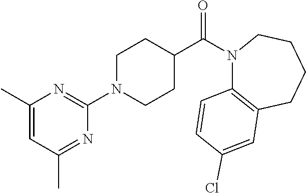 [SMILES: Cc1cc(C)nc(N2CCC(C(=O)N3CCCCc4cc(Cl)ccc43)CC2)n1] | 0.002 | 399.0 |
| 166 | 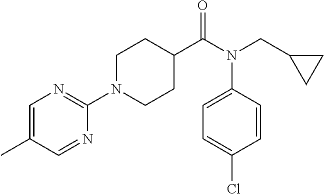 [SMILES: Cc1cnc(N2CCC(C(=O)N(CC3CC3)c3ccc(Cl)cc3)CC2)nc1] | 0.038 | 385.0 |
| 167 | 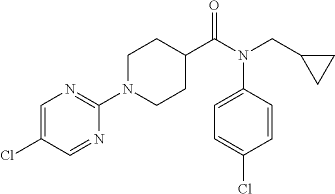 [SMILES: O=C(C1CCN(c2ncc(Cl)cn2)CC1)N(CC1CC1)c1ccc(Cl)cc1] | 0.195 | 405.0 |
| 168 | 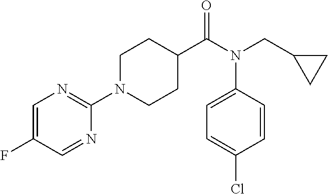 [SMILES: O=C(C1CCN(c2ncc(F)cn2)CC1)N(CC1CC1)c1ccc(Cl)cc1] | 0.091 | 389.0 |
| 169 | 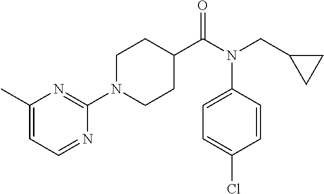 [SMILES: Cc1ccnc(N2CCC(C(=O)N(CC3CC3)c3ccc(Cl)cc3)CC2)n1] | 0.051 | 385.0 |
| 170 | 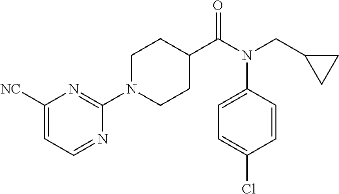 [SMILES: N#Cc1ccnc(N2CCC(C(=O)N(CC3CC3)c3ccc(Cl)cc3)CC2)n1] | 0.096 | 396.0 |
| 171 | 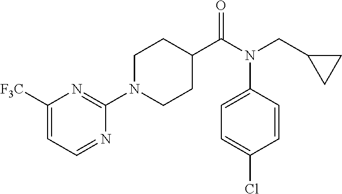 [SMILES: O=C(C1CCN(c2nccc(C(F)(F)F)n2)CC1)N(CC1CC1)c1ccc(Cl)cc1] | 0.146 | 439.0 |
| 172 | 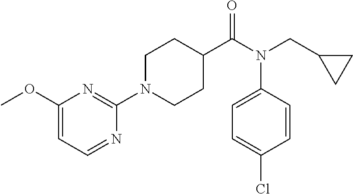 [SMILES: COc1ccnc(N2CCC(C(=O)N(CC3CC3)c3ccc(Cl)cc3)CC2)n1] | 0.017 | 401.0 |
| 173 | 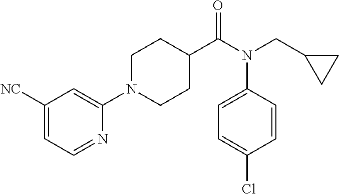 [SMILES: N#Cc1ccnc(N2CCC(C(=O)N(CC3CC3)c3ccc(Cl)cc3)CC2)c1] | 0.063 | 395.0 |
| 174 | 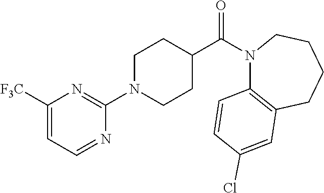 [SMILES: O=C(C1CCN(c2nccc(C(F)(F)F)n2)CC1)N1CCCCc2cc(Cl)ccc21] | 0.082 | 439.0 |
| 175 | 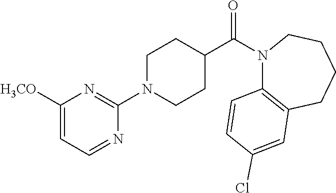 [SMILES: COc1ccnc(N2CCC(C(=O)N3CCCCc4cc(Cl)ccc43)CC2)n1] | 0.011 | 401.0 |
| 176 | 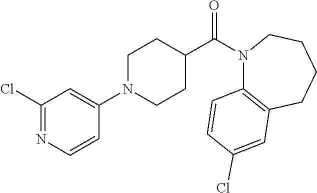 [SMILES: O=C(C1CCN(c2ccnc(Cl)c2)CC1)N1CCCCc2cc(Cl)ccc21] | 0.216 | 404.0 |
| 177 | 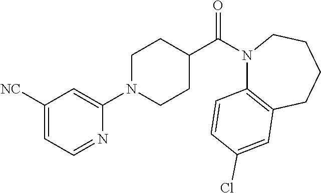 [SMILES: N#Cc1ccnc(N2CCC(C(=O)N3CCCCc4cc(Cl)ccc43)CC2)c1] | 0.028 | 395.0 |
| 178 | 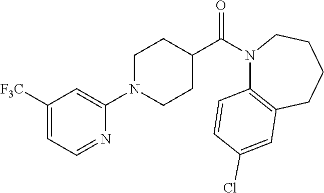 [SMILES: O=C(C1CCN(c2cc(C(F)(F)F)ccn2)CC1)N1CCCCc2cc(Cl)ccc21] | 0.017 | 438.0 |
| 179 | 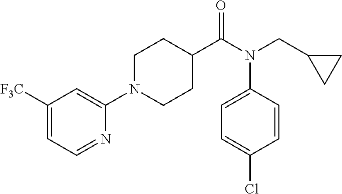 [SMILES: O=C(C1CCN(c2cc(C(F)(F)F)ccn2)CC1)N(CC1CC1)c1ccc(Cl)cc1] | 0.037 | 438.0 |
| 180 | 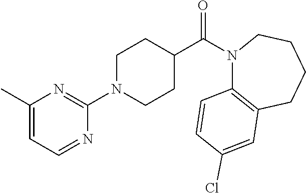 [SMILES: Cc1ccnc(N2CCC(C(=O)N3CCCCc4cc(Cl)ccc43)CC2)n1] | 0.017 | 385.0 |
| 181 | 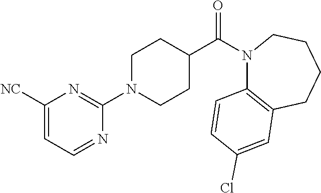 [SMILES: N#Cc1ccnc(N2CCC(C(=O)N3CCCCc4cc(Cl)ccc43)CC2)n1] | 0.081 | 396.0 |
| 182 | 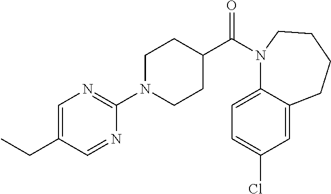 [SMILES: CCc1cnc(N2CCC(C(=O)N3CCCCc4cc(Cl)ccc43)CC2)nc1] | 0.049 | 399.0 |
| 183 | 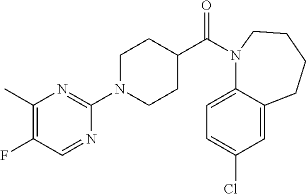 [SMILES: Cc1nc(N2CCC(C(=O)N3CCCCc4cc(Cl)ccc43)CC2)ncc1F] | 0.016 | 403.0 |
| 184 | 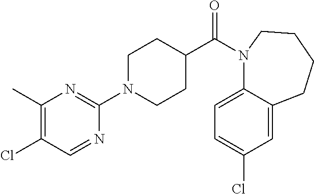 [SMILES: Cc1nc(N2CCC(C(=O)N3CCCCc4cc(Cl)ccc43)CC2)ncc1Cl] | 0.026 | 419.0 |
| 185 | 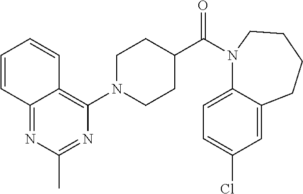 [SMILES: Cc1nc(N2CCC(C(=O)N3CCCCc4cc(Cl)ccc43)CC2)c2ccccc2n1] | 0.032 | 435.0 |
| 186 | 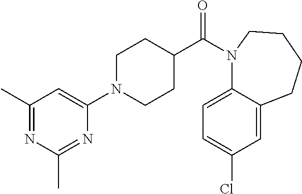 [SMILES: Cc1cc(N2CCC(C(=O)N3CCCCc4cc(Cl)ccc43)CC2)nc(C)n1] | 0.405 | 399.0 |
| 187 | 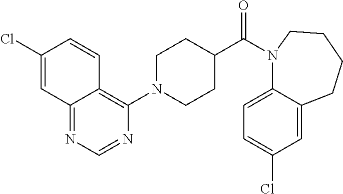 [SMILES: O=C(C1CCN(c2ncnc3cc(Cl)ccc23)CC1)N1CCCCc2cc(Cl)ccc21] | 0.066 | 455.0 |
| 188 | 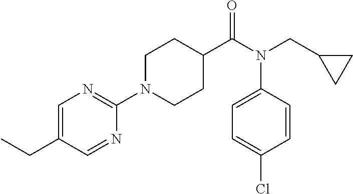 [SMILES: CCc1cnc(N2CCC(C(=O)N(CC3CC3)c3ccc(Cl)cc3)CC2)nc1] | 0.227 | 399.0 |
| 189 | 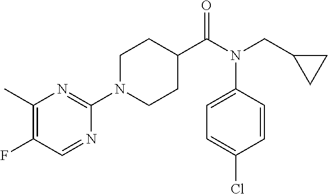 [SMILES: Cc1nc(N2CCC(C(=O)N(CC3CC3)c3ccc(Cl)cc3)CC2)ncc1F] | 0.048 | 403.0 |
| 190 | 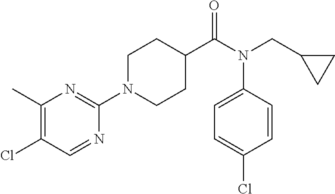 [SMILES: Cc1nc(N2CCC(C(=O)N(CC3CC3)c3ccc(Cl)cc3)CC2)ncc1Cl] | 0.120 | 419.0 |
| 191 | 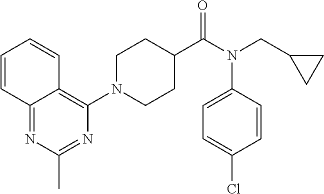 [SMILES: Cc1nc(N2CCC(C(=O)N(CC3CC3)c3ccc(Cl)cc3)CC2)c2ccccc2n1] | 0.074 | 435.0 |
| 192 | 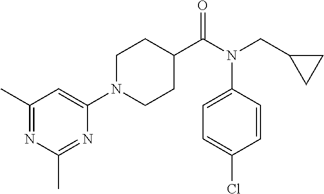 [SMILES: Cc1cc(N2CCC(C(=O)N(CC3CC3)c3ccc(Cl)cc3)CC2)nc(C)n1] | 1.247 | 399.0 |
| 193 | 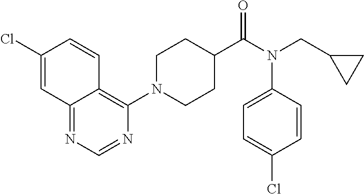 [SMILES: O=C(C1CCN(c2ncnc3cc(Cl)ccc23)CC1)N(CC1CC1)c1ccc(Cl)cc1] | 0.861 | 455.0 |
| 194 | 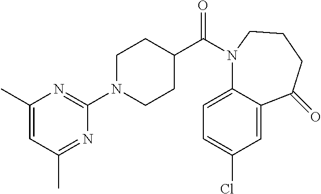 [SMILES: Cc1cc(C)nc(N2CCC(C(=O)N3CCCC(=O)c4cc(Cl)ccc43)CC2)n1] | 0.158 | 413.0 |
| 195 | 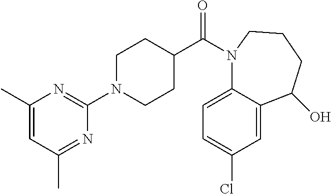 [SMILES: Cc1cc(C)nc(N2CCC(C(=O)N3CCCC(O)c4cc(Cl)ccc43)CC2)n1] | 0.026 | 415.0 |
| 196 | 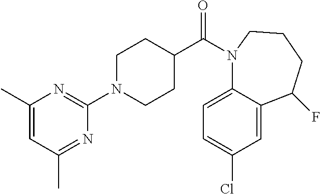 [SMILES: Cc1cc(C)nc(N2CCC(C(=O)N3CCCC(F)c4cc(Cl)ccc43)CC2)n1] | 0.084 | 417.0 |
| 197 | 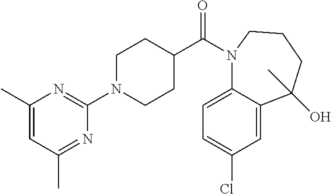 | 0.134 | 429.0 |
| 198 | 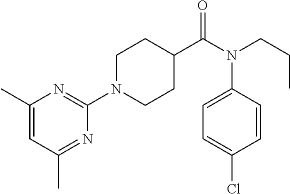 [SMILES: CCCN(C(=O)C1CCN(c2nc(C)cc(C)n2)CC1)c1ccc(Cl)cc1] | 0.284 | 387.0 |
| 199 | 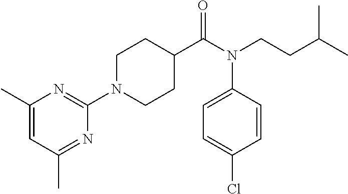 [SMILES: Cc1cc(C)nc(N2CCC(C(=O)N(CCC(C)C)c3ccc(Cl)cc3)CC2)n1] | 0.794 | 415.0 |
| 200 | 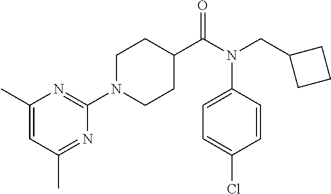 [SMILES: Cc1cc(C)nc(N2CCC(C(=O)N(CC3CCC3)c3ccc(Cl)cc3)CC2)n1] | 0.229 | 413.0 |
| 201 | 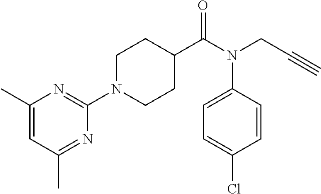 [SMILES: C#CCN(C(=O)C1CCN(c2nc(C)cc(C)n2)CC1)c1ccc(Cl)cc1] | 0.028 | 383.0 |
| 202 | 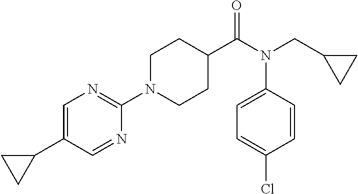 [SMILES: O=C(C1CCN(c2ncc(C3CC3)cn2)CC1)N(CC1CC1)c1ccc(Cl)cc1] | 0.348 | 411.0 |
| 203 | 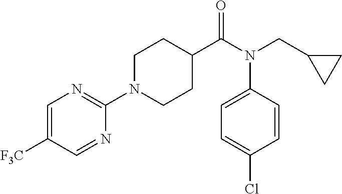 [SMILES: O=C(C1CCN(c2ncc(C(F)(F)F)cn2)CC1)N(CC1CC1)c1ccc(Cl)cc1] | 0.570 | 439.0 |
| 204 | 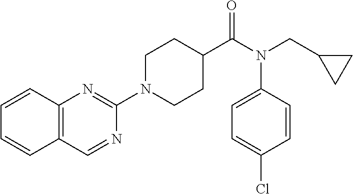 [SMILES: O=C(C1CCN(c2ncc3ccccc3n2)CC1)N(CC1CC1)c1ccc(Cl)cc1] | 0.097 | 421.0 |
| 205 | 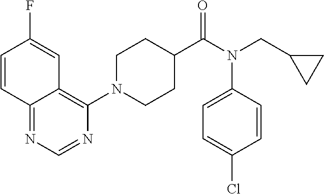 [SMILES: O=C(C1CCN(c2ncnc3ccc(F)cc23)CC1)N(CC1CC1)c1ccc(Cl)cc1] | 0.074 | 439.0 |
| 206 | 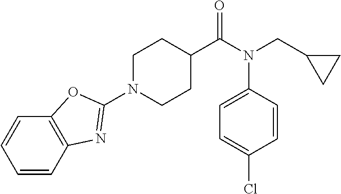 [SMILES: O=C(C1CCN(c2nc3ccccc3o2)CC1)N(CC1CC1)c1ccc(Cl)cc1] | 0.130 | 410.0 |
| 207 | 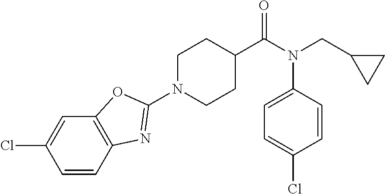 [SMILES: O=C(C1CCN(c2nc3ccc(Cl)cc3o2)CC1)N(CC1CC1)c1ccc(Cl)cc1] | 0.350 | 444.0 |
| 208 | 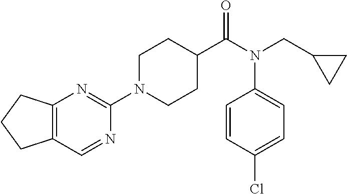 [SMILES: O=C(C1CCN(c2ncc3c(n2)CCC3)CC1)N(CC1CC1)c1ccc(Cl)cc1] | 0.033 | 411.0 |
| 209 | 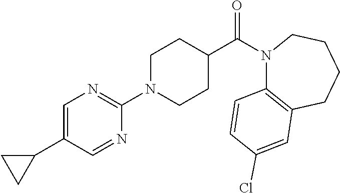 [SMILES: O=C(C1CCN(c2ncc(C3CC3)cn2)CC1)N1CCCCc2cc(Cl)ccc21] | 0.069 | 411.0 |
| 210 | 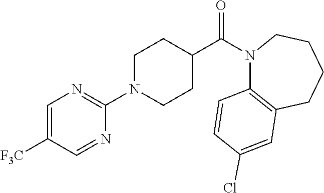 [SMILES: O=C(C1CCN(c2ncc(C(F)(F)F)cn2)CC1)N1CCCCc2cc(Cl)ccc21] | 0.089 | 439.0 |
| 211 | 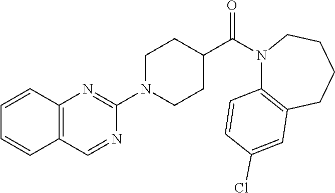 [SMILES: O=C(C1CCN(c2ncc3ccccc3n2)CC1)N1CCCCc2cc(Cl)ccc21] | 0.016 | 421.0 |
| 212 | 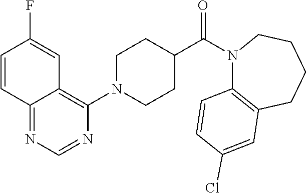 [SMILES: O=C(C1CCN(c2ncnc3ccc(F)cc23)CC1)N1CCCCc2cc(Cl)ccc21] | 0.049 | 439.0 |
| 213 | 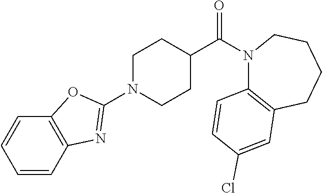 [SMILES: O=C(C1CCN(c2nc3ccccc3o2)CC1)N1CCCCc2cc(Cl)ccc21] | 0.024 | 410.0 |
| 214 | 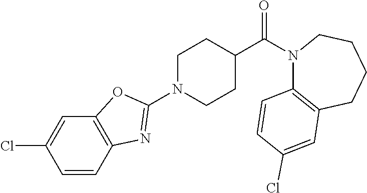 [SMILES: O=C(C1CCN(c2nc3ccc(Cl)cc3o2)CC1)N1CCCCc2cc(Cl)ccc21] | 0.045 | 444.0 |
| 215 | 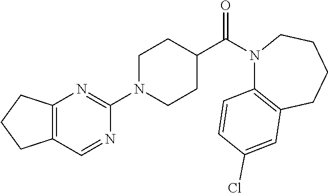 [SMILES: O=C(C1CCN(c2ncc3c(n2)CCC3)CC1)N1CCCCc2cc(Cl)ccc21] | 0.007 | 411.0 |
| 216 | 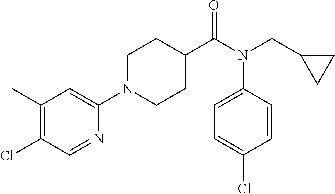 [SMILES: Cc1cc(N2CCC(C(=O)N(CC3CC3)c3ccc(Cl)cc3)CC2)ncc1Cl] | 0.041 | 418.0 |
| 217 | 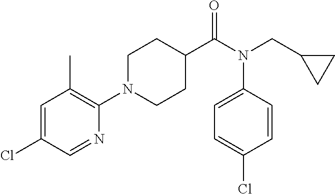 [SMILES: Cc1cc(Cl)cnc1N1CCC(C(=O)N(CC2CC2)c2ccc(Cl)cc2)CC1] | 0.050 | 418.0 |
| 218 | 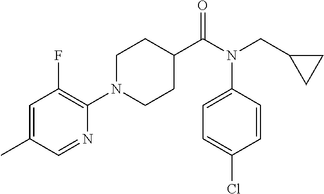 [SMILES: Cc1cnc(N2CCC(C(=O)N(CC3CC3)c3ccc(Cl)cc3)CC2)c(F)c1] | 0.020 | 402.0 |
| 219 | 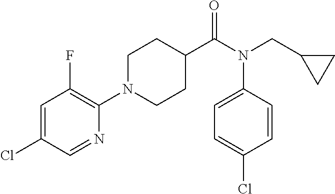 [SMILES: O=C(C1CCN(c2ncc(Cl)cc2F)CC1)N(CC1CC1)c1ccc(Cl)cc1] | 0.061 | 422.0 |
| 220 | 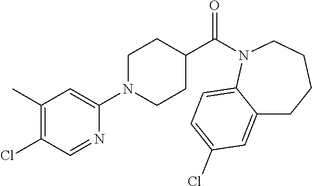 [SMILES: Cc1cc(N2CCC(C(=O)N3CCCCc4cc(Cl)ccc43)CC2)ncc1Cl] | 0.007 | 418.0 |
| 221 | 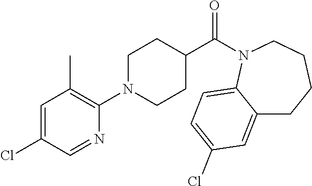 [SMILES: Cc1cc(Cl)cnc1N1CCC(C(=O)N2CCCCc3cc(Cl)ccc32)CC1] | 0.007 | 418.0 |
| 222 | 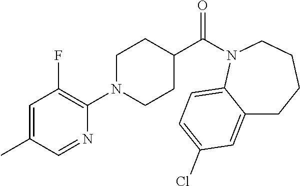 [SMILES: Cc1cnc(N2CCC(C(=O)N3CCCCc4cc(Cl)ccc43)CC2)c(F)c1] | 0.005 | 402.0 |
| 223 | 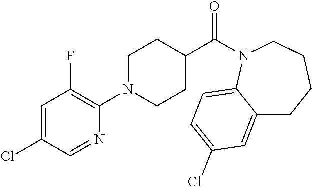 [SMILES: O=C(C1CCN(c2ncc(Cl)cc2F)CC1)N1CCCCc2cc(Cl)ccc21] | 0.019 | 424.0 |
| 224 | 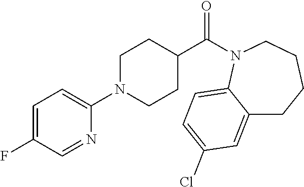 [SMILES: O=C(C1CCN(c2ccc(F)cn2)CC1)N1CCCCc2cc(Cl)ccc21] | 0.011 | 388.0 |
| 225 | 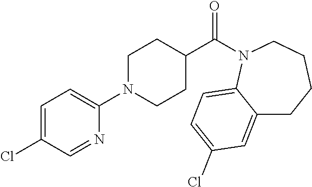 [SMILES: O=C(C1CCN(c2ccc(Cl)cn2)CC1)N1CCCCc2cc(Cl)ccc21] | 0.011 | 404.0 |
| 226 | 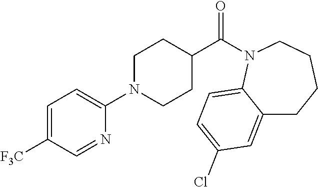 [SMILES: O=C(C1CCN(c2ccc(C(F)(F)F)cn2)CC1)N1CCCCc2cc(Cl)ccc21] | 0.020 | 438.0 |
| 227 | 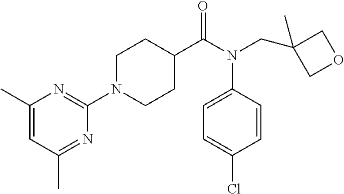 [SMILES: Cc1cc(C)nc(N2CCC(C(=O)N(CC3(C)COC3)c3ccc(Cl)cc3)CC2)n1] | 0.517 | 429.0 |
| 228 | 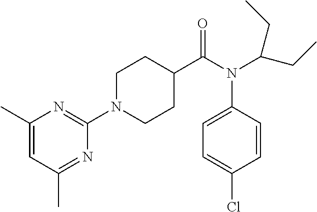 [SMILES: CCC(CC)N(C(=O)C1CCN(c2nc(C)cc(C)n2)CC1)c1ccc(Cl)cc1] | 4.938 | 415.0 |

TABLE 5
Receptor Binding and functional data for Compounds of Formula (V)
Each row represents data for a single compound. Data are shown in sequence
for compounds V: 138-143, 146-157, 159-167, 169-179, 180-246, and 254-259

| | | | OT | V1a | | |
|---|---|---|---|---|---|---|
| OT | V1a | V2 | bind. | bind. | OT | V1a |
| bind. | bind. @ | bind. | IC50 | IC50 | Antag.- | Antag.- |
| @ 10 μM | 10 μM | @ 10 μM | (nM) | (nM) | 10 μM | 10 μM |
| −18.2 | −7.9 | −8.5 | nt | nt | nt | nt |
| −6.1 | −13.5 | −11.4 | nt | nt | nt | nt |
| 79.0 | 79.4 | 72.1 | 458 | 173.3 | nt | nt |
| −17.4 | −5.1 | 11.5 | >10,000 | >10,000 | nt | nt |
| 10.4 | 57.8 | 4.3 | nt | >10,000 | 33.5 | −21.1 |
| 6.9 | 55.2 | −6.8 | nt | >10,000 | 51 | −0.9 |
| 73.1 | 94.3 | 6.8 | 1998 | 202.7 nM | 6.4 | 53.30% |
| −3.0 | 27.8 | −1.6 | nt | nt | 31.9 | −36 |
| 1.7 | 19.5 | −3.7 | nt | nt | −8 | −43.7 |
| 98.8 | 92 | 77.1 | 37.2 | 132.1 | 100.4 | 37.2 |
| 39.1 | 88.3 | −12.4 | nt | 430.7 nM | 41.4 | 57.40% |
| 42.8 | 88.7 | 9.2 | nt | 746.4 | 20.7 | −12.8 |
| 8.4 | 8.6 | 2.8 | nt | nt | 12.7 | −17.6 |
| 9.6 | 64.8 | −1.1 | nt | 3715 | 4.8 | 4.5 |
| −2.3 | 96.6 | −6.1 | nt | 216.9 nM | 43 | 100.30% |
| 3.3 | 94.5 | −10.3 | nt | 362.9 nM | 38.2 | 96.10% |
| −2.2 | 88.8 | −17.1 | nt | 881.7 nM | 44.6 | 100.30% |
| 5.7 | 94.2 | −9.3 | nt | 271.9 nM | 23.9 | 100.30% |
| 1.5 | 97.8 | −10.1 | nt | 16.8 nM | 6.4 | 100.30% |
| 6.6 | 99.1 | −12.8 | nt | 36.7 nM | 94 | 100.30% |
| 9.6 | 80.2 | −2.1 | nt | >10,000 | 44.6 | −15.2 |
| 11.0 | 97.7 | −1.5 | nt | 251.7 nM | 3.2 | 99.10% |
| 23.2 | 92.5 | −0.6 | nt | 4606 nM | 60.6 | 94.90% |
| 37.7 | 98 | 14.3 | nt | 416.9 nM | 35.1 | 100.30% |
| 22.7 | 100.9 | 15.4 | nt | 649.2 nM | 39.8 | 100.30% |
| 37.6 | 96.8 | 19.5 | nt | 230.3 nM | 9.6 | 100.90% |
| 22.5 | 91.4 | −8.5 | nt | 2654 | 11.2 | 30.6 |
| 23.1 | 58.6 | −12.6 | nt | >10,000 | 62.2 | −16.4 |
| 22.1 | 97.1 | −7.3 | nt | 293.6 | 49.4 | 99.10% |
| 25.1 | 94.7 | 2 | nt | 1387 | −14.3 | 43.7 |
| 18.2 | 78.3 | 0.1 | nt | >10,000 | 33.5 | −18.2 |
| 10.4 | 92.9 | 14 | nt | 2037 | 31.9 | 65.80% |
| 2.0 | 11.8 | −3.4 | nt | nt | 25.5 | −24.1 |
| 1.1 | 92.2 | −9.9 | nt | 1309 | 12.7 | 20.5 |
| −2.5 | 86.4 | −9.5 | nt | 2331 | −4.8 | 11 |
| 7.7 | 55 | −8.5 | nt | >10,000 | 14.3 | −29.5 |
| 24.9 | 65 | 17.2 | nt | >10,000 | 0.59 | −17 |
| 99.1 | 96.7 | 77.3 | 236.1 | 164.6 | 84.5 | 100.3 |
| nt | nt | nt | nt | nt | 31.1 | 37.9 |
| nt | nt | nt | nt | nt | 35.1 | −70.7 |
| nt | nt | nt | nt | 176 | 66.9 | 101.40% |
| nt | nt | nt | nt | 411 | 15.2 | 95.50% |
| nt | nt | nt | nt | >10,000 | 37.1 | 43.1 |
| nt | nt | nt | nt | 243 | 59 | 89.60% |
| nt | nt | nt | nt | 234 | 3.2 | 61.50% |
| nt | nt | nt | nt | >10,000 | −12.7 | 4.7 |
| nt | nt | nt | nt | 553 | 43 | 77.10% |
| nt | nt | nt | nt | 165 | 35.1 | 100.70% |
| nt | nt | nt | nt | >10,000 | 7.2 | −44.1 |
| nt | nt | nt | nt | 92 | 41 | 100.70% |
| nt | nt | nt | nt | 16 | −2.7 | 101.40% |
| nt | nt | nt | nt | 6.4 | 1.2 | 97% |
| nt | nt | nt | nt | 854 | 80.8 | 99.20% |
| nt | nt | nt | nt | 1,905 | −4.7 | −1.2 |
| nt | nt | nt | nt | 184 | 39.1 | 100.70% |
| nt | nt | nt | nt | 874 | 13.2 | 50.50% |
| nt | nt | nt | nt | >10,000 | 15.2 | −8.6 |
| nt | nt | nt | nt | >10,000 | −16.7 | −4.2 |
| nt | nt | nt | nt | 486 | 3.2 | 74.10% |
| nt | nt | nt | nt | 278 | −28.6 | 93.30% |
| nt | nt | nt | nt | 146 | −42.5 | 98.50% |
| nt | nt | nt | nt | 197 | 1.2 | 96.30% |
| nt | nt | nt | nt | 986 | 37.1 | 69.70% |
| nt | nt | nt | nt | nt | 19.2 | −35.2 |
| nt | nt | nt | nt | nt | 31.1 | −51.5 |
| nt | nt | nt | nt | nt | 3.2 | 6.9 |
| nt | nt | nt | nt | nt | −2.7 | −52.9 |
| nt | nt | nt | nt | 1,657 | −0.7 | 33.5 |
| nt | nt | nt | nt | 184 | 21.1 | 100.70% |
| 6.2% | 75.10% | nt | nt | 75 | 37.1 | 100% |
| 13.5% | 83.90% | nt | nt | 297 | 29.1 | 54.2 |
| 8.0% | 93.60% | nt | nt | 244 | 43 | 101.40% |
| 3.3% | 98.60% | nt | nt | 30 | 27.1 | 101.40% |
| −5.8% | 33.70% | nt | nt | nt | 3.2 | −13 |
| 11.0% | 81.90% | nt | nt | 130 | 33.1 | 100.70% |
| −3.9% | 66.60% | nt | nt | 500 | 19.2 | 79.30% |
| −9.7% | 77.10% | nt | nt | 15 | 59 | 100.70% |
| −4.6% | 66.80% | nt | nt | 175 | 49 | 99.20% |
| 0.2% | 64.80% | nt | nt | 210 | 21.1 | 83.70% |
| 2.3% | 52.60% | nt | nt | 655 | 9.2 | 6.9 |
| 2.3% | 95.70% | nt | nt | 557 | 9.2 | 91.80% |
| −13.2% | 15.20% | nt | nt | nt | −24.6 | −58.8 |
| 5.8% | 69.30% | nt | nt | 167 | 9.2 | 69.7 |
| −5.9% | 84.50% | nt | nt | 50 | 21.1 | 100.70% |
| −11.2% | 43.80% | nt | nt | nt | −12.7 | −15.3 |
| −11.6% | 50% | nt | nt | >10,000 | 7.2 | 44.6 |
| −7.4% | 65.30% | nt | nt | 482 | 47 | 76.3 |
| −0.6% | 76% | nt | nt | 79 | 35.1 | 63 |
| 6.9% | 85% | nt | nt | 127 | 41 | 100% |
| 4.7% | 93.30% | nt | nt | 35 | 15.2 | 100.70% |
| 14.0% | 96.90% | nt | nt | 408 | −28.6 | 90.30% |
| 34.0% | 104.70% | nt | nt | 119 | 49 | 100% |
| 15.5% | 64.50% | nt | nt | 1,393 | −22.6 | 35 |
| 11.0% | 54% | nt | nt | 1,472 | 53 | 56.4 |
| 8.3% | 38.70% | nt | nt | nt | 43 | 26.8 |
| 7.4% | 70.70% | nt | nt | 160 | 21.1 | 10070.00% |
| 9.7% | 23.20% | nt | nt | nt | 45 | 34.2 |
| 5.9% | 4.10% | nt | nt | nt | 27.1 | −30.8 |
| 21.0% | 42.20% | nt | nt | nt | 21.1 | 36.4 |
| 7.8% | 10.10% | nt | nt | nt | 0.988 | 54.9 |
| 8.4% | 19.10% | nt | nt | nt | 13.2 | −8.6 |
| 13.4% | 12.50% | nt | nt | nt | 0.868 | 43.1 |
| 23.3% | 19.10% | nt | nt | nt | 86.8 | 51.9 |
| nt | nt | nt | nt | nt | −76.4 | −38.2 |
| nt | nt | nt | nt | nt | 45.1 | 0.9 |
| nt | nt | nt | nt | nt | −0.7 | 1.2 |
| nt | nt | nt | nt | nt | −14.1 | 0.2 |
| nt | nt | nt | nt | nt | 16.5 | 1.7 |
| nt | nt | nt | nt | nt | 60.4 | 1.4 |
| nt | nt | nt | nt | nt | 41.3 | 0.2 |
| nt | nt | nt | nt | nt | 20.3 | −0.3 |
| nt | nt | nt | nt | nt | −2.6 | −0.1 |
| nt | nt | nt | nt | nt | −10.3 | −0.1 |
| nt | nt | nt | nt | nt | −54.2 | −0.1 |
| nt | nt | nt | nt | nt | −29.4 | −0.1 |
| nt | nt | nt | nt | nt | −38.9 | −0.1 |
| nt | nt | nt | nt | nt | 20.3 | −0.3 |
| nt | nt | nt | nt | nt | 20.3 | −0.3 |

TABLE 6
Biodata for Additional Compounds of Formula (V)

| | | V1a-R | |
|---|---|---|---|
| Cpd. | | IC₅₀ | Observed |
| # | Structure | (μM) | LC-MS |
| V:292 | 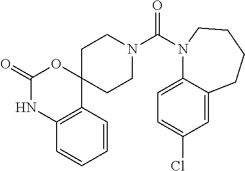 [SMILES: O=C1Nc2ccccc2C2(CCN(C(=O)N3CCCCc4cc(Cl)ccc43)CC2)O1] | 0.305 | 426.0 |
| V:293 | 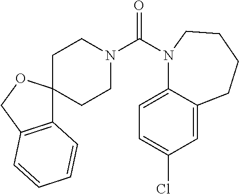 [SMILES: O=C(N1CCC2(CC1)OCc1ccccc12)N1CCCCc2cc(Cl)ccc21] | 0.078 | 397.0 |

### Methods of Synthesis of Compounds of the Invention

Compounds of formulas (I) and (v) can be prepared according to the schemes and synthetic procedures provided below in conjunction with knowledge of the person of ordinary skill and the known disclosures of the chemical literature. Specific exemplary synthetic procedures are provided in the Examples, below.

The following abbreviations are used throughout this document:
anhyd. anhydrous
Boc Tert-butoxycarbonyl
Cbz Carbobcnzyloxy
CDI Carbonyl diimidazole
DBU Diazabicycloundecane
DCM Dichloromethane
DIPEA, ⁱPr₂EtN N,N-Diisopropylethylamine
DMAP 4-(N,N-dimethylamino)pyridine
DMF N,N-Dimethylformamide
DMSO Dimethylsulfoxide
EDAC 1-Ethyl-3-(3-dimethylaminopropyl)carbodiimide hydrochloride
eq Equivalents
Et₂O Diethyl ether
EtOAc Ethyl acetate
h Hours
HATU O-(7-Azabenzotriazole-1-yl)-N, N,N′N′-tetramethyluronium hexafluorophosphate
HCl Hydrochloric acid
HOAT Hydroxyazabenztriazole
HOBT Hydroxybenzotriazole
LiHDMS Lithium hexamethyldisilazide
LiOH Lithium hydroxide
mg Milligrams
min Minutes
mL Milliliters
μL Microliters
mmole Millimoles
MS Mass spectroscopy
MeOH Methanol
Mw, MV microwave heating
NaBH₃CN Sodium cyanoborohydride
NEt3 Triethylamine
NaH Sodium hydride
NaIO₄ Sodium periodate
NMM N-Methylmorpholine
rb Round-bottom
r.t. Room temperature
sat. Saturated
TEA Triethylamine
TFA Trifluoroacetic acid
THF Tetrahydrofuran

### General Schemes and Procedures for Synthesis of Compounds of Formula (I)

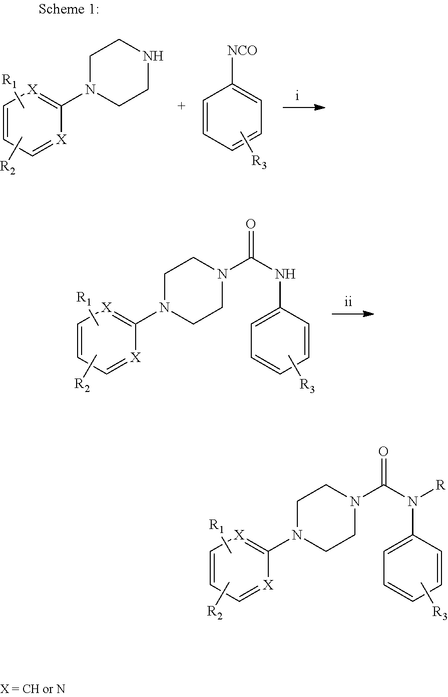

Reagents and conditions: a) CH₂Cl₂, NEt₃, r.t., 3-4 hrs, (60-80%); b) RX, NaH, DMF, r.t., overnight (40-72%).

### General Procedure for the Preparation of Urea Derivatives (Step i):

To a stirred solution of aryl piperazine (1 equiv.), in 3 mL of dry CH₂Cl₂, was added an appropriate phenyl isocyanate in 1 mL of dry CH₂Cl₂, and the mixture was stirred at room temperature for 3-4 hrs. The mixture was diluted with CH₂Cl₂, washed with H₂O and saturated brine solution. The organic layer was dried over anhydrous Na₂SO₄, solvents removed in vacuo and the crude product was purified by column chromatography to obtain desired products in 60-80% yields.

### General Procedure for the Alkylation of Urea Derivatives (Step ii):

To a stirred solution of N,4-diaryl or heteroaryl piperazine-1-carboxamide (0.5 mmol), in 2 mL of dry DMF, was added NaH (60% suspension in oil) and the mixture was stirred for 10 min. Then, alkyl halide (0.65 mmol) was added and the mixture was stirred overnight (in case of alkyl bromides or chlorides 0.1 equiv. of NaI was added). The reaction mixture was partitioned between ethyl acetate and water, organic layer was separated, washed with H₂O, saturated brine solution, dried over anhydrous Na₂SO₄, solvents removed in vacuo and the crude was purified by column chromatography to obtain the desired products in 40-72% yields.

### Preparation of Compounds 7, 8 and 24

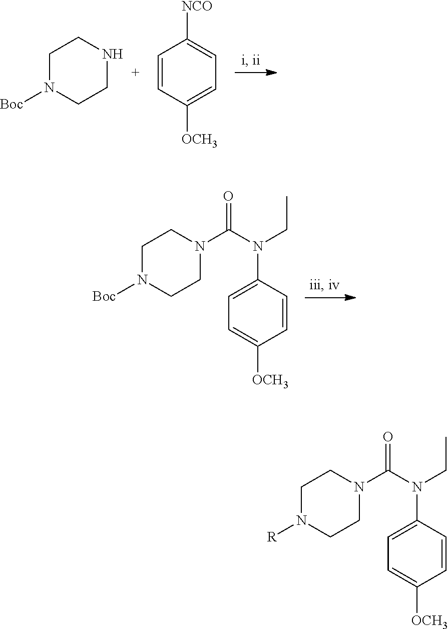

Reagents and conditions: i) CH₂Cl₂, NEt₃, r.t., 3-4 hrs; ii) C₂H₅I, NaH, DMF, r.t., overnight; iii) TFA/CH₂Cl₂, 3 hrs.; iv) acid chloride, CH₂Cl₂, NEt₃, overnight or benzyl bromide, K₂CO₃, DMF, overnight.

### Step i: Preparation of tert-butyl 4-((4-methoxyphenyl)carbamoyl)piperazine-1-carboxylate

The title compound was prepared following the general procedure (above) for the preparation of urea derivatives.

### Step ii: Preparation of tert-butyl 4-(ethyl(4-methoxyphenyl)carbamoyl)piperazine-1-carboxylate

The title compound was prepared following the general procedure (above) for the alkylation of urea derivatives.

### Step iii: Deprotection of Boc group

To a stirred solution of text-butyl 4-(ethyl(4-methoxyphenyl)carbamoyl)piperazine-1-carboxylate (2.75 mmol) in 8 mL of dry CH₂Cl₂, was added 8 mL of TFA at 0° C. and the mixture was brought to room temperature (r.t.), and stirred for 2 hrs at r.t. The solvents were concentrated in vacuo, the residue azeotroped with toluene (4×) and dried under high vacuum to obtain the desired product in quantitative yield, which was used for next step without purification.

### Step iv: Preparation of Compounds 7 and 24

To a stirred solution of N-ethyl-N-(4-methoxyphenyl)piperazine-1-carboxamide (1 equiv.) in dry CH₂Cl₂, was added NEt₃ at 0° C. followed by the addition of corresponding acid chloride (1.2 equiv.) in dry CH₂Cl₂ and the mixture was stirred at r.t., overnight. The mixture was diluted with CH₂Cl₂, washed with 1N HCl, H₂O and saturated NaHCO₃ solution. The organic layer was dried over anhydrous Na₂SO₄, solvents removed in vacuo and the crude product was purified by column chromatography to obtain desired products.

### Step iv: Preparation of Compound 8

To a stirred solution of N-ethyl-N-(4-methoxyphenyl)piperazine-1-carboxamide (0.57 mmol) in dry DMF, was added K₂CO₃ followed by the addition of benzyl bromide (0.684) and the mixture was stirred at r.t., overnight. The mixture was diluted with EtOAc, washed with H₂O and sat. brine solution. The organic layer was dried over anhyd.Na₂SO₄, solvents removed in vacuo and the crude product was purified by column chromatography to obtain the desired product.

### Preparation of Compound 23

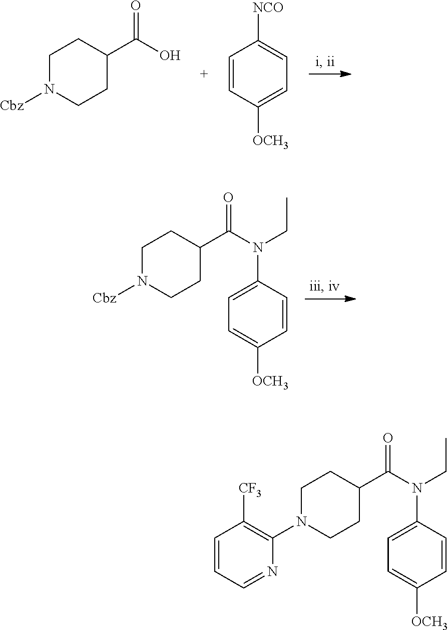

Reagents and conditions: i) CH₂Cl₂, HATU, NEt₃, r.t., overnight, (78%); ii) iodoethane, NaH, DMF, r.t., overnight (88%); iii) 10% Pd—C, H₂, MeOH, 3 hrs, quant.; iv) 2-chloro-3-trifluormethyl pyridine, DIEA, DMF, microwave, 180° C., 30 min.

### Step i: Preparation of benzyl-4-(4-methoxyphenyl)carbamoyl)piperidine-1-carboxylate

To a stirred solution of 1-(benzyloxycarbonyl) piperidine-4-carboxylic acid (2 mmol), in 6 mL of dry CH₂Cl₂, was added HATU (2 mmol) followed by the addition of NEt₃ (4 mmol) and the mixture was stirred for 5 min. Then, p-anisidine (2.5 mmol), in 2 mL of dry CH₂Cl₂, was added and the mixture was stirred under N₂, at r.t., overnight. The mixture was diluted with CH₂Cl₂, washed sequentially with 2N HCl, H₂O and saturated NaHCO₃ solution. The organic layer was dried over anhydrous Na₂SO₄, solvents removed in vacuo and the crude product was purified by column chromatography to obtain the desired product in 78% yield.

### Step ii: Preparation of benzyl-4-(ethyl-4-methoxyphenyl)carbamoyl)piperidine-1-carboxylate

To a stirred solution of benzyl-4-(4-methoxyphenyl)carbamoyl)piperidine-1-carboxylate (1.56 mmol), in 2 mL of dry DMF, was added NaH (60% suspension in oil) and the mixture was stirred for 10 min. Then, alkyl halide (1.95 mmol) was added and the mixture was stirred overnight. The reaction mixture was partitioned between ethyl acetate and water, organic layer was separated, washed with H₂O, saturated brine solution, dried over anhydrous Na₂SO₄, solvents removed in vacuo and the crude was purified by column chromatography to obtain the desired product in 88% yield.

### Step iii: Deprotection of Cbz Group

A sample of benzyl-4-(ethyl-4-methoxyphenyl)carbamoyl)piperidine-1-carboxylate (550 mg), 10% Pd—C (110 mg), in 6 mL of MeOH, was stirred under H₂ atmosphere until the starting material was disappeared completely (˜3 hrs). The mixture was filtered through celite, washed with MeOH, filtrates concentrated in vacuo and the crude product was dried under high vacuo which was used for next step without purification.

### Step iv: Preparation of N-ethyl-N-(4-methoxyphenyl)-1-(3-trifluormethyl)pyridine-2-yl)piperidine-4-carboxamide

A sample of N-ethyl-N-(4-methoxyphenyl)piperidine-4-carboxamide (175 mg, 0.75 mmol), and 2-chloro-3-trifluormethyl pyridine was taken in 2-5 mL of microwave vial and was added 3 mL of dry DMF followed by the addition of DIEA (0.26 mL, 1.5 mmol). The reaction mixture was heated to 180° C. for 30 min in microwave. The reaction mixture was diluted with EtOAc, washed with H₂O and sat. brine solution. The organic layer was dried over anhyd.Na₂SO₄, solvents removed in vacuo and the crude product was purified by column chromatography to obtain the desired product compound 23.

### Preparation of Compounds 33 and 34

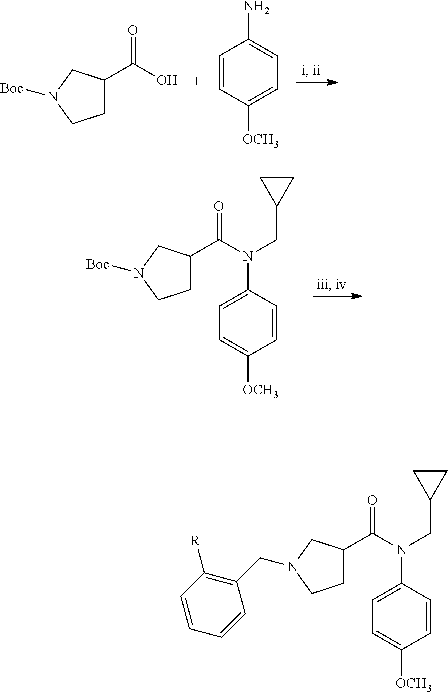

Reagents and conditions: i) CH₂Cl₂, HATU, NEt₃, r.t., overnight, (78%); ii) cyclopropylmethyl bromide, NaH, DMF, r.t., overnight (88%); iii) TFA/CH₂Cl₂, 3 hrs; iv) benzyl bromide, K₂CO₃, DMF, overnight

### Step i: Preparation of tert-butyl-3-(4-methoxyphenyl)carbamoyl)pyrrolidine-1-carboxylate

The title compound was prepared, following the procedure used for the preparation of benzyl-4-(4-methoxyphenyl)carbamoyl)piperidine-1-carboxylate, from 1-(tert-butoxycarbonyl)pyrrolidine-3-carboxylic acid and p-anisidine.

### Step ii: Preparation of tert-butyl-3-((cyclopropylmethyl)(4-methoxyphenyl)carbamoyl)pyrrolidine-1-carboxylate

The title compound was prepared following the procedure described for the preparation of benzyl-4-(ethyl-4-methoxyphenyl)carbamoyl)piperidine-1-carboxylate.

### Step iii: Preparation of N-(cyclopropylmethyl)-N-(4-methoxyphenyl)pyrrolidine-1-carboxamide

The title compound was prepared following the procedure described for the preparation of N-ethyl-N-(4-methoxyphenyl)piperazine-1-carboxamide.

### Step iv: Preparation of Compounds 33 and 34

The title compounds were prepared following the procedure described for the preparation of compound 7.

### Preparation of benzimidazolyl-2-piperazines and -piperidines, e.g., compounds 37-39, 41-49, 51-52, 53-56 and 59-77

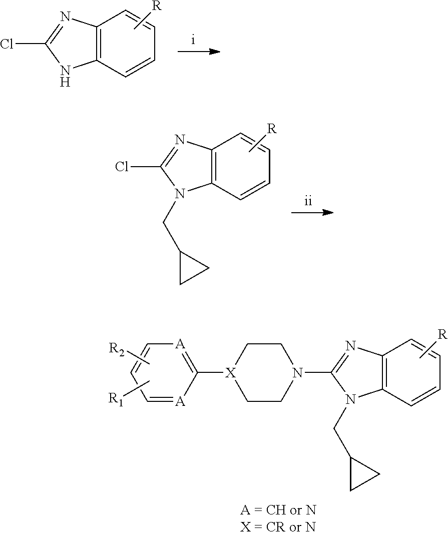

Reagents and conditions: i) Cyclopropylmethyl bromide, K₂CO₃, CH₃CN, reflux, overnight, 81%; ii) Aryl or heteroaryl piperazines, DIEA, DMF, microwave, 185° C., 3 hrs (11-30%).

### Step i

To a stirred solution of commercially available 2-chloro benzimidazole (13.1 mmol), in 20 mL of dry CH₃CN, was added K₂CO₃ (13.1 mmol) followed by the addition of cyclopropylmethyl bromide (13.1 mmol) and the mixture was heated to reflux overnight. The mixture was cooled to r.t., solvents concentrated in vacuo, the residue partitioned between ethyl acetate and water, organic layer was separated, washed with H₂O, saturated brine solution, dried over anhydrous Na₂SO₄, solvents removed in vacuo to obtain the pure product in 81% yield which was used for next step without purification.

### Step ii

A sample of 2-chloro-1-(cyclopropylmethyl)-1H-benzimidazole (1 equiv.) and aryl piperazine/piperidine or heteroaryl piperazine (1.2 equiv.) was taken in microwave vial and was added dry DMF followed by the addition of DIEA (2 equiv.). The reaction mixture was heated to 185° C. for 3 hrs in microwave. The reaction mixture was diluted with EtOAc, washed with H₂O and saturated brine solution. The organic layer was dried over anhydrous Na₂SO₄, and the solvents were removed in vacuo and the crude product was purified by column chromatography to obtain the desired products in 11-30% yields.

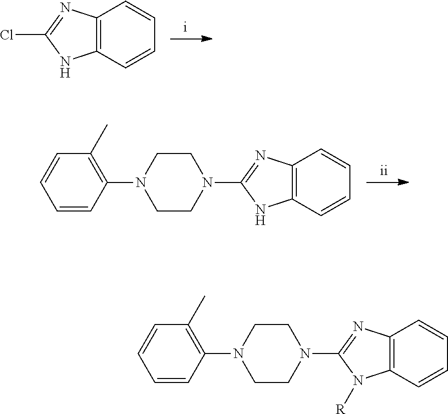

Reagents and conditions: i) 1-(2-Methylphenyl)piperazine, NEt₃, DMF, microwave, 185° C., 1 hr, 65%; ii) R—X, NaH, DMF, r.t, overnight, NaI when X═Cl or Br. (50-86%)

### Step i

A sample of 2-chloro-1H-benzimidazole (0.762 g, 5 mmol) and 1-(2-methylphenyl)piperazine (1.0 g, 5.67 mmol) was taken in 10-20 mL of microwave vial and was added 12 mL of dry DMF followed by the addition of NEt₃ (1.39 mL, 10 mmol). The reaction mixture was heated to 185° C. for 1 hr in microwave. The reaction mixture was added to 150 mL of ice-cold water, the resulting solids collected by filtration, washed with H2O, EtOAc and dried under high vacuum to obtain 1.1 g of pure product, confirmed by LC-MS.

### Step ii

To a stirred solution of 2-(4-(o-tolyl)piperazin-1-yl)-1H-benzimidazole (1 equiv.), in 1 mL of dry DMF (dissolves on warming), was added NaH (60% suspension in oil, 1.2 equiv.) and the mixture was stirred for 10 min. Then, alkyl or benzyl halide (1.2 equiv.) was added and the mixture was stirred overnight (in case of alkyl or benzyl bromides or chlorides 0.1 equiv. of NaI was added). The reaction mixture was partitioned between ethyl acetate and water, organic layer was separated, washed with H₂O, saturated brine solution, dried over anhydrous Na₂SO₄, solvents removed in vacuo and the crude was purified by column chromatography to obtain the desired products in 50-86% yields.

### Preparation of Compounds 35, 36, 57, and 58

These compounds are prepared following the procedure described for the preparation of 2-(4-(o-tolyl) piperazin-1-yl)-1H-benzimidazole as shown in the scheme directly above. The crude products, after work up, are purified by column chromatography to obtain pure final products.

### Preparation of Compound 12

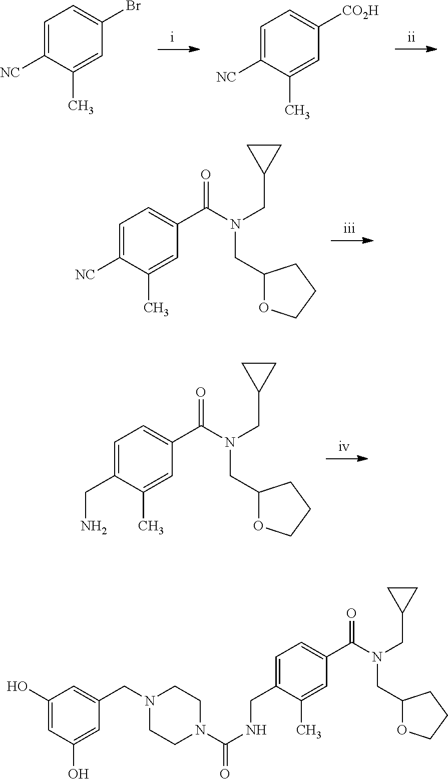

Reagents and conditions: (i) n-BuLi, THF, −78° C., 1 hr, then CO₂; 60%; (ii) amine, HATU, NEt₃, CH₂Cl₂, overnight, 75%; (iii) NaBH₄, CoCl₂.6H₂O, MeOH, 1 hr, 79%; (iv) CDI, DMF, DIEA, 1 hr, then 5-(piperazin-1-ylmethyl)benzene-1,3-diol, r.t., overnight, 49%.

### Step i

The compound was prepared following the literature procedure provided in published PCT application WO 2005/023812, incorporated by reference herein.

### Step ii

To a stirred solution of 4-cyano-3-methylbenzoic acid (1 mmol) in DMF was added HATU (1 mmol), followed by the addition of NEt₃ (2 mmol) and the mixture was stirred for 5 min. Then, corresponding amine (1.2 mmol), in dry DMF, was added and the mixture was stirred under N₂, at r.t., overnight. The mixture was poured on water, extracted with CH₂Cl₂, combined organic layers were washed with brine, dried over anhydrous Na₂SO₄ and solvents removed in vacuo and the crude product was purified by column chromatography to obtain the desired product in 75% yield.

### Step iii

Cobalt (II) chloride hexahydrate (2 equiv.) was added to an ice-cold solution of 4-cyano-N-(cyclopropylmethyl)-3-methyl-N-((tetrahydrofuran-2-yl)methyl)benzamide (1 equiv.) in methanol (35 ml). Sodium borohydride (10 equiv.) was added portionwise at 0° C. and the mixture was stirred at room temperature for 1 hr, then quenched with IM KHSO₄ and concentrated in vacuo. The aqueous residue was diluted with 1M KHSO₄ (40 ml), filtered through celite, the filtrate washed with diethyl ether (2×) then basified with 2M NaOH and extracted with CH₂Cl₂. The organic layer was dried over Na₂SO₄ and concentrated in vacuo to give the desired product in 79% yield, which was used for next step without purification.

### Step iv

1,1′-Carbonyldiimidazole (1 equiv.) and DIEA (2 equiv.) were added to a solution of 4-(aminomethyl)-N-(cyclopropylmethyl)-3-methyl-N-((tetrahydrofuran-2-yl)methyl)benzamide (1 equiv.) in DMF and stirred for 1 h. 5-Piperazin-1-ylmethyl-benzene-1,3-diol dihydrochloride (1.1 equiv.), prepared according to the procedure of WO 2005/023812, and DIEA (2 equiv.) were added and the mixture was stirred for 18 h. The mixture was reduced in vacuo and the residue was taken up in EtOAc and a minimum amount of methanol. The mixture was washed with brine, reduced in vacuo and taken up again in EtOAc and a minimum amount of methanol. The mixture was washed with sat. NaHCO3 and brine, dried and reduced in vacuo. The residue was purified twice by column chromatography to obtain the target compound in 49% yield.

### Preparation of Compound 13

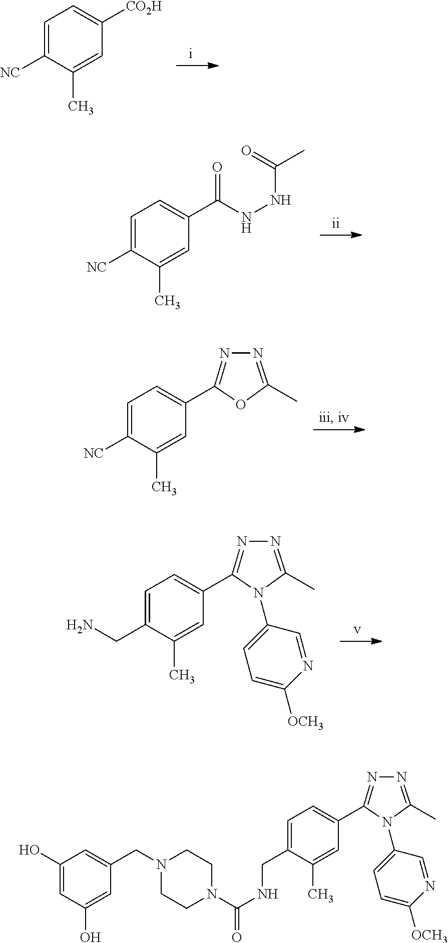 [SMILES: Cc1cc(C(=O)O)ccc1C#N ; CC(=O)NNC(=O)c1ccc(C#N)c(C)c1 ; Cc1nnc(-c2ccc(C#N)c(C)c2)o1 ; COc1ccc(-n2c(C)nnc2-c2ccc(CN)c(C)c2)cn1 ; COc1ccc(-n2c(C)nnc2-c2ccc(CNC(=O)N3CCN(Cc4cc(O)cc(O)c4)CC3)c(C)c2)cn1]

Reagents and conditions: (i) Acetic hydrazide, HATU, DIEA, CH₂Cl₂, overnight, 62%; (ii) Burgess reagent, THF, sealed tube, 78° C., overnight, 79%; (iii) 5-amino-2-methoxy pyridine, TFA, toluene, sealed tube, 120° C., overnight, 31%; (iv) NaBH₄, CoCl₂.6H₂O, MeOH, 1 hr, 86%; (e) CDI, DMF, DIEA, 1 hr, then 5-(piperazin-1-ylmethyl)benzene-1,3-diol, r.t., overnight, 57%.

### Step i

To a stirred solution of 4-cyano-3-methylbenzoic acid (1 mmol) in 4 ml of dry CH₂Cl₂ was added HATU (1 mmol), followed by the addition of DIEA (2 mmol) and the mixture was stirred for 5 min. Then, acetic hydrazide (1.2 mmol) was added and the mixture was stirred under N₂, at r.t., overnight. The solvents were concentrated in vacuo and the residue was purified by column chromatography to obtain the desired product in 76% yield.

### Step ii

A sample of N′-acetyl-4-cyano-3-methylbenzohydrazide (1 mmol) and burgess reagent (4 mmol) was taken in 4 mL of dry THF and the mixture was heated to 78° C. in a sealed tube overnight. The reaction mixture was cooled to r.t, absorbed on silica gel and purified by column chromatography to obtain the desired product in 79% yield.

### Step iii

A sample of 2-methyl-4-(5-methyl-1,3,4-oxadiazol-2-yl)benzonitrile (1 mmol) and 5-amino-2-methoxy pyridine (1.2 mmol) was taken in 6 ml of toluene, added TFA (2 mmol) and the mixture was heated to 120° C. overnight in a sealed tube. The mixture was cooled to r.t., solvents removed in vacuo, the residue taken in CH₂Cl₂, washed with sat.NaHCO₃ solution, H₂O and sat. brine solution. The organic layer was dried over anhyd.Na₂SO₄, solvents removed in vacuo and the crude product was purified by column chromatography to obtain the desired product in 31% yield.

### Step iv

The reaction was carried out as described for the preparation of compound 12 (step iii), to obtain the desired product in 86% yield.

### Step v

The reaction was carried out as described for the preparation of compound 12 (step iv), to obtain the final product in 57% yield.

### General Procedure for Preparation of the Compounds 78-93, 109-121, 162-165, 174-178, 180-187, 209-215 and 220-226

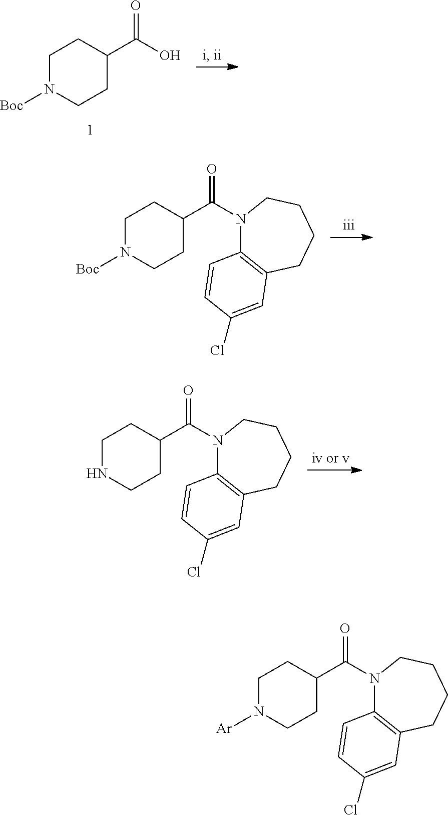

Reagents and conditions: (i) 1 (1 equiv.), oxalyl chloride (1.2 equiv., 2M solution in CH₂Cl₂), NEt₃ (1.2 equiv.), CH₂Cl₂, 0° C., overnight; (ii) 7-chloro-2,3,4,5-tetrahydro-1H-benzo[b]azepine (1.05 equiv.), CH₂Cl₂, 0° C. to r.t., 2 h; (iii) TFA/CH₂Cl₂ (1:1), 0° C. to r.t., 1 h; (iv) 2-halo pyridine or 2-halo pyrimidines c, DIEA, ⁱPrOH, 150° C., microwave, 2-6 h (v) R—COOH, EDCI, HOBt, CH₂Cl₂, rt, 16 h or R—COCl, NEt₃, CH₂Cl₂, 2 h.
i) A solution of piperidine-1,4-dicarboxylic acid mono-text-butyl ester 1 and triethylamine in CH₂Cl₂ were cooled to 0° C. under N₂. Oxalyl chloride was added, followed by few drops of DMF, and the mixture was stirred at 0° C. for 2 hours. The solvents were concentrated under vacuo, azeotroped twice with toluene and the crude acid chloride was used for the next step.
ii) To a stirred solution of 7-chloro-2,3,4,5-tetrahydro-1H-benzo[b]azepine in CH₂Cl₂, NEt₃ was added, followed by the drop-wise addition of the acid chloride (obtained from above) in CH₂Cl₂ at 0° C. The reaction mixture was stirred for 2 hours, diluted with CH₂Cl₂ and washed sequentially (2×) with H₂O, saturated NaHCO₃ and brine solution. The organic layer was separated, dried over anhydrous Na₂SO₄, concentrated under vacuo and the crude product was purified by column chromatography using ethyl acetate/hexanes.
iii) To a stirred solution of tert-butyl 4-(7-chloro-2,3,4,5-tetrahydro-1H-benzo[b]azepine-1-carbonyl)piperidine-1-carboxylate obtained above in CH₂Cl₂ was added TFA at 0° C. and the reaction mixture was stirred for 1 hour. The mixture was concentrated under reduced pressure, diluted with CH₂Cl₂ and washed with saturated Na₂CO₃. The organic layer was separated, dried over anhydrous. Na₂SO₄, concentrated under vacuo and the crude product was used for next step.
iv) A solution of 7-chloro-2,3,4,5-tetrahydro-1H-benzo[b]azepine, 2-halo heteroaryl, DIEA in isopropanol was heated at 150° C. for 2-6 hours under microwave irradiation. The solvents were concentrated under vacuo, the residue taken in EtOAc, washed with H₂O and brine solution. The organic layer was separated, dried over anhydrous Na₂SO₄, concentrated under vacuo and the crude product was purified by column chromatography ethyl acetate/hexanes.
v) A solution of R—COOH, EDCI and HOBt in CH₂Cl₂ were stirred for 30 min. Then, a solution of 7-chloro-2,3,4,5-tetrahydro-1H-benzo[b]azepine in CH₂Cl₂ was added and the mixture was stirred at room temperature for 16 h. The reaction mixture was diluted with CH₂Cl₂, washed sequentially (2×) with H₂O, saturated NaHCO₃ and brine solution. The organic layer was separated, dried over anhydrous Na₂SO₄, concentrated under vacuo and the crude product was purified by column chromatography; or, alternatively,
to a stirred solution of 7-chloro-2,3,4,5-tetrahydro-1H-benzo[b]azepine in CH₂Cl₂, NEt₃ was added, followed by the drop-wise addition of the R—COCl in CH₂Cl₂ at 0° C. The reaction mixture was stirred for 2 hours, diluted with CH₂Cl₂ and washed sequentially (2×) with H₂O, saturated NaHCO₃ and brine solution. The organic layer was separated, dried over anhydrous Na₂SO₄, concentrated under vacuo and the crude product was purified by column chromatography.

### General Procedure for Preparation of the Compounds 140-154, 166-173, 179, 188-193, 202-208, and 216-219

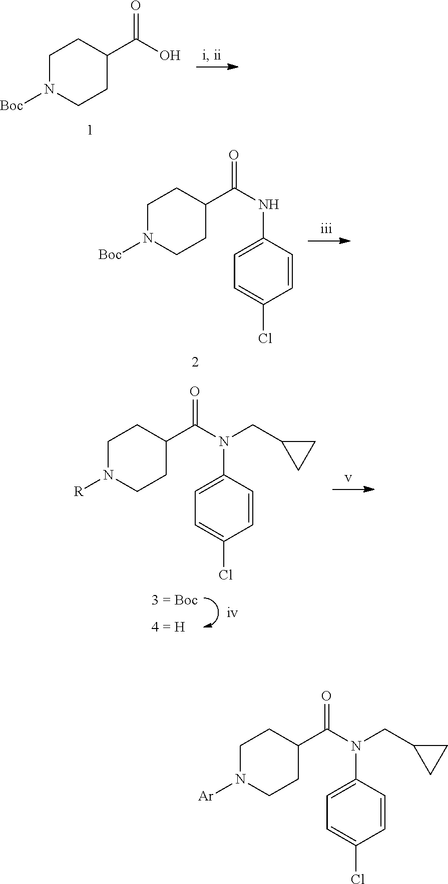

Reagents and conditions: (i) 1 (1 equiv.), oxalyl chloride (1.2 equiv., 2M solution in CH₂Cl₂), NEt₃ (1.2 equiv.), CH₂Cl₂, 0° C., overnight; (ii) 4-chloro aniline (1.05 equiv.), CH₂Cl₂, 0° C. to r.t., 2 h (iii) 2 (1 equiv.), cyclopropylmethyl bromide (1.2 equiv.), NaH (1.2 equiv., 60% suspension in oil), DMF, 0° C. to r.t., overnight (iv) 3 (1 equiv.), TFA (1 mL), CH₂Cl₂ (1 mL), 0° C. to r.t., 1 h; (v) 4, DIEA, ⁱPrOH, 145° C., microwave, 2-6 h.
i) A solution of piperidine-1,4-dicarboxylic acid mono-text-butyl ester and triethylamine in CH₂Cl₂ were cooled to 0° C. under N₂. Oxalyl chloride was added, followed by few drops of DMF, and the mixture was stirred at 0° C. for 2 hours. The solvents were concentrated under vacuo, azeotroped twice with toluene and the crude acid chloride was used for the next step.
ii) To a stirred solution of 4-chloro aniline in CH₂Cl₂, NEt₃ was added, followed by the drop-wise addition of the acid chloride (obtained from above) in CH₂Cl₂ at 0° C. The reaction mixture was stirred for 2 hours, diluted with CH₂Cl₂ and washed sequentially (2×) with H₂O, saturated NaHCO₃ and brine solution. The organic layer was separated, dried over anhydrous Na₂SO₄, concentrated under vacuo and the crude product was purified by column chromatography using ethyl acetate/hexanes (4:6).
iii) To a stirred solution of the product from the previous step in DMF at 0° C., NaH was added portion-wise. After 15 minutes, cyclopropylmethyl bromide was added and the reaction mixture was stirred overnight under N₂. The reaction mixture was poured into H₂O and extracted with ethyl acetate. The organic layer was washed with H₂O, brine solution, separated, dried over anhydrous Na₂SO₄, concentrated under vacuo and the crude product was purified by column chromatography ethyl acetate/hexanes (3:7).
iv) To a stirred solution of the product from the previous step in CH₂Cl₂ was added TFA at 0° C. and the reaction mixture was stirred for 1 hour. The mixture was concentrated under reduced pressure, diluted with CH₂Cl₂ and washed with saturated Na₂CO₃. The organic layer was separated, dried over anhydrous. Na₂SO₄, concentrated under vacuo and the crude product was used for next step.
v) A solution of the product from the previous step, 2-halo heteroaryl and DIEA in isopropanol was heated at 150° C. for 2 hours under microwave irradiation. The solvents were concentrated under vacuo, the residue taken in EtOAc, washed with H₂O and brine solution. The organic layer was separated, dried over anhydrous Na₂SO₄, concentrated under vacuo and the crude product was purified by column chromatography.

### Synthetic Schemes for Compounds of Formula (V)

Compounds of formula (V) can be prepared according to the schemes and synthetic procedures provided below in conjunction with knowledge of the person of ordinary skill and the known disclosures of the chemical literature. Specific exemplary synthetic procedures are provided in the Examples, below.

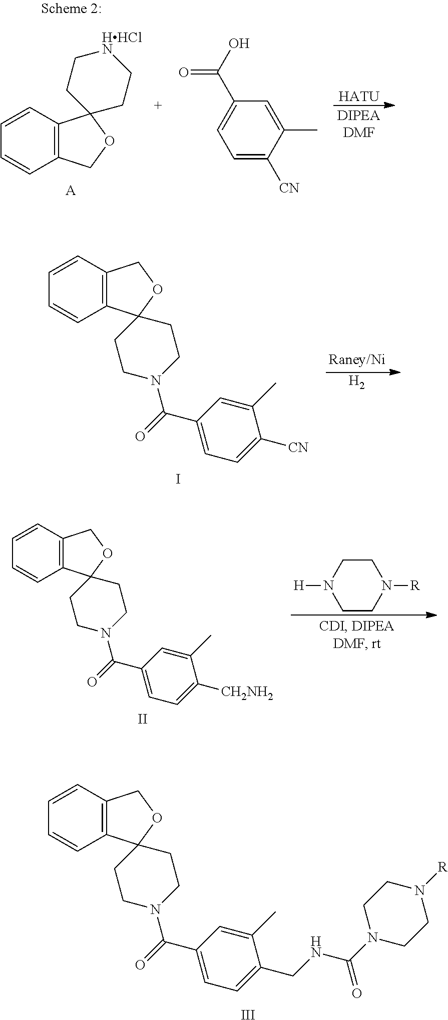

### General Procedure

To a stirred solution of a 4-cyano-3-methylbenzoic acid (1 mmol) in DMF was added HATU (1.3 mmol), DIPEA (2.2 mmol) and 1 mmol of spiro-4-(benzofuryl)piperidine A. The mixture was stirred overnight at r.t. and then poured onto water and extracted with CH₂Cl₂. The combined organic phases were washed with brine, dried and concentrated in vacuo. Flash chromatography afforded the title compound I.

Raney Nickel (10% wt) was added to a solution of compound I in ethanol. Under H₂ atmosphere, the mixture was stirred at room temperature overnight. When the reaction was complete, the solution was filtered through Celite® filter agent. The filtrate concentrated in vacuo to give brown oil and used to next step without purification.

1,1′-Carbonyldiimidazole (1.1 mmol) and DIPEA (1.2 mmol) were added to a solution of Compound 11 (1 mmol) in DMF and stirred for 1 h. Piperazine derivatives (1 mmol) and DIPEA (1.2 mmol) were added and the mixture stirred for 18 h. The mixture was reduced in vacuo to remove most of DMF and the residue was taken up in EtOAc and a minimum amount of methanol. The mixture was washed with brine, reduced in vacuo and taken up again in EtOAc and a minimum amount of methanol. The mixture was washed with NaHCO₃ and brine, dried and reduced in vacuo. The residue was purified by flash column chromatography on silica gel to get the compound III.

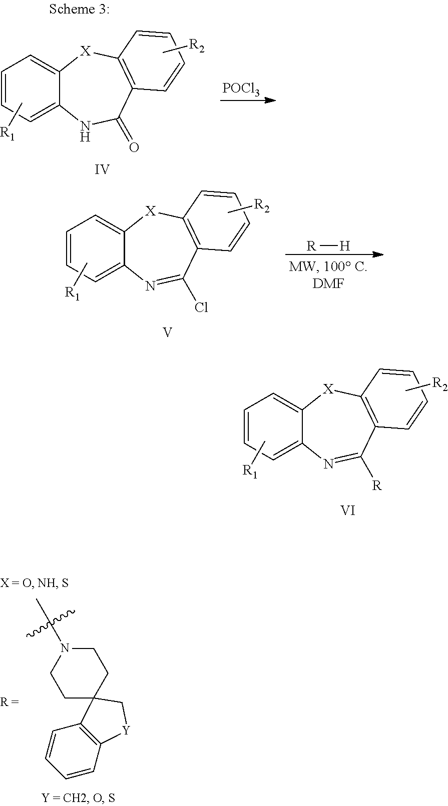

Tricyclic compounds (IV) are either commercially available or readily prepared using a procedure described in the chemical literature. Added excess POCl₃ to Compound IV (1 mmol) and heated at 90° C. for 2 hr, then cooled to room temperature and poured onto sat. brine, then extracted with EtOAc. The combined organic phases were washed with brine, dried over Na₂SO₄ and concentrated in vacuo. Flash chromatography afforded the title compound V.

Piperazine, morpholine, thiomorpholine, or spiropiperidine derivatives R—H (1 mmol) were added to a solution of Compound V (1 mmol) and DIPEA (1.2 mmol) in DMF and stirred under Microwave conditions (heated at 100° C. for 1 h). After cooled to room temperature, the mixture was reduced in vacuo to remove most of DMF and the residue was taken up in EtOAc. The organic phase was washed with brine, dried and reduced in vacuo. The residue was purified by flash column chromatography on silica gel to get the compound VI.

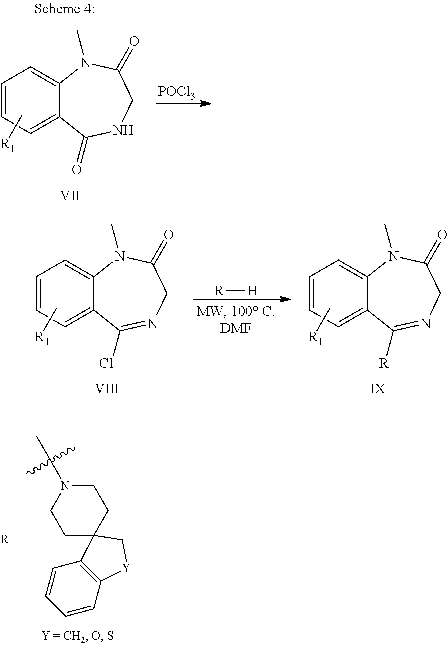

Bicyclic compounds (VII) are either commercially available or readily prepared using a procedure described in the chemical literature. Added excess POCl₃ to Compound VII (1 mmol) and heated at 90° C. for 2 hr, then cooled to room temperature and poured onto sat brine, then extracted with EtOAc. The combined organic phases were washed with brine, dried over Na₂SO₄ and concentrated in vacuo. Flash chromatography afforded the title compound VIII.

Piperazine, morpholine, thiomorpholine, or spiropiperidine derivatives R—H (1 mmol) were added to a solution of Compound VIII (1 mmol) and DIPEA (1.2 mmol) in DMF and stirred under Microwave conditions (heated at 100° C. for 1 h). After cooled to room temperature, the mixture was reduced in vacuo to remove most of DMF and the residue was taken up in EtOAc. The organic phrase was washed with brine, dried and reduced in vacuo. The residue was purified by flash column chromatography on silica gel to get the compound IX.

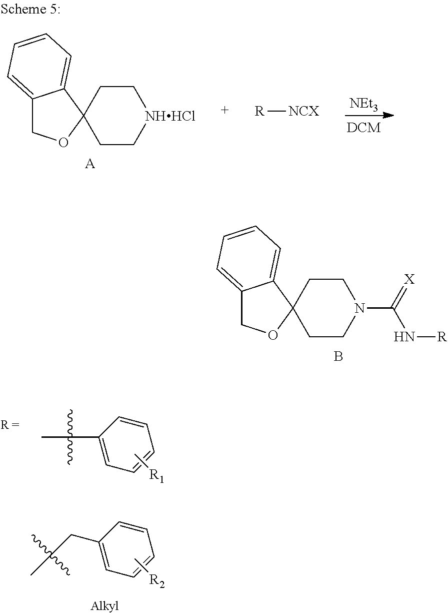

Added the alkyl or aryl isocyanates or isothiocyanate (1.1 mmol) to 3H-spiro[isobenzofuran-1,4′-piperidine] hydrochloride A (1 mmol) in DCM solution. The mixture was stirred overnight. When the reaction was complete, the solvent was reduced in vacuo and the residue was purified by flash column chromatography on silica gel or reverse HPLC preparation column to yield compounds B.

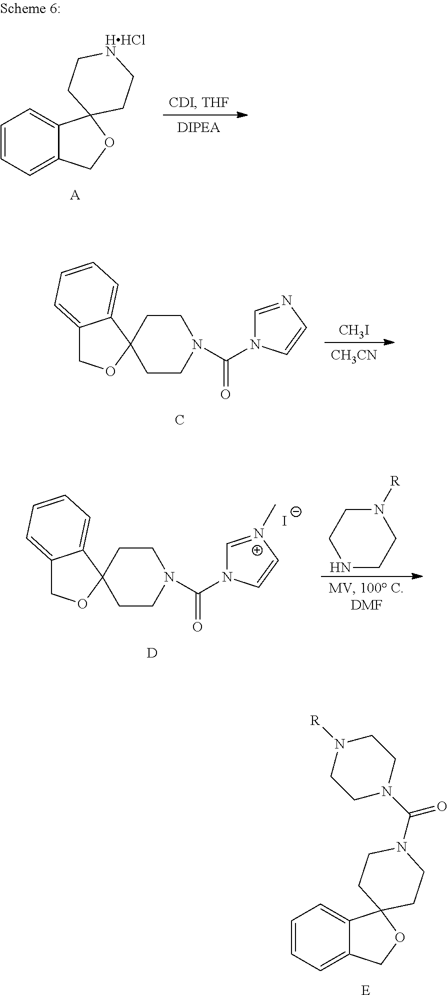

1,1′-Carbonyldiimidazole (1.1 mmol) and DIPEA (1.2 mmol) were added to a solution of 3H-spiro[isobenzofuran-1,4′-piperidine] hydrochloride A (1 mmol) in THF and refluxed overnight. The solvents were reduced in vacuo and the residue was purified by flash column chromatography on silica gel to get the compound C.

Methyl iodide (4 mmol) was added to a solution of Compound C (1 mmol) in CH₃CN and stirred at 50° C. overnight. When the reaction was complete, the solution was concentrated in vacuo to give brown oil and used to next step as salt D without purification.

Piperazine derivatives (1 mmol) were added to a solution of Compound D (1 mmol) and DIPEA (2.2 mmol) in DMF and stirred under Microwave conditions (heated at 100° C. for 1 h). After being cooled to room temperature, the mixture was reduced in vacuo to remove most of DMF and the residue was taken up in DCM. The organic phase was washed with brine, dried and reduced in vacuo. The residue was purified by flash column chromatography on silica gel to yield compounds E.

### General Procedure for the Synthesis of Spirocyclic Amides

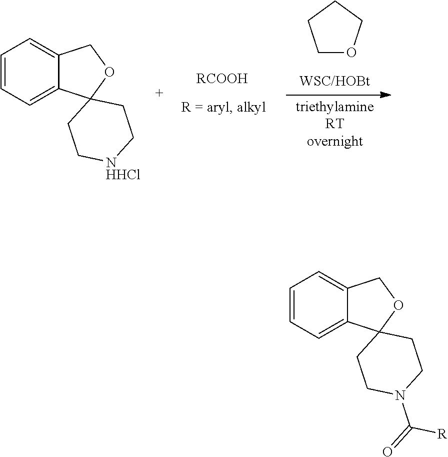

Spirocyclic compound 3H-spiro[isobenzofuran-1,4′-piperidine] hydrochloride (1 eq), 1-ethyl-3-(3-dimethylaminopropyl)carbodiimide hydrochloride (1.1 eq), HOBt (1.1 eq), acid (1.1 eq) and Et₃N (2 eq) were combined in THF; the resulting solution was stirred at room temperature overnight. Water (10 mL) was added to the reaction solution and the mixture was extracted with EtOAc (10 mL) two times. The combined organic layer was stripped under vacuum and the residue was purified by preparative TLC to afford spirocyclic amide compounds.

### Preparation of Compound V:139

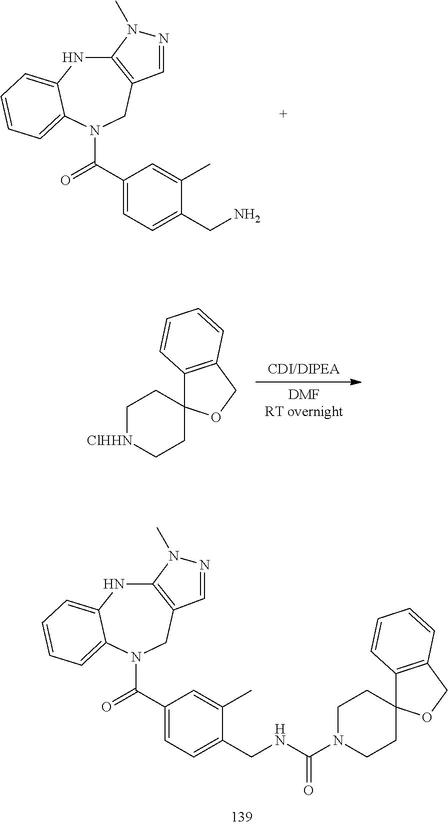

To a stirred solution of the tricyclic compound (20 mg, 0.057 mmol; prepared following the procedure described in J. Med. Chem, 2010, 53, 1546) in 1 mL of dry DMF was added DIPEA (11.6 mg, 0.115 mmol) followed by the addition of CDI (9.3 mg, 0.057 mmol) and the resulting solution was stirred at room temperature for 1 hour. The LC-MS showed all of the starting material (m/e=348[M+H]⁺) has been transformed into the intermediate (m/e=442[M+H]⁺). The spirocyclic compound 3H-spiro[isobenzofuran-1,4′-piperidine] hydrochloride was added and the mixture was stirred overnight at room temperature. Water (10 mL) was added to the reaction solution and the mixture was extracted by EtOAc (10 mL) two times. The combined organic layers were evaporated and the residue was purified by preparative TLC to afford 20 mg of the compound V:139, Yield: 62.5%.

### Preparation of Compounds V:145 and V:154

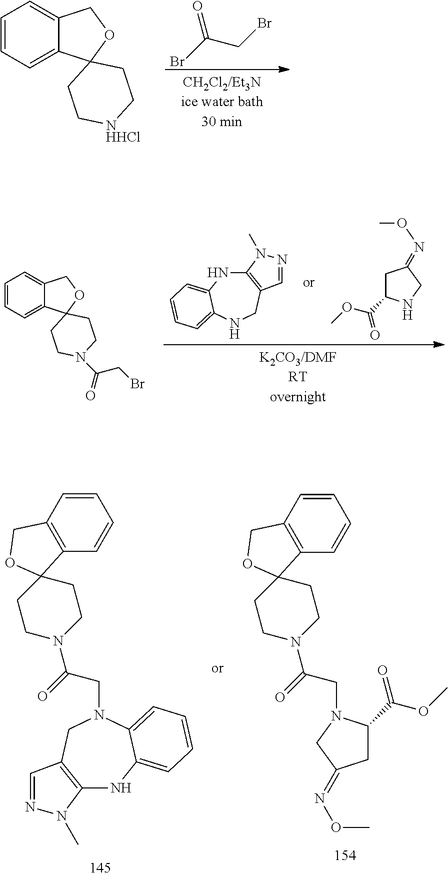

To a stirred suspension of 3H-spiro[isobenzofuran-1,4′-piperidine] hydrochloride (20 mg, 0.089 mmol) in CH₂Cl₂, was added triethylamine (18 mg, 0.178 mmol) at 0° C. followed by the addition of bromoacetyl bromide (22 mg, 0.11 mmol) and the mixture was stirred at 0° C. for 30 min. The reaction solution was washed with water, dried over Na₂SO₄, evaporated under vacuum and the crude was purified by preparative TLC to afford 23 mg of the α-bromo carbonyl intermediate, Yield: 70%.

The intermediate (10 mg, 0.032 mmol), tricyclic compound (7 mg, 0.035 mmol; prepared following the procedure described in J. Med. Chem, 2010, 53, 1546) and K₂CO₃ (5 mg, 0.036 mmol) were combined in DMF and the mixture was stirred at room temperature overnight. Water (10 mL) was added to the reaction solution and the mixture was extracted by EtOAc (10 mL) for two times. Vacuum evaporation of the combined organic layer and preparative TLC purification afforded 9 mg of compound V:145, Yield: 65.2%. Following the same procedure, 10 mg of the α-bromo carbonyl intermediate reacted with oxime compound (prepared following the procedure described in published patent WO2002/074741) to afford 8 mg of compound V:154, Yield: 61%.

### Preparation of Compound V:146

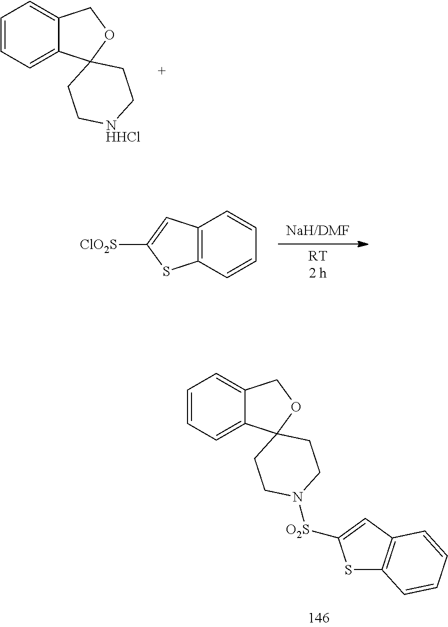

To a stirred solution of 3H-spiro[isobenzofuran-1,4′-piperidine] hydrochloride (20 mg, 0.089 mmol) in dry DMF, at 0° C., was added NaH (60% in mineral oil, 5 mg, 0.125 mmol) and the mixture was stirred for 20 min, then sulfonamide compound was added, allowed to warm to room temperature and the mixture was stirred for 2 hours. Water (10 mL) was added to the reaction solution and the mixture was extracted by EtOAc (10 mL) two times. The combined organic layers were evaporated and the residue was purified by preparative TLC to afford 16 mg of compound V:146, Yield: 50%.

### Preparation of Compound V:147

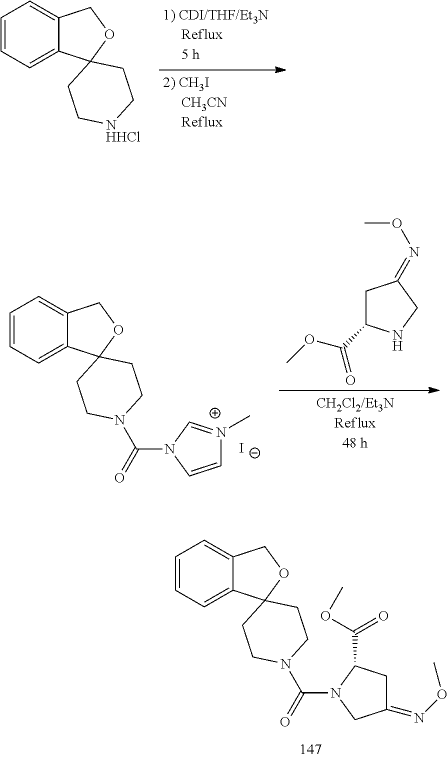

To a stirred suspension of 3H-spiro[isobenzofuran-1,4′-piperidine] hydrochloride (20 mg, 0.089 mmol) in dry THF, was added triethylamine (18 mg, 0.178 mmol) followed by the addition of CDI (15 mg, 0.090 mmol). The resulting solution was heated to reflux for 5 hours, the solvent removed under vacuum and the residue was purified by preparative TLC to afford 22 mg of the imidazole intermediate (m/e=284[M+1-1]⁺), Yield: 88%.

The above imidazole intermediate (22 mg, 0.078 mmol) was dissolved in CH₃CN and CH₃I (55 mg, 0.39 mmol) was added and the resulting solution was heated to reflux overnight. The reaction solution was evaporated to afford the crude imidazole salt as pale-yellow oil.

The above crude imidazole salt compound (11 mg, 0.078 mmol), the oxime, and Et₃N (16 mg, 0.156 mmol) were combined in CH₂Cl₂ and heated to reflux for two days. The solvent was evaporated and the residue was purified by preparative TLC to afford 16 mg of compound V:147 (m/e=350[M+H]⁺), Yield=60%.

### Preparation of Compound V:148

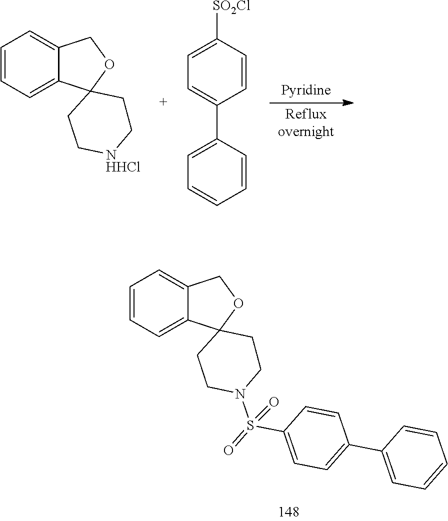

The compound 3H-spiro[isobenzofuran-1,4′-piperidine] hydrochloride (10 mg, 0.044 mmol) and sulfonyl chloride (12 mg, 0.047 mmol) were combined in pyridine (0.5 ml) and stirred at 80° C. overnight, The solvent was removed and the residue was purified by preparative TLC to afford 14 mg of compound V:148 (m/e=406[M+H]⁺), Yield: 78%.

### Preparation of Compounds V:149 and V:150

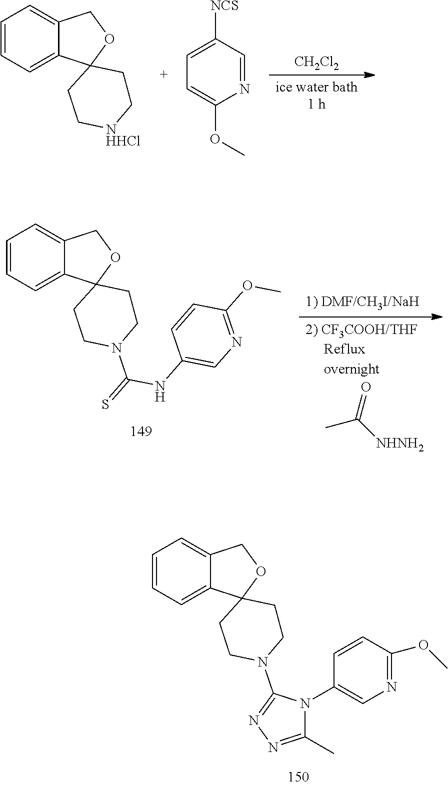

The compound 3H-spiro[isobenzofuran-1,4′-piperidine] hydrochloride (20 mg, 0.088 mmol) and the isothiocyanate (15 mg, 0.09 mmol) were combined in CH₂Cl₂ at 0° C., stirred at this temperature for 1 hour, the reaction solution washed with water and the solvent was evaporated to afford the residue, which was purified by preparative TLC to yield 30 mg of compound V:149 (m/e=356[M+H]⁺), Yield: 86%.

Compound V:149 (30 mg, 0.085 mmol) was dissolved in DMF and cooled to 0° C., NaH (5 mg, 0.090 mmol) was added and stirred for 30 min. CH₃I (12 mg, 0.085 mmol) was added and the resulting solution was warmed to room temperature and stirred for 1 hr. The reaction was quenched with water (10 mL) and extracted with EtOAc (2×10 mL), the organic layers combined, dried over anhyd. Na₂SO₄, evaporated under vacuum and purified by preparative TLC to obtain 25 mg of the sulfur methylation product (m/e=370[M+H]⁺), Yield: 78%.

The sulfur methylation product (25 mg, 0.068 mmol) was dissolved in THF and CF₃COOH (two drops) was added and the resulting solution was heated to reflux overnight. The solvent was removed under vacuum and the residue was purified by preparative TLC to afford 10 mg of product V:150, Yield: 40%.

### Preparation of Compound V:153

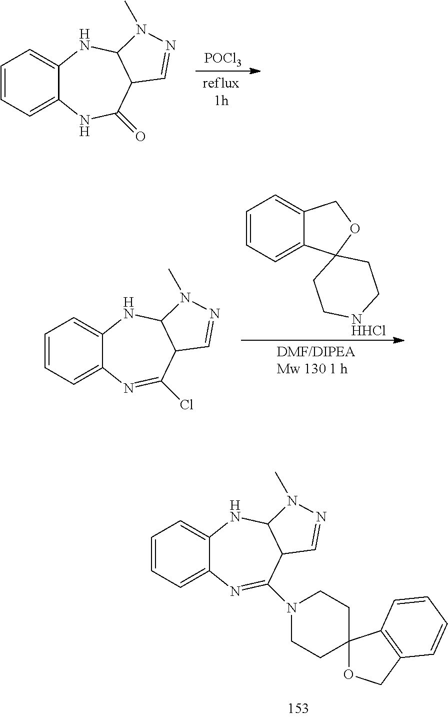

The tricyclic compound (J. Med. Chem, 2010, 53, 1546; 20 mg, 0.093 mmol) and POCl₃ (0.5 mL) were combined in a small sealed tube flushed with nitrogen, which was then was heated to 100° C. for one hour. The reaction mixture was stripped to afford the crude chloro intermediate. The crude chloro intermediate was dissolved in DMF (1 mL) and DIPEA (37 mg, 0.372 mmol) and 3H-spiro[isobenzofuran-1,4′-piperidine] hydrochloride (21 mg, 0.093 mmol) were added, then the resulting solution was heated to 130° C. in a microwave reactor for one hour. Water (10 mL) was added to the reaction solution and the mixture was extracted by EtOAc (10 mL) for two times. The combined organic layers were evaporated and the residue was purified by preparative TLC to afford 6 mg of compound V:153, Yield: 17%.

### General Procedure for the Synthesis of Compounds V:197, V:198, V:234, V:235, and V:236

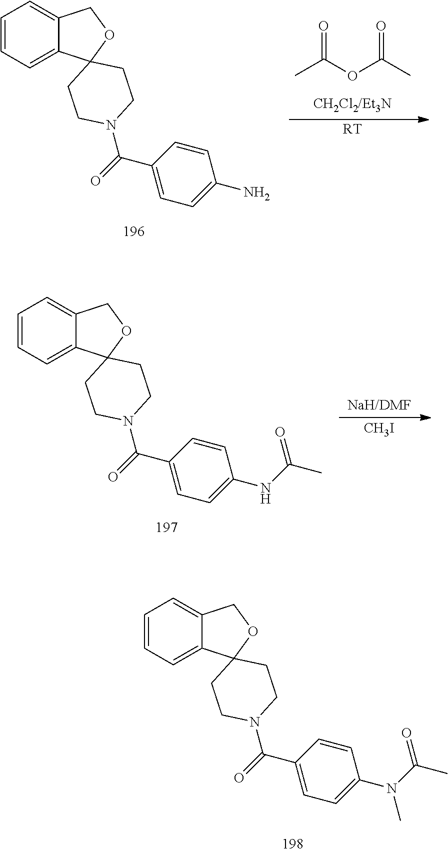

Aniline compound V:196 (20 mg, 0.065 mmol) was dissolved in CH₂Cl₂ then Et₃N (13 mg, 0.13 mmol) and acetic anhydride (10 mg, 0.10 mmol) were added; the resulting solution was stirred at room temperature for 3 hours. The reaction mixture was stripped under vacuum and the residue was purified by preparative TLC to afford 20 mg of compound V:197, Yield: 88%. Following the same procedure, the compound V:196 was reacted with trifluoroacetic anhydride, cyclopentane carboxylic acid chloride and with cyclopropanecarboxylic acid to afford the compounds V:234, V:235 and V:236 respectively.

Compound V:197 (10 mg, 0.028 mmol) was dissolved in DMF and cooled to 0° C., then NaH (2 mg, 0.050 mmol) was added and stirred for 30 min. CH₃T (5 mg, 0.035 mmol) was added and the resulting solution was warmed to room temperature and stirred for 1 hr, quenched the reaction with water (10 mL) and extracted with EtOAc (2×10 mL). The combined organic layers were dried over Na₂SO₄, vacuum evaporated, and purified by preparative TLC to obtain 8 mg of compound V:198 (m/e=365[M+H]⁺), Yield: 77%.

### General Procedure for Dimethylation of Anilines V:200 and V:209

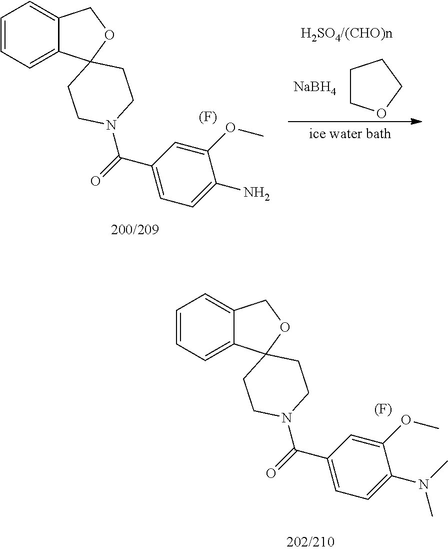

Aniline compound V:200 or V:209 (1 eq) was dissolved in THF and paraformaldehyde (5 eq) and H₂SO₄ (3M, 1 eq) were added, then cooled to 0° C. NaBH₄ (6 eq) was added and stirred at room temperature for three hours. The yield of the respective N,N-dimethylation product V:202 or V:210 is around 90%.

### Preparation of Compound V:203

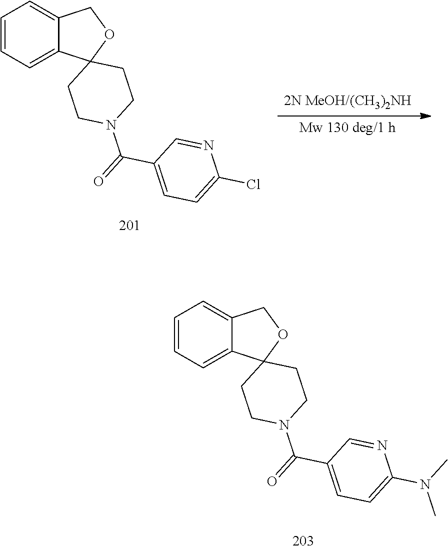 [SMILES: O=C(c1ccc(Cl)nc1)N1CCC2(CC1)OCc1ccccc12 ; CN(C)c1ccc(C(=O)N2CCC3(CC2)OCc2ccccc23)cn1]

Compound V:201 was dissolved in 2N (CH₃)₂NH/MeOH, then the resulting solution was heated to 130° C. in a microwave reactor for one hour. The solvent was removed under vacuum and the residue was purified by preparative TLC afford 9 mg of compound V:203, Yield: 90%.

### Preparation of Compounds V:241, V:243-V:245

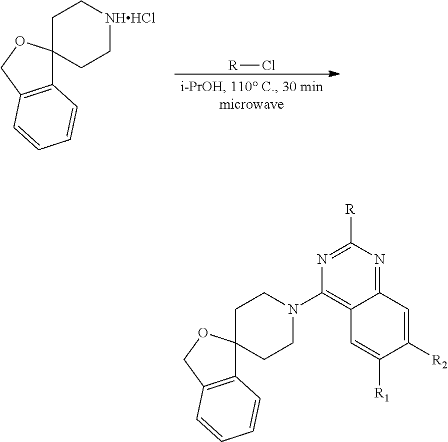

A sample of 3H-spiro[isobenzofuran-1,4′-piperidine] hydrochloride (1.2 equiv), corresponding 4-chloro quinazolines (1 equiv.) and DIEA (2 equiv.) was taken in a microwave vial and was added i-PrOH. The reaction mixture was heated to 110° C. for 30 min in microwave. The solvents were concentrated in vacuo, the residue partitioned between EtOAc and H₂O, organic layer separated, washed with sat. brine solution, dried over anhyd. Na₂SO₄, solvents removed in vacuo and the crude was purified by column chromatography to obtain desired products in 35-70% yields.

### Bioassay Procedures

### Agonist Assays:

Data represent the percent efficacy (relative to cognate agonist) of the test compound at 10 micromolar. The response to a saturating concentration of cognate/reference agonist is set to 100%; the response to a saturating concentration of a reference antagonist or to vehicle (usually none or negligible) is set to 0%.

### Antagonist Assays:

Data represent the percent inhibition of the response to an EC₉₀ concentration (empirically determined immediately prior to assay) of cognate/reference agonist by the test compound at 10 micromolar. The response inhibition elicited by a saturating concentration of reference antagonist is set to 100%; the response inhibition (usually none or negligible) elicited by vehicle is set to 0%.

### Receptors:

Vasopressin V1, V2, V3;

### Assay Buffer:

1× Hanks Balanced Salt Solution, 20 mM HEPES, 2.5 mM probenecid, pH 7.4 is used to reconstitute lyophilized Calcium Plus Assay Kit dye (Molecular Devices)

Protocol adapted from Davies et al. Psychopharmacology 178(4):451-460 (2005).

### Experimental Procedure and Data Analysis:

Receptor-expressing cell lines are seeded in glass-bottom 96- or 384-well, poly-L-lysine-coated plates 48 hours prior to the assay (40,000 cells per well or 6,700 cells, respectively) in DMEM containing 5% dialyzed serum. Twenty hours prior to the assay, the medium is changed to serum-free DMEM. Then, the cells are preincubated in 30 μl (96-well plates) or 20 μl (384-well plates) of calcium dye-containing assay buffer (the lyophilized dye is reconstituted with 15 ml of assay buffer) at 37 degrees centigrade for 75 min in a humidified incubator. During that time, serial dilutions of the reference and test compounds are made at 2× assay concentration (final assay concentrations ranging from 0.1 nM to 10 μM). Just prior to the assay, the plates are allowed to cool to room temperature for 10 min and then are transferred to a FLIPR Tetra fluorescence image plate reader (Molecular Devices). Basal fluorescence (excitation 488 nm, emission 510-570 nm) is measured for 20 sec, then test compound or reference agonist dilutions (2× assay concentration) are added (30 μl for 96-well plates, 20 μl for 384-well plates, each concentration assayed in triplicate) and fluorescence is measured for 60 sec. The maximum fluorescence values during the baseline and test compound or reference agonist addition phases (for agonist assays) are exported for analysis.

For agonist tests, raw data (maximum fluorescence, fluorescence units) for each concentration of test compound or reference are normalized to the baseline fluorescence (reported as fold increase over basal) and plotted as a function of the logarithm of the molar concentration of the drug (i.e., test or reference compound). Non-linear regression of the normalized data is performed in Prism 4.0 (GraphPad Software) using the built-in three parameter logistic model (i.e., sigmoidal concentration-response) describing agonist-stimulated activation of one receptor population:

y=bottom+[(top−bottom)/(1+10×−log EC50)]

where bottom equals the best-fit basal fluorescence and top equals the best-fit maximal fluorescence stimulated by the test compound or reference agonist. The log EC₅₀ (i.e., the log of the drug concentration that increases fluorescence by 50% of the maximum fluorescence observed for the test compound or reference agonist) is thus estimated from the data, and the EC50 (agonist potency) is obtained. To obtain an estimate of the relative efficacy of the test compound (Rel. Emax), its best-fit top is compared to and expressed as a ratio of that for the reference agonist (Rel. Emax of the reference agonist is 1.00).

To ascertain whether test compounds are antagonists, a double-addition paradigm is employed. After measuring baseline fluorescence for 20 seconds, 30 μl of test compound (20 μM) is added (10 μM final concentration, assayed in triplicate) and fluorescence is measured for an additional 15 min. Then, 30 μl of reference agonist (3×; EC₉₀) is added (final concentration of agonist is EC₃₀) and fluorescence is measured for 60 sec. Maximum baseline-normalized fluorescence evoked by the reference agonist in the presence of test compound is compared to the maximum baseline-normalized fluorescence elicited by the reference agonist following addition of vehicle instead of test compound and expressed as a ratio. ‘Hits’ (compounds that antagonize reference agonist-stimulated increases in baseline-normalized fluorescence by at least 50%) are then characterized by a modified Schild analysis.

For modified Schild analysis, a family of reference agonist concentration-response isotherms is generated in the absence and presence of graded concentrations of test compound (added 15 min prior to reference agonist). Theoretically, compounds that are competitive antagonists cause a dextral shift of agonist concentration-response isotherms without reducing the maximum response to agonist (i.e., surmountable antagonism). However, on occasion, factors such as non-competitive antagonism, hemiequilibria, and/or receptor reserve cause apparent insurmountable antagonism. To account for such deviations, we apply the modified Lew-Angus method to ascertain antagonist potency (Christopoulos et al., 1999). Briefly, equieffective concentrations of agonist (concentrations of agonist that elicit a response equal to the EC₂₅% of the agonist control curve) are plotted as a function of the test compound concentration present in the wells in which they were measured. Non-linear regression of the baseline-normalized data is performed in Prism 4.0 using the following equation:

pEC25%=−log([B]+10−pK)−log c

where EC_{25%} equals the concentration of agonist that elicits a response equal to 25% of the maximum agonist control curve response and [B] equals the antagonist concentration; K, c, and s are fit parameters. The parameter s is equal to the Schild slope factor. If s is not significantly different from unity, pK equals pKB; otherwise, pA2 is calculated (pA2=pK/s). The parameter c equals the ratio EC₂₅%/[B].

### Vasopressin V1a Receptor Binding Assay.

A cell line expressing the human V1a receptor in CHO cells is used. The hV1a cDNA sequence is described by Thibonnier et al., (1994), and the expression method is the same as described by Morel et al. (1992). The hV1a cell line is grown in alpha-MEM with 10% fetal bovine serum and 250 ug/ml G418 (Gibco, Grand Island, N.Y., USA). For competitive binding assay, hV1a cells are plated into 6-well culture plate at 1:10 dilution from a confluency flask, and maintained in culture for at least two days. Culture medium is then removed, cells washed with 2 ml binding buffer (35 mM Hepes, 0.25% BSA, 1× DMEM, PH-7.0). To each well, 990 μl binding buffer containing 1 nM 3H-AVP is added, followed by 10 μl series diluted Example compounds dissolved in DMSO. All incubations are done in triplicate and dose-inhibition curves consisting of total binding (DMSO) and 5 concentrations (0.1, 1.0, 10, 100, and 1000 nM) of test agents encompassing the IC50. 100 nM cold AVP (Sigma) is used to assess non-specific binding. Cells are incubated for 45 minutes at 37° C., assay mixture is removed and each well is washed three times with PBS (pH=7.4). 1 ml 2% SDS was added per well, plates were let sit for 30 minutes. The whole content of a well is transferred to a scintillation vial. Each well is rinsed with 0.5 ml PBS which is then added to the corresponding vial. Scintillation fluid (Ecoscint) is then added at 3 ml per vial. Samples are counted in a liquid scintillation counter (Beckman LS3801). IC50 values are calculated by Prism Curve fitting software.

### V1a-R Antagonist Assay

### Assay Overview:

The purpose of this assay is to determine the potency of test synthesized compounds. This assay uses AVPR1a-NFAT-bla CHO-K1 cells, which contain the human Arginine Vasopressin Receptor 1a (AVPR1a) stably integrated into the CellSensor® NFAT-bla CHO-K1 cell line. The cells also express a beta-lactamase reporter gene under control of the Nuclear Factor of Activated T-cells (NEAT) response element. Stimulation of the AVPR1a receptor by agonist results in increased transcription of the NFAT-beta lactamase reporter gene. BLA expression is monitored by measuring fluorescence resonance energy transfer (FRET) of a cleavable, fluorogenic, cell-permeable BLA substrate. As designed, test compounds that act as AVPR1a antagonists will inhibit agonist activation and thus prevent BLA transcription, leading to no increase in well FRET. Compounds were tested in triplicate in a 10-point, 1:3 dilution series starting at a nominal concentration of either 50 micromolar, or 500 nM for selected compounds.

### Protocol Summary:

The AVPR1a-BLA CHO-K1 dividing cell line was routinely cultured in T175 Flasks at 37° C., 5% CO₂ and 95% relative humidity (RH). The growth medium consisted of DMEM Media supplemented with 10% v/v dialyzed fetal bovine serum, 25 mM HEPES, 0.1 mM non-essential amino acids, and 1× antibiotic mix (penicillin streptomycin). On day 1 of the assay, 10,000 cells in 10 μL of assay media (DMEM with 1% dialyzed fetal bovine serum, 25 mM HEPES, 0.1 mM non-essential amino acids, and antibiotic mix (penicillin streptomycin) were seeded into each well of a 384-well plate. On Day 2, 50 nL of test compound in DMSO were added to the appropriate wells and plates were incubated for 30 minutes at 37° C., 5% CO₂ and 95% RH. Next, 0.1 uL of 300 nM OXT peptide (EC₉₀ Challenge; final concentration 0.27 μM) or DMSO in assay medium was added to appropriate wells and incubated 5 hours at 37° C., 5% CO₂ and 95% RH. 2.2 μL of LiveBLazertrade mark FRET B/G (CCF4-AM) loading mix prepared according to manufacturer's instructions were added to each well, and plates incubated at room temperature in the dark for 2 hours. Well fluorescence was measured on Perkin Elmer's Envision using an Excitation filter 409 nm, Emission filters at 460 nm and 530 nm, bottom read. Assay Cutoff: Compounds that exhibited an IC₅₀ for V1a-R of ≦10 μM were considered active.

### DOCUMENTS CITED

Aakerlund, Mats (2006) Targeting the oxytocin receptor to relax the myometrium, Expert Opinion on Therapeutic Targets 10(3), 423-427.
Abraham, William T.; Shamshirsaz, Alireza A.; McFann, Kim; Oren, Ron M.; Schrier, Robert W. (2006) Aquaretic effect of lixivaptan, an oral, non-peptide, selective V2 receptor vasopressin antagonist, in New York heart association functional class II and III chronic heart failure patients Journal of the American College of Cardiology 47(8), 1615-1621
Albright, J. Donald; Reich, Marvin F.; Delos Santos, Efren G.; Dusza, John P.; Sum, Fuk-Wah; Venkatesan, Aranapakam M.; Coupet, Joseph; Chan, Peter S.; Ru, Xun; Mazandarani, Hossein; et al (1998) 5-Fluoro-2-methyl-N-[4-(5H-pyrrolo[2,1-c]-[1,4]benzodiazepin-10(11H)-ylcarbonyl)-3-chlorophenyl]benzamide (VPA-985): an orally active arginine vasopressin antagonist with selectivity for V2 receptors. Journal of Medicinal Chemistry 41(14), 2442-2444.
Barberis, C.; Morin, D.; Durroux, T.; Mouillac, B.; Guillon, G.; Seyer, R.; Hibert, M.; Tribollet, E.; Manning, M. (1999) Molecular pharmacology of AVP and OT receptors and therapeutic potential. Drug News & Perspectives 12(5), 279-292.
Bernier Virginie; Morello Jean-Pierre; Zarruk Alexandro; Debrand Nicolas; Salahpour Ali; Lonergan Michele; Arthus Marie-Francoise; Laperriere Andre; Brouard Remi; Bouvier Michel; et al (2006) Pharmacologic chaperones as a potential treatment for X-linked nephrogenic diabetes insipidus Journal of the American Society of Nephrology 17(1), 232-43.
Blanchard, Robert J, et al (2005) AVP V1b selective antagonist SSR149415 blocks aggressive behaviors in hamsters Pharmacol Biochem Behav, 80:189-94.
Cawley, Michael J. (2007) Hyponatremia: current treatment strategies and the role of vasopressin antagonists. Annals of Pharmacotherapy 41(5), 840-850.
Chen, Sheldon; Jalandhara, Nishant; Batllc, Daniel (2007) Evaluation and management of hyponatremia: an emerging role for vasopressin receptor antagonists. Nature Clinical Practice Nephrology 3(2), 82-95.
Craighead, Mark; Milne, Rachel; Campbell-Wan, Leigh; Watson, Lynn; Presland, Jeremy; Thomson, Fiona J.; Marston, Hugh M.; MacSweeney, Cliona P. (2008) Characterization of a novel and selective V1B receptor antagonist. Progress in Brain Research 170 (Advances in Vasopressin and Oxytocin), 527-535.
Ferris, Craig F.; Lu, Shi-fang; Messenger, Tara; Guillon, Christophe D.; Heindel, Ned; Miller, Marvin; Koppel, Gary; Bruns, F. Robert; Simon, Neal G. (2006) Orally active vasopressin V1a receptor antagonist, SRX251, selectively blocks aggressive behavior Pharmacology, Biochemistry and Behavior 83(2), 169-174.
Flouret, George; Chaloin, Olivier; Borovickova, Lenka; Slaninova, Jirina (2006) Design of novel bicyclic analogues derived from a potent oxytocin antagonist. Journal of Peptide Science 12(6), 412-419.
Ghali, Jalal K.; Koren, Michael J.; Taylor, James R.; Brooks-Asplund, Esther; Fan, Kaisheng; Long, Walker A.; Smith, Neila; The Conivaptan Study Group (2006) Efficacy and safety of oral conivaptan: a V1A/V2 vasopressin receptor antagonist, assessed in a randomized, placebo-controlled trial in patients with euvolemic or hypervolemic hyponatremia Journal of Clinical Endocrinology and Metabolism 91(6), 2145-2152.
Gimpl, Gerald; Fahrenholz, Falk (2001) The oxytocin receptor system: structure, function, and regulation Physiological Reviews 81(2), 629-683
Gines, Pere (2007) Vaptans: A promising therapy in the management of advanced cirrhosis

### Journal of Hepatology 46(6), 1150-1152.

Greenberg, A.; Verbalis, J. G. (2006) Vasopressin receptor antagonists Kidney International 69(12), 2124-2130.
Griebel, Guy; Simiand, Jacques; Serradeil-Le Gal, Claudine; Wagnon, Jean; Pascal, Marc; Scatton, Bernard; Maffrand, Jean-Pierre; Soubrie, Philippe (2002) Anxiolytic- and antidepressant-like effects of the non-peptide vasopressin V1b receptor antagonist, SSR149415, suggest an innovative approach for the treatment of stress-related disorders. Proceedings of the National Academy of Sciences of the United States of America 99(9), 6370-6375.
Guillon, Christophe D.; Koppel, Gary A.; Brownstein, Michael J.; Chancy, Michael O.; Ferris, Craig F.; Lu, Shi-Fang; Fabio, Karinc M.; Miller, Marvin J.; Heindel, Ned D.; Hunden, David C.; et al (2007) Azetidinones as vasopressin V1a antagonists. Bioorganic & Medicinal Chemistry 15(5), 2054-2080.
Hays Richard M (2006) Vasopressin antagonists progress and promise The New England journal of medicine 355(20), 2146-8.
Hirsch, Alan T.; Dzau, Victor J.; Majzoub, Joseph A.; Creager, Mark A. (1989) Vasopressin-mediated forearm vasodilation in normal humans. Evidence for a vascular vasopressin V2 receptor Journal of Clinical Investigation 84(2), 418-26.
Hollander, E., Novotny, S., Hanratty, M., Yaffe, R., DeCaria, C. M., Aronowitz, B. R. and Mosovich, S. (2003) Oxytocin infusion reduces repetitive behaviors in adults with autistic and Asperger's disorders. Neuropsychopharmacology, 28: 193-198.
Hollander, E., Bartz, J., Chaplin, W., Phillips, A., Sumner, J., Soorya, L., Anagnostou, E. and Wasserman, S. (2007) Oxytocin increases retention of social cognition in autism. Biol. Psychiatry, 61: 498-503.
Holmes Cheryl L; Landry Donald W; Granton John T (2003) Science review: Vasopressin and the cardiovascular system part 1—receptor physiology Critical care (London, England) 7(6), 427-34
Holmes Cheryl L, Landry Donald W, Granton John T. (2004) Science Review: Vasopressin and the cardiovascular system part 2—clinical physiology. Crit Care 8: 15-23.
Huber, D., Veinante, P. and Stoop, R. (2005) Vasopressin and oxytocin excite distinct neuronal populations in the central amygdala. Science, 308: 245-248
Insel, T. R. and Young, L. J. (2001) The neurobiology of attachment. Nat. Rev. Neurosci., 2: 129-136.
Jard, Serge (1998) Vasopressin receptors: a historical survey. Advances in Experimental Medicine and Biology, 449 (Vasopressin and Oxytocin), 1-13.
Kirsch, P., Esslinger, C., Chen, Q., Mier, D., Lis, S., Siddhanti, S., Gruppe, H., Mattay, V. S., Gallhofer, B. and Meyer-Lindenberg, A. (2005) Oxytocin modulates neural circuitry for social cognition and fear in humans. J. Neurosci., 25: 11489-11493.
Landgraf Rainer (2006) The involvement of the vasopressin system in stress-related disorders
   CNS & neurological disorders drug targets 5(2), 167-79.
Liard, Jean Francois (1989) Vasopressin reduces oxygen uptake in intact dogs but not in sinoaortic-denervated dogs. American Journal of Physiology 257(1, Pt. 2), R1-R9.
M. Manning, L. L. Cheng, S. Stoev, N.C. Wo, W. Y. Chan, H. H. Szeto, T. Durroux, B. Mouillac and C. Barberis (2005) Design of Peptide Oxytocin Antagonists with Strikingly Higher Affinities and Selectivities for the Human Oxytocin Receptor than Atosiban. J. Peptide Sci. 11:593-608
Melin, P.; Trojnar, J.; Johansson, B.; Vilhardt, H.; Aakerlund, M. (1986) Synthetic antagonists of the myometrial response to vasopressin and oxytocin. Journal of Endocrinology 111(1), 125-31
Munger, Mark A. (2007) New agents for managing hyponatremia in hospitalized patients American Journal of Health-System Pharmacy 64(3), 253-265.
Oliver J. Bosch, Simone L. Meddle, Daniela I. Beiderbeck, Alison J. Douglas, and Inga D. Neumann (2005) Brain Oxytocin Correlates with Maternal Aggression: Link to Anxiety The Journal of Neuroscience, 25(29):6807-6815
Palm, Catrin; Pistrosch, Frank; Herbrig, Kay; Gross, Peter (2006) Vasopressin antagonists as aquaretic agents for the treatment of hyponatremia American Journal of Medicine 119(7A), S87-S92.
Parker, K. J., Buckmaster, C. L., Schatzberg, A. F. and Lyons, D. M. (2005) Intranasal oxytocin administration attenuates the ACTH stress response in monkeys. Psychoneuroendocrinology, 30: 924-929.
Reinheimer, Torsten M.; Bee, Walter H.; Resendez, John C.; Meyer, Julie K.; Haluska, George J.; Chellman, Gary J. (2005) Barusiban, a new highly potent and long-acting oxytocin antagonist: Pharmacokinetic and pharmacodynamic comparison with atosiban in a cynomolgus monkey model of preterm labor. Journal of Clinical Endocrinology and Metabolism 90(4), 2275-2281.
Ring R H (2005) The central vasopressinergic system: examining the opportunities for psychiatric drug development. Curr Pharm Des 11: 205-225.
Ring, R. H., Malberg, J. E., Potestio, L., Ping, J., Boikess, S., Luo, B., Schechter, L. E., Rizzo, S., Rahman, Z., Rosenzweig-Lipson, S., (2006). Anxiolytic-like activity of oxytocin in male mice: behavioral and autonomic evidence, therapeutic implications. Psychopharmacology (Berl.) 185, 218-225.
Robben, J. H.; Sze, M.; Knoers, N. V. A. M.; Deen, P. M. T. (2007) Functional rescue of vasopressin V2 receptor mutants in MDCK cells by pharmacochaperones: relevance to therapy of nephrogenic diabetes insipidus American Journal of Physiology 292(1, Pt. 2), F253-F260.
Romergo, Roberto; Sibai, Baha M.; Sanchez-Ramos, Luis; Valenzuela, Guillermo J.; Veille, Jean-Claude; Tabor, Bannie; Perry, Kenneth G.; Varner, Michael; Goodwin, T. Murphy; Lane, Rosanne; et al (2000) An oxytocin receptor antagonist (atosiban) in the treatment of preterm labor: a randomized, double-blind, placebo-controlled trial with tocolytic rescue. American Journal of Obstetrics and Gynecology 182(5), 1173-1183.
Schrier, Robert W.; Gross, Peter; Gheorghiade, Mihai; Berl, Tomas; Verbalis, Joseph G.; Czerwiec, Frank S.; Orlandi, Cesare; Tolvaptan, a selective oral vasopressin V2-receptor antagonist, for hyponatremia (2006) New England Journal of Medicine, 355(20), 2099-2112
Schwarz, Ernst R.; Sanghi, Pramod (2006) Conivaptan: a selective vasopressin antagonist for the treatment of heart failure Expert Review of Cardiovascular Therapy 4(1), 17-23
Serradeil-Le Gal, Claudine; Lacour, Colette; Valette, Gerard; Garcia, Georges; Foulon, Loiec; Galindo, Gerard; Bankir, Lise; Pouzet, Brigitte; Guillon, Gilles; Barberis, Claude; et al (1996) Characterization of SR 121463A, a highly potent and selective, orally active vasopressin V2 receptor antagonist. Journal of Clinical Investigation 98(12), 2729-2738.
Serradeil-Le Gal, C.; Wagnon, J.; Valette, G.; Garcia, G.; Pascal, M.; Maffrand, J. P.; Le Fur, G. (2002) Nonpeptide vasopressin receptor antagonists: development of selective and orally active V1a, V2 and V1b receptor ligands. Progress in Brain Research 139 (Vasopressin and Oxytocin: From Genes to Clinical Applications), 197-210.
Serradeil-Le Gal, Claudine; Raufastc, Daniele; Dcrick, Sylvain; Blankenstein, Jorg; Allen, John; Pouzet, Brigitte; Pascal, Marc; Wagnon, Jean; Ventura, Maria Angeles (2007) Biological characterization of rodent and human vasopressin V1b receptors using SSR-149415, a nonpeptide V1b receptor ligand American Journal of Physiology 293(2, Pt. 2), R938-R949.
Soupart, Alain; Gross, Peter; Legros, Jean-Jacques; Alfoldi, Sandor; Annane, Djillali; Heshmati, Hassan M.; Decaux, Guy (2006) Successful long-term treatment of hyponatremia in syndrome of inappropriate antidiuretic hormone secretion with satavaptan (SR121463B), an orally active nonpeptide vasopressin V2-receptor antagonist Clinical Journal of the American Society of Nephrology 1(6), 1154-1160.
Streefkerk, J. O.; van Zwieten, P. A. (2006) Vasopressin receptor antagonists: pharmacological tools and potential therapeutic agents Autonomic & Autacoid Pharmacology 26(2), 141-148.
Stymiest, Jake L.; Mitchell, Bryan F.; Wong, Susan; Vederas, John C. (2005) Synthesis of Oxytocin Analogues with Replacement of Sulfur by Carbon Gives Potent Antagonists with Increased Stability. Journal of Organic Chemistry 70(20), 7799-7809.
Tagawa, Tatsuya; Imaizumi, Tsutomu; Shiramoto, Masanari; Endo, Toyonari; Hironaga, Kiyoshi; Takeshita, Akira (1995) V2 receptor-mediated vasodilation in healthy humans. Journal of Cardiovascular Pharmacology 25(3), 387-92.
Tahara, Atsuo; Tomura, Yuichi; Wada, Koh-ichi; Kusayama, Toshiyuki; Tsukada, Junko; Ishii, Noe; Yatsu, Takeyuki; Uchida, Wataru; Tanaka, Akihiro (1997) Effect of YM087, a potent nonpeptide vasopressin antagonist, on vasopressin-induced hyperplasia and hypertrophy of cultured vascular smooth-muscle cells. Journal of Cardiovascular Pharmacology 30(6), 759-766.
Tahara, Atsuo; Tomura, Yuichi; Wada, Koh-ichi; Kusayama, Toshiyuki; Tsukada, Junko; Ishii, Noe; Yatsu, Takeyuki; Uchida, Wataru; Tanaka, Akihiro (1998) Effect of YM087, a potent nonpeptide vasopressin antagonist, on vasopressin-induced protein synthesis in neonatal rat cardiomyocyte Cardiovascular Research 38(1), 198-205.
Thibonnier, M.; Coles, P.; Thibonnier, A.; Shoham, M. (2001) The basic and clinical pharmacology of nonpeptide vasopressin receptor antagonists. Annual Review of Pharmacology and Toxicology 41, 175-202.
Treschan, Tanja A.; Peters, Juergen (2006) The Vasopressin System: Physiology and Clinical Strategies Anesthesiology 105(3), 599-612.
Tsatsaris, V.; Carbonne, B.; Cabrol, D. Atosiban for Preterm Labour. (2004) Drugs 64, 375-382.
Valenzuela, Guillermo J.; Sanchez-Ramos, Luis; Romero, Roberto; Silver, Helayne M.; Koltun, William D.; Millar, Lynnae; Hobbins, John; Rayburn, William; Shangold, Gary; Wang, Julianne; et al (2000) Maintenance treatment of preterm labor with the oxytocin antagonist atosiban. American Journal of Obstetrics and Gynecology 182(5), 1184-1190.
Van Lieburg, Angenita F.; Knoers, Nine V. A. M.; Monnens, Leo A. H.; Smits, Paul (1995) Effects of arginine vasopressin and 1-desamino-8-D arginine vasopressin on forearm vasculature of healthy subjects and patients with a V2 receptor defect. Journal of Hypertension 13(12, Pt. 2), 1695-700.
Verbalis, Joseph G. Edited by Kastin, Abba J (2006) Renal effects of neurohypophyseal peptides Handbook of Biologically Active Peptides 1227-1233.
Walker, Benjimen R.; Haynes, Johnson, Jr.; Wang, Huai Liang; Voelkel, Norbert F. (1989) Vasopressin-induced pulmonary vasodilation in rats. American Journal of Physiology 257(2, Pt. 2), H415-H422.
Yamamura, Yoshitaka; Nakamura, Shigeki; Itoh, Shuji; Hirano, Takahiro; Onogawa, Toshiyuki; Yamashita, Tatsuya; Yamada, Yoshihisa; Tsujimae, Kenji; Aoyama, Masashi; Kotosai, Kounori; et al (1998) OPC-41061, a highly potent human vasopressin V2-receptor antagonist: pharmacological profile and aquaretic effect by single and multiple oral dosing in rats. Journal of Pharmacology and Experimental Therapeutics 287(3), 860-867.
Yang, Jun; Chen, Jian-min; Liu, Wen-yan; Song, Chao-you; Lin, Bao-cheng (2006) Through V2, not V1 receptor relating to endogenous opiate peptides, arginine vasopressin in periaqueductal gray regulates antinociception in the rat Regulatory Peptides 137(3),156-161.

All patents and publications referred to herein are incorporated by reference herein to the same extent as if each individual publication was specifically and individually indicated to be incorporated by reference in its entirety.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A compound of formula (I) 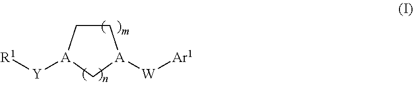
wherein
each A is independently N or CR, provided that at least one A is N;
m and n are independently 0, 1, 2, or 3;
wherein the ring comprising A optionally comprises a double bond; and,
the ring comprising A is substituted with 0-3 J;
W and Y are each independently a bond, (CHR)₁₋₄, (CH₂)₀₋₂O, (CH₂)₀₋₂C(O)(CH₂)₀₋₂, (CH₂)₀₋₂CR₂(CH₂)₀₋₂, (CH₂)₀₋₂C(OR)(R)(CH₂)₀₋₂, O(CR₂)₁₋₄O, (CH₂)₀₋₂N(R)(CH₂)₀₋₂, (CH₂)₀₋₂S(CH₂)₀₋₂, (CH₂)₀₋₂SO(CH₂)₀₋₂, (CH₂)₀₋₂SO₂(CH₂)₀₋₂, (CH₂)₀₋₂SO₂N(R)(CH₂)₀₋₂, (CH₂)₀₋₂SO₃(CH₂)₀₋₂, (CH₂)₀₋₂C(O)C(O)(CH₂)₀₋₂, (CH₂)₀₋₂C(O)CH₂C(O)(CH₂)₀₋₂, (CH₂)₀₋₂C(S)(CH₂)₀₋₂, (CH₂)₀₋₂C(O)O(CH₂)₀₋₂, (CH₂)₀₋₂OC(O)(CH₂)₀₋₂, (CH₂)₀₋₂OC(O)O(CH₂)₀₋₂, (CH₂)₀₋₂C(O)N(R)(CH₂)₀₋₂, (CH₂)₀₋₂OC(O)N(R)(CH₂)₀₋₂, (CH₂)₀₋₂C(S)N(R)(CH₂)₀₋₂, (CH₂)₀₋₂NHC(O)(CH₂)₀₋₂, (CH₂)₀₋₂N(R)N(R)C(O)(CH₂)₀₋₂, (CH₂)₀₋₂N(R)N(R)C(O)O(CH₂)₀₋₂, (CH₂)₀₋₂N(R)N(R)CON(R)(CH₂)₀₋₂, (CH₂)₀₋₂N(R)SO₂ (CH₂)₀₋₂ (CH₂)₀₋₂N(R)SO₂N(R)(CH₂)₀₋₂, (CH₂)₀₋₂N(R)C(O)O(CH₂)₀₋₂, (CH₂)₀₋₂N(R)C(O)(CH₂)₀₋₂, (CH₂)₀₋₂N(R)C(S)(CH₂)₀₋₂, (CH₂)₀₋₂N(R)C(O)N(R)(CH₂)₀₋₂, (CH₂)₀₋₂N(R)C(S)N(R)(CH₂)₀₋₂, (CH₂)₀₋₂N(COR)CO(CH₂)₀₋₂, (CH₂)₀₋₂N(OR)(CH₂)₀₋₂, (CH₂)₀₋₂C(═NH)N(R)(CH₂)₀₋₂, (CH₂)₀₋₂C(O)N(OR)(CH₂)₀₋₂, or (CH₂)₀₋₂C(═NOR)(CH₂)₀₋₂;
wherein R is independently at each occurrence hydrogen or an alkyl, heteroalkyl, acyl, cycloalkyl, cycloalkylalkyl, aryl, aralkyl, heterocyclyl, heterocyclylalkyl, heteroaryl, or heteroarylalkyl, wherein any alkyl, heteroalkyl, acyl, cycloalkyl, cycloalkylalkyl, aryl, aralkyl, heterocyclyl, heterocyclylalkyl, heteroaryl, or heteroarylalkyl is substituted with 0-3 J; or wherein two R groups together with a nitrogen atom or with two adjacent nitrogen atoms to which they are bonded can together form a 3-8 membered heterocyclyl substituted with 0-3 J, optionally further comprising 1-3 additional heteroatoms selected from the group consisting of O, NR, S, S(O) and S(O)₂;
R¹ is hydrogen, alkyl, cycloalkyl, cycloalkylalkyl, aryl, aralkyl, heterocyclyl, heterocyclylalkyl, heteroaryl, or heteroarylalkyl, wherein any alkyl, cycloalkyl, cycloalkylalkyl, aryl, aralkyl, heterocyclyl, heterocyclylalkyl, heteroaryl, or heteroarylalkyl is substituted with 0-3 J;
Ar¹ is cycloalkyl, aryl, heterocyclyl, or heteroaryl, wherein any cycloalkyl, aryl, heterocyclyl, or heteroaryl, is mono- or independently multi-substituted with J, (C₁-C₆)alkyl, (C₂-C₆)alkenyl, (C₂-C₆) alkynyl, (C₁-C₆)haloalkyl, (C₁-C₆)alkoxy, (C₁-C₆)haloalkoxy, cycloalkyl-(C₀-C₆)alkyl, heterocyclyl-(C₀-C₆)alkyl, aryl-(C₀-C₆)alkyl, or heteroaryl-(C₀-C₆)alkyl, wherein any alkyl, alkenyl, alkynyl, haloalkyl, alkoxy, haloalkoxy, cycloalkyl, heterocyclyl, aryl, or heteroaryl is optionally mono- or independently multi-substituted with J;
J is independently at each occurrence F, Cl, Br, I, OR^{J}, CN, CF₃, OCF₃, O, S, C(O), S(O), methylenedioxy, ethylenedioxy, N(R^{J})₂, SR^{J}, SOR^{J}, SO₂R^{J}, SO₂N(R^{J})₂, SO₃R^{J}, C(O)R^{J}, C(O)C(O)R^{J}, C(O)CH₂C(O)R^{J}, C(S)R^{J}, C(O)OR^{J}, OC(O)R^{J}, OC(O)OR^{J}, C(O)N(R^{J})₂, OC(O)N(R^{J})₂, C(S)N(R^{J})₂, (CH₂)₀₋₂NHC(O)R^{J}, N(R^{J})N(R^{J})C(O)R^{J}, N(R^{J})N(R^{J})C(O)OR^{J}, N(R^{J})N(R^{J})CON(R^{J})₂, N(R^{J})SO₂R^{J}, N(R^{J})SO₂N(R^{J})₂, N(R^{J})C(O)OR^{J}, N(R^{J})C(O)R^{J}, N(R^{J})C(S)R^{J}, N(R^{J})C(O)N(R^{J})₂, N(R^{J})C(S)N(R^{J})₂, N(COR^{J})COR^{J}, N(OR)R^{J}, C(═NH)N(R^{J})₂, C(O)N(OR^{J})R^{J}, or C(═NOR^{J})R; wherein R^{J} is hydrogen, alkyl, cycloalkyl, cycloalkylalkyl, aryl, aralkyl, heterocyclyl, heterocyclylalkyl, heteroaryl, or heteroarylalkyl; or J is Ar² wherein Ar² is cycloalkyl, aryl, heterocyclyl, or heteroaryl, wherein any cycloalkyl, aryl, heterocyclyl, or heteroaryl, is mono- or independently multi-substituted with J, (C₁-C₆)alkyl, (C₂-C₆)alkenyl, (C₂-C₆)alkynyl, (C₁-C₆)haloalkyl, (C₁-C₆)alkoxy, (C₁-C₆)haloalkoxy, cycloalkyl-(C₀-C₆)alkyl, heterocyclyl-(C₀-C₆)alkyl, aryl-(C₀-C₆)alkyl, or heteroaryl-(C₀-C₆)alkyl, wherein any alkyl, alkenyl, alkynyl, haloalkyl, alkoxy, haloalkoxy, cycloalkyl, heterocyclyl, aryl, or heteroaryl is optionally mono- or independently multi-substituted with J;
wherein any cycloalkyl, aryl, heterocyclyl, or heteroaryl comprised by formula (I) can be fused, bridged, or in a spiro configuration with one or more additional optionally substituted cycloalkyl, aryl, heterocyclyl, and heteroaryl, monocyclic, bicyclic or polycyclic, saturated, partially unsaturated, or aromatic rings; and wherein any heterocyclyl or heteroaryl comprising nitrogen can be an N-oxide or N-metho salt thereof;
or a pharmaceutically acceptable isomer, racemate, hydrate, solvate, isotope, or salt thereof.

2. The compound of formula (I) of claim 1 comprising a compound of formula (IA) 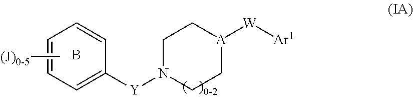
wherein Y is a bond, (CHR)₁₋₄, (CH₂)₀₋₂C(O)(CH₂)₀₋₂, or (CH₂)₀₋₂OC(O)(CH₂)₀₋₂, and ring B can comprise one or two N atoms, and wherein A, J, W, and Ar¹ are as defined in claim 1.

3. The compound of formula (I) of claim 1 comprising a compound of formula (IB) 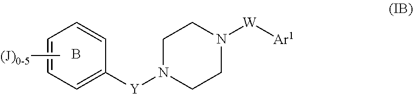
wherein Y is a bond, (CHR)₁₋₄, (CH₂)₀₋₂C(O)(CH₂)₀₋₂, or (CH₂)₀₋₂OC(O)(CH₂)₀₋₂, and ring B can comprise one or two N atoms, and wherein J, W, and Ar¹ are as defined in claim 1.

4. The compound of formula (I) of claim 1 comprising a compound of formula (IC) 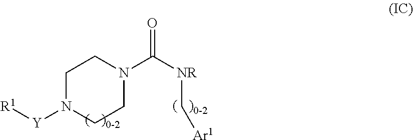
wherein R, Y, and Ar¹ are as defined in claim 1.

5. The compound of formula (I) of claim 1 comprising a compound of formula (ID) 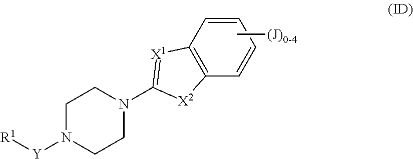
wherein J, R¹, and Y are as defined in claim 1, and wherein X¹ is CR or N, and X² is NR, S, or O.

6. The compound of formula (I) of claim 1, wherein Ar¹ is any of the following 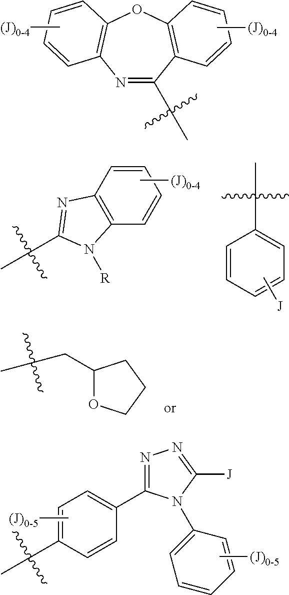
wherein J is as defined in claim 1 and a wavy line indicates a point of attachment.

7. The compound of formula (I) of claim 1, wherein R¹ is unsubstituted or substituted phenyl or pyridyl.

8. The compound of formula (I) of claim 1, wherein W is C(O) or C(O)NR, or wherein Y is a bond, or both.

9. The compound of formula (I) of claim 1, wherein the group of formula 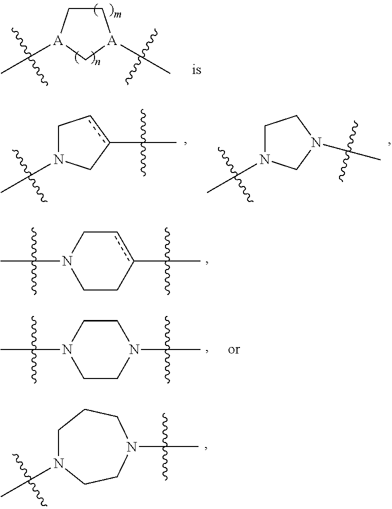
wherein a wavy line indicates a point of attachment, wherein a dashed line indicates that a single or a double bond can be present.

10. The compound of formula (I) of claim 1, wherein the compound is any of the following: 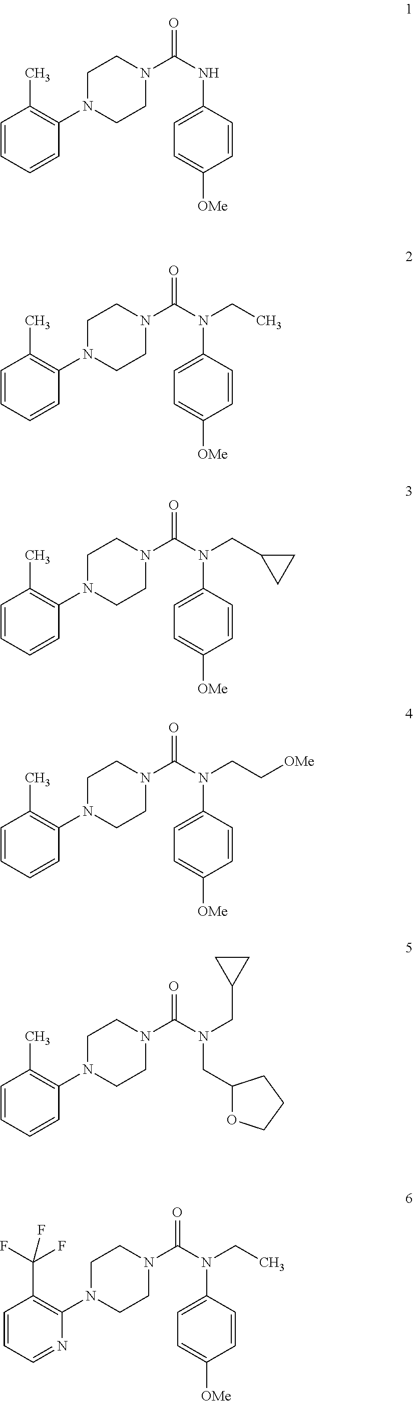 [SMILES: COc1ccc(NC(=O)N2CCN(c3ccccc3C)CC2)cc1 ; CCN(C(=O)N1CCN(c2ccccc2C)CC1)c1ccc(OC)cc1 ; COc1ccc(N(CC2CC2)C(=O)N2CCN(c3ccccc3C)CC2)cc1 ; COCCN(C(=O)N1CCN(c2ccccc2C)CC1)c1ccc(OC)cc1 ; Cc1ccccc1N1CCN(C(=O)N(CC2CC2)CC2CCCO2)CC1 ; CCN(C(=O)N1CCN(c2ncccc2C(F)(F)F)CC1)c1ccc(OC)cc1] 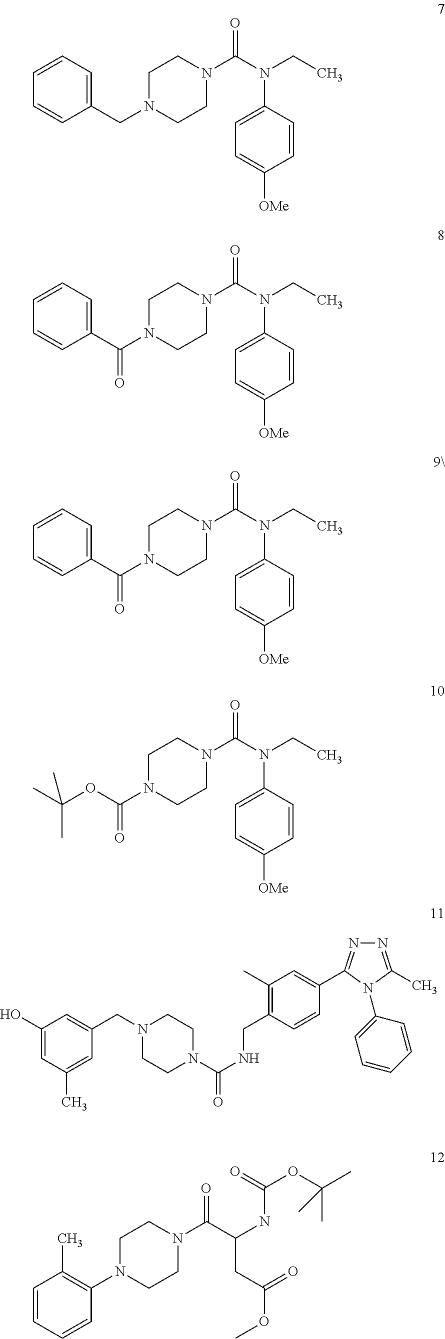 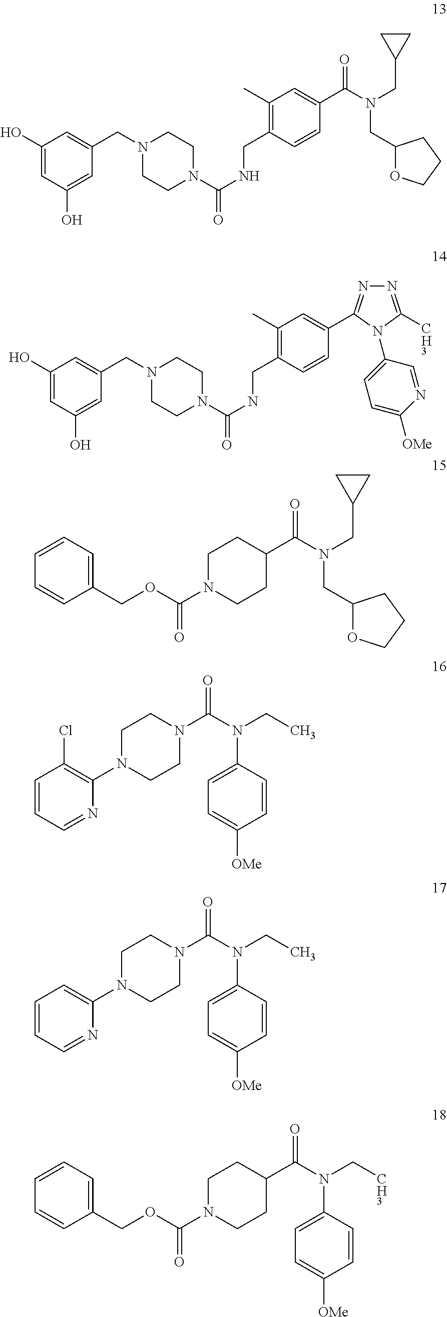 [SMILES: Cc1cc(C(=O)N(CC2CC2)CC2CCCO2)ccc1CNC(=O)N1CCN(Cc2cc(O)cc(O)c2)CC1 ; COc1ccc(-n2c(C)nnc2-c2ccc(CNC(=O)N3CCN(Cc4cc(O)cc(O)c4)CC3)c(C)c2)cn1 ; O=C(OCc1ccccc1)N1CCC(C(=O)N(CC2CC2)CC2CCCO2)CC1 ; CCN(C(=O)N1CCN(c2ncccc2Cl)CC1)c1ccc(OC)cc1 ; CCN(C(=O)N1CCN(c2ccccn2)CC1)c1ccc(OC)cc1 ; CCN(C(=O)C1CCN(C(=O)OCc2ccccc2)CC1)c1ccc(OC)cc1] 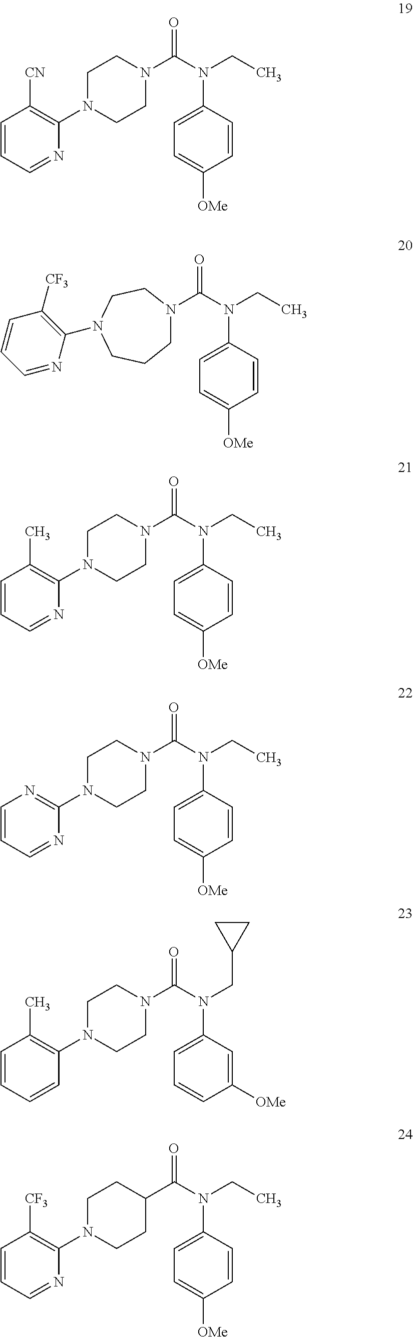 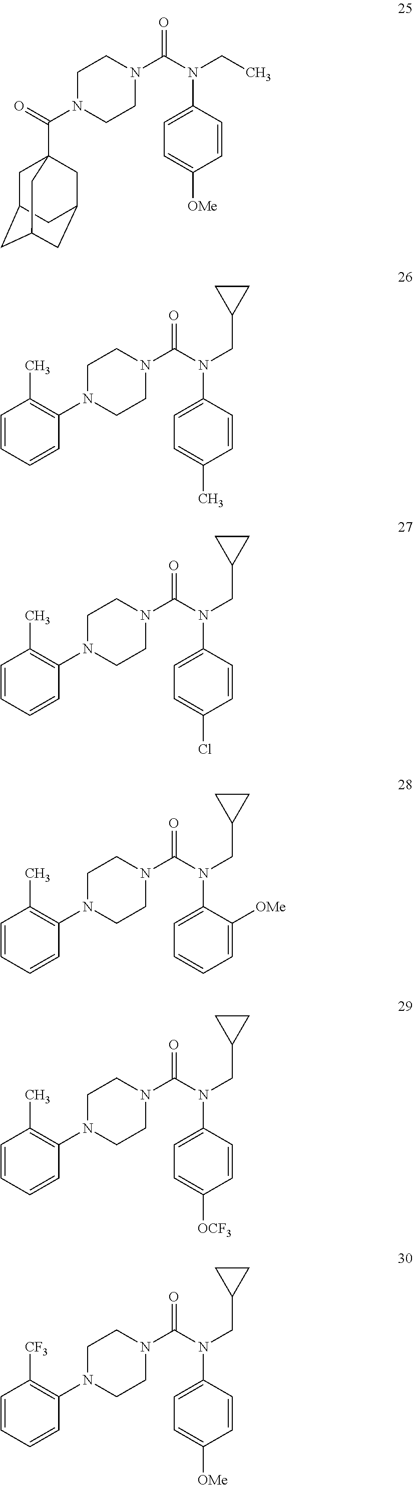 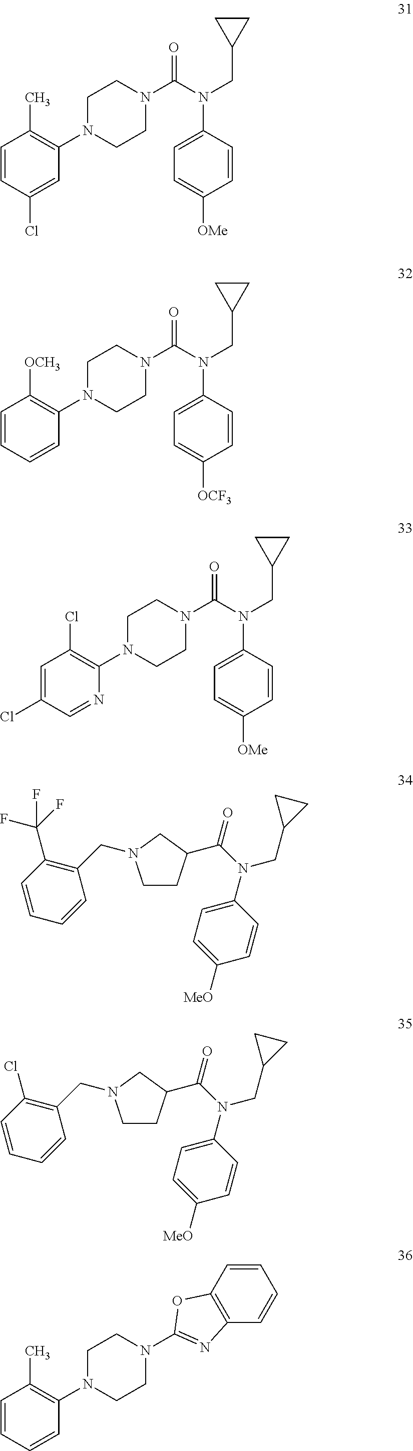 [SMILES: COc1ccc(N(CC2CC2)C(=O)N2CCN(c3cc(Cl)ccc3C)CC2)cc1 ; COc1ccccc1N1CCN(C(=O)N(CC2CC2)c2ccc(OC(F)(F)F)cc2)CC1 ; COc1ccc(N(CC2CC2)C(=O)N2CCN(c3ncc(Cl)cc3Cl)CC2)cc1 ; COc1ccc(N(CC2CC2)C(=O)C2CCN(Cc3ccccc3C(F)(F)F)C2)cc1 ; COc1ccc(N(CC2CC2)C(=O)C2CCN(Cc3ccccc3Cl)C2)cc1 ; Cc1ccccc1N1CCN(c2nc3ccccc3o2)CC1] 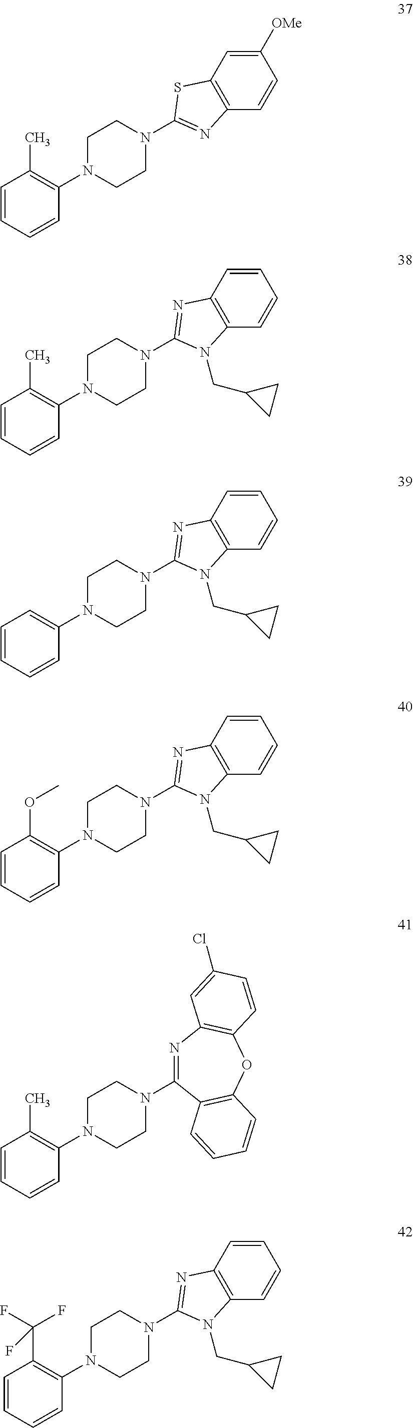 [SMILES: COc1ccc2nc(N3CCN(c4ccccc4C)CC3)sc2c1 ; Cc1ccccc1N1CCN(c2nc3ccccc3n2CC2CC2)CC1 ; c1ccc(N2CCN(c3nc4ccccc4n3CC3CC3)CC2)cc1 ; COc1ccccc1N1CCN(c2nc3ccccc3n2CC2CC2)CC1 ; Cc1ccccc1N1CCN(C2=Nc3cc(Cl)ccc3Oc3ccccc32)CC1 ; FC(F)(F)c1ccccc1N1CCN(c2nc3ccccc3n2CC2CC2)CC1] 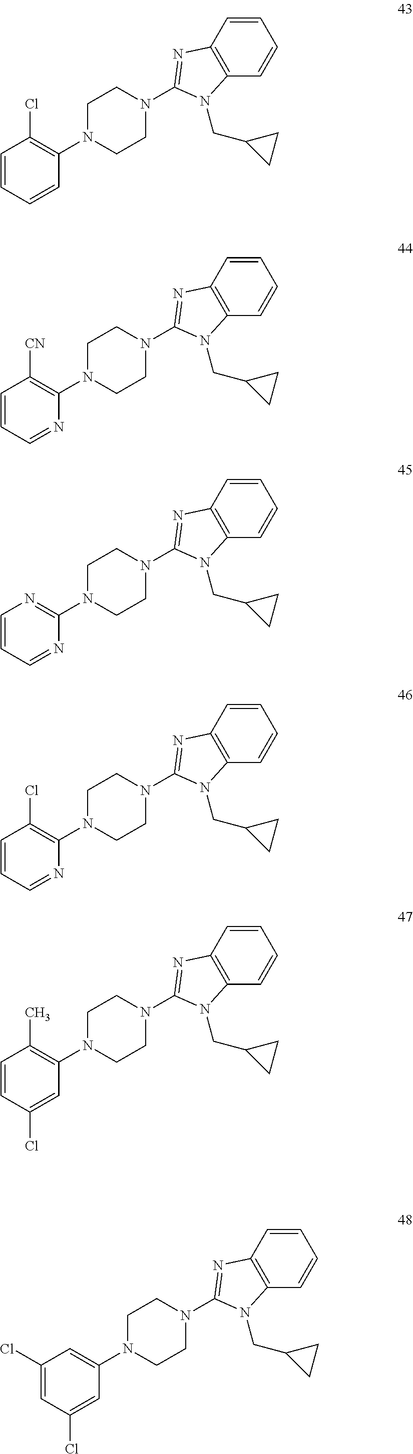 [SMILES: Clc1ccccc1N1CCN(c2nc3ccccc3n2CC2CC2)CC1 ; N#Cc1cccnc1N1CCN(c2nc3ccccc3n2CC2CC2)CC1 ; c1cnc(N2CCN(c3nc4ccccc4n3CC3CC3)CC2)nc1 ; Clc1cccnc1N1CCN(c2nc3ccccc3n2CC2CC2)CC1 ; Cc1ccc(Cl)cc1N1CCN(c2nc3ccccc3n2CC2CC2)CC1 ; Clc1cc(Cl)cc(N2CCN(c3nc4ccccc4n3CC3CC3)CC2)c1] 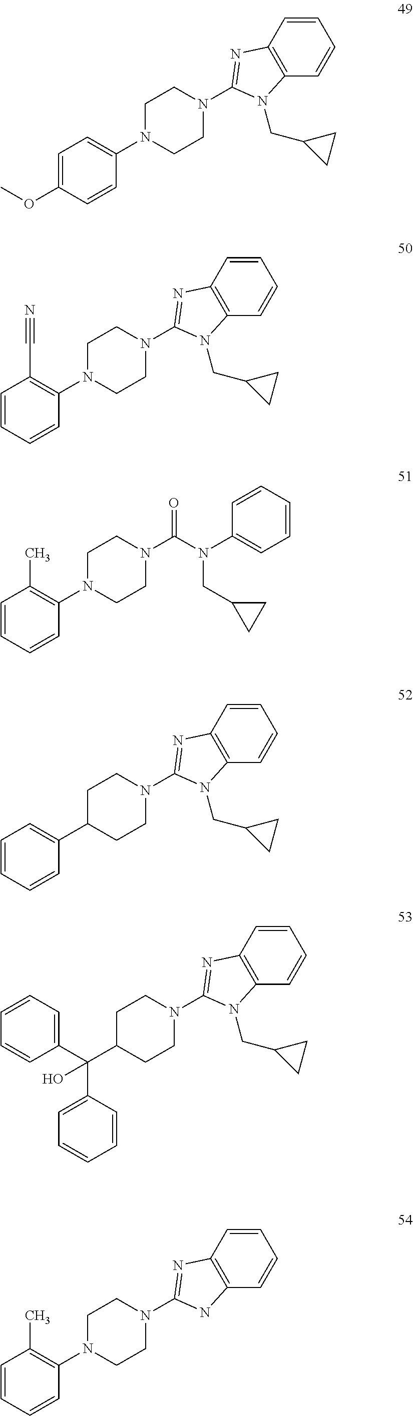 [SMILES: COc1ccc(N2CCN(c3nc4ccccc4n3CC3CC3)CC2)cc1 ; N#Cc1ccccc1N1CCN(c2nc3ccccc3n2CC2CC2)CC1 ; Cc1ccccc1N1CCN(C(=O)N(CC2CC2)c2ccccc2)CC1 ; c1ccc(C2CCN(c3nc4ccccc4n3CC3CC3)CC2)cc1 ; OC(c1ccccc1)(c1ccccc1)C1CCN(c2nc3ccccc3n2CC2CC2)CC1 ; Cc1ccccc1N1CCN(c2nc3ccccc3[nH]2)CC1] 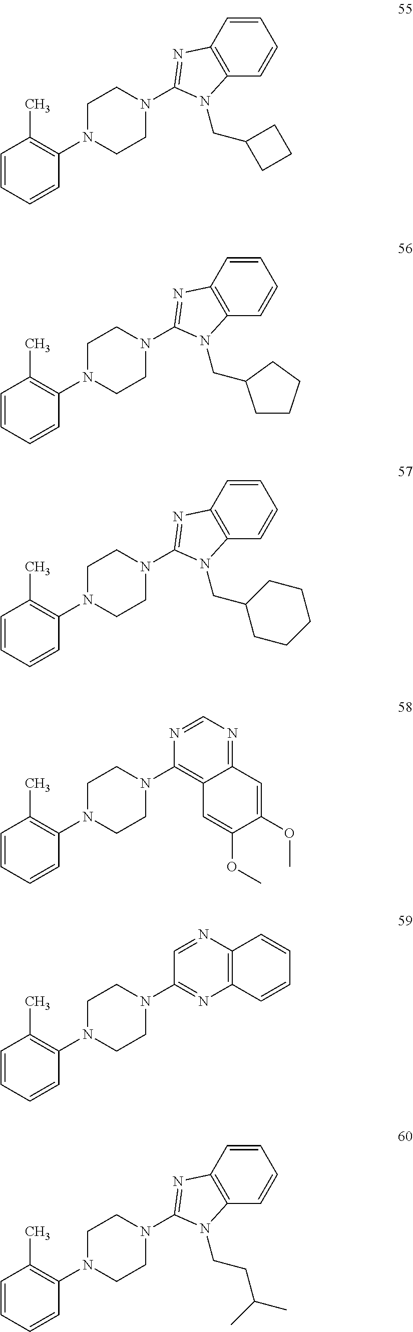 [SMILES: Cc1ccccc1N1CCN(c2nc3ccccc3n2CC2CCC2)CC1 ; Cc1ccccc1N1CCN(c2nc3ccccc3n2CC2CCCC2)CC1 ; Cc1ccccc1N1CCN(c2nc3ccccc3n2CC2CCCCC2)CC1 ; COc1cc2ncnc(N3CCN(c4ccccc4C)CC3)c2cc1OC ; Cc1ccccc1N1CCN(c2cnc3ccccc3n2)CC1 ; Cc1ccccc1N1CCN(c2nc3ccccc3n2CCC(C)C)CC1] 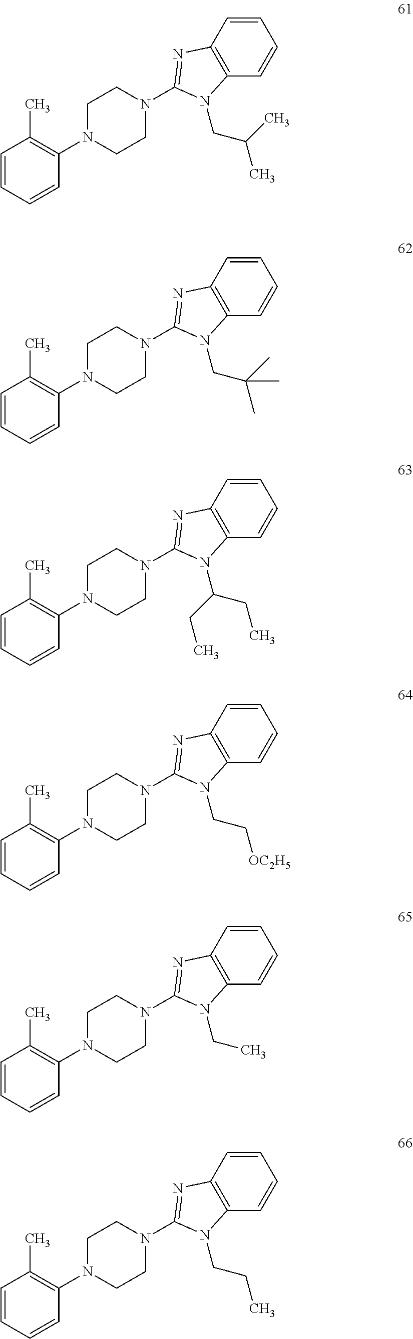 [SMILES: Cc1ccccc1N1CCN(c2nc3ccccc3n2CC(C)C)CC1 ; Cc1ccccc1N1CCN(c2nc3ccccc3n2CC(C)(C)C)CC1 ; CCC(CC)n1c(N2CCN(c3ccccc3C)CC2)nc2ccccc21 ; CCOCCn1c(N2CCN(c3ccccc3C)CC2)nc2ccccc21 ; CCn1c(N2CCN(c3ccccc3C)CC2)nc2ccccc21 ; CCCn1c(N2CCN(c3ccccc3C)CC2)nc2ccccc21] 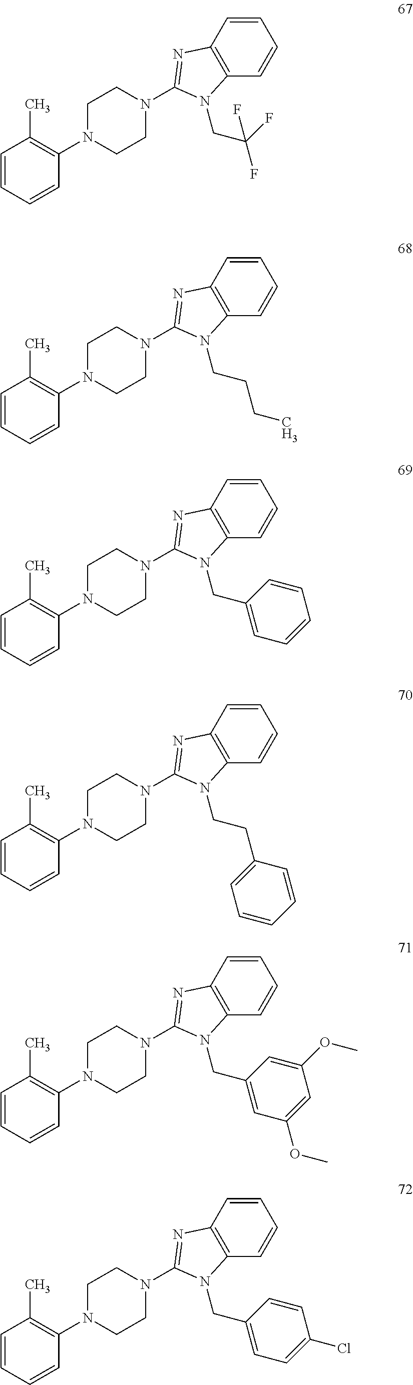 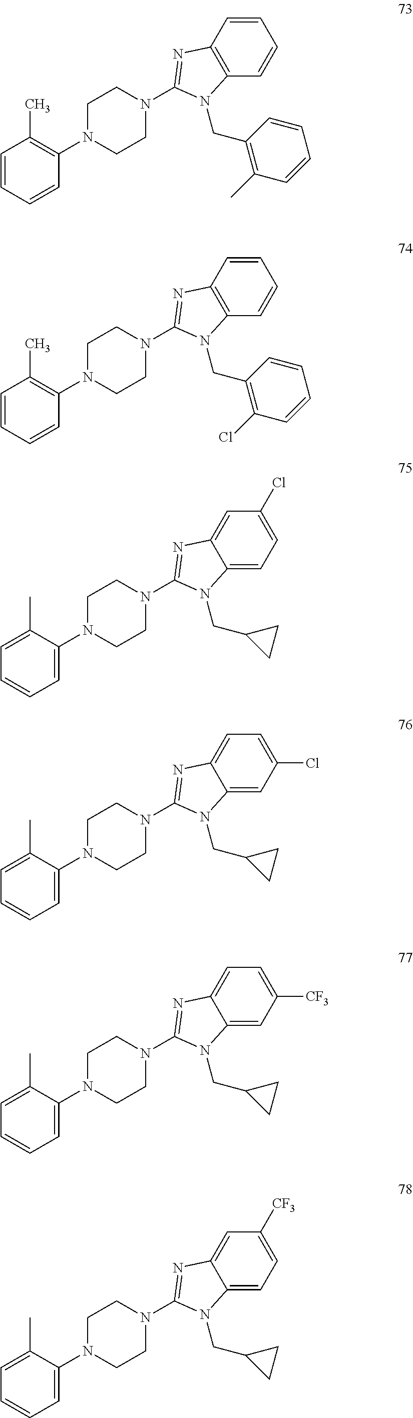 [SMILES: Cc1ccccc1Cn1c(N2CCN(c3ccccc3C)CC2)nc2ccccc21 ; Cc1ccccc1N1CCN(c2nc3ccccc3n2Cc2ccccc2Cl)CC1 ; Cc1ccccc1N1CCN(c2nc3cc(Cl)ccc3n2CC2CC2)CC1 ; Cc1ccccc1N1CCN(c2nc3ccc(Cl)cc3n2CC2CC2)CC1 ; Cc1ccccc1N1CCN(c2nc3ccc(C(F)(F)F)cc3n2CC2CC2)CC1 ; Cc1ccccc1N1CCN(c2nc3cc(C(F)(F)F)ccc3n2CC2CC2)CC1] 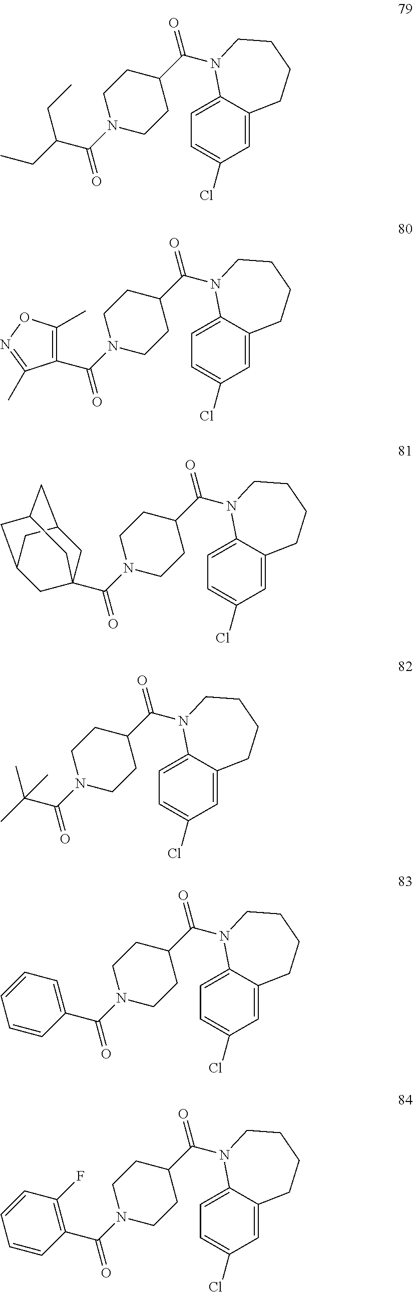 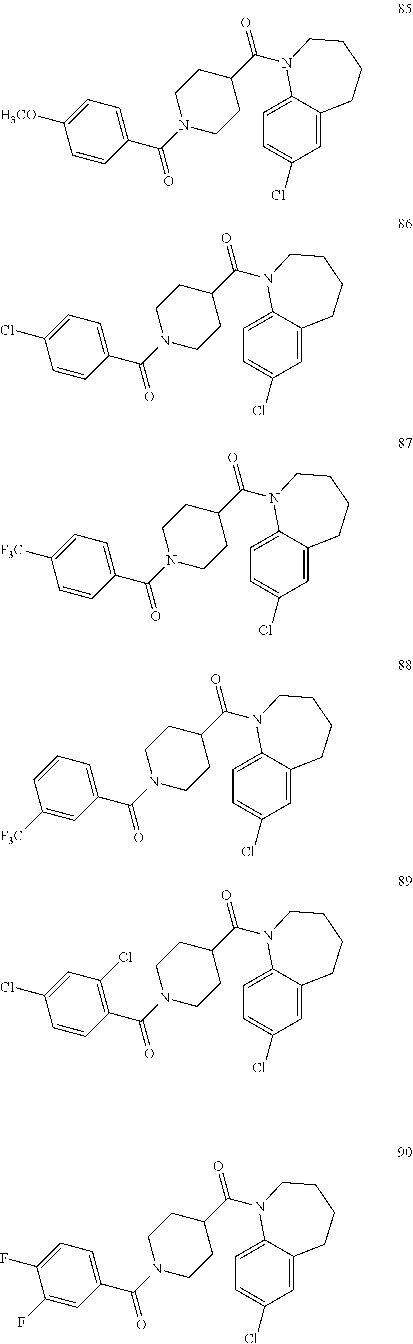 [SMILES: COc1ccc(C(=O)N2CCC(C(=O)N3CCCCc4cc(Cl)ccc43)CC2)cc1 ; O=C(c1ccc(Cl)cc1)N1CCC(C(=O)N2CCCCc3cc(Cl)ccc32)CC1 ; O=C(c1ccc(C(F)(F)F)cc1)N1CCC(C(=O)N2CCCCc3cc(Cl)ccc32)CC1 ; O=C(c1cccc(C(F)(F)F)c1)N1CCC(C(=O)N2CCCCc3cc(Cl)ccc32)CC1 ; O=C(c1ccc(Cl)cc1Cl)N1CCC(C(=O)N2CCCCc3cc(Cl)ccc32)CC1 ; O=C(c1ccc(F)c(F)c1)N1CCC(C(=O)N2CCCCc3cc(Cl)ccc32)CC1] 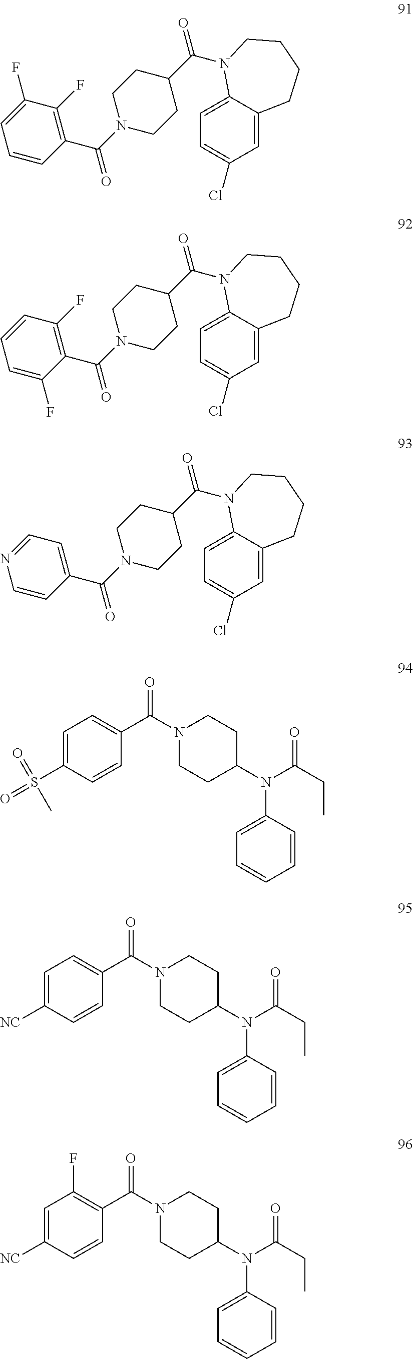 [SMILES: O=C(c1cccc(F)c1F)N1CCC(C(=O)N2CCCCc3cc(Cl)ccc32)CC1 ; O=C(c1c(F)cccc1F)N1CCC(C(=O)N2CCCCc3cc(Cl)ccc32)CC1 ; O=C(c1ccncc1)N1CCC(C(=O)N2CCCCc3cc(Cl)ccc32)CC1 ; CCC(=O)N(c1ccccc1)C1CCN(C(=O)c2ccc(S(C)(=O)=O)cc2)CC1 ; CCC(=O)N(c1ccccc1)C1CCN(C(=O)c2ccc(C#N)cc2)CC1 ; CCC(=O)N(c1ccccc1)C1CCN(C(=O)c2ccc(C#N)cc2F)CC1] 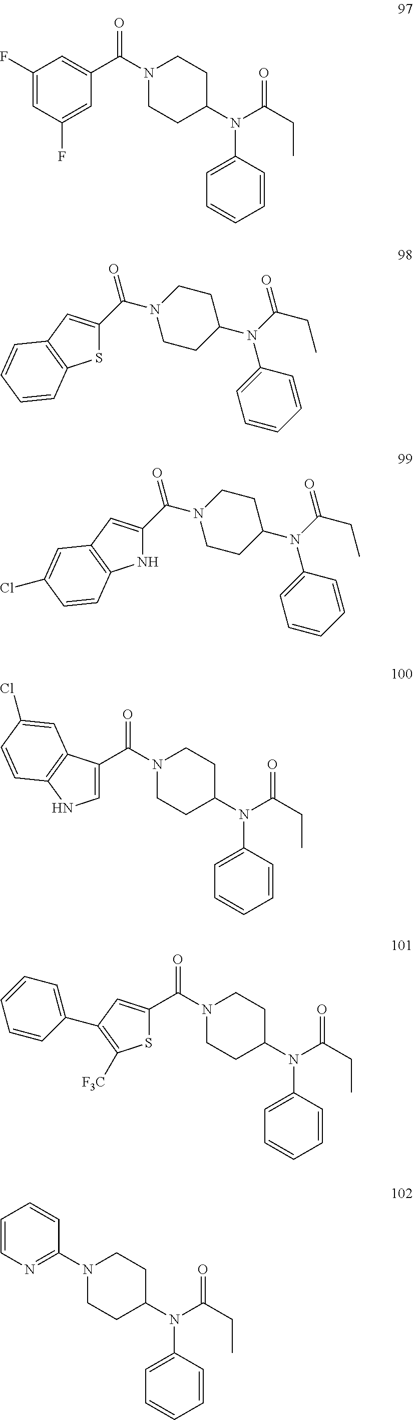 [SMILES: CCC(=O)N(c1ccccc1)C1CCN(C(=O)c2cc(F)cc(F)c2)CC1 ; CCC(=O)N(c1ccccc1)C1CCN(C(=O)c2cc3ccccc3s2)CC1 ; CCC(=O)N(c1ccccc1)C1CCN(C(=O)c2cc3cc(Cl)ccc3[nH]2)CC1 ; CCC(=O)N(c1ccccc1)C1CCN(C(=O)c2c[nH]c3ccc(Cl)cc23)CC1 ; CCC(=O)N(c1ccccc1)C1CCN(C(=O)c2cc(-c3ccccc3)c(C(F)(F)F)s2)CC1 ; CCC(=O)N(c1ccccc1)C1CCN(c2ccccn2)CC1] 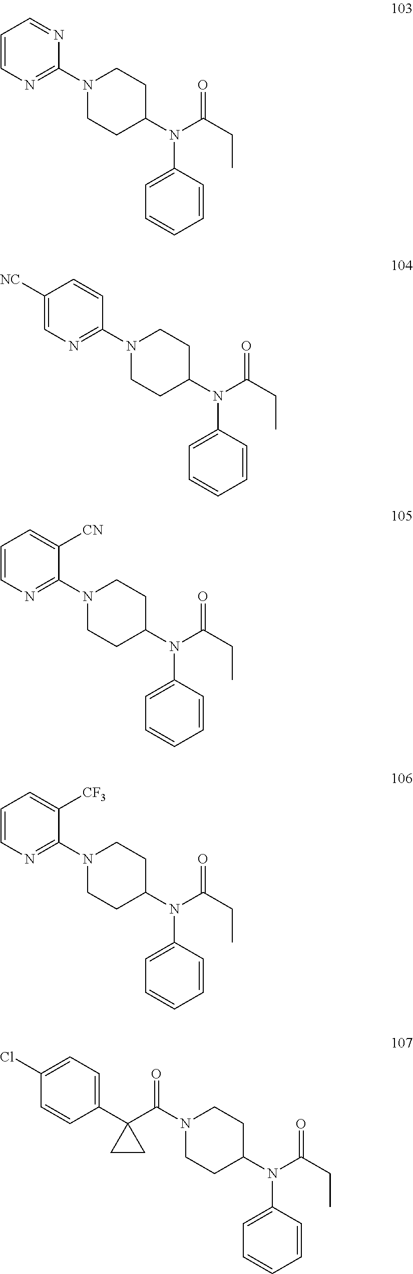 [SMILES: CCC(=O)N(c1ccccc1)C1CCN(c2ncccn2)CC1 ; CCC(=O)N(c1ccccc1)C1CCN(c2ccc(C#N)cn2)CC1 ; CCC(=O)N(c1ccccc1)C1CCN(c2ncccc2C#N)CC1 ; CCC(=O)N(c1ccccc1)C1CCN(c2ncccc2C(F)(F)F)CC1 ; CCC(=O)N(c1ccccc1)C1CCN(C(=O)C2(c3ccc(Cl)cc3)CC2)CC1] 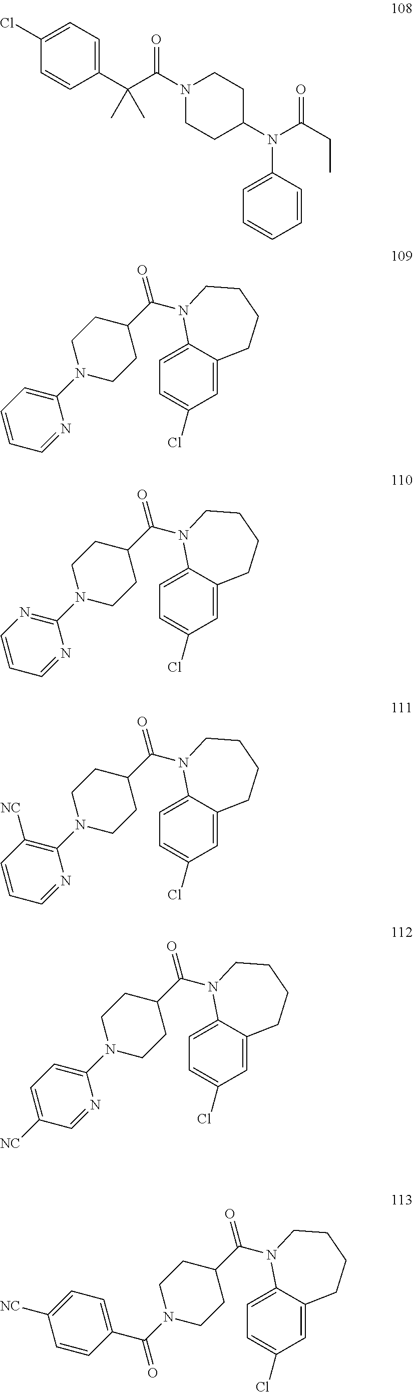 [SMILES: CCC(=O)N(c1ccccc1)C1CCN(C(=O)C(C)(C)c2ccc(Cl)cc2)CC1 ; O=C(C1CCN(c2ccccn2)CC1)N1CCCCc2cc(Cl)ccc21 ; O=C(C1CCN(c2ncccn2)CC1)N1CCCCc2cc(Cl)ccc21 ; N#Cc1cccnc1N1CCC(C(=O)N2CCCCc3cc(Cl)ccc32)CC1 ; N#Cc1ccc(N2CCC(C(=O)N3CCCCc4cc(Cl)ccc43)CC2)nc1 ; N#Cc1ccc(C(=O)N2CCC(C(=O)N3CCCCc4cc(Cl)ccc43)CC2)cc1] 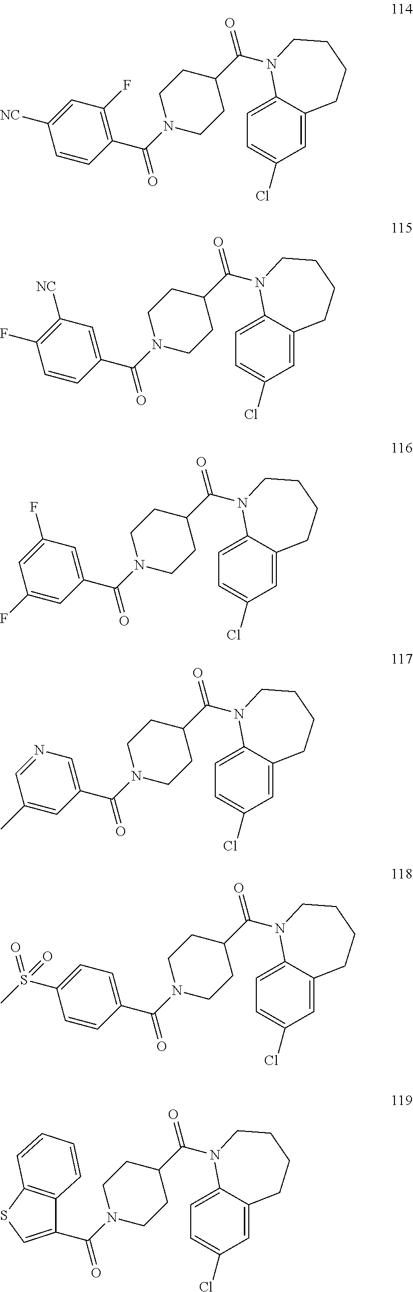 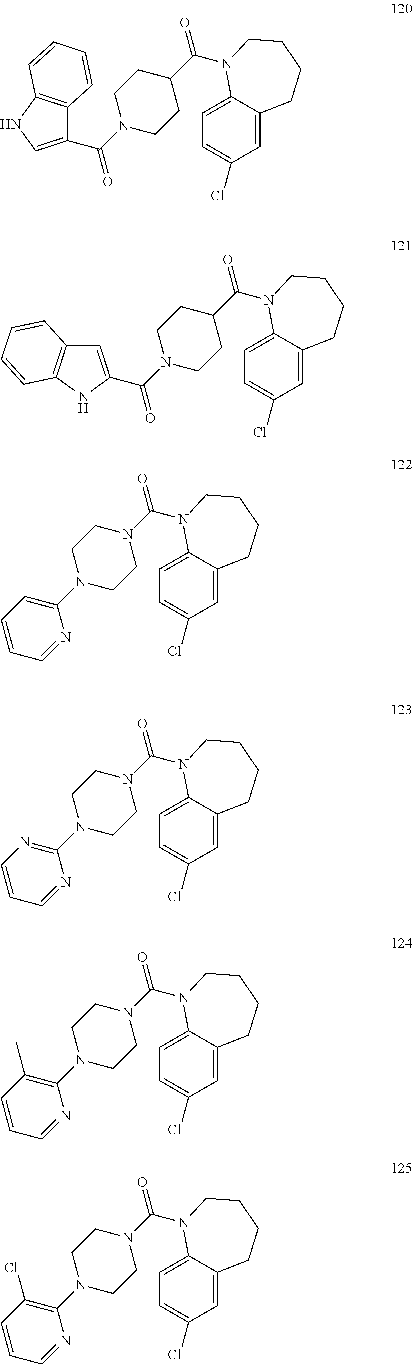 [SMILES: O=C(c1c[nH]c2ccccc12)N1CCC(C(=O)N2CCCCc3cc(Cl)ccc32)CC1 ; O=C(c1cc2ccccc2[nH]1)N1CCC(C(=O)N2CCCCc3cc(Cl)ccc32)CC1 ; O=C(N1CCN(c2ccccn2)CC1)N1CCCCc2cc(Cl)ccc21 ; O=C(N1CCN(c2ncccn2)CC1)N1CCCCc2cc(Cl)ccc21 ; Cc1cccnc1N1CCN(C(=O)N2CCCCc3cc(Cl)ccc32)CC1 ; O=C(N1CCN(c2ncccc2Cl)CC1)N1CCCCc2cc(Cl)ccc21] 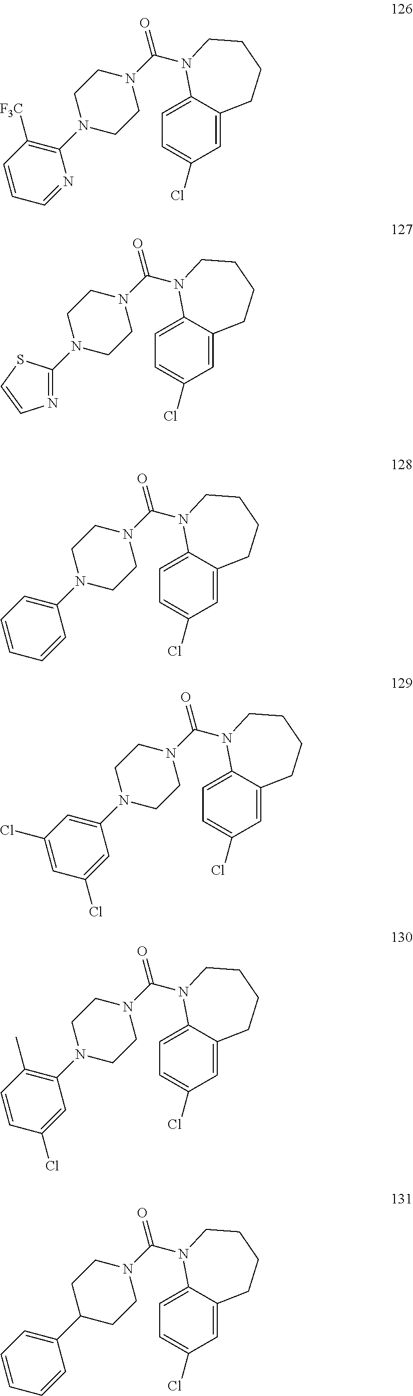 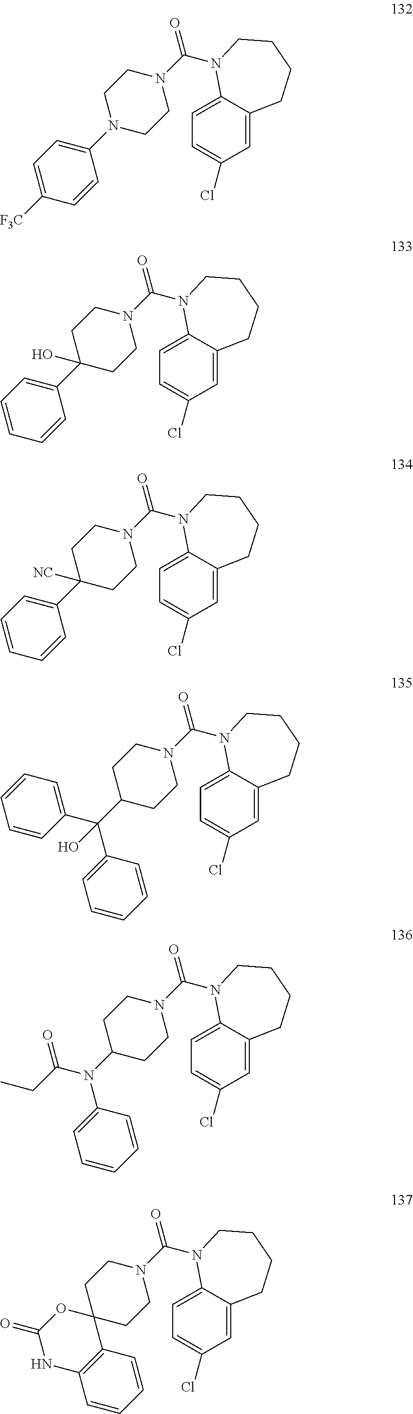 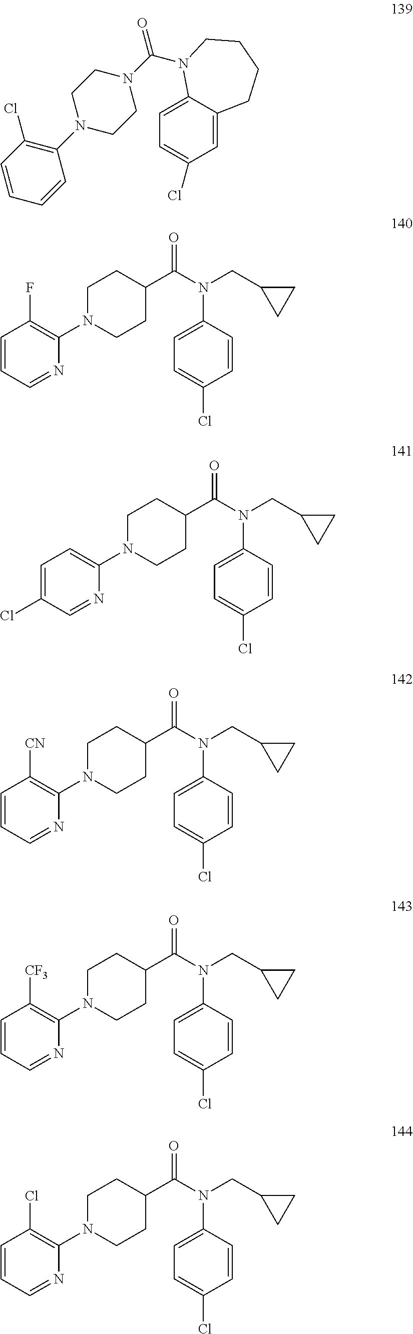 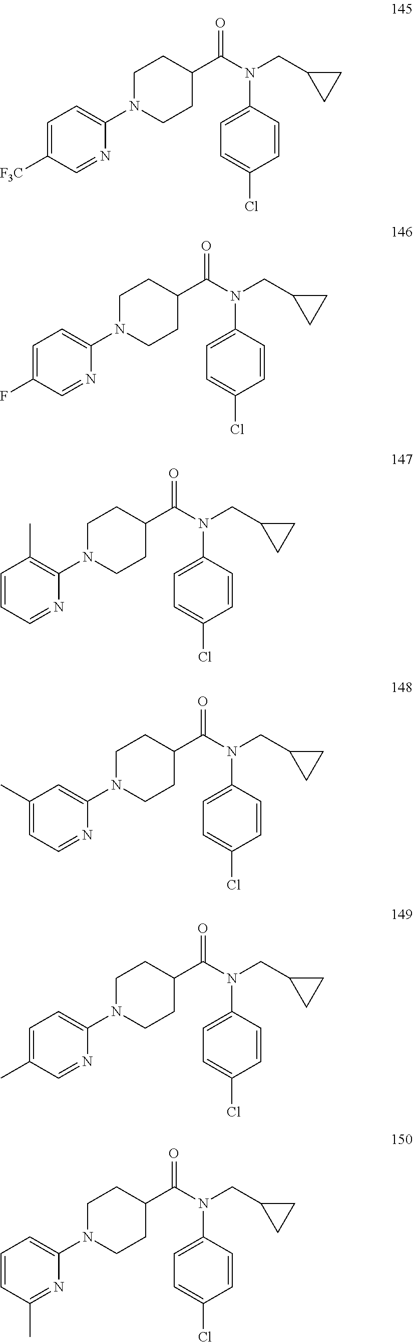 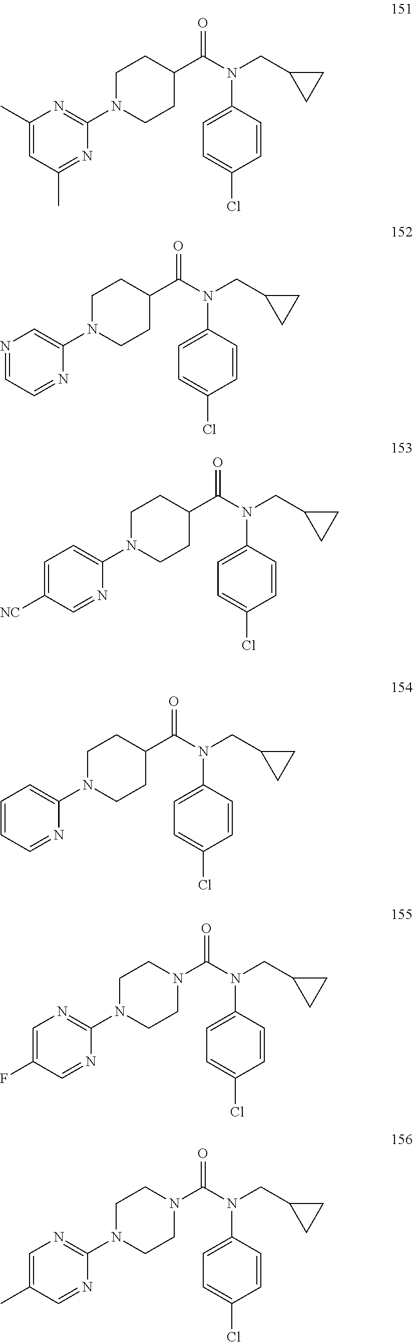 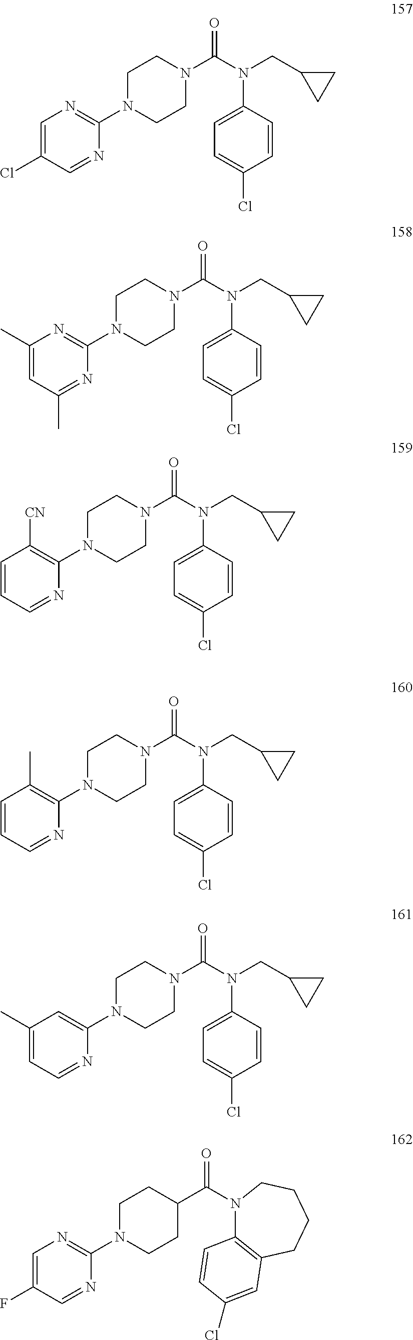 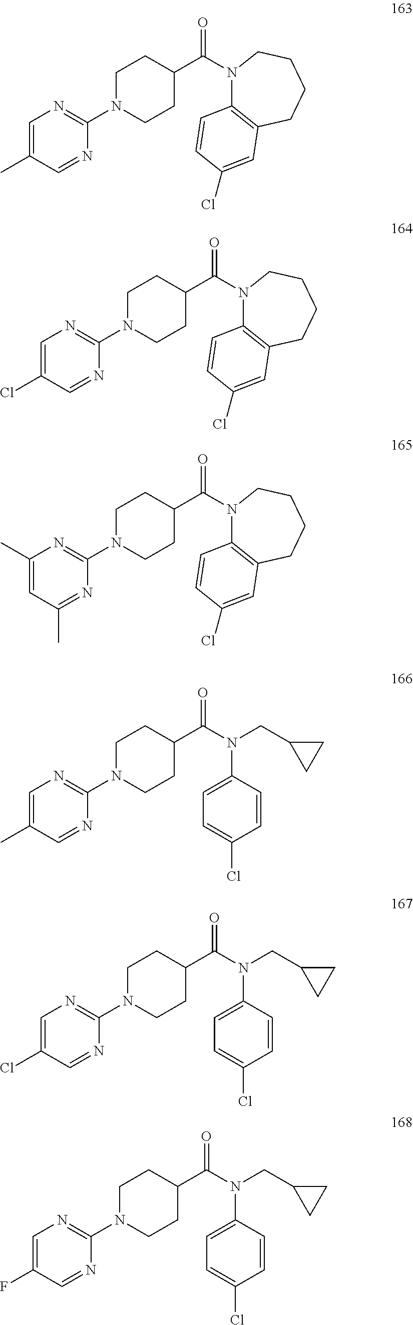 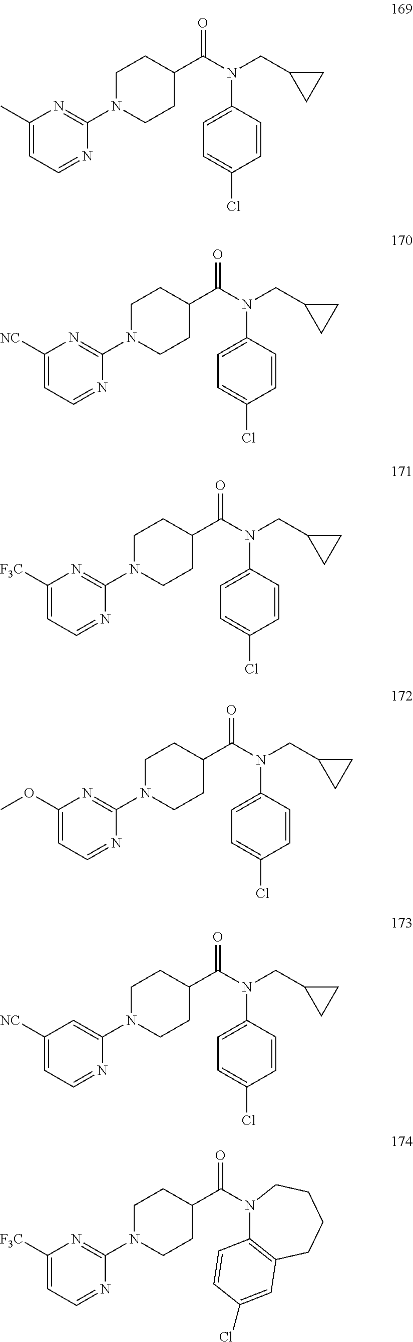 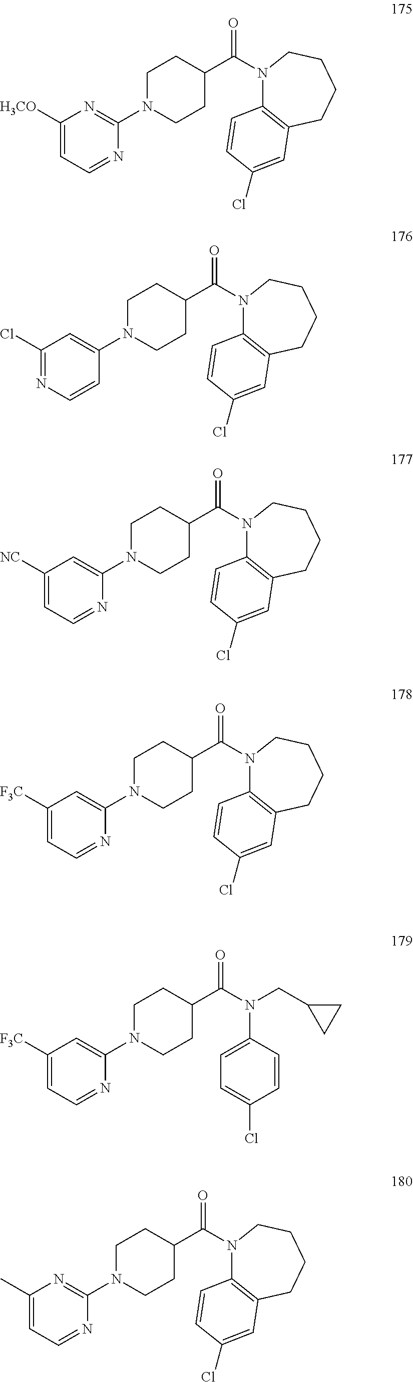 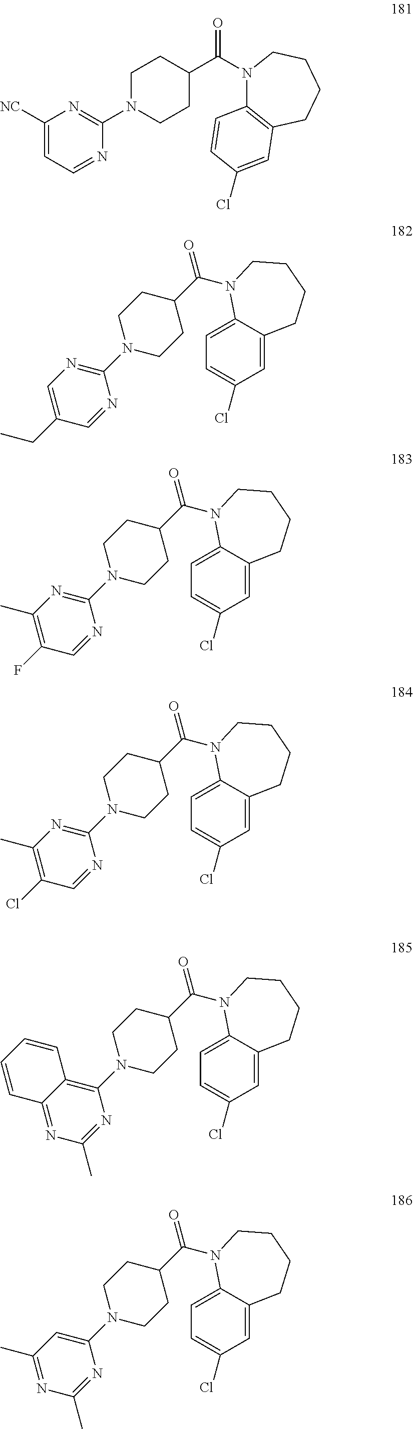 [SMILES: N#Cc1ccnc(N2CCC(C(=O)N3CCCCc4cc(Cl)ccc43)CC2)n1 ; CCc1cnc(N2CCC(C(=O)N3CCCCc4cc(Cl)ccc43)CC2)nc1 ; Cc1nc(N2CCC(C(=O)N3CCCCc4cc(Cl)ccc43)CC2)ncc1F ; Cc1nc(N2CCC(C(=O)N3CCCCc4cc(Cl)ccc43)CC2)ncc1Cl ; Cc1nc(N2CCC(C(=O)N3CCCCc4cc(Cl)ccc43)CC2)c2ccccc2n1 ; Cc1cc(N2CCC(C(=O)N3CCCCc4cc(Cl)ccc43)CC2)nc(C)n1] 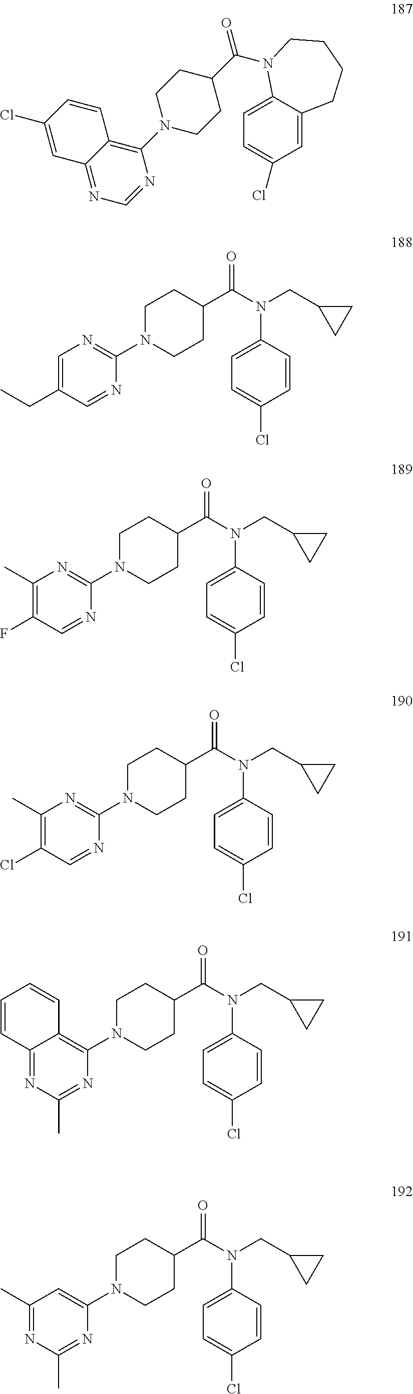 [SMILES: O=C(C1CCN(c2ncnc3cc(Cl)ccc23)CC1)N1CCCCc2cc(Cl)ccc21 ; CCc1cnc(N2CCC(C(=O)N(CC3CC3)c3ccc(Cl)cc3)CC2)nc1 ; Cc1nc(N2CCC(C(=O)N(CC3CC3)c3ccc(Cl)cc3)CC2)ncc1F ; Cc1nc(N2CCC(C(=O)N(CC3CC3)c3ccc(Cl)cc3)CC2)ncc1Cl ; Cc1nc(N2CCC(C(=O)N(CC3CC3)c3ccc(Cl)cc3)CC2)c2ccccc2n1 ; Cc1cc(N2CCC(C(=O)N(CC3CC3)c3ccc(Cl)cc3)CC2)nc(C)n1] 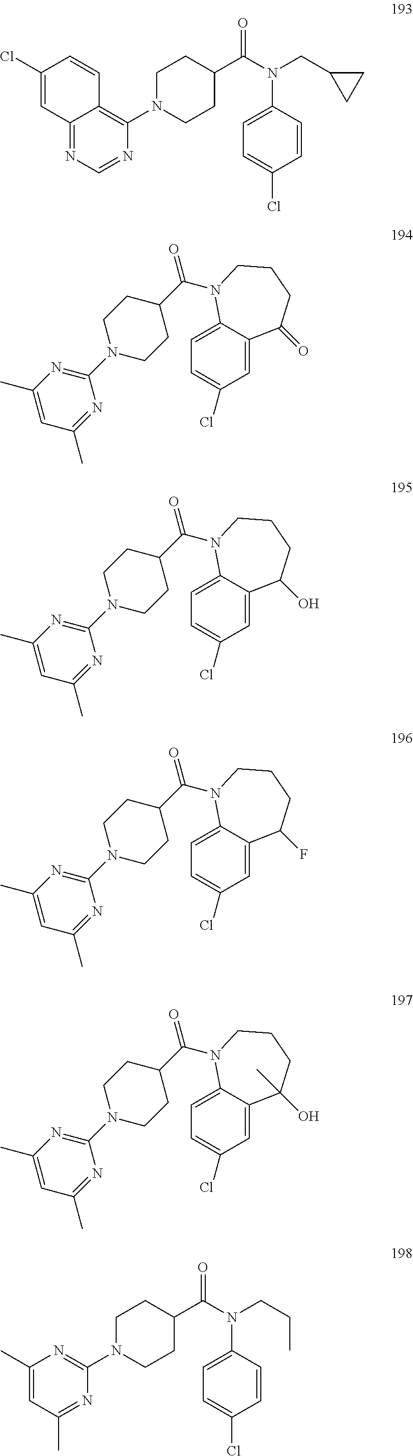 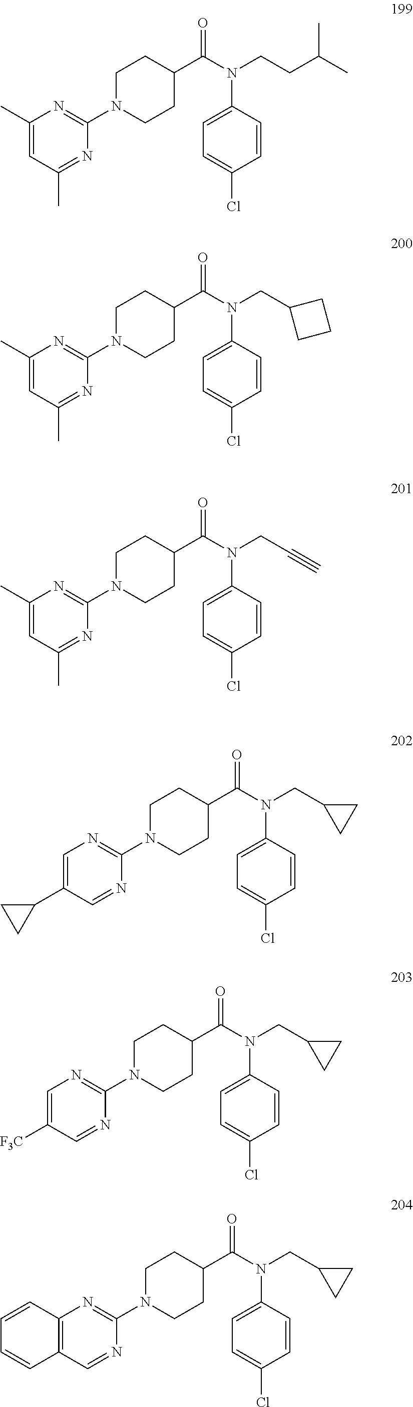 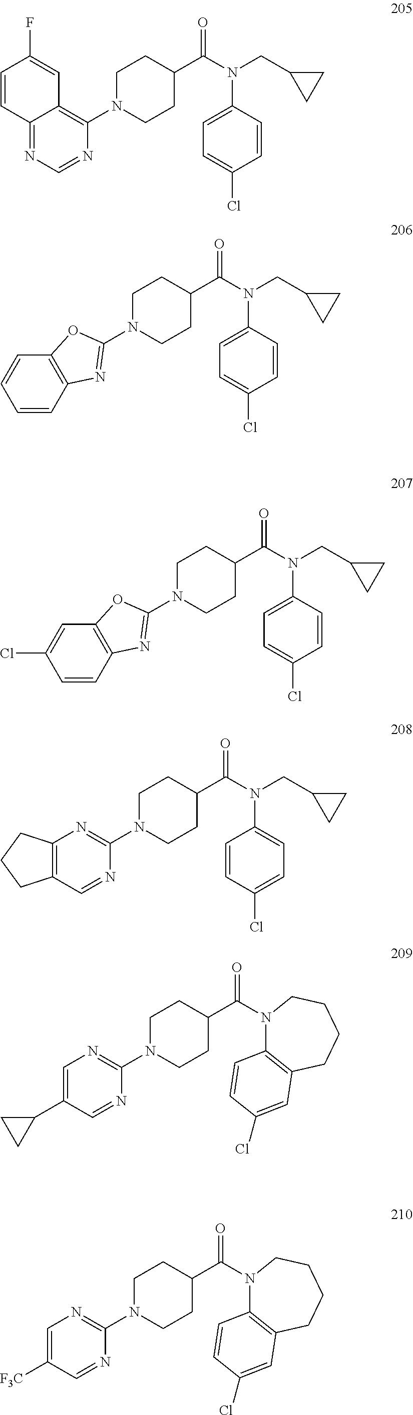 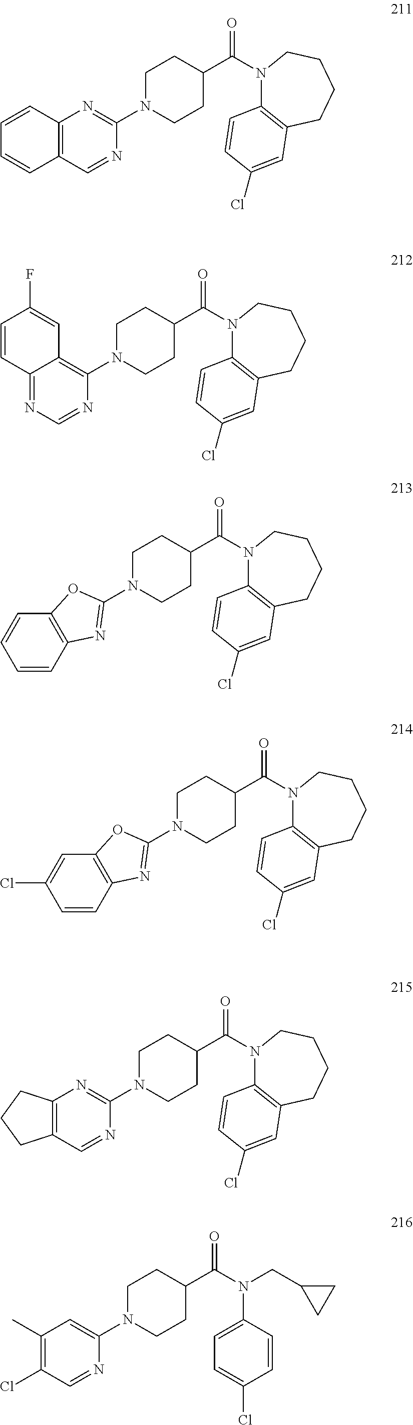 [SMILES: O=C(C1CCN(c2ncc3ccccc3n2)CC1)N1CCCCc2cc(Cl)ccc21 ; O=C(C1CCN(c2ncnc3ccc(F)cc23)CC1)N1CCCCc2cc(Cl)ccc21 ; O=C(C1CCN(c2nc3ccccc3o2)CC1)N1CCCCc2cc(Cl)ccc21 ; O=C(C1CCN(c2nc3ccc(Cl)cc3o2)CC1)N1CCCCc2cc(Cl)ccc21 ; O=C(C1CCN(c2ncc3c(n2)CCC3)CC1)N1CCCCc2cc(Cl)ccc21 ; Cc1cc(N2CCC(C(=O)N(CC3CC3)c3ccc(Cl)cc3)CC2)ncc1Cl] 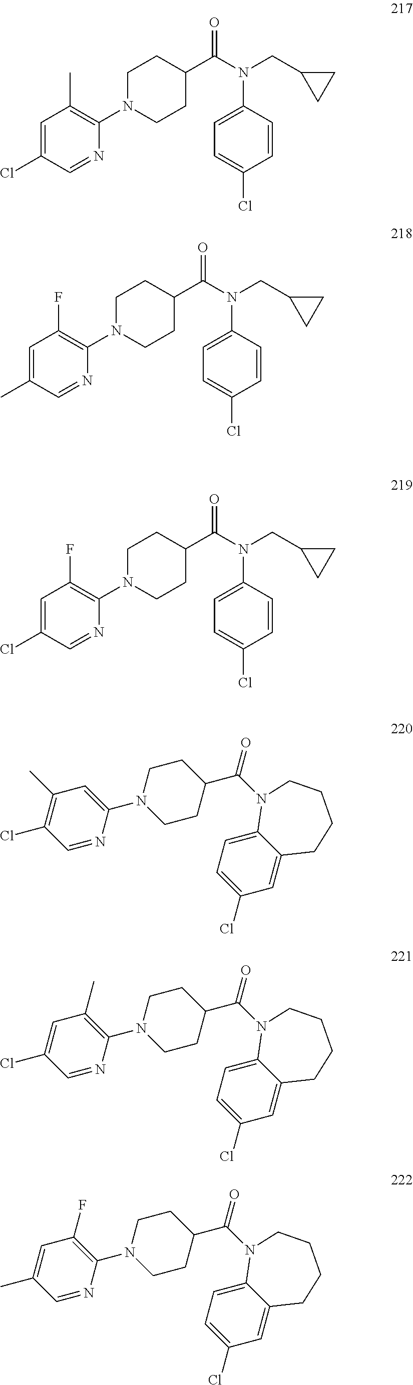 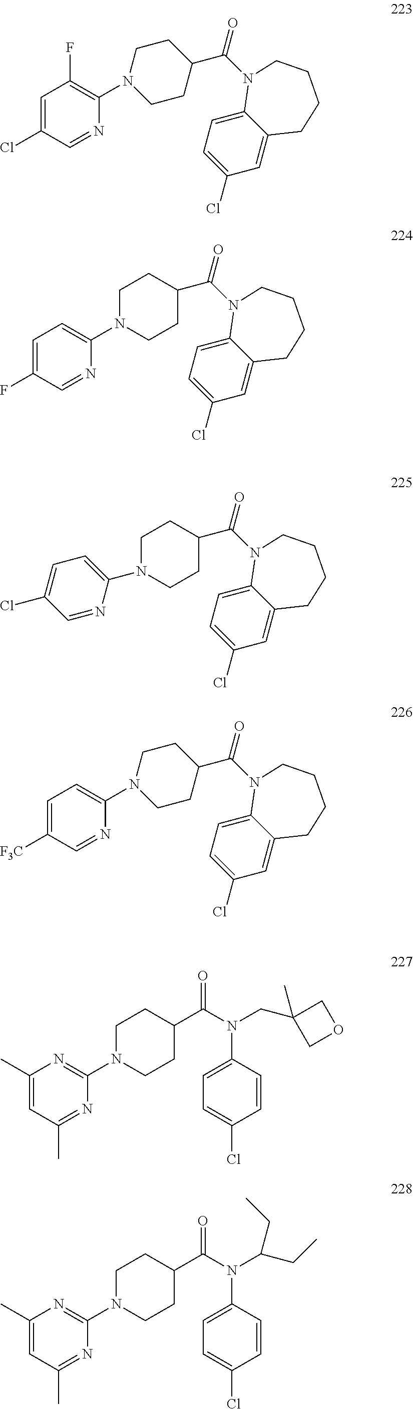 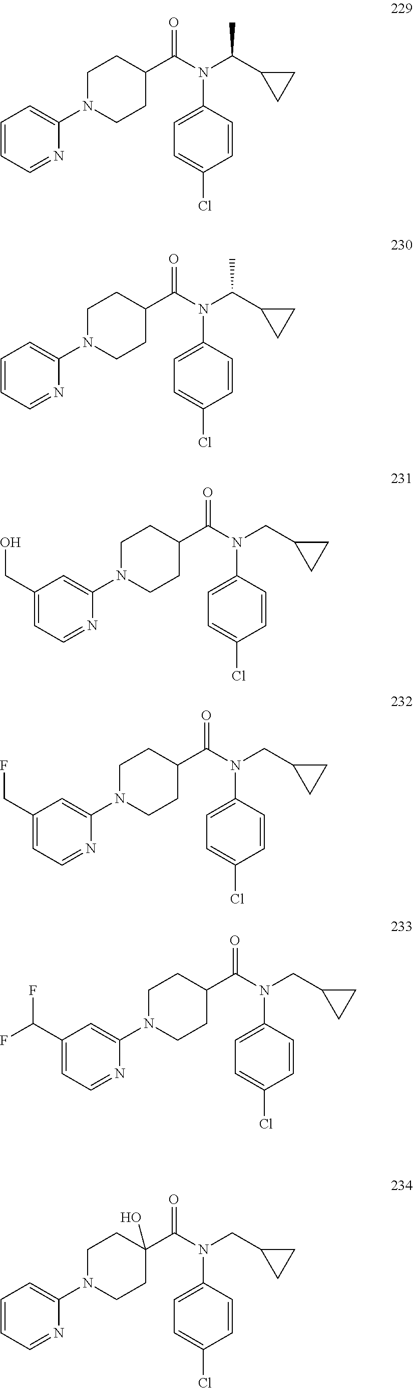 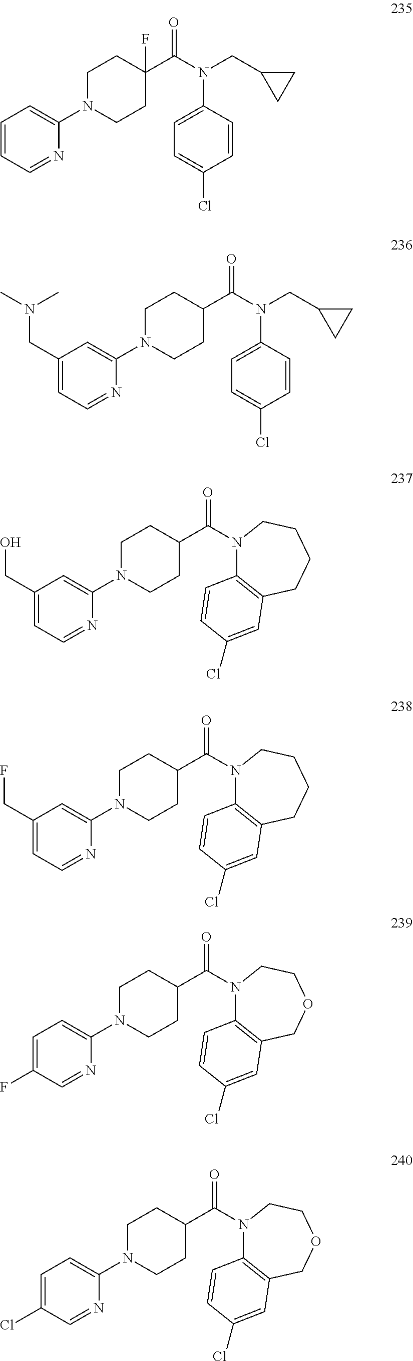 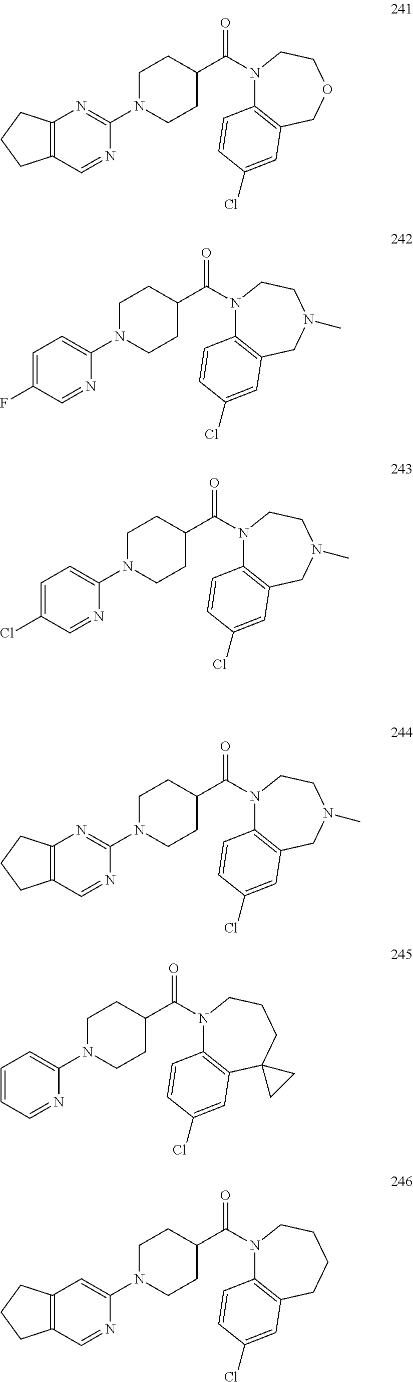 [SMILES: O=C(C1CCN(c2ncc3c(n2)CCC3)CC1)N1CCOCc2cc(Cl)ccc21 ; CN1CCN(C(=O)C2CCN(c3ccc(F)cn3)CC2)c2ccc(Cl)cc2C1 ; CN1CCN(C(=O)C2CCN(c3ccc(Cl)cn3)CC2)c2ccc(Cl)cc2C1 ; CN1CCN(C(=O)C2CCN(c3ncc4c(n3)CCC4)CC2)c2ccc(Cl)cc2C1 ; O=C(C1CCN(c2ccccn2)CC1)N1CCCC2(CC2)c2cc(Cl)ccc21 ; O=C(C1CCN(c2cc3c(cn2)CCC3)CC1)N1CCCCc2cc(Cl)ccc21] 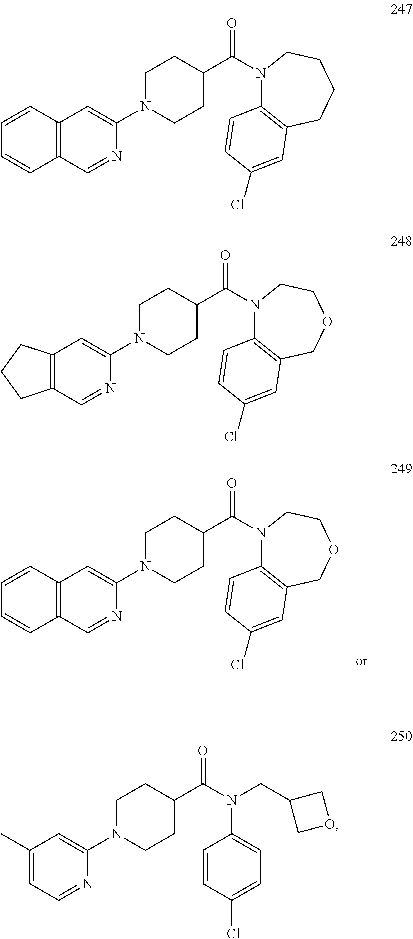 [SMILES: O=C(C1CCN(c2cc3ccccc3cn2)CC1)N1CCCCc2cc(Cl)ccc21 ; O=C(C1CCN(c2cc3c(cn2)CCC3)CC1)N1CCOCc2cc(Cl)ccc21 ; O=C(C1CCN(c2cc3ccccc3cn2)CC1)N1CCOCc2cc(Cl)ccc21 ; Cc1ccnc(N2CCC(C(=O)N(CC3COC3)c3ccc(Cl)cc3)CC2)c1]
or any salt thereof.

11. A compound of formula (V): 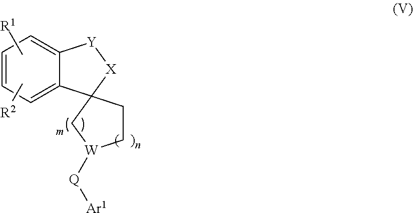
wherein:
X and Y are independently NR³, CR⁴R⁵, O, S, SO, SO₂, CO, or CO₂;
provided that when X and Y are both CR⁴R⁵, one each of R⁵ on respective X and Y can optionally be absent and a double bond can optionally be present between X and Y; and provided that when one of X and Y is NR³ and the other of X and Y is CR⁴R⁵, R³ and R⁵ on respective X and Y can optionally be absent and a double bond can optionally be present between X and Y; and when X and Y are both NR³, both R³ can optionally be absent and a double bond can optionally be present between X and Y;
m and n are both independently 0, 1, 2, or 3;
W is N or CR⁴;
Ar¹ is cycloalkyl, aryl, heterocyclyl, or heteroaryl, wherein any cycloalkyl, aryl, heterocyclyl, or heteroaryl is optionally mono- or independently multi-substituted with J, (C₁-C₆)alkyl, (C₂-C₆)alkenyl, (C₂-C₆)alkynyl, (C₁-C₆)acyl, (C₁-C₆)haloalkyl, (C₁-C₆)alkoxy, (C₁-C₆)haloalkoxy, cycloalkyl(C₀-C₆)alkyl, heterocyclyl(C₀-C₆)alkyl, aryl(C₀-C₆)alkyl, heteroaryl(C₀-C₆)alkyl; or Ar¹ is -Q²-Ar², and Ar² is cycloalkyl, aryl, heterocyclyl, or heteroaryl;
wherein any cycloalkyl, aryl, heterocyclyl, or heteroaryl of Ar¹ or Ar² can be further optionally independently substituted with 1-5 J;
Q and Q² each independently is a bond, (CH₂)₀₋₂O, (CH₂)₀₋₂C(O)(CH₂)₀₋₂, (CH₂)₀₋₂C(O)CR═CR(CH₂)₀₋₂, (CH₂)₀₋₂CR₂(CH₂)₀₋₂, (CH₂)₀₋₂C(OR)(R)(CH₂)₀₋₂, O(CR₂)₁₋₄O, (CH₂)₀₋₂N(R)(CH₂)₀₋₂, (CH₂)₀₋₂S(CH₂)₀₋₂, (CH₂)₀₋₂SO(CH₂)₀₋₂, (CH₂)₀₋₂SO₂(CH₂)₀₋₂ (CH₂)₀₋₂SO₂N(R)(CH₂)₀₋₂, (CH₂)₀₋₂SO₃ (CH₂)₀₋₂, (CH₂)₀₋₂C(O)C(O)(CH₂)₀₋₂, (CH₂)₀₋₂C(O)CH₂C(O)(CH₂)₀₋₂, (CH₂)₀₋₂C(S)(CH₂)₀₋₂, (CH₂)₀₋₂C(O)O(CH₂)₀₋₂, (CH₂)₀₋₂ OC(O)(CH₂)₀₋₂, (CH₂)₀₋₂ OC(O)O(CH₂)₀₋₂, (CH₂)₀₋₂C(O)N(R)(CH₂)₀₋₂ (CH₂)₀₋₂ OC(O)N(R)(CH₂)₀₋₂, (CH₂)₀₋₂C(S)N(R)(CH₂)₀₋₂, (CH₂)₀₋₂NHC(O)(CH₂)₀₋₂, (CH₂)₀₋₂N(R)N(R)C(O)(CH₂)₀₋₂, (CH₂)₀₋₂N(R)N(R)C(O)O(CH₂)₀₋₂, (CH₂)₀₋₂N(R)N(R)CON(R)(CH₂)₀₋₂, (CH₂)₀₋₂N(R)SO₂ (CH₂)₀₋₂, (CH₂)₀₋₂N(R) SO₂N(R)(CH₂)₀₋₂, (CH₂)₀₋₂N(R)C(O)O(CH₂)₀₋₂, (CH₂)₀₋₂N(R)C(O)(CH₂)₀₋₂, (CH₂)₀₋₂N(R)C(S)(CH₂)₀₋₂, (CH₂)₀₋₂N(R)C(O)N(R)(CH₂)₀₋₂, (CH₂)₀₋₂N(R)C(S)N(R)(CH₂)₀₋₂, (CH₂)₀₋₂N(COR)CO(CH₂)₀₋₂, (CH₂)₀₋₂N(OR)(CH₂)₀₋₂, (CH₂)₀₋₂C(═NH)N(R)(CH₂)₀₋₂, (CH₂)₀₋₂C(O)N(OR)(CH₂)₀₋₂, or (CH₂)₀₋₂C(═NOR)(CH₂)₀₋₂;
R¹, R², R³, R⁴, and R⁵ are each independently H, J, (C₁-C₆)alkyl, (C₂-C₆)alkenyl, (C₂-C₆)alkynyl, (C₁-C₆)acyl, (C₁-C₆)haloalkyl, (C₁-C₆)haloalkoxy, cycloalkyl(C₁-C₆)alkyl, heterocyclyl(C₁-C₆)alkyl, aryl(C₁-C₆)alkyl, or heteroaryl(C₁-C₆)alkyl, wherein any alkyl, alkenyl, alkynyl, acyl, haloalkyl, haloalkoxy, cycloalkyl, heterocyclyl, aryl, or heteroaryl of R¹, R², R³, R⁴, or R⁵, can be mono- or independently multi-substituted with J;
or R¹ and R² taken together can be methylenedioxy or ethylenedioxy;
J is independently at each occurrence F, Cl, Br, I, OR^{J}, CN, CF₃, OCF₃, O, S, C(O), S(O), methylenedioxy, ethylenedioxy, N(R^{J})₂, SR^{J}, SOR^{J}, SO₂R^{J}, SO₂N(R^{J})₂, SO₃R^{J}, C(O)R^{J}, C(O)C(O)R^{J}, C(O)CH₂C(O)R^{J}, C(S)R^{J}, C(O)OR^{J}, OC(O)R^{J}, OC(O)OR′, C(O)N(R^{J})₂, OC(O)N(R^{J})₂, C(S)N(R^{J})₂, (CH₂)₀₋₂NHC(O)R^{J}, N(R^{J})N(R^{J})C(O)R^{J}, N(R^{J})N(R^{J})C(O)OR^{J}, N(R^{J})N(R^{J})CON(R^{J})₂, N(R^{J})SO₂R^{J}, N(R^{J})SO₂N(R^{J})₂, N(R^{J})C(O)OR^{J}, N(R^{J})C(O)R^{J}, N(R^{J})C(S)R^{J}, N(R^{J})C(O)N(R^{J})₂, N(R^{J})C(S)N(R^{J})₂, N(COR^{J})COR^{J}, N(OR^{J})R^{J}, C(═NH)N(R^{J})₂, C(O)N(OR^{J})R^{J}, or C(═NOR^{J})R^{J};
wherein R and R^{J} are independently at each occurrence hydrogen or an alkyl, cycloalkyl, cycloalkylalkyl, aryl, aralkyl, heterocyclyl, heterocyclylalkyl, heteroaryl, or heteroarylalkyl; or wherein two R groups together with a nitrogen atom or with two adjacent nitrogen atoms to which they are bonded can together form a 3-8 membered heterocyclyl; optionally further comprising 1-3 additional heteroatoms selected from the group consisting of O, NR, S, S(O) and S(O)₂;
wherein any cycloalkyl, aryl, heterocyclyl, or heteroaryl of formula (V) throughout can be fused, bridged, or in a spiro configuration with one or more additional optionally substituted cycloalkyl, aryl, heterocyclyl, and heteroaryl, monocyclic, bicyclic or polycyclic, saturated, partially unsaturated, or aromatic rings; and wherein any heterocyclyl or heteroaryl comprising nitrogen can be an N-oxide or N-metho salt thereof;
or a pharmaceutically acceptable isomer, racemate, hydrate, solvate, isotope, or salt thereof.

12. The compound of formula (V) of claim 11 wherein the compound is of formula (VA) 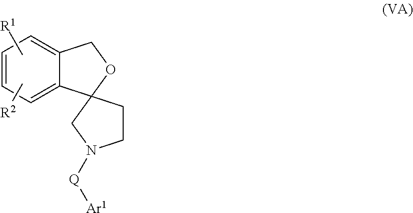
or (VB) 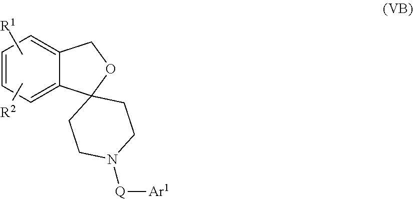
wherein R¹, R², Q, and Ar¹ are as defined in claim 11.

13. The compound of formula (V) of claim 11 wherein the compound is of formula (VC) 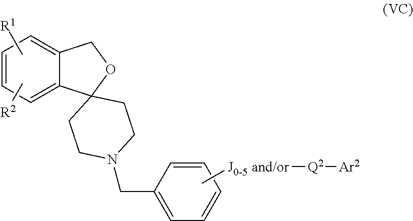
or (VD) 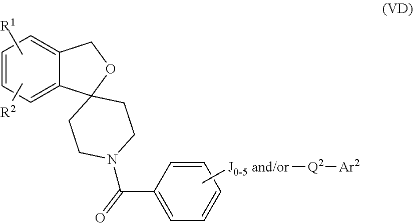
or (VE) 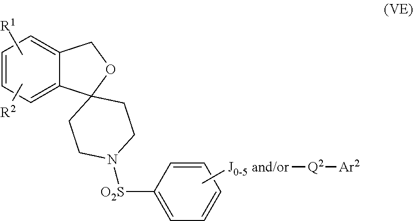
or (VF) 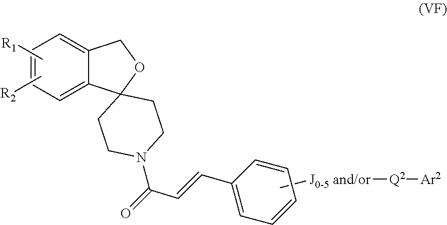
wherein R¹, R², J, Q² and Ar² are as defined in claim 11.

14. The compound of formula (V) of claim 11 wherein Q is a bond, CH₂, C(O), or SO₂.

15. The compound of formula (V) of claim 11 wherein Ar¹ is unsubstituted or substituted phenyl, or comprises a pyrazole, benzimidazole, benzthiophene, oxazepine or diazepine.

16. A compound of formula (V) of claim 11 wherein the compound is any of 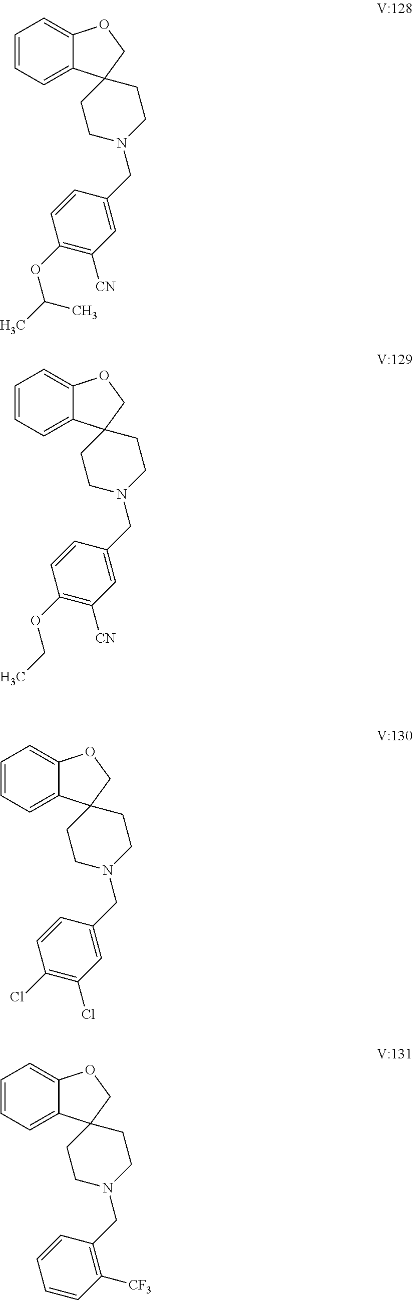 [SMILES: CC(C)Oc1ccc(CN2CCC3(CC2)COc2ccccc23)cc1C#N ; CCOc1ccc(CN2CCC3(CC2)COc2ccccc23)cc1C#N ; Clc1ccc(CN2CCC3(CC2)COc2ccccc23)cc1Cl ; FC(F)(F)c1ccccc1CN1CCC2(CC1)COc1ccccc12] 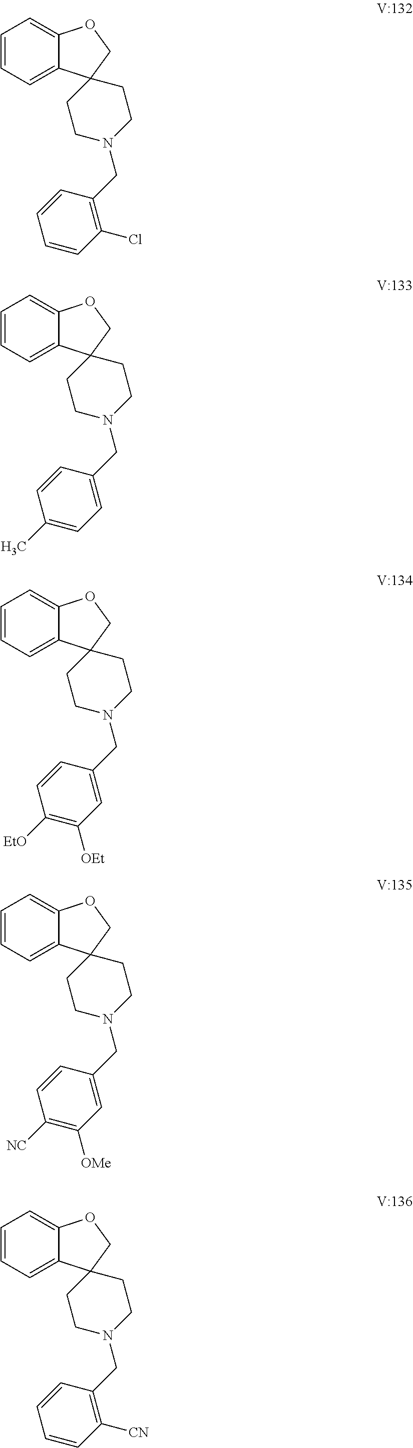 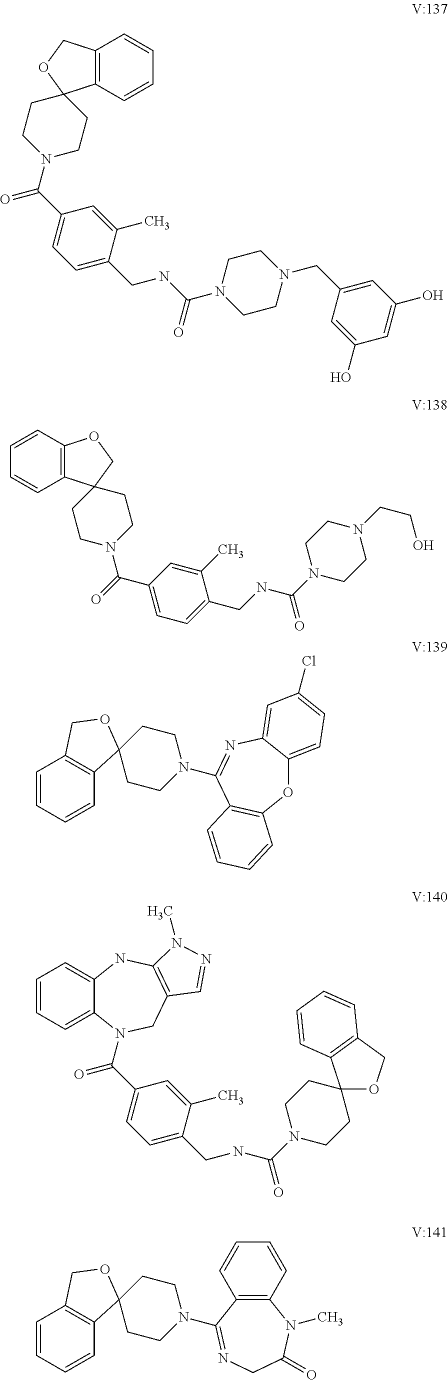 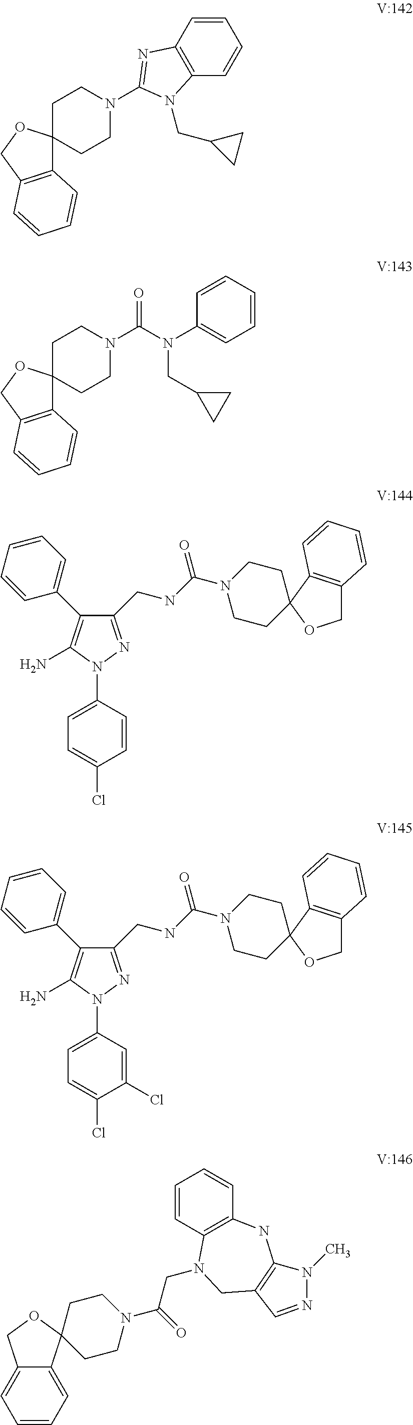 [SMILES: c1ccc2c(c1)COC21CCN(c2nc3ccccc3n2CC2CC2)CC1 ; O=C(N1CCC2(CC1)OCc1ccccc12)N(CC1CC1)c1ccccc1 ; Nc1c(-c2ccccc2)c(CNC(=O)N2CCC3(CC2)OCc2ccccc23)nn1-c1ccc(Cl)cc1 ; Nc1c(-c2ccccc2)c(CNC(=O)N2CCC3(CC2)OCc2ccccc23)nn1-c1ccc(Cl)c(Cl)c1 ; Cn1ncc2c1Nc1ccccc1N(CC(=O)N1CCC3(CC1)OCc1ccccc13)C2] 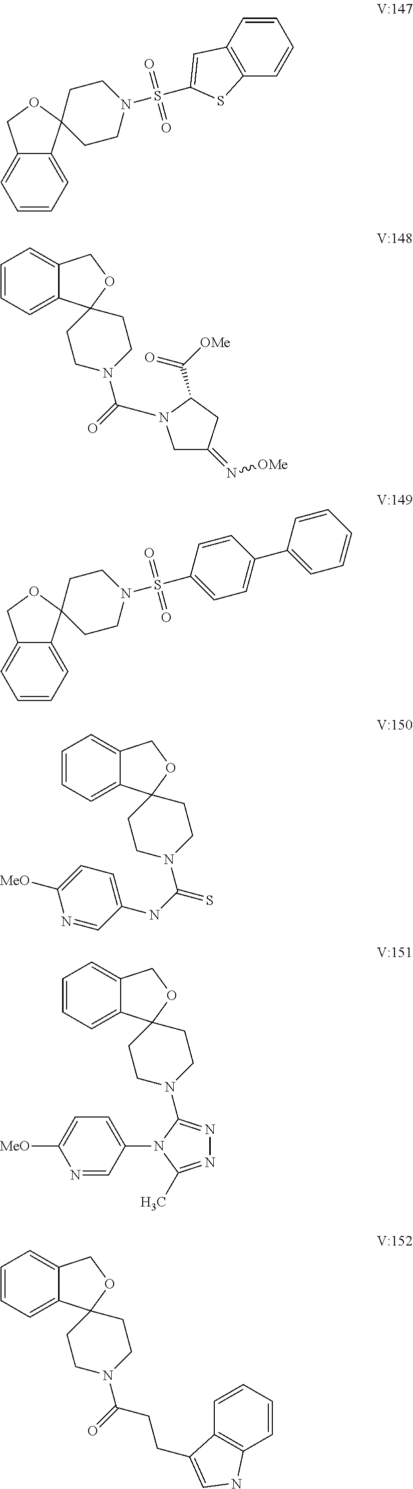 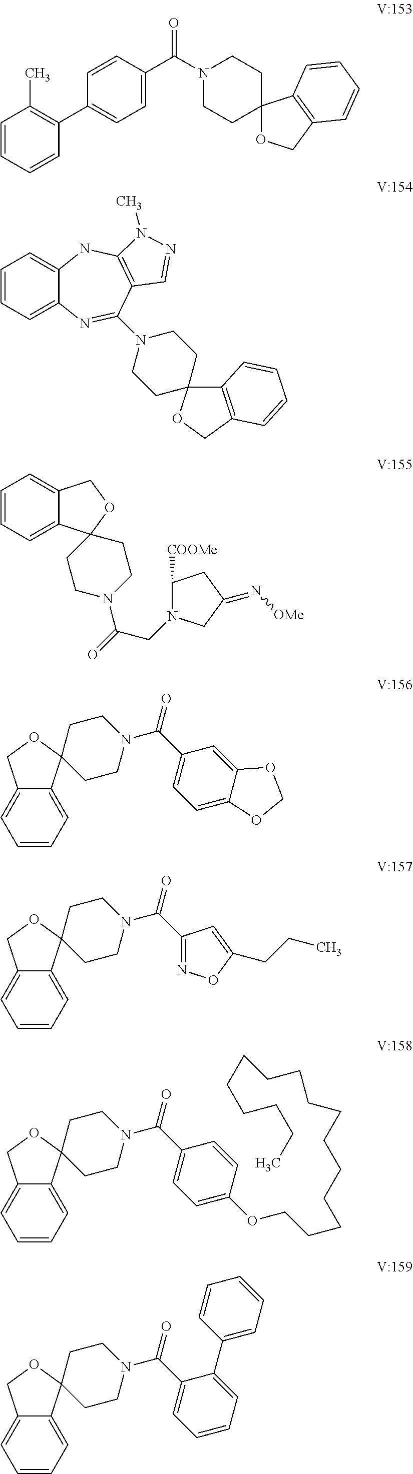 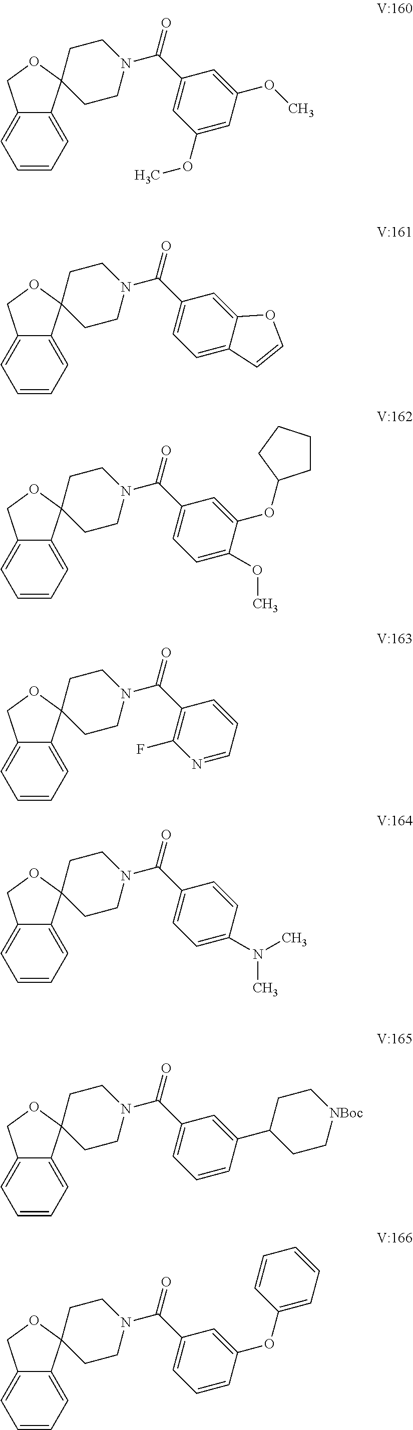 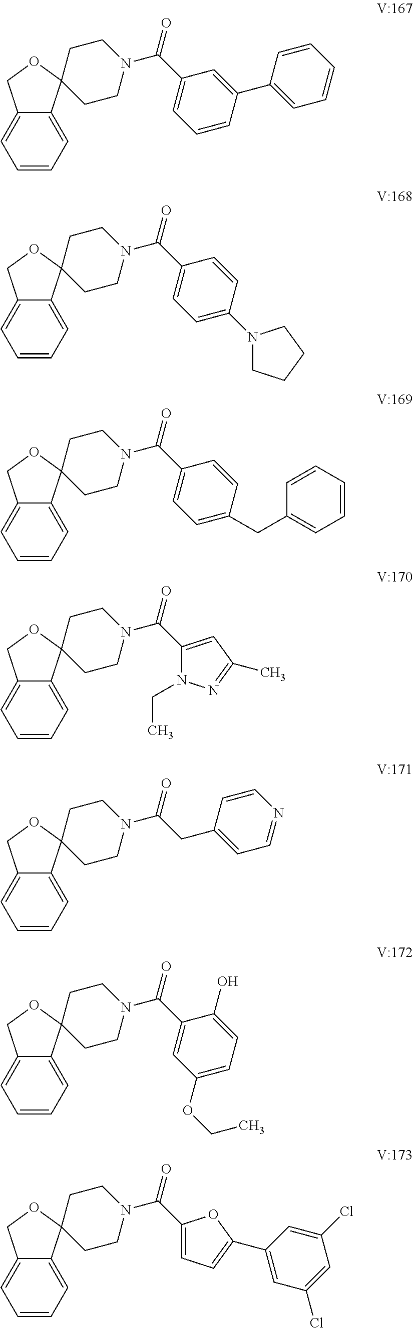 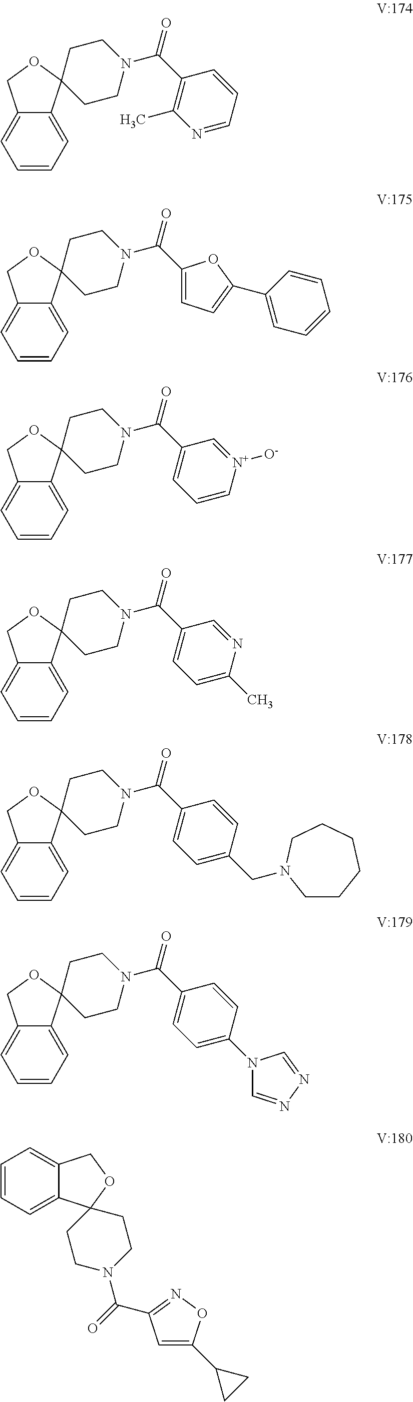 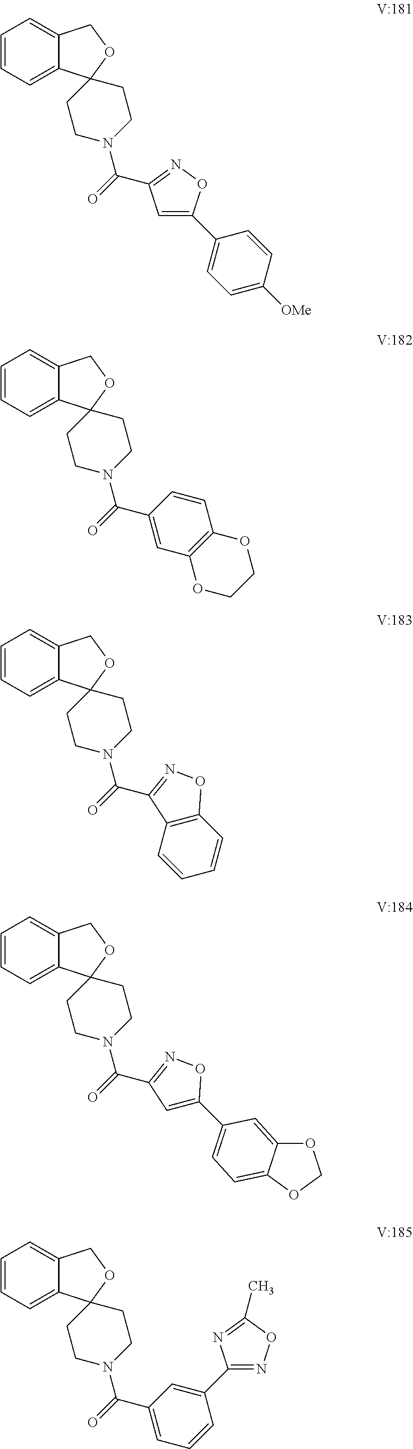 [SMILES: COc1ccc(-c2cc(C(=O)N3CCC4(CC3)OCc3ccccc34)no2)cc1 ; O=C(c1ccc2c(c1)OCCO2)N1CCC2(CC1)OCc1ccccc12 ; O=C(c1noc2ccccc12)N1CCC2(CC1)OCc1ccccc12 ; O=C(c1cc(-c2ccc3c(c2)OCO3)on1)N1CCC2(CC1)OCc1ccccc12 ; Cc1nc(-c2cccc(C(=O)N3CCC4(CC3)OCc3ccccc34)c2)no1] 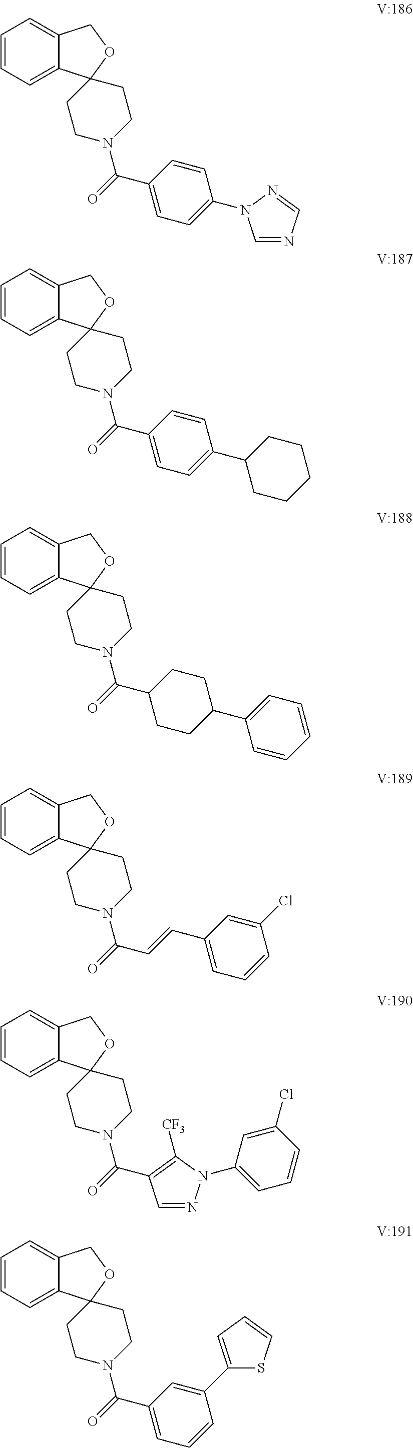 [SMILES: O=C(c1ccc(-n2cncn2)cc1)N1CCC2(CC1)OCc1ccccc12 ; O=C(c1ccc(C2CCCCC2)cc1)N1CCC2(CC1)OCc1ccccc12 ; O=C(C1CCC(c2ccccc2)CC1)N1CCC2(CC1)OCc1ccccc12 ; O=C(/C=C/c1cccc(Cl)c1)N1CCC2(CC1)OCc1ccccc12 ; O=C(c1cnn(-c2cccc(Cl)c2)c1C(F)(F)F)N1CCC2(CC1)OCc1ccccc12 ; O=C(c1cccc(-c2cccs2)c1)N1CCC2(CC1)OCc1ccccc12] 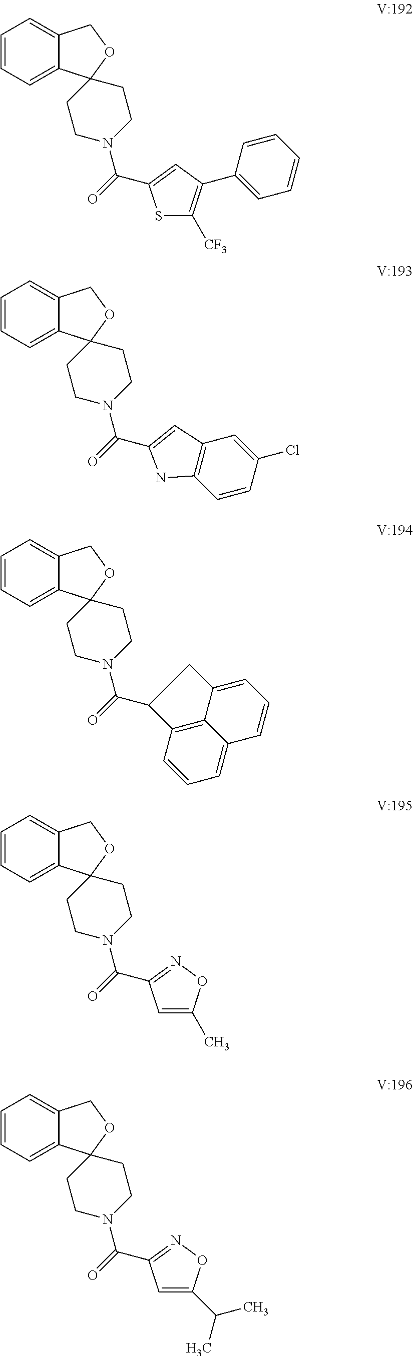 [SMILES: O=C(c1cc(-c2ccccc2)c(C(F)(F)F)s1)N1CCC2(CC1)OCc1ccccc12 ; O=C(c1cc2cc(Cl)ccc2[nH]1)N1CCC2(CC1)OCc1ccccc12 ; O=C(C1Cc2cccc3cccc1c23)N1CCC2(CC1)OCc1ccccc12 ; Cc1cc(C(=O)N2CCC3(CC2)OCc2ccccc23)no1 ; CC(C)c1cc(C(=O)N2CCC3(CC2)OCc2ccccc23)no1] 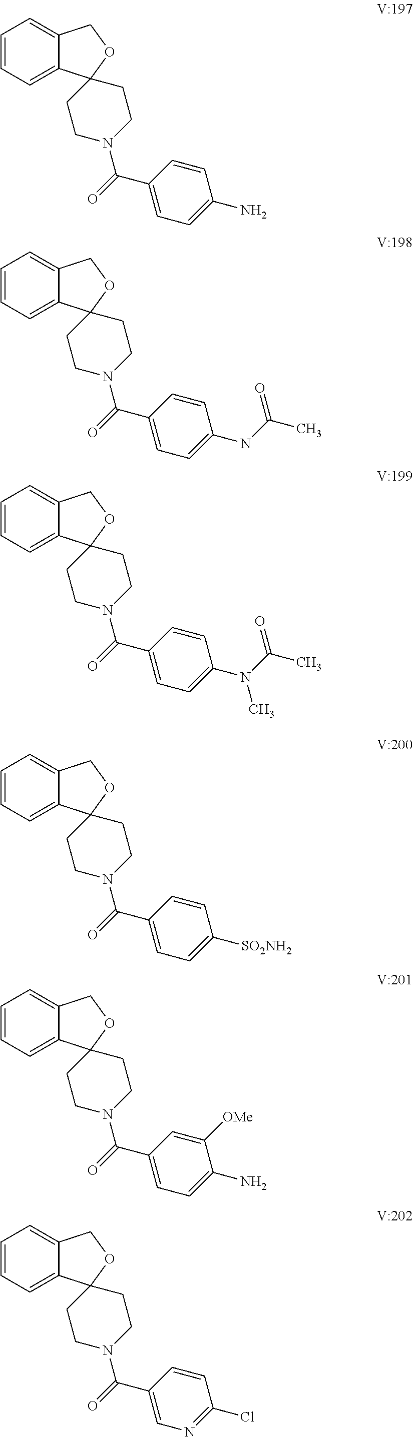 [SMILES: Nc1ccc(C(=O)N2CCC3(CC2)OCc2ccccc23)cc1 ; CC(=O)Nc1ccc(C(=O)N2CCC3(CC2)OCc2ccccc23)cc1 ; CC(=O)N(C)c1ccc(C(=O)N2CCC3(CC2)OCc2ccccc23)cc1 ; NS(=O)(=O)c1ccc(C(=O)N2CCC3(CC2)OCc2ccccc23)cc1 ; COc1cc(C(=O)N2CCC3(CC2)OCc2ccccc23)ccc1N ; O=C(c1ccc(Cl)nc1)N1CCC2(CC1)OCc1ccccc12] 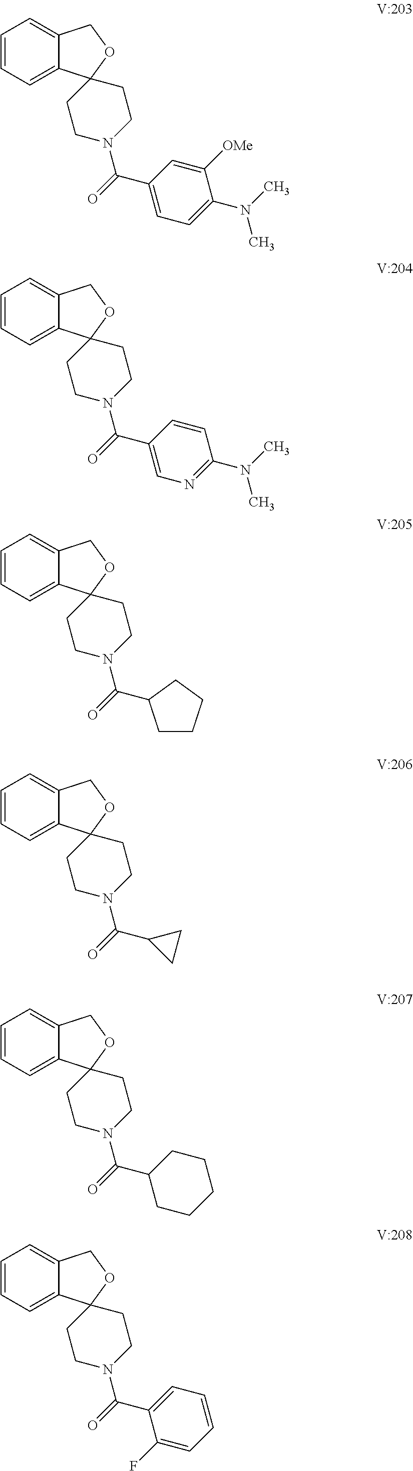 [SMILES: COc1cc(C(=O)N2CCC3(CC2)OCc2ccccc23)ccc1N(C)C ; CN(C)c1ccc(C(=O)N2CCC3(CC2)OCc2ccccc23)cn1 ; O=C(C1CCCC1)N1CCC2(CC1)OCc1ccccc12 ; O=C(C1CC1)N1CCC2(CC1)OCc1ccccc12 ; O=C(C1CCCCC1)N1CCC2(CC1)OCc1ccccc12 ; O=C(c1ccccc1F)N1CCC2(CC1)OCc1ccccc12] 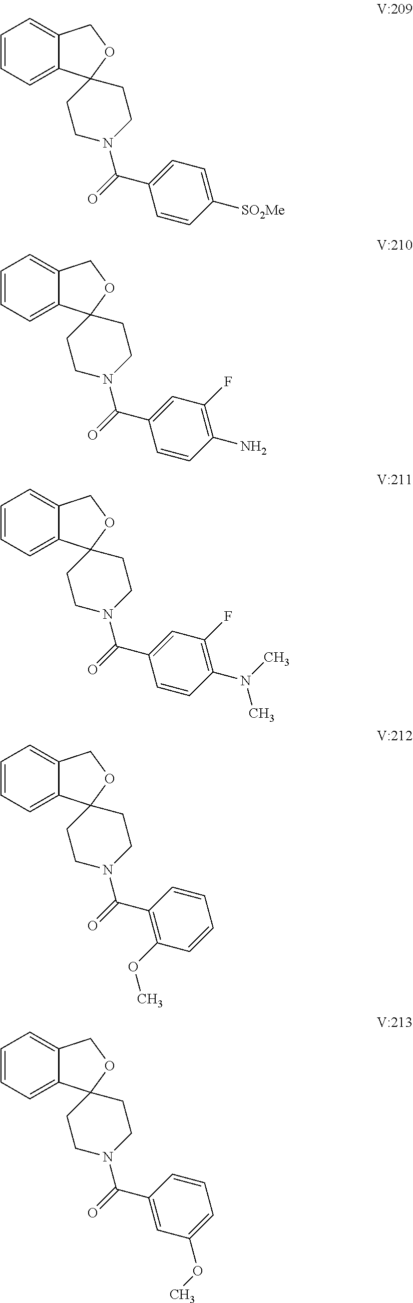 [SMILES: CS(=O)(=O)c1ccc(C(=O)N2CCC3(CC2)OCc2ccccc23)cc1 ; Nc1ccc(C(=O)N2CCC3(CC2)OCc2ccccc23)cc1F ; CN(C)c1ccc(C(=O)N2CCC3(CC2)OCc2ccccc23)cc1F ; COc1ccccc1C(=O)N1CCC2(CC1)OCc1ccccc12 ; COc1cccc(C(=O)N2CCC3(CC2)OCc2ccccc23)c1] 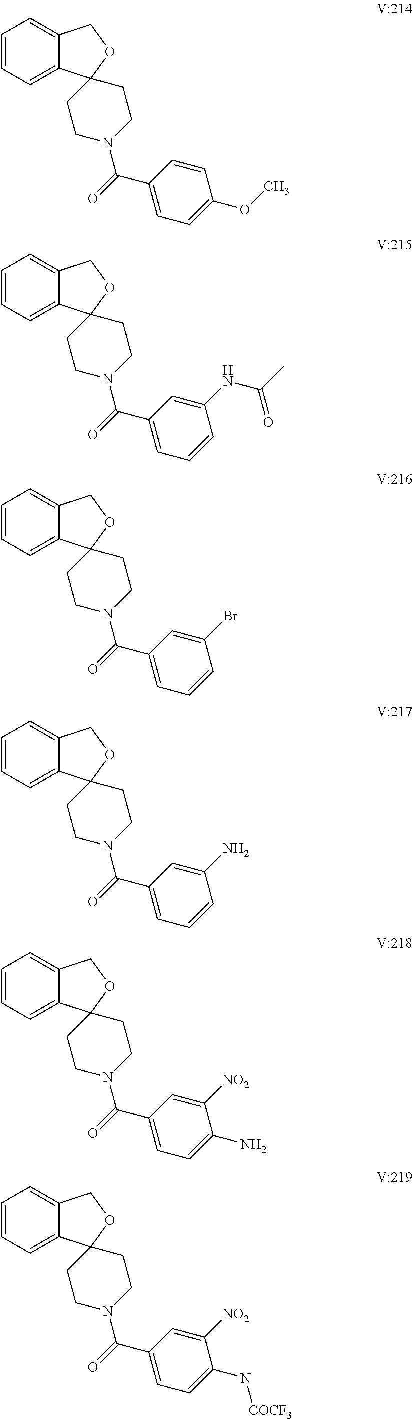 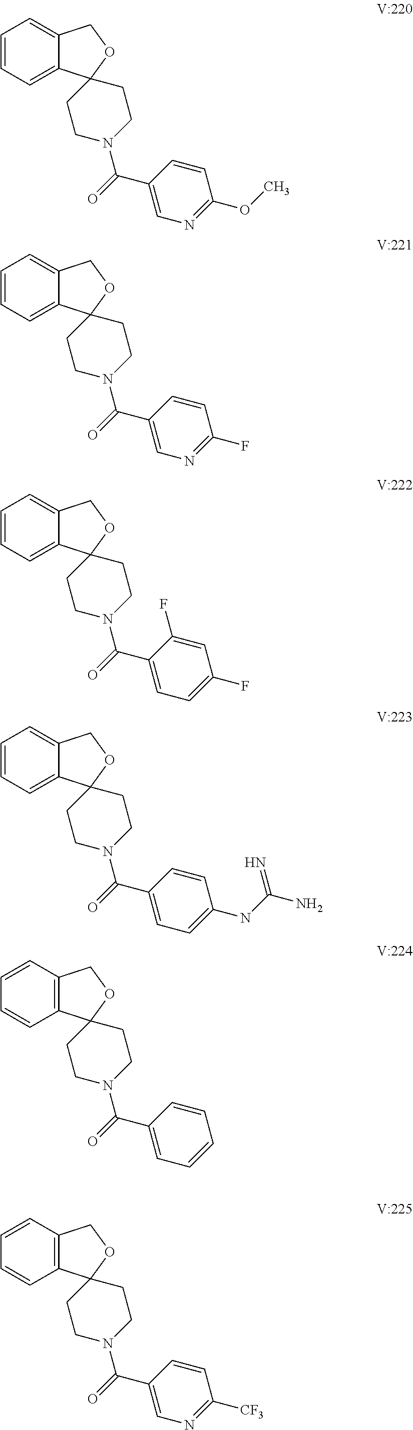 [SMILES: COc1ccc(C(=O)N2CCC3(CC2)OCc2ccccc23)cn1 ; O=C(c1ccc(F)nc1)N1CCC2(CC1)OCc1ccccc12 ; O=C(c1ccc(F)cc1F)N1CCC2(CC1)OCc1ccccc12 ; N=C(N)Nc1ccc(C(=O)N2CCC3(CC2)OCc2ccccc23)cc1 ; O=C(c1ccccc1)N1CCC2(CC1)OCc1ccccc12 ; O=C(c1ccc(C(F)(F)F)nc1)N1CCC2(CC1)OCc1ccccc12] 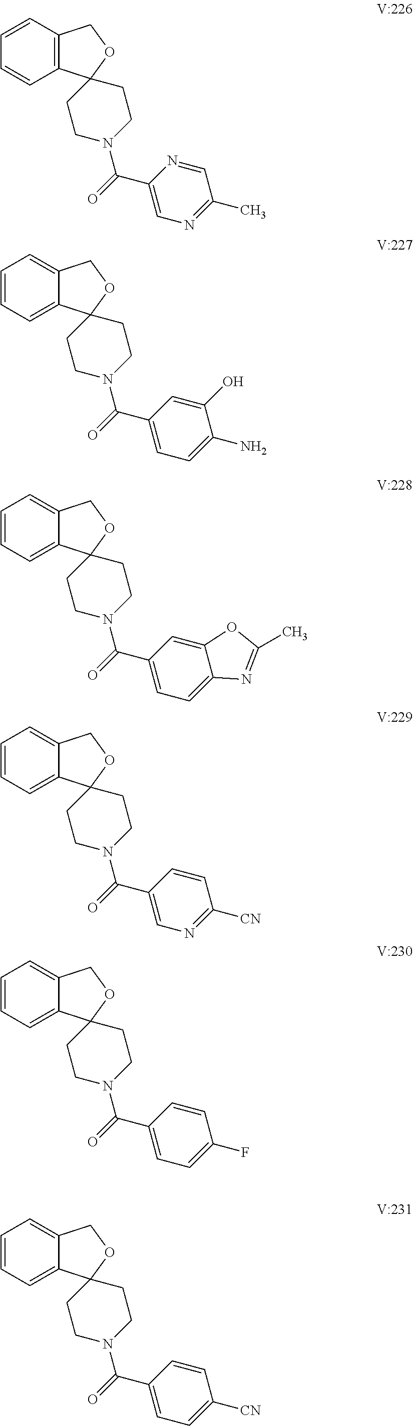 [SMILES: Cc1cnc(C(=O)N2CCC3(CC2)OCc2ccccc23)cn1 ; Nc1ccc(C(=O)N2CCC3(CC2)OCc2ccccc23)cc1O ; Cc1nc2ccc(C(=O)N3CCC4(CC3)OCc3ccccc34)cc2o1 ; N#Cc1ccc(C(=O)N2CCC3(CC2)OCc2ccccc23)cn1 ; O=C(c1ccc(F)cc1)N1CCC2(CC1)OCc1ccccc12 ; N#Cc1ccc(C(=O)N2CCC3(CC2)OCc2ccccc23)cc1] 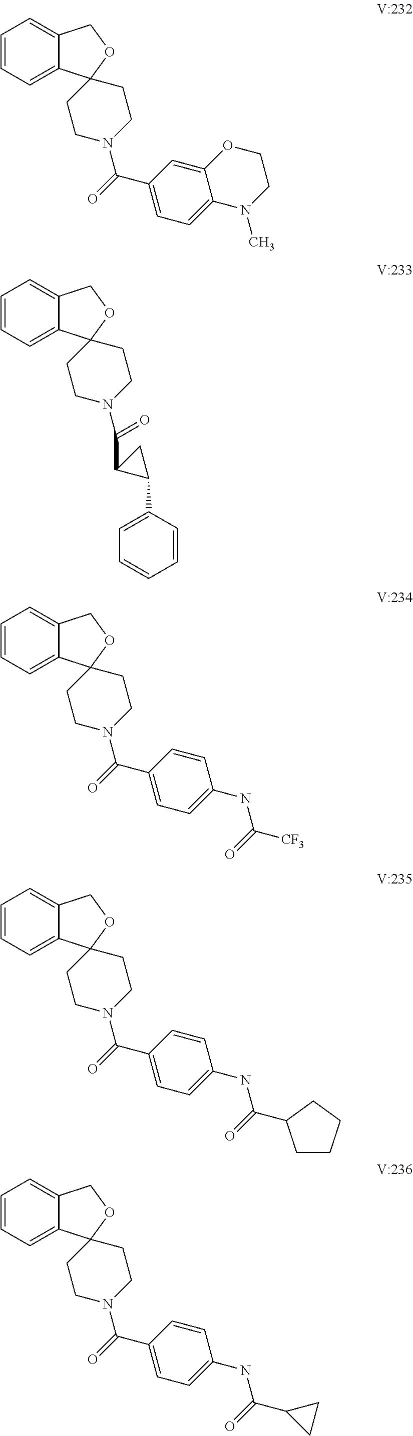 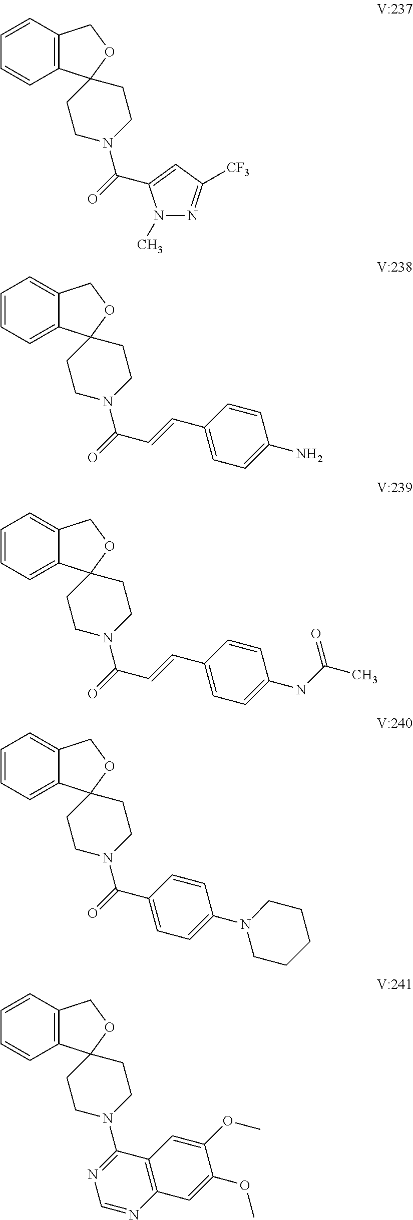 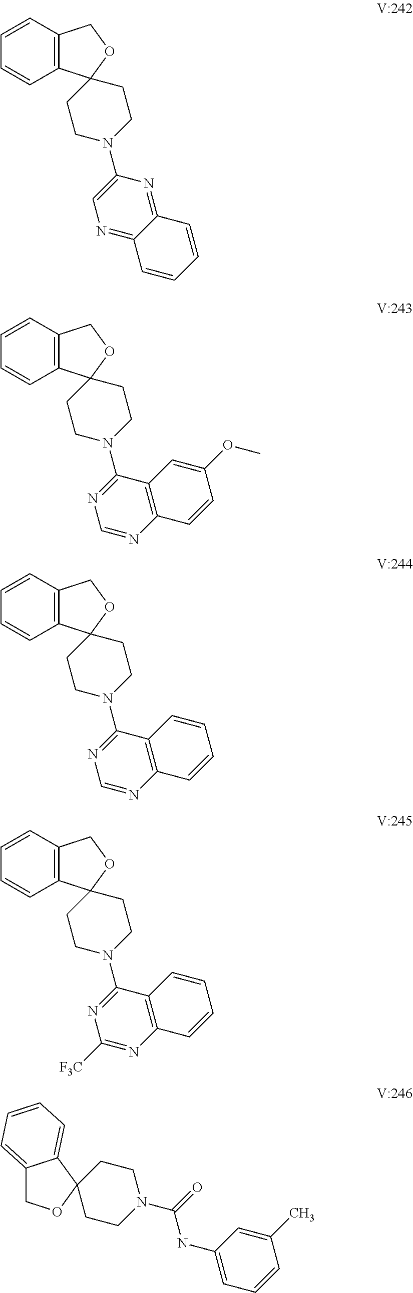 [SMILES: c1ccc2c(c1)COC21CCN(c2cnc3ccccc3n2)CC1 ; COc1ccc2ncnc(N3CCC4(CC3)OCc3ccccc34)c2c1 ; c1ccc2c(c1)COC21CCN(c2ncnc3ccccc23)CC1 ; FC(F)(F)c1nc(N2CCC3(CC2)OCc2ccccc23)c2ccccc2n1 ; Cc1cccc(NC(=O)N2CCC3(CC2)OCc2ccccc23)c1] 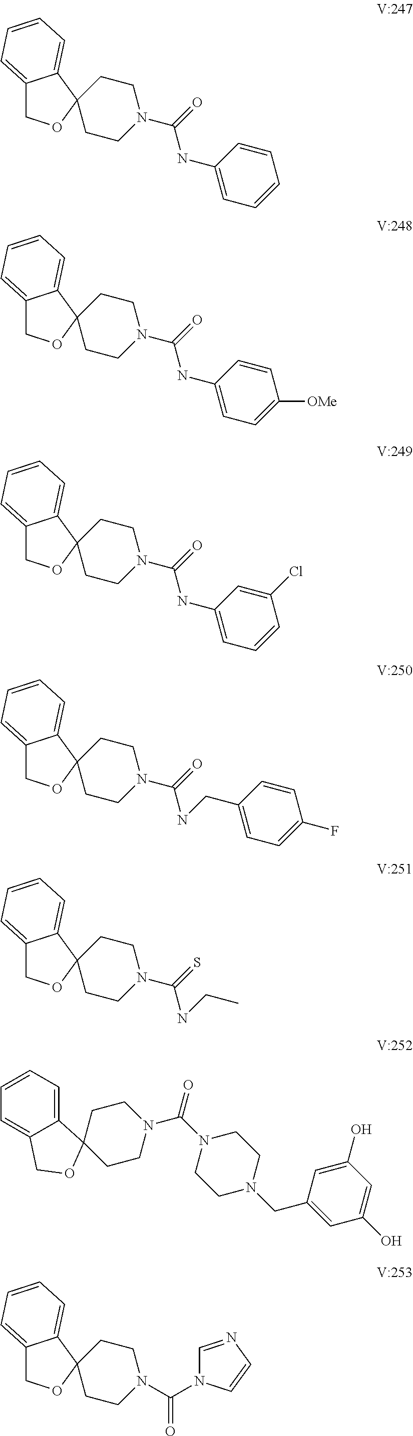 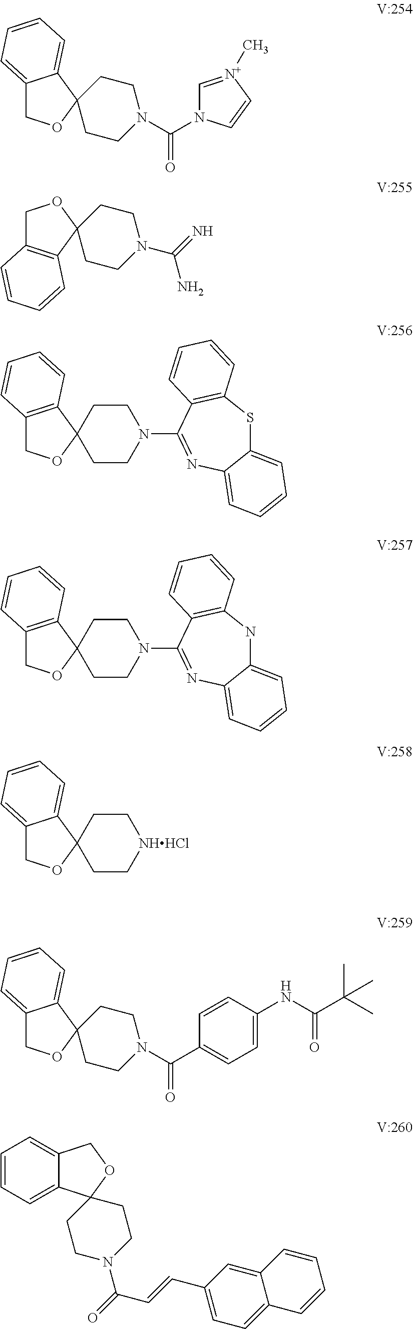 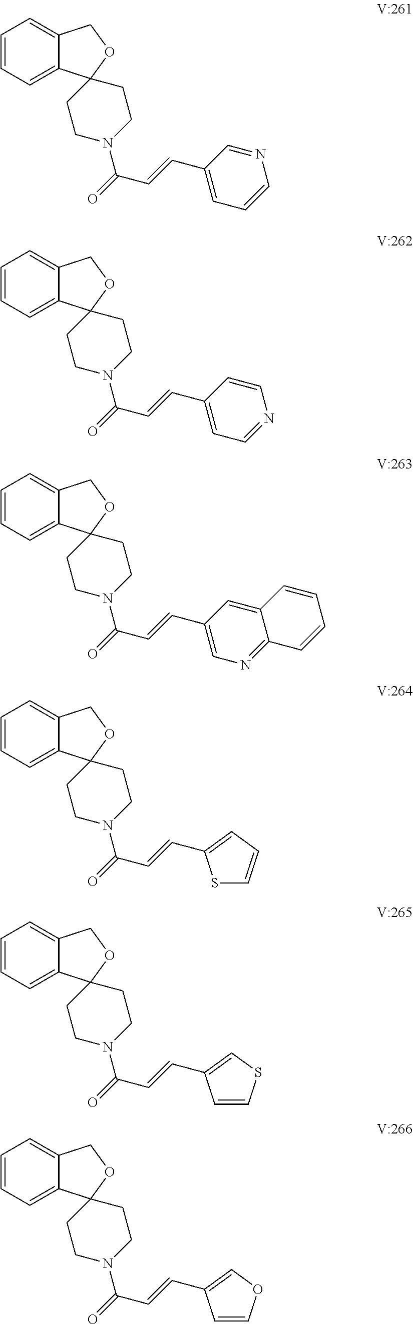 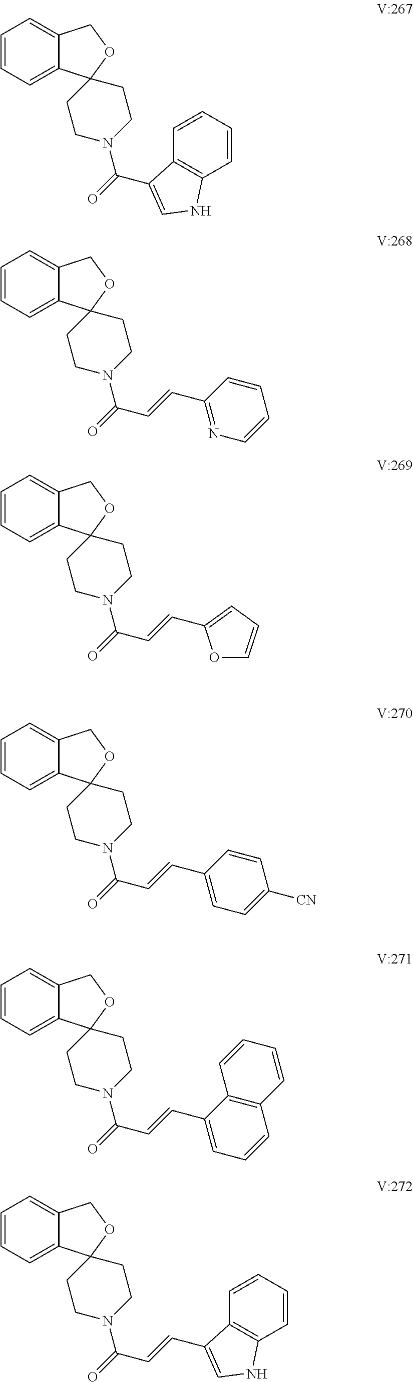 [SMILES: O=C(c1c[nH]c2ccccc12)N1CCC2(CC1)OCc1ccccc12 ; O=C(/C=C/c1ccccn1)N1CCC2(CC1)OCc1ccccc12 ; O=C(/C=C/c1ccco1)N1CCC2(CC1)OCc1ccccc12 ; N#Cc1ccc(/C=C/C(=O)N2CCC3(CC2)OCc2ccccc23)cc1 ; O=C(/C=C/c1cccc2ccccc12)N1CCC2(CC1)OCc1ccccc12 ; O=C(/C=C/c1c[nH]c2ccccc12)N1CCC2(CC1)OCc1ccccc12] 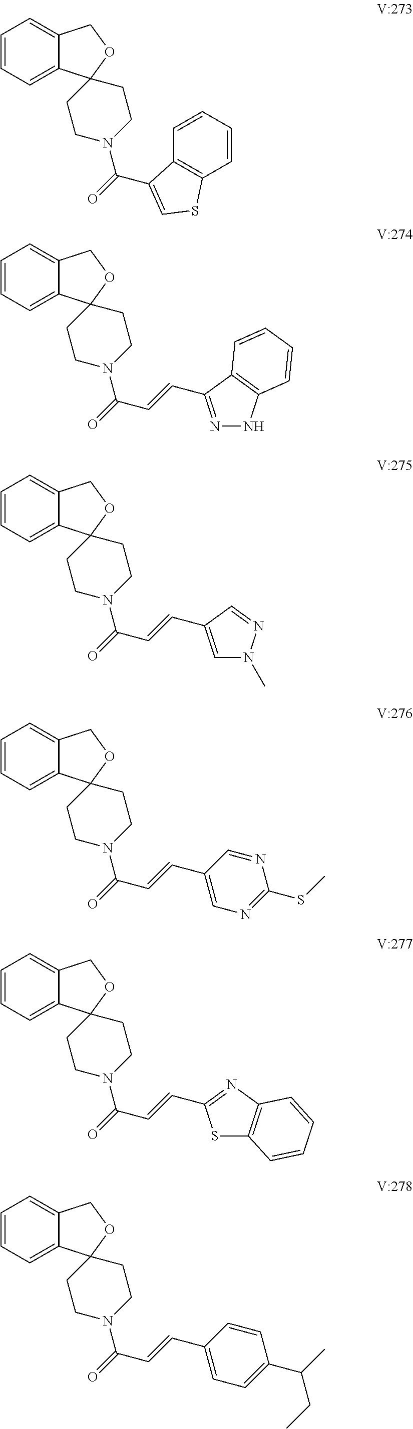 [SMILES: O=C(c1csc2ccccc12)N1CCC2(CC1)OCc1ccccc12 ; O=C(/C=C/c1n[nH]c2ccccc12)N1CCC2(CC1)OCc1ccccc12 ; Cn1cc(/C=C/C(=O)N2CCC3(CC2)OCc2ccccc23)cn1 ; CSc1ncc(/C=C/C(=O)N2CCC3(CC2)OCc2ccccc23)cn1 ; O=C(/C=C/c1nc2ccccc2s1)N1CCC2(CC1)OCc1ccccc12 ; CCC(C)c1ccc(/C=C/C(=O)N2CCC3(CC2)OCc2ccccc23)cc1] 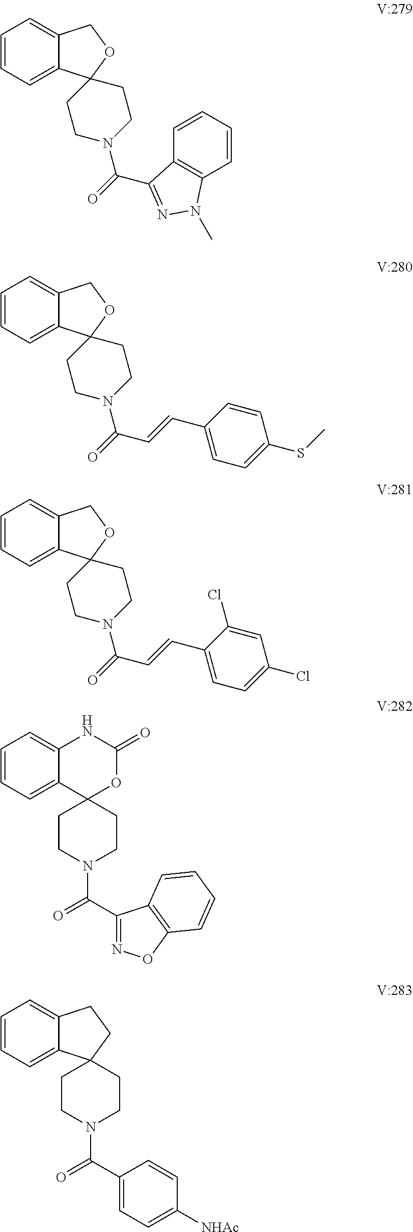 [SMILES: Cn1nc(C(=O)N2CCC3(CC2)OCc2ccccc23)c2ccccc21 ; CSc1ccc(/C=C/C(=O)N2CCC3(CC2)OCc2ccccc23)cc1 ; O=C(/C=C/c1ccc(Cl)cc1Cl)N1CCC2(CC1)OCc1ccccc12 ; O=C1Nc2ccccc2C2(CCN(C(=O)c3noc4ccccc34)CC2)O1 ; CC(=O)Nc1ccc(C(=O)N2CCC3(CCc4ccccc43)CC2)cc1] 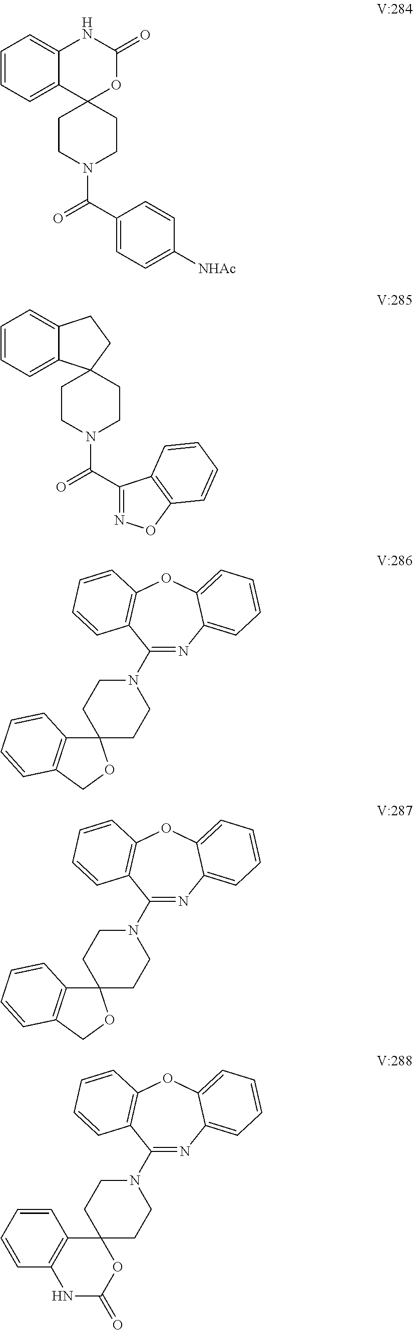 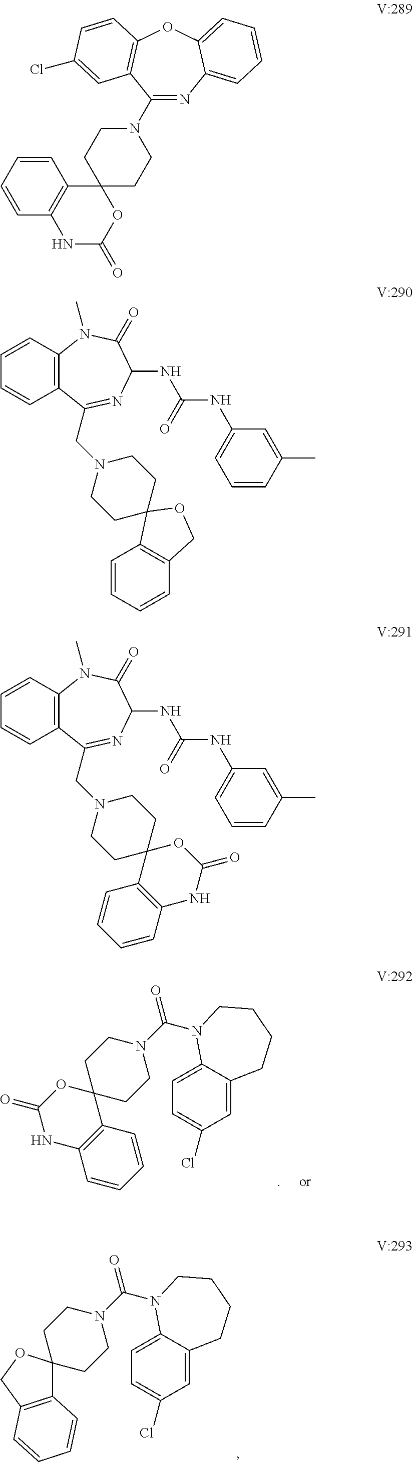
or any salt thereof.

17. A pharmaceutical composition comprising a compound of claim 1, or a pharmaceutically acceptable isomer, racemate, hydrate, solvate, isotope, or salt thereof, and a pharmaceutically acceptable carrier, diluent, or excipient.

18-26. (canceled)

27. A method for treating vasopressin-dependent conditions or conditions related to inappropriate secretion of vasopressin, comprising administering to a subject in need thereof an effective amount of a compound of claim 1, or a pharmaceutically acceptable isomer, racemate, hydrate, solvate, isotope, or salt thereof, or a pharmaceutical composition comprising the same, at a frequency and for a duration sufficient to provide a beneficial effect to the subject.

28. A pharmaceutical composition comprising a compound of claim 11, or a pharmaceutically acceptable isomer, racemate, hydrate, solvate, isotope, or salt thereof, and a pharmaceutically acceptable carrier, diluent, or excipient.

29. A method for treating vasopressin-dependent conditions or conditions related to inappropriate secretion of vasopressin, comprising administering to a subject in need thereof an effective amount of a compound of claim 11, or a pharmaceutically acceptable isomer, racemate, hydrate, solvate, isotope, or salt thereof, or a pharmaceutical composition comprising the same, at a frequency and for a duration sufficient to provide a beneficial effect to the subject.
